(19) United States
(12) Patent Application Publication
(10) Pub. No.: US 2025/0325628 A1
(43) Pub. Date: Oct. 23, 2025

(54) REVERSING AGING OF THE CENTRAL NERVOUS SYSTEM

(71) Applicant: President and Fellows of Harvard College, Cambridge, MA (US)

(72) Inventors: David A. Sinclair, Cambridge, MA (US); Xiao Tian, Cambridge, MA (US)

(73) Assignee: President and Fellows of Harvard College, Cambridge, MA (US)

(21) Appl. No.: 18/854,506

(22) PCT Filed: Apr. 5, 2023
(86) PCT No.: PCT/US2023/065374

§ 371 Date: Oct. 4, 2024

Related U.S. Application Data
(60) Provisional application No. 63/328,069, filed on Apr. 6, 2022.

(51) Int. Cl. A61K 38/17; A61K 48/00; A61P 25/28; C12N 5/0793; C12N 15/86

(52) U.S. Cl. CPC A61K 38/1709; A61K 48/005; A61P 25/28; C12N 5/0619; C12N 15/86; C12N 2501/602; C12N 2501/603; C12N 2501/604; C12N 2510/00; C12N 2740/15011; C12N 2750/14143

(57) ABSTRACT

Provided herein are engineered nucleic acids (e.g., expression vectors, including viral vectors, such as lentiviral vectors, adenoviral vectors, AAV vectors, herpes viral vectors, and retroviral vectors) that encode OCT4; KLF4; SOX2; or any combination thereof that are useful, for example, in inducing cellular reprogramming, tissue repair, tissue regeneration, organ regeneration, reversing aging, or any combination thereof in the central nervous system or ex vivo. Also provided herein are recombinant viruses (e.g., lentiviruses, alphaviruses, vaccinia viruses, adenoviruses, herpes viruses, retroviruses, or AAVs) comprising the engineered nucleic acids (e.g., engineered nucleic acids), engineered cells, compositions comprising the engineered nucleic acids, the recombinant viruses, engineered cells, engineered proteins, chemical agents that are capable of activating expression of OCT4; KLF4; SOX2; or any combination thereof, an engineered protein selected from the group consisting of OCT4; KLF4; SOX2; or any combination thereof, an antibody capable of activating expression of OCT4; KLF4; SOX2; or any combination thereof, and methods of treating a disease (e.g., a neurological disease), preventing a disease (e.g., neurological disease), regulating (e.g., inducing or inducing and then stopping) cellular reprogramming, regulating tissue repair, regulating tissue regeneration, or any combination thereof.

### CROSS-REFERENCE TO RELATED APPLICATIONS

This application claims the benefit under 35 U.S.C. § 119 (e) of U.S. Provisional Application, U.S. Ser. No. 63/328,069, filed Apr. 6, 2022, which is incorporated by reference herein in its entirety.

### GOVERNMENT SUPPORT

This invention was made with government support under AG068303 awarded by National Institutes of Health (NIH). The government has certain rights in this invention.

### REFERENCE TO AN ELECTRONIC SEQUENCE LISTING

The contents of the electronic sequence listing (H082470401WO00-SEQ-FL.xml; Size: 278,541 bytes; and Date of Creation: Apr. 5, 2023) is herein incorporated by reference in its entirety.

### BACKGROUND OF THE INVENTION

In many animals, including vertebrates, vital organs have a limited intrinsic capacity for regeneration and repair. Acute injury and chronic disorders can damage vital organs and tissues, including the brain. Mature somatic cells, however, often cannot survive these insults, and even if they do, they are unable to self-renew and transdifferentiate to replace damaged cells. Furthermore, cells that are capable of self-renewal can be limited in quantity, have limited capacity and are susceptible to damage, especially with age. In contrast to somatic cells from adults, cells from individuals that are chronologically closer to fertilization, such as those from embryos and infants, display cellular youthfulness and have a greater capacity to resist injury and stress, to heal, renew, and regenerate organs and tissues. Thus, compositions and methods directed at rejuvenating cells, thereby restoring them from an aged, mature state to a younger, more vital state, have long been sought to treat certain injuries and diseases, as well as generally reverse and prevent aging in entire organisms.

There are two types of information in the body: digital and analog. DNA is digital information and the epigenome is analog information. Analog information never lasts as long as digital, nor can analog information be copied with high fidelity compared to digital information. This has consequences for how long organisms live and thrive. Aging was once thought of as a process driven by mutations in the genetic material of a cell. This has largely been abandoned as an explanation. A major cause of aging is now thought to be due to epigenetic changes that cause cells to transcribe the wrong genes at the wrong time, a process that becomes more dysfunctional over time, leading to diseases, an inability to heal and eventually to death. The Yamanaka factors (OCT4, SOX2, c-Myc, and KLF4) have previously been shown to induce pluripotency in vitro (Takahashi et al., Cell. 2006 Aug. 25; 126 (4): 663-76) and reverse the DNA methylation clock of aging (Horvath, Genome Biol. 2013). Nanog and Lin28 can help induce pluripotency together with Yamanaka factors. And Tet1, NR5A-2, Sall4, and NKX3-1 can replace Oct4 (Gao et al., Cell Stem Cell 12, 1-17, Apr. 4, 2013; and Mai et al., Nature Cell Biology 20, 900-908, 2018). Expression of the original four transcription factors in transgenic mice, however, induces teratomas in vivo, along with other acute toxicities like dysplasia in the intestinal epithelium, that can kill an animal in a few days (Abad et al., Nature. 2013 Oct. 17; 502 (7471): 340-5). Therefore, non-toxic and efficient methods of cellular reprogramming are needed.

### SUMMARY OF THE INVENTION

The cellular aging process has been postulated to be caused by the loss of both genetic and epigenetic information. While previous studies have hypothesized that aging is caused primarily by the loss of genetic information (most commonly in the form of genetic mutations such as substitutions, and deletions in an organism's genome), the systems, compositions, uses, kits, and methods of the present disclosure are informed by the unexpected finding that aging in the central nervous system is primarily driven by a loss in the particular epigenetic information that is established closer to fertilization and final differentiation of particular cells. Epigenetic information, which commonly takes the form of covalent modifications to DNA, such as 5-methylcytosine (5mC), hydroxymethylcytosine (5hmeC), 5-formylcytosine (fC), 5-carboxylcytosine (caC), and adenine methylation, and to certain proteins, such as lysine acetylation, lysine and arginine methylation, serine and threonine phosphorylation, and lysine ubiquitination and sumoylation of histone proteins, is sometimes referred to as the “analog” information of the cell. The loss of this analog information can result in dysregulation of vital cellular processes, such as the processes that maintain cell identity, causing cells to exhibit traits that are typically associated with aging, such as senescence.

Aging of the brain is often characterized by a progressive loss of neuronal plasticity and function, leading to cognitive decline and increased vulnerability to neurodegeneration. Many of the clinically approved treatments for neurological disorders attempt to modulate neurotransmitter activity by targeting proteins rather than reversing the cellular causes of aging. For example, memantine is a NMDA receptor antagonist and has been approved by the Food and Drug Administration in the United States and Europe for patients with Alzheimer's disease. Other treatments for Alzheimer's disease include cholinesterase inhibitors. However, these inhibitors fail to address the underlying disease.

Surprisingly, as disclosed herein, it was found that epigenetic reprogramming with three transcription factors was sufficient to improve neuronal and cognitive function in vivo. The methods, compositions, uses, and kits of the present disclosure rejuvenate cells by preventing and reversing the cellular causes of aging in the central nervous system (e.g., brain). In some embodiments, the central nervous system does not include the eye. In some embodiments, the central nervous system does not include the retina, uvea, pupil, lens, cornea, and/or sclera.

The present disclosure stems from the unexpected discovery that, in some embodiments, precise expression of OCT4, SOX2, and KLF4 in the absence of exogenous c-Myc expression can be used to promote reprogramming and tissue regeneration in the central nervous system (e.g., the brain) in vivo without acute toxicity. Surprisingly, in some embodiments, the compositions disclosed herein can permeate the blood-brain barrier and results disclosed herein demonstrate that OCT4, SOX2, and KLF4 expression can be used to reverse aging of the brain in the absence of exogenous c-Myc expression. The expression vectors provided herein, in certain embodiments, allow for precise control and targeting of OCT4, SOX2, and KLF4 (OSK) expression to the central nervous system, incorporation into viruses (e.g., adeno-associated virus (AAV) at a high viral titer (e.g., more than 2×10¹² particles per preparation, 1×10¹³ particles per mL), reversing aging, and treating diseases, including neurological diseases.

Aspects of the present disclosure provide several systems, compositions, uses, kits, and methods that may be useful for efficient rejuvenation of a cell, tissue, and/or organ in the central nervous system. In some embodiments, the central nervous system does not include the retina. In some embodiments, the cell, tissue, and/or organ in the central nervous system is a brain cell, brain tissue, and/or the brain. For example, aspects of the present disclosure provide nucleic acids that may be useful for efficient rejuvenation of the central nervous system by promoting expression of OCT4, SOX2, and/or KLF4 without inducing c-Myc expression. In some embodiments, such a nucleic acid encodes an inducing agent (e.g., tetracycline transactivor or reverse tetracycline transactivator) operably linked to a CaMKIIα promoter. In some embodiments, the CaMKIIα promoter is at least 70% (e.g., at least 75%, 80%, 85%, 90%, 95%, 98%, 99%, or 100%) identical to SEQ ID NO: 149. In some embodiments, the nucleic acid encoding an inducing agent does not comprise a Synapsin-I promoter or a CaMKII-gamma promoter. In some embodiments, the Synapsin-I promoter is at least 70% (e.g., at least 75%, 80%, 85%, 90%, 95%, 98%, 99%, or 100%) identical to SEQ ID NO: 157. In some embodiments, a nucleic acid encoding an inducing agent is a viral vector. In some embodiments, the viral vector is packaged in AAV.PHP.eB virus. In some embodiments, a nucleic acid encoding an inducing agent comprises a sequence that is at least 70% (e.g., at least 75%, 80%, 85%, 90%, 95%, 98%, 99%, or 100%) identical to rtTA2S-M2 (SEQ ID NO: 14), pLVX-rtTA-hOSK-all-in-one (human) (SEQ ID NO: 123), pAAV-CaMKIIα-tTA2 (SEQ ID NO: 124), pAAV-CaMKIIα-rtTA2S-M2 (SEQ ID NO: 125), pAAV-CaMKIIα-rtTA3 (SEQ ID NO: 126), CaMKIIα promoter (SEQ ID NO: 146, 149, or 154); rtTA Advanced in reverse complement (SEQ ID NO: 128), tTA Advanced (SEQ ID NO: 137), and/or TA (SEQ ID NO: 158). In some embodiments, a nucleic acid encoding an inducing agent does not comprise a sequence that is at least 70% (e.g., at least 75%, 80%, 85%, 90%, 95%, 98%, 99%, or 100%) identical to pAAV-ihSyn1-tTA (SEQ ID NO: 127). In some embodiments, an inducing agent comprises a sequence that is at least 70% (e.g., at least 75%, 80%, 85%, 90%, 95%, 98%, 99%, or 100%) identical to rtTA Advanced (SEQ ID NO: 129), (TA Advanced (SEQ ID NO: 138), rtTA2S-M2 (SEQ ID NO: 15), and/or tTA (SEQ ID NO: 159).

Aspects of the present disclosure provide nucleic acids, which may be useful in inducing expression of OCT4, KLF4, and/or SOX2 in the absence of inducing c-Myc expression in a cell, tissue, or organ of the central nervous system. These nucleic acids may be useful in rejuvenating the cell, tissue, or organ of the central nervous system. In some embodiments, the nucleic acid is a nucleic acid with (a) a nucleic acid sequence that encodes OCT4, KLF4, and SOX2 operably linked to a TRE promoter; and (b) a nucleic acid sequence that encodes rtTA operably linked to a UbC promoter. In some embodiments, the nucleic acid sequence that encodes OCT4, KLF4, and SOX2 further encodes a 2A peptide. In some embodiments, the nucleic acid with (a) and (b) further encodes a neomycin resistance gene and/or comprises a WPRE sequence. In some embodiments, the neomycin resistance gene is operably linked to a PGK promoter. In some embodiments, the nucleic acid with (a) and (b) encodes at least one protein sequence that is at least 70% identical (e.g., at least 75%, 80%, 85%, 90%, 95%, 98%, 99%, or 100% identical) to a sequence selected from: rtTA Advanced (SEQ ID NO: 129); human OCT4 (SEQ ID NO: 41); P2A (SEQ ID NO: 118); human SOX2 (SEQ ID NO: 43); T2A (SEQ ID NO: 9); human KLF4 (SEQ ID NO: 45); and neomycin resistance gene (SEQ ID NO: 134). In some embodiments, the nucleic acid with (a) and (b) comprises at least one sequence that is at least 70% identical to a sequence selected from: rtTA Advanced in reverse complement (SEQ ID NO: 128); UbC promoter in reverse complement (SEQ ID NO: 130); P tight TRE promoter (SEQ ID NO: 24); human OCT4 (SEQ ID NO: 40); P2A (SEQ ID NO: 119); human SOX2 (SEQ ID NO: 42); T2A (SEQ ID NO: 120); human KLF4 (SEQ ID NO: 131); PGK promoter (SEQ ID NO: 132); neomycin resistance gene (SEQ ID NO: 133); and WPRE (SEQ ID NO: 135). In some embodiments, the nucleic acid with (a) and (b) comprises a sequence that is at least 70% identical (e.g., at least 75%, 80%, 85%, 90%, 95%, 98%, 99%, or 100% identical) to pLVX-rtTA-hOSK-all-in-one (human) (SEQ ID NO: 123).

In some embodiments, the nucleic acid encodes a tTA, wherein the tTA comprises a sequence that is at least 70% identical (e.g., at least 75%, 80%, 85%, 90%, 95%, 98%, 99%, or 100% identical) to SEQ ID NO: 138. In some embodiments, the nucleic acid encoding the tTA is at least 70% identical (e.g., at least 75%, 80%, 85%, 90%, 95%, 98%, 99%, or 100% identical) to SEQ ID NO: 137. In some embodiments, the nucleic acid encoding the tTA comprises a hGH pA sequence and/or a CMV promoter. In some embodiments, the hGH pA sequence is at least 70% identical (e.g., at least 75%, 80%, 85%, 90%, 95%, 98%, 99%, or 100% identical) to SEQ ID NO: 139. In some embodiments, the CMV promoter is at least 70% identical (e.g., at least 75%, 80%, 85%, 90%, 95%, 98%, 99%, or 100% identical) to SEQ ID NO: 136. In some embodiments, the nucleic acid encoding a tTA comprises a sequence that is at least 70% identical (e.g., at least 75%, 80%, 85%, 90%, 95%, 98%, 99%, or 100% identical) to pAAV-CMV-tTA (Advanced) (SEQ ID NO: 32).

In some embodiments, the nucleic acid comprises a nucleic acid encoding OCT4, SOX2, and KLF4 operably linked to a TRE promoter. In some embodiments, the nucleic acid sequence encoding OCT4, SOX2, and KLF4 operably linked to a TRE promoter further encodes a 2A peptide and/or SV40 pA. In some embodiments, the 2A peptide comprises a sequence that is at least 70% identical (e.g., at least 75%, 80%, 85%, 90%, 95%, 98%, 99%, or 100% identical) to T2A (SEQ ID NO: 9) or P2A (SEQ ID NO: 118). In some embodiments, the nucleic acid sequence encoding OCT4, SOX2, and KLF4 encodes at least one sequence that is 70% identical to a sequence selected from: mouse OCT4 (SEQ ID NO: 2); human OCT4 (SEQ ID NO: 40); mouse SOX2 (SEQ ID NO: 4); human SOX2 (SEQ ID NO: 42); human KLF4 (SEQ ID NO: 131); and mouse KLF4 (SEQ ID NO: 6). In some embodiments, the TRE promoter operably linked to the nucleic acid sequence encoding OCT4, SOX2, and KLF4 is at least 70% identical (e.g., at least 75%, 80%, 85%, 90%, 95%, 98%, 99%, or 100% identical) to SEQ ID NO: 7. In some embodiments, the nucleic acid sequence encoding OCT4, SOX2, and KLF4 operably linked to the TRE promoter comprises at least one sequence that is 70% identical to a sequence selected from: TRE3G (SEQ ID NO: 7): mouse Oct4 (SEQ ID NO: 1); P2A (SEQ ID NO: 144); mouse Klf4 (SEQ ID NO: 145); SV40 pA (SEQ ID NO: 143); mouse Sox2 (SEQ ID NO: 3); and T2A (SEQ ID NO: 120), optionally wherein the sequence is at least 70% identical (e.g., at least 75%, 80%, 85%, 90%, 95%, 98%, 99%, or 100% identical) to pAAV-TRE3G-OSK (mouse) SEQ ID NO: 16.

In some embodiments, the nucleic acid comprises a nucleic acid that encodes an inducing agent and the nucleic acid encoding the inducing agent is operably linked to a CaMKIIα promoter. In some embodiments, the inducing agent is a tTA or rtTA. In some embodiments, the inducing agent comprises a sequence that is at least 70% identical (e.g., at least 75%, 80%, 85%, 90%, 95%, 98%, 99%, or 100% identical) to tTA Advanced (SEQ ID NO: 138), rtTA2S-M2 (SEQ ID NO: 15), or rtTA3 (SEQ ID NO: 11). In some embodiments, the CaMKIIα promoter comprises a sequence that is at least 70% identical (e.g., at least 75%, 80%, 85%, 90%, 95%, 98%, 99%, or 100% identical) to SEQ ID NO: 146, SEQ ID NO: 149, or SEQ ID NO: 154. In some embodiments, the nucleic acid encoding the inducing agent further comprises a WPRE and/or hGH pA sequence. In some embodiments, the WPRE sequence is at least 70% identical (e.g., at least 75%, 80%, 85%, 90%, 95%, 98%, 99%, or 100% identical) to SEQ ID NO: 147, 152, or 155. In some embodiments, the hGH pA sequence is at least 70% identical (e.g., at least 75%, 80%, 85%, 90%, 95%, 98%, 99%, or 100% identical) to SEQ ID NO: 148, 153, or 156.

In some embodiments, the nucleic acid encoding the inducing agent comprises a sequence that is at least 70% identical (e.g., at least 75%, 80%, 85%, 90%, 95%, 98%, 99%, or 100% identical) to a sequence selected from tTA-Advanced (SEQ ID NO: 137), rtTA2S-M2 (SEQ ID NO: 14), or rtTA3 (SEQ ID NO: 10). In some embodiments, the nucleic acid encoding the inducing agent comprises a sequence that is at least 70% identical (e.g., at least 75%, 80%, 85%, 90%, 95%, 98%, 99%, or 100% identical) to a sequence selected from pAAV-CaMKIIα-tTA2 (SEQ ID NO: 124), pAAV-CaMKIIα-rtTA2S-M2 (SEQ ID NO: 125), or pAAV-CaMKIIα-rtTA3 (SEQ ID NO: 126).

In some embodiments, the nucleic acid does not comprise a Synapsin-I promoter operably linked to a nucleic acid sequence encoding an inducing agent. In some embodiments, the nucleic acid does not comprise a sequence that is at least 70% identical (e.g., at least 75%, 80%, 85%, 90%, 95%, 98%, 99%, or 100% identical) to SEQ ID NO: 157. In some embodiments, the nucleic acid does not comprise a sequence that is at least 70% identical (e.g., at least 75%, 80%, 85%, 90%, 95%, 98%, 99%, or 100% identical) to pAAV-ihSyn1-tTA (SEQ ID NO: 127).

In some embodiments, OCT4, SOX2, and/or KLF4 is expressed in the central nervous system (e.g., the brain) for at most one month. Without being bound by a particular theory, expression of OCT4, SOX2, and/or KLF4 for two months may fail to rejuvenate the central nervous system.

Further aspects of the present disclosure provide recombinant viruses (e.g., lentivirus, adenovirus, alphavirus, vaccinia virus, retrovirus, herpes virus, human papillomavirus, or AAV) comprising a nucleic acid encoding OCT4, KLF4, and/or SOX2 and/or an inducing agent disclosed herein for delivery to the central nervous system.

In yet another aspect, the present disclosure provides methods of regulating (e.g., inducing) cellular reprogramming, tissue repair, tissue regeneration, organ regeneration, reversing aging, or any combination thereof. In certain embodiments, one or more expression vectors (e.g., AAV comprising an expression vector) is administered to a cell, tissue, or organ in the central nervous system, or to a subject with a neurological disease. The subject may have an injury or condition, is suspected of having a condition or injury, or is at risk for a condition or injury. Without being bound by a particular theory, expression of the transcription factors OCT4, SOX2, and KLF4 induces cellular reprogramming. In some embodiments, when the nucleic acid (e.g., engineered nucleic acid) encoding OCT4, SOX2, KLF4, or a combination thereof is operably linked to an inducible promoter, administration of an inducing agent (e.g., chemical, a protein, a nucleic acid (e.g., engineered nucleic acid) encoding an inducing agent) under the appropriate conditions (e.g., in the presence or absence of tetracycline). In certain embodiments, an inducing agent (e.g., rtTA) is capable of binding a promoter and driving expression of an operably linked nucleic acid (e.g., engineered nucleic acid) only when the inducing agent is bound to tetracycline. In certain embodiments, an inducing agent (e.g., (TA) cannot bind a promoter and drive expression of an operably linked nucleic acid (e.g., engineered nucleic acid) when the inducing agent is bound to tetracycline. The condition may be a neurological disease (e.g., a disease affecting the brain and/or neurodegenerative disease), cancer, aging, an age-related disease, injury, or a a disease affecting neurons and/or nerves. In certain embodiments, the cell, tissue, or organ is from the central nervous system, e.g., brain or spinal cord. In some embodiments, the cell or tissue is a neuronal cell or nervous tissue. In some embodiments, the cell is a neuron. In some embodiments, the neuron is an excitatory neuron. In some embodiments, a brain cell is a neuron or a glial cell. In some embodiments, a cell from the central nervous system is a neuron, glial cell, or choroid plexus cell. In some embodiments, a glial cell is an astrocyte, oligodendrocyte, ependymal cell, or microglia cell. In some embodiments, a neuron is a sensory neuron, a motor neuron or an interneuron.

In certain embodiments, the method comprises further regulation of a biological process in the central nervous system of a subject. In some embodiments, the methods described herein comprise regulating any biological process, including, cellular reprogramming, tissue repair, tissue survival, tissue regeneration, tissue growth, tissue function, organ regeneration, organ survival, organ function, or any combination thereof. In some embodiments, the methods comprise inducing cellular reprogramming, reversing aging, improving tissue function, improving organ function, promoting tissue repair, promoting tissue survival, promoting tissue regeneration, promoting tissue growth, promoting angiogenesis, reducing scar formation, promoting organ regeneration, promoting organ survival, treating a disease, or any combination thereof, in vivo or in vitro. For example, the method may induce cellular reprogramming, cell survival, organ regeneration, tissue regeneration, or a combination thereof. In certain embodiments, the method comprises inducing and then stopping cellular reprogramming, cell survival, tissue regeneration, organ regeneration, aging, or a combination thereof. In certain embodiments, the method reverses aging of a cell, tissue, organ, or subject. In some embodiments, the method does not induce cancer. Cancer formation may include teratoma formation and/or uncontrolled cell growth. In some embodiments, the method does not induce unwanted cell proliferation. In some embodiments, the method does not induce malignant cell growth. In some embodiments, the method does not induce tumor growth or tumor formation. In some embodiments, the method does not induce glaucoma or macular degeneration.

In some embodiments, a method described herein reverses the epigenetic clock of a cell, a tissue, and/or an organ from the central nervous system, or any combination thereof. In some embodiments, the epigenetic clock is determined using a DNA methylation-based (DNAm) age estimator. In some embodiments, the method alters the expression of one or more genes associated with ageing. In some embodiments, the method reduces expression of one or more genes associated with ageing. In some embodiments, the method alters the expression of one or more genes associated with ageing. In some embodiments, the one or more genes is a gene expressed by a cell in the central nervous system. In some embodiments, the one or more genes is a gene expressed in the brain or spinal cord. In some embodiments, the one or more genes is expressed in excitatory neurons or glial cells. In some embodiments, the one or more genes is a gene expressed by a brain cell, e.g., a neuron or a glial cell. In some embodiments, the one or more genes is a gene expressed by a neuron, glial cell, or choroid plexus cell. In some embodiments, the one or more genes is a gene expressed by an astrocyte, oligodendrocyte, ependymal cell, or microglia cell. In some embodiments, the one or more genes is a gene expressed by a sensory neuron, a motor neuron or an interneuron.

Another aspect of the present disclosure provides engineered cells generated by any of the methods described herein. In some embodiments, the engineered cell is a cell of the central nervous system. In some embodiments, the engineered cell is a neural cell. The engineered cells of the present disclosure may be produced ex vivo and the methods may further comprise generating an engineered tissue or engineered organ. In some embodiments, the methods of the present disclosure comprise administering an engineered cell, engineered tissue, and/or engineered organ of the present disclosure to a subject in need thereof. In some embodiments, the method further comprises treating a neurological disease.

Aspects of the present disclosure also provide compositions comprising any of the nucleic acids, any of the engineered proteins described herein, any of the chemical agents, any of the recombinant viruses, cells, and/or agents described herein, alone, or in combination. In some embodiments, the composition is a pharmaceutical composition. In some embodiments, the pharmaceutical compositions of the present disclosure further comprise a pharmaceutically acceptable carrier. In some embodiments, the pharmaceutical composition comprises an engineered nucleic acid encoding OCT4, SOX2, and/or KLF4. In some embodiments, a pharmaceutical composition comprises an engineered nucleic acid (e.g., engineered nucleic acid) (e.g., expression vector including viral vector) encoding an inducing agent (e.g., rtTA or (TA).

Aspects of the present disclosure also provide kits comprising any of the nucleic acids, viruses, cells, compositions, and agents disclosed herein and instructions for instructions for rejuvenating a cell, tissue, or organ from the central nervous system.

The details of one or more embodiments of the invention are set forth herein. Other features, objects, and advantages of the invention will be apparent from the Detailed Description, Examples, Figures, and Claims.

References cited in this application are incorporated herein by reference.

### Definitions

Definitions of specific terms are described in more detail below. The disclosure is not intended to be limited in any manner by the exemplary listing of substituents described herein.

“AAV” or “adeno-associated virus” is a nonenveloped virus that is capable of carrying and delivering nucleic acids (e.g., engineered nucleic acids) (e.g., nucleic acids (e.g., engineered nucleic acids) encoding OCT4; KLF4; SOX2; or any combination thereof) and belongs to the genus Dependoparvovirus. In some instances, an AAV is capable of delivering a nucleic acid encoding an inducing agent. In general, AAV does not integrate into the genome. The tissue-specific targeting capabilities of AAV is often determined by the AAV capsid serotype (see, e.g., Table 1 below for examples of AAV serotypes and their utility in tissue-specific delivery). Non-limiting serotypes of AAV include AAV1, AAV2, AAV3, AAV4, AAV5, AAV6, AAV7, AAV8, AAV9, AAV10, AAV11, AAV PHP.b, and variants thereof. In certain embodiments, the AAV serotype is a variant of AAV9 (e.g., AAV PHP.b). In certain embodiments, the AAV serotype is AAV PHP.eB. In some embodiments, the AAV serotype is not AAV2 or AAV9. In some embodiments, an AAV capsid comprises a targeting moiety for one or more cells of the central nervous system. See, e.g., WO2023004367 and WO2022232327. In some embodiments, the AAV capsid is AAV-BI30. See, e.g., Krolak et al., Nat Cardiovasc Res. 2022 April; 1 (4): 389-400.

A “recombinant virus” is a virus (e.g., lentivirus, adenovirus, retrovirus, herpes virus, human papillomavirus, alphavirus, vaccinia virus or adeno-associated virus (AAV)) that has been isolated from its natural environment (e.g., from a host cell, tissue, or a subject) or is artificially produced.

The term “AAV vector” as used herein is a nucleic acid (e.g., engineered nucleic acid) that comprises AAV inverted terminal repeats (ITRs) flanking an expression cassette (e.g., an expression cassette comprising a nucleic acid (e.g., engineered nucleic acid) encoding OCT4, KLF4, and SOX2, each alone or in combination, or an expression cassette encoding rtTA or (TA). An AAV vector may further comprise a promoter sequence.

The terms “administer.” “administering.” or “administration,” as used herein, refers to introduction of any of the compositions described herein, any of the nucleic acids described herein, any of the engineered proteins described herein, any of the chemical agents activating (e.g., inducing expression of) OCT4, KLF4, and/or SOX2, any of the chemical agents activating (e.g., inducing expression of) one or more transcription factors selected from OCT4; KLF4; SOX2; and any combinations thereof, any of the antibodies activating (e.g., inducing expression of) OCT4, KLF4, and/or SOX2, any of the antibodies activating (e.g., inducing expression of) one or more transcription factors selected from OCT4; KLF4; SOX2; and any combinations thereof, and/or any of the recombinant viruses (e.g., lentivirus, adenovirus, alphavirus, vaccinia virus, retrovirus, herpes virus, human papillomavirus, or AAV) described herein, alone, or in combination to any cell, tissue, organ, and/or subject. In some embodiments, a nucleic acid (e.g., engineered nucleic acid) encoding an inducing agent, an engineered protein encoding an inducing agent, a chemical agent capable of modulating (e.g., activating or inhibiting) the activity of an inducing agent, and/or a recombinant virus encoding an inducing agent is also administered to the cell, tissue, organ and/or subject. Any of the compositions described herein, comprising any of the nucleic acids (e.g., engineered nucleic acid) capable of inducing OCT4, KLF4, and/or SOX2 expression (e.g., expression vector), comprising any of the nucleic acids (e.g., engineered nucleic acid) (e.g., expression vector) capable of inducing expression of one or more transcription factors selected from OCT4; KLF4; SOX2; and any combinations thereof, any of the engineered proteins described herein, any of the chemical agents activating (e.g., inducing expression of) OCT4, KLF4, and/or SOX2, any of the engineered proteins encoding OCT4, SOX2, KLF4, or any combinations thereof, any of the chemical agents activating (e.g., inducing expression of) OCT4; KLF4; SOX2; or any combination thereof, any of the antibodies activating (e.g., inducing expression of) OCT4, KLF4, and/or SOX2, any of the antibodies activating (e.g., inducing expression of) OCT4; KLF4; SOX2; or any combination thereof, and/or any of the recombinant viruses (e.g., lentivirus, adenovirus, alphavirus, vaccinia virus, retrovirus, herpes virus, human papillomavirus, or AAV) described herein, alone, or in combination may be administered intravenously, intradermally, intraarterially, intralesionally, intratumorally, intracranially, intraarticularly, intraprostatically, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes), or by other method or any combination of the forgoing as would be known to one of ordinary skill in the art (see, for example, Remington's Pharmaceutical Sciences (1990), incorporated herein by reference). In some embodiments, a composition comprising a nucleic acid (e.g., engineered nucleic acid) encoding an inducing agent, an engineered protein encoding an inducing agent, a chemical agent capable of modulating (e.g., activating or inhibiting) the activity of an inducing agent, and/or a recombinant virus encoding an inducing agent is also administered to the cell, tissue, organ and/or subject is administered using any suitable method.

The term “epigenome” or “epigenetics” refers to the modification and structural changes within a cell that control the expression of nucleic acids (e.g., engineered nucleic acids) or genomic information in a cell. Changes to the epigenome occur during, and drive the processes of embryonic development, disease progression, and aging.

The term “epigenetic clock” may refer to an age estimator or an innate biological process. In some embodiments, rejuvenating or reversing the epigenetic clock refers to reducing the estimated age of a cell, tissue, organ, or a subject. The epigenetic clock may be partially or fully reversed or rejuvenated by any of the methods described herein. In some embodiments, an age estimator is an epigenetic age estimator. For example, an epigenetic age estimator may be sets of CpG dinucleotides that when used in combination with a mathematical algorithm may be used to estimate age of a DNA source, including cells, organs, or tissues. In some embodiments, an age estimator is a DNA methylation-based (DNAm) age estimator. In some embodiments, a DNAm age estimator is calculated as an age correlation using Pearson correlation coefficient r, between DNA methylation-based (DNam) age (also known as estimated age) and chronological age. In some embodiments, the DNA methylation-based (DNAm) age estimator is a single-tissue DNA methylation-based age estimator. In some embodiments, the DNA methylation-based age estimator is a multi-tissue DNA methylation-based age estimator. In some embodiments, the DNAm age estimator is DNAm PhenoAge. See, e.g., Horvath and Raj, Nat Rev Genet. 2018 June; 19 (6): 371-384 and Levine et al., Aging (Albany NY). 2018 Apr. 18; 10 (4): 573-591.

“Epigenetic information” as used herein includes covalent modifications to DNA, such as 5-methylcytosine (5mC), hydroxymethylcytosine (5hmeC), 5-formylcytosine (fC), 5-carboxylcytosine (caC), and adenine methylation and to certain proteins, such as lysine acetylation, lysine and arginine methylation, serine and threonine phosphorylation, and lysine ubiquitination and sumoylation of histone proteins, and the 3D architecture of cells, including TADs (topologically associated domains) and compartments. Epigenetic information is sometimes referred to as the “analog” information of the cell.

“Restoring the expression” of at least one gene to youthful levels is meant to include increasing the expression of a downregulated gene or decreasing the expression of an upregulated gene that changes during aging. In some embodiments, the at least one gene is at least one gene selected from the group consisting of RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

As used herein, the term “cell” is meant not only to include an individual cell but refers also to the particular tissue or organ from which it originates.

The term “cellular senescence” refers to a cell that has exited the cell cycle, displays epigenetic markers consistent with senescence, or expressing senescence cell markers (e.g., senescence-associated beta-galactosidase, or inflammatory cytokines). Cellular senescence may be partial or complete.

The term “gene expression” refers to the degree to which certain genes or all genes in a cell or tissue are transcribed into RNA. In some instances, the RNA is translated by the cell into a protein. The epigenome dictates gene expression patterns.

The term “cellular reprogramming” refers to the process of altering the epigenome of a cell using reprogramming factors (e.g., reversing or preventing epigenetic changes in cells that are causes of dysfunction, deterioration, cell death, senescence, or aging). Cellular reprogramming may be complete reprogramming, such that a differentiated cell (e.g., somatic cell) is reprogrammed to a pluripotent stem cell. Cellular reprogramming may be incomplete, such that a differentiated cell (e.g., somatic cell) retains its cellular identity (e.g., lineage-specific stem cell). Cellular reprogramming may be incomplete, e.g., a stem cell is not created, such that a cell is rejuvenated, or takes on more youthful attributes (e.g., increased survival, reduced inflammation, or ability to divide). Cellular reprogramming may provide additional cellular functions, or prevent cellular aging (e.g., transdifferentiation, or transition into cellular senescence). Cellular reprogramming may induce temporary or permanent gene expression changes. In some embodiments, incomplete cellular reprogramming is shown by the lack of Nanog expression. In some embodiments, cellular reprogramming prevents senescence from occurring.

The term “rejuvenating a cell” as used herein is meant to include preventing or reversing the cellular causes of aging without inducing a pluripotent state. A rejuvenated cell as used herein includes for example a central nervous system cell that is not a diseased cell.

A “pluripotent state” as used herein is meant to include a state in which the cell expresses at least one stem cell marker, such as, but not limited to, Esrrb, Nanog, Lin28, TRA-1-60/TRA-1-81/TRA-2-54, SSEA1, or SSEA4. Methods of measuring the expression of stem cell markers on the cell are known in the art and include the methods described herein.

The term “transdifferentiation” refers to a process in which one cell type is changed into another cell type without entering a pluripotent state. Transdifferentiation may also be referred to as lineage reprogramming or lineage conversion. See, e.g., Cieślar-Pobuda et al., Biochim Biophys Acta Mol Cell Res. 2017 July; 1864 (7): 1359-1369, which is incorporated herein by reference in its entirety.

The term “central nervous system” refers to the part of the nervous system that includes the brain, chochlea, the spinal cord, the medulla, the pons, the cerebellum, the midbrain, the diencephalon, and the cerebral hemispheres. In some embodiments, the central nervous system includes the cranial nerves. In some embodiments, the central nervous system excludes the eye. In some embodiments, the central nervous system excludes the retina, uvea, pupil, lens, cornea, and/or sclera. In some embodiments, a cell or tissue is derived from the central nervous system. Non-limiting examples of cells from the central nervous system include neurons and glial cells. In some embodiments, a neuron is an excitatory neuron. In some embodiments, a cell from the central nervous system is a brain cell. In some embodiments, a brain cell is a neuron or a glial cell. In some embodiments, a cell from the central nervous system is a neuron, glial cell, or choroid plexus cell. In some embodiments, a glial cell is an astrocyte, oligodendrocyte, ependymal cell, or microglia cell. In some embodiments, a neuron is a sensory neuron, a motor neuron or an interneuron.

The terms “condition,” “disease,” and “disorder” are used interchangeably. Non-limiting examples of conditions, diseases, and disorders include acute injuries, neurodegenerative diseases, chronic diseases, proliferative diseases, cardiovascular diseases, genetic diseases, inflammatory diseases, autoimmune diseases, neurological diseases, hematological diseases, painful conditions, psychiatric disorders, metabolic disorders, chronic diseases, cancers, aging, age-related diseases, and diseases affecting any tissue in a subject. For example, age-related conditions include, heart failure, stroke, heart disease, atherosclerosis, neurodegenerative diseases (e.g., Alzheimer's Disease, Parkinson's Disease, dementia, Friedreich ataxia, amyotrophic lateral sclerosis, or vascular dementia.), cognitive decline, memory loss, diabetes, osteoporosis, arthritis, muscle loss, hearing loss (partial or total), eye-related conditions (e.g., poor eye sight or retinal disease), glaucoma, a progeroid syndrome (e.g., Hutchinson-Gilford progeria syndrome), and cancer. In certain embodiments, the disease is a retinal disease (e.g., macular degeneration). In some embodiments, an age-related condition is senescence. As a non-limiting example, senescence of glial cells may be a cause of Alzheimer's disease. Sec e.g., Bussian, et al., Nature. 2018 October; 562 (7728): 578-582. In some instances, the disease is nerve damage. In some embodiments, the nerve damage is neurapraxia, axonotmesis, or neurotmesis. In some embodiments, the disease is not an ocular disease, ophthalmic disease, or eye disease.

In some instances, the condition is nerve damage. In some instances, the condition is damage in the central nervous system (CNS). In some instances, the nerve damage is a spinal cord injury. In some instances, the nerve damage is neurapraxia, axonotmesis, or neurotmesis.

In some instances, a condition increases the DNA methylation-based age of a cell, a tissue, an organ, and/or a subject relative to a control. In some embodiments, the cell is a cell of the central nervous system. In some instances, a condition increases the DNA methylation-based age of a cell, a tissue, an organ, and/or a subject by at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% relative to a control. In some instances, the control is a cell, a tissue, an organ, and/or a subject that does not have the condition. In some instances, the control is the same cell, tissue, organ, and/or subject prior to having the condition. Without being bound by a particular theory, any of the methods described herein may be useful in decreasing the DNA methylation-based age of a diseased cell, a diseased tissue, a diseased organ, and/or a subject who has, is at risk for, or is suspected of having a disease. In some instances, the disease increases the DNA-methylation-based age of the cell, tissue, organ, and/or subject. In some instances, the disease is an injury.

In some instances, the condition is ageing. In some instances, aging is driven by epigenetic noise. See, e.g., Oberdoerffer and Sinclair. Nat Rev Mol Cell Biol 8, 692-702. doi: 10.1038/nrm2238 (2007); Oberdoerffer et al. Cell 135, 907-918, doi: 10.1016/j.cell.2008.10.025 (2008). Without being bound by a particular theory, mammalian cells may retain a faithful copy of epigenetic information from earlier in life, analogous to Shannon's “observer” system in Information Theory, essentially a back-up copy of the original signal to allow for its reconstitution at the receiving end if information is lost or noise is introduced during transmission. See, e.g., Shannon, The Bell System Technical Journal 27, 379-423 (1948) for a description of the observer system.

As used herein, an “ocular disease,” “ophthalmic disease,” or “eye disease” is a disease or condition of the eye. Non-limiting examples of conditions that affect the eye include Ectropion, Lagophthalmos, Blepharochalasis, Ptosis, Stye, Xanthelasma, Dermatitis, Demodex, leishmaniasis, loiasis, onchocerciasis, phthiriasis, (herpes simplex), leprosy, molluscum contagiosum, tuberculosis, yaws, zoster, impetigo, Dacryoadenitis, Epiphora, exophthalmos, Conjunctivitis, Scleritis, Keratitis, Corneal ulcer/Corneal abrasion, Snow blindness/Arc eye, Thygeson's superficial punctate keratopathy, Corneal neovascularization, Fuchs' dystrophy, Keratoconus, Keratoconjunctivitis sicca, Iritis, iris, Uveitis, Sympathetic ophthalmia, Cataract, lens, Chorioretinal inflammation, Focal chorioretinal inflammation, chorioretinitis, choroiditis, retinitis, retinochoroiditis, Disseminated chorioretinal inflammation, exudative retinopathy, Posterior cyclitis, Pars planitis, chorioretinal inflammations, Harada's disease, Chorioretinal inflammation, choroid, Chorioretinal scars, Macula scars, posterior pole (postinflammatory) (post-traumatic), Solar retinopathy, Choroidal degeneration, Atrophy, Sclerosis, angioid streaks, choroidal dystrophy, Choroideremia, choroidal, arcolar, (peripapillary), Gyrate atrophy, choroid, ornithinacmia, Choroidal haemorrhage, Choroidal detachment, Chorioretinal, Chorioretinal inflammation, infectious and parasitic diseases, Chorioretinitis, syphilitic, toxoplasma, tuberculosis, chorioretinal, Retinal detachment, retina, choroid, distorted vision, Retinoschisis, Hypertensive retinopathy, Diabetic retinopathy, Retinopathy, Retinopathy of prematurity, Age-related macular degeneration, macula, Macular degeneration, Bull's Eye Maculopathy, Epiretinal membrane, Peripheral retinal degeneration, Hereditary retinal dystrophy, Retinitis pigmentosa, Retinal haemorrhage, retinal layers, Central serous retinopathy, Retinal detachment, retinal disorders, Macular edema, macula, Retinal disorder, Diabetic retinopathy, Glaucoma, optic neuropathy, ocular hypertension, open-angle glaucoma, angle-closure glaucoma, Normal Tension glaucoma, open-angle glaucoma, angle-closure glaucoma, Floaters, Leber's hereditary optic neuropathy, Optic disc drusen, Strabismus, Ophthalmoparesis, eye muscles, Progressive external ophthalmoplegia, Esotropia, Exotropia, Disorders of refraction, accommodation, Hypermetropia, Myopia, Astigmatism, Anisometropia, Presbyopia, ophthalmoplegia, Amblyopia, Leber's congenital amaurosis, Scotoma, Anopsia, Color blindness, Achromatopsia/Maskun, cone cells, Nyctalopia, Blindness, River blindness, Micropthalmia/coloboma, optic nerve, brain, spinal cord, Red eye, Argyll Robertson pupil, pupils, Keratomycosis, Xerophthalmia, and Aniridia. In some embodiments, the ocular disease is acute or chronic eye injury. The methods disclosed herein exclude treatment of ocular disease, ophthalmic disease, or eye disease.

In some embodiments, the ocular disease is a scratched cornea.

In some embodiments, the ocular disease is glaucoma.

In some embodiments, an ocular disease is a corneal disease (e.g., a disease affecting the cornea or corneal cells). In some embodiments, an ocular disease is acanthamoeba keratitis, ectropion, lagoph amblyopia, anisocoria, astigmatism, Bell's Palsy, blepharitis, blurry vision, burning eyes, cataracts, macular degeneration, age-related macular degeneration, diabetic eye disease, glaucoma, dry eye, poor vision (e.g., low vision), astigmatism, blepharitis, cataract, chalazion, conjunctivitis, diabetic retinopathy, dry eye, glaucoma, keratitis, keratonconus, macular degeneration, ocular hypertension, pinquecula, pterygium, retinitis pigmentosa, or ocular cancer (e.g., retinoblastoma, melanoma of the eye, lymphoma of the eye, medulloepithelioma, squamous cell cancer of the conjunctiva). Examples of corneal diseases include, but are not limited to, corneal neovascularization (NV), corneal dystrophy, corneal inflammation, corneal abrasion, and corneal fibrosis. In some embodiments, the ocular disease is Keritaconus. In some embodiments, an ocular disease is macular degeneration. Additional non-limiting examples of eye diseases may be found in the International Statistical Classification of Diseases and Related Health Problems (e.g., VII Diseases of the eye and adnexa).

An ocular disease may affect any part of the eye and/or adnexa. In some embodiments, the ocular disease is a disorder of the eyelid, lacrimal system, and/or orbit. In some embodiments, the ocular disease is a disorder of the conjunctiva. In some embodiments, the ocular disease is a disorder of sclera, cornea, iris, and/or ciliary body. In some embodiments, the ocular disease is a disorder of the lens. In some embodiments, the ocular disease is a disorder of the choroid and/or retina. In some embodiments, the ocular disease is glaucoma. In some embodiments, the ocular disease is a disorder of vitreous body and/or globe. In some embodiments, the ocular disease is a disorder of optic nerve and/or visual pathways. In some embodiments, the ocular disease is a disorder of ocular muscles, binocular movement, accommodation, and/or refraction. In some embodiments, the ocular disease is a visual disturbance and/or blindness. In some embodiments, the ocular disease is associated with aging, for example, vision loss associated with aging, decline in visual acuity associated with aging, and/or decline in retinal function.

Any suitable method may be used to measure ocular function. Non-limiting examples include visual acuity tests, pattern electroretinograms, and pathology. The methods disclosed herein exclude treatment of ocular disease, ophthalmic disease, or eye disease.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroncal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, a progeroid syndrome (e.g., Hutchinson-Gilford progeria syndrome), and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “Nanog” refers to a DNA binding homeobox transcription factor that has been implicated in embryonic stem (ES) cell proliferation, renewal, and pluripotency. Non-limiting examples of amino acid sequences encoding Nanog include the amino acid sequences under UniProtKB Accession Nos. Q9H9S0, Q4JM65, Q80Z64, and A7Y7W3. In some embodiments, an amino acid sequence encoding Nanog comprises a sequence that is at least 70% (e.g., at least 75%, 80%, 85%, 90%, 95%, 98%, 99%, or 100%) identical to a sequence as described in UniProtKB Accession No. Q9H9S0, Q4JM65, Q80Z64, or A7Y7W3.

The term “neurological disease” refers to any disease of the nervous system, including diseases and injuries that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, dementia, Friedreich ataxia, amyotrophic lateral sclerosis, tauopathies (including frontotemporal dementia), and Huntington's disease. Examples of neurological diseases include, but are not limited to, vascular dementias, stroke, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illnesses include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing fect syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; parancoplastic diseases; paroxysmal attacks; Parry Romberg syndrome; Pelizacus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome. In some embodiments, a neurological disorder affects the central nervous system, which includes the brain and the spinal cord. In some embodiments, a neurological disorder affects neurons. In some embodiments, a neurological disorder affects nerves. In some embodiments, a neurological disorder affects the spinal cord. In some embodiments, a neurological disorder is a brain disease (i.e., a disease affecting the brain). In some embodiments, the brain disease is Alzheimer's disease.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g., nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D.C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

In some embodiments, a disease is characterized by cellular dysfunction. For example, a disease may be a mitochondrial disease. Non-limiting mitochondrial diseases include Freidrich's ataxia, alphers disease, barth syndrome, beta-oxidation defects, carnitine deficiency, CPT I deficiency, and mitochondrial DNA depletion. Cellular dysfunction may include mitochondria dysfunction, RNA replication dysfunction, DNA replication dysfunction, translation dysfunction, and/or protein folding dysfunction.

In some embodiments, the disease or condition is caused by a wound, bleeding out, injuries (e.g., broken bones, gunshot wound, cut, scarring during surgery (e.g., cesarean). In some embodiments, the disease or condition is caused by a spinal cord injury. In some embodiments, the disease or condition is caused by a brain injury.

“Cellular causes of aging” as used herein include loss or modification of epigenetic information.

The terms “c-Myc” or “Myc” refer to a nuclear phosphoprotein that has been implicated in cell cycle progression. C-Myc is capable of forming a heterodimer with the transcription factor MAX and the heterodimer is capable of binding to an E box consequence sequence on nucleic acids (e.g., engineered nucleic acids) to regulate transcription of target genes. In certain embodiments, a nucleotide sequence encoding c-Myc comprises a sequence that is at least 70% (e.g., at least 75%, 80%, 85%, 90%, 95%, 98%, 99%, or 100%) identical to a sequence as described in the NCBI RefSeq database under accession number NM_001354870.1 or NM_002467.5. In certain embodiments, an amino acid sequence encoding c-Myc comprises a sequence that is at least 70% (e.g., at least 75%, 80%, 85%, 90%, 95%, 98%, 99%, or 100%) identical to NP_002458.2 or NP_001341799.1. In certain embodiments, the methods comprise inducing expression of OCT4; KLF4; SOX2; or any combination thereof in the absence of inducing c-Myc expression or in the absence of activating c-Myc. Absence of inducing c-Myc expression may refer to absence of substantial induction of c-Myc expression over endogenous levels of c-Myc expression in a cell, tissue, subject, or any combination thereof. Absence of substantial induction of c-Myc expression as compared to endogenous levels of c-Myc expression in a cell, tissue, subject, or any combination thereof, may refer to increasing c-Myc expression by less than 70%, less than 60%, less than 50%, less than 40%, less than 30%, less than 20%, less than 10%, or any values in between as compared to endogenous levels of c-Myc expression in the cell, tissue, subject, or any combination thereof. Absence of activating c-Myc expression may refer to absence of substantial activation of c-Myc (e.g., activity) over endogenous c-Myc activity in a cell, tissue, subject, or any combination thereof. Absence of substantial induction of c-Myc activity as compared to endogenous c-Myc activity in a cell, tissue, subject, or any combination thereof, may refer to increasing c-Myc activity by less than 70%, less than 60%, less than 50%, less than 40%, less than 30%, less than 20%, less than 10%, or any values in between as compared to endogenous c-Myc activity in the cell, tissue, subject, or any combination thereof.

The terms “effective amount” and “therapeutically effective amount,” as used herein, refer to the amount or concentration of an inventive compound, that, when administered to a subject, is effective to at least partially treat a condition from which the subject is suffering.

As used herein, a protein that is “functional” or “active” is one that retains its biological activity (e.g., capable of acting as a transcription factor or as an inducing agent). Conversely, a protein that is not functional or is inactive is one that is not capable of performing one or more of its wild-type functions.

The term “gene” refers to a nucleic acid (e.g., engineered nucleic acid) fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

“Homolog” or “homologous” refers to sequences (e.g., nucleic acid (e.g., engineered nucleic acid) or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity). Homologous sequences include but are not limited to paralogous or orthologous sequences. Paralogous sequences arise from duplication of a gene within a genome of a species, while orthologous sequences diverge after a speciation event. A functional homolog retains one or more biological activities of a wild-type protein. In certain embodiments, a functional homolog of OCT4, KLF4, or SOX2 retains at least 10%, at least 20%, at least 30%, at least 40%, at least 50%, at least 60%, at least 70%, at least 80%, at least 90%, or at least 100% of the biological activity (e.g., transcription factor activity) of a wild-type counterpart.

“KLF4” may also be referred to as Kruppel-like factor 4, EZF, or GKLF and is a zinc-finger transcription factor. KLF4 has been implicated in regulation of differentiation and proliferation and is capable of interacting with co-activators, including members of the p300-CBP coactivator family. A KLF4 transcription factor, homolog (e.g., functional homolog), or variant thereof, as used herein, may be derived from any species, including humans. In certain embodiments, the nucleic acid (e.g., engineered nucleic acid) encoding human KLF4 comprises a sequence that is at least 70% (e.g., at least 75%, 80%, 85%, 90%, 95%, 98%, 99%, or 100%) identical to a nucleic acid (e.g., engineered nucleic acid) described in the NCBI RefSeq database under accession number NM_004235.5, NM_001314052.1. SEQ ID NO: 131, or SEQ ID NO: 145. Non-limiting examples of KLF4 variants include Krueppel-like factor 4 transcript variant 1 and Krueppel-like factor 4 transcript variant 2. In certain embodiments, KLF4 comprises a nucleic acid (e.g., engineered nucleic acid) sequence that is at least 70% (e.g., at least 75%, 80%, 85%, 90%, 95%, 98%, 99%, or 100%) identical to SEQ ID NO: 5, SEQ ID NO: 44, SEQ ID NO: 131, SEQ ID NO: 145. SEQ ID NOs: 5 and 145 are non-limiting examples of a nucleotide sequence encoding KLF4 from Mus musculus. SEQ ID NO: 44 is a non-limiting example of a nucleotide sequence encoding human KLF4. In certain embodiments, KLF4 comprises an amino acid sequence that is at least 70% (e.g., at least 75%, 80%, 85%, 90%, 95%, 98%, 99%, or 100%) identical to NP_001300981.1 or NP_004226.3. In certain embodiments, KLF4 comprises an amino acid sequence that is at least 70% (e.g., at least 75%, 80%, 85%, 90%, 95%, 98%, 99%, or 100%) identical to SEQ ID NO: 6. In certain embodiments, KLF4 comprises an amino acid sequence that is at least 70% (e.g., at least 75%, 80%, 85%, 90%, 95%, 98%, 99%, or 100%) identical to SEQ ID NO: 45. SEQ ID NO: 6 is a non-limiting example of an amino acid sequence encoding KLF4 from Mus musculus. SEQ ID NO: 45 is a non-limiting example of an amino acid sequence encoding human KLF4. Other KLF4 transcription factors (e.g., from other species) are known and nucleic acids (e.g., engineered nucleic acids) encoding KLF4 transcription factors can be found in publically available databases, including GenBank

“Inverted terminal repeats” or “ITRs” are nucleic acid (e.g., engineered nucleic acid) sequences that are reverse complements of one another. In general, in an AAV vector, ITRs are found on either side of a cassette (e.g., an expression cassette comprising a nucleic acid (e.g., engineered nucleic acid) encoding OCT4; KLF4; SOX2; or any combination thereof). In some instances, the cassette encodes an inducing agent. AAV ITRs include ITRs from AAV1, AAV2, AAV3, AAV4, AAV5, AAV6, AAV7, AAV8, AAV9, AAV10, AAV11, AAV.PHP.b (e.g., AAV.PHP.eB), and AAV variants thereof. In some embodiments, an AAV ITR is from an AAV that targets the central nervous system (e.g., AAV.PHP.b (e.g., AAV.PHP.eB. In some embodiments, the AAV serotype is not AAV2 or AAV9. In some embodiments, the AAV serotype corresponds to an AAV capsid that targets the central nervous system.

The terms “nucleic acid,” “polynucleotide”, “nucleotide sequence”, “nucleic acid (e.g., engineered nucleic acid) molecule”, “nucleic acid (e.g., engineered nucleic acid) sequence”, and “oligonucleotide” refer to a series of nucleotide bases (also called “nucleotides”) in DNA and RNA, and mean any chain of two or more nucleotides. The terms “nucleic acid” or “nucleic acid (e.g., engineered nucleic acid) sequence”, “nucleic acid (e.g., engineered nucleic acid) molecule”, “nucleic acid (e.g., engineered nucleic acid) fragment” or “polynucleotide” may be used interchangeably with “gene,” “mRNA encoded by a gene,” and “cDNA”.

The nucleic acids (e.g., engineered nucleic acids) can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids (e.g., engineered nucleic acids)” (PNAs) formed by conjugating bases to an amino acid backbone. This also includes nucleic acids (e.g., engineered nucleic acids) containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

The nucleic acids (e.g., engineered nucleic acids) described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters, such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore, a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The nucleic acids (e.g., engineered nucleic acids) may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids (e.g., engineered nucleic acids) may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, epitope tags, isotopes (e.g., radioactive isotopes), biotin, and the like.

A “recombinant nucleic acid (e.g., engineered nucleic acid) molecule” or “engineered nucleic acid” is a nucleic acid (e.g., engineered nucleic acid) molecule that has undergone a molecular biological manipulation, i.e., non-naturally occurring nucleic acid (e.g., engineered nucleic acid) molecule or genetically engineered nucleic acid (e.g., engineered nucleic acid) molecule. Furthermore, the terms “recombinant DNA molecule” or “engineered nucleic acid” refer to a nucleic acid (e.g., engineered nucleic acid) sequence, which is not naturally occurring, or can be made by the artificial combination of two otherwise separated segments of nucleic acid (e.g., engineered nucleic acid) sequence, i.e., by ligating together pieces of DNA that are not normally continuous. By “recombinantly produced” is meant artificial combination often accomplished by either chemical synthesis means, or by the artificial manipulation of isolated segments of nucleic acids (e.g., engineered nucleic acids), e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid (e.g., engineered nucleic acid) segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design.

“OCT4” may also be referred to as Octamer-binding transcription factor 4, OCT3, OCT3/4, POU5F1, or POU class 5 homeobox 1 and is a transcription factor that has been implicated in embryonic development and determination of cell fate. Similar to other OCT transcription factors, OCT4 is characterized by a bipartite DNA binding domain called a POU domain. An OCT4 transcription factor, homolog, or variant thereof, as used herein, may be derived from any species, including humans. In certain embodiments, the nucleic acid (e.g., engineered nucleic acid) encoding human OCT4 is at least 70% (e.g., at least 75%, 80%, 85%, 90%, 95%, 98%, 99%, or 100%) identical to a nucleic acid (e.g., engineered nucleic acid) described in the NCBI RefSeq under accession number NM_002701, NM_203289, NM_001173531, NM_001285986, or NM_001285987. In certain embodiments, the nucleic acid (e.g., engineered nucleic acid) encoding an OCT4 comprises a sequence that is at least 70% (e.g., at least 75%, 80%, 85%, 90%, 95%, 98%, 99%, or 100%) identical to a nucleic acid (e.g., engineered nucleic acid) sequence provided as SEQ ID NO: 1. SEQ ID NO: 1 is a non-limiting example of a nucleotide sequence encoding OCT4 from Mus musculus. In certain embodiments, the nucleic acid (e.g., engineered nucleic acid) encoding a human OCT4 comprises a sequence that is at least 70% (e.g., at least 75%, 80%, 85%, 90%, 95%, 98%, 99%, or 100%) identical to a nucleic acid (e.g., engineered nucleic acid) sequence provided as SEQ ID NO: 40. SEQ ID NO: 40 is a non-limiting example of a nucleotide sequence encoding human OCT4. Non-limiting examples of OCT4 variants encompassed herein include POU5F1, transcript variant 1, POU5F1, transcript variant 2, POU5F1, transcript variant 3, POU5F1, transcript variant 4, and POU5F1 transcript variant 5. In certain embodiments, the amino acid sequence encoding human OCT4 is at least 70% (e.g., at least 75%, 80%, 85%, 90%, 95%, 98%, 99%, or 100%) identical to a nucleic acid (e.g., engineered nucleic acid) described in the NCBI RefSeq under accession number NP_001167002.1, NP_001272915.1, NP_001272916.1, NP_002692.2, or NP_976034.4. In certain embodiments, an OCT4 comprises an amino acid sequence that is at least 70% (e.g., at least 75%, 80%, 85%, 90%, 95%, 98%, 99%, or 100%) identical to SEQ ID NO: 2. SEQ ID NO: 2 is a non-limiting example of an amino acid sequence encoding OCT4 from Mus musculus. In certain embodiments, an OCT4 comprises an amino acid sequence that is at least 70% (e.g., at least 75%, 80%, 85%, 90%, 95%, 98%, 99%, or 100%) identical to SEQ ID NO: 41. SEQ ID NO: 41 is a non-limiting example of an amino acid sequence encoding human OCT4. Other OCT4 transcription factors (e.g., from other species) are known and nucleic acids (e.g., engineered nucleic acids) encoding OCT4 transcription factors can be found in publically available databases, including GenBank.

The term “promoter” refers to a control region of a nucleic acid (e.g., engineered nucleic acid) sequence at which initiation and rate of transcription of the remainder of a nucleic acid (e.g., engineered nucleic acid) sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid (e.g., engineered nucleic acid) sequence that it regulates. Herein, a promoter is considered to be “operably linked” when it is in a correct functional location and orientation in relation to a nucleic acid (e.g., engineered nucleic acid) sequence it regulates to control (“drive”) transcriptional initiation of that sequence, expression of that sequence, or a combination thereof. A promoter may comprise a sequence that is at least 70% (e.g., at least 75%, 80%, 85%, 90%, 95%, 98%, 99%, or 100%) identical to any promoter sequence disclosed herein.

A promoter may promote ubiquitous expression or tissue-specific expression of an operably linked nucleic acid (e.g., engineered nucleic acid) sequence from any species, including humans. In some embodiments, the promoter is a eukaryotic promoter. Non-limiting examples of eukaryotic promoters include TDH3, PGK1, PKC1, TDH2, PYK1, TPI1, AT1, CMV, EF1 alpha, SV40, PGK1 (human or mouse), Ubc, human beta actin, CAG, TRE, UAS, Ac5, Polyhedrin, CaMKIIα, GAL1, GAL10, TEF1, GDS, ADH1, CaMV35S, Ubi, H1, and U6, as would be known to one of ordinary skill in the art (see, e.g., Addgene website: blog.addgene.org/plasmids-101-the-promoter-region).

Non-limiting examples of ubiquitous promoters include tetracycline-responsive promoters (under the relevant conditions), CMV (e.g., SEQ ID NO: 48 or 136), EF1 alpha, a SV40 promoter, PGK1 (SEQ ID NO: 132), Ubc (SEQ ID NO: 130), CAG, human beta actin gene promoter, a RSV promoter (e.g., SEQ ID NO: 47), an EFS promoter (e.g., SEQ ID NO: 49), and a promoter comprising an upstream activating sequence (UAS). In certain embodiments, the promoter is a mammalian promoter.

Non-limiting examples of tissue-specific promoters include central nervous system-specific (e.g., brain-specific, nerve cell-specific, microglia-specific, or astrocyte-specific), liver-specific, muscle-specific, lung-specific, heart-specific, bone-specific, intestine-specific, skin-specific promoters, brain-specific promoters, and eye-specific promoters. As an example, a muscle-specific promoter is a desmin promoter (e.g., a sequence that is at least 70% (e.g., at least 75%, 80%, 85%, 90%, 95%, 98%, 99%, or 100%) identical to SEQ ID NO: 29). Non-limiting examples of eye-specific promoters include human GRK1 (rhodopsin kinase) promoter (e.g., SEQ ID NO: 50), human CRX (cone rod homeobox transcription factor) promoter (e.g., SEQ ID NO: 51), and human NRL promoter (neural retina leucine zipper transcription factor enhancer upstream of the human TK terminal promoter). In some embodiments, a tissue-specific promoter does not comprise an eye-specific promoter. In some embodiments, a tissue-specific promoter is a promoter that is capable of inducing gene expression in the central nervous system. In some embodiments, a tissue-specific promoter is a promoter that is capable of inducing gene expression in the nerve cells. In some embodiments, a tissue-specific promoter is a promoter that is capable of inducing gene expression in the brain.

Non-limiting examples of central nervous system-specific promoters include: Tal α-tubulin promoter, CaMKIIα, neuron-specific enolase (NSE) promoter, Synapsin I (SYN) promoter, Nestin (NES) promoter, GFAP promoter, F4/80 promoter, and Cx3cr1 promoter. In some embodiments, a neuron-specific promoter is selected from the group consisting of: Tal a-tubulin promoter, CaMKIIα, NSE, SYN, and NES. In some embodiments, an astrocyte-specific promoter is the GFAP promoter. In some embodiments, a microglia-specific promoter is F4/80 or Cx3cr1. In some embodiments, a brain-specific promoter is a Tal a-tubulin promoter, CaMKIIα, neuron-specific enolase (NSE) promoter, Synapsin I (SYN) promoter, Nestin (NES) promoter, GFAP promoter, F4/80 promoter, or Cx3cr1 promoter. In some embodiments, a brain-specific promoter does not comprise a Synapsin I (SYN) promoter sequence.

In some embodiments, a tissue-specific promoter comprises a CaMKIIα promoter. In some embodiments, a CaMKIIα promoter comprises a sequence that is at least 70% (e.g., at least 75%, 80%, 85%, 90%, 95%, 98%, 99%, or 100%) identical to SEQ ID NO: 146, 149, or 154.

In some embodiments, a tissue-specific promoter does not comprise a Synapsin-I promoter, does not comprise a CaMKII-gamma promoter, or a combination thereof. In some embodiments, an expression vector does not comprise a sequence that is at least 70% (e.g., at least 75%, 80%, 85%, 90%, 95%, 98%, 99%, or 100%) identical to SEQ ID NO: 157, which encodes the Synapsin-I promoter.

In some embodiments, a promoter is specific for senescent cells. For example, a promoter may specifically induce expression of an operably linked nucleic acid in a senescent cell and not in non-senescent cells. As a non-limiting example, the p16 promoter may be used to promote expression of a operably linked nucleic acid in senescent cells.

In some embodiments, a promoter of the present disclosure is suitable for use in AAV vectors. See, e.g., U.S. Patent Application Publication No. 2018/0155789, which is herein incorporated by reference in its entirety.

Non-limiting examples of constitutive promoters include CaMKIIα, Synapsin-I, CP1, CMV, EF1 alpha, SV40, PGK1, Ubc, human beta actin, beta tubulin, CAG, Ac5, Rosa26 promoter, COL1A1 promoter, polyhedrin, TEF1, GDS, CaM3 5S, Ubi, H1, U6, red opsin promoter (red promoter), rhodopsin promoter (rho promoter), cone arrestin promoter (car promoter), rhodopsin kinase promoter (rk promoter). An Ubc promoter may comprise a sequence that is at least 70% (e.g., at least 75%, 80%, 85%, 90%, 95%, 98%, 99%, or 100%) identical to SEQ ID NO: 18. In some instances, the constitutive promoter is a Rosa26 promoter. In some instances, the constitutive promoter is a COL1A1 promoter. A red opsin promoter may comprise a sequence that is at least 70% (e.g., at least 75%, 80%, 85%, 90%, 95%, 98%, 99%, or 100%) identical to SEQ ID NO: 101. A rho promoter may comprise a sequence that is at least 70% (e.g., at least 75%, 80%, 85%, 90%, 95%, 98%, 99%, or 100%) identical to SEQ ID NO: 102. A cone arrestin promoter may comprise a sequence that is at least 70% (e.g., at least 75%, 80%, 85%, 90%, 95%, 98%, 99%, or 100%) identical to SEQ ID NO: 103. A rhodopsin kinase promoter may comprise a sequence that is at least 70% (e.g., at least 75%, 80%, 85%, 90%, 95%, 98%, 99%, or 100%) identical to SEQ ID NO: 104. A tissue-specific promoter may be used to drive expression of an engineered nucleic acid, including e.g., a nucleic acid encoding a rtTA, (TA, OCT4, KLF4, SOX2, or any combination thereof. In some embodiments, a tissue-specific promoter is used to drive expression of a rtTA or a rTA. In some embodiments, a tissue-specific promoter is used to drive expression of OCT4, KLF4, and SOX2. In some embodiments, the tissue-specific promoter is not a muscle-specific promoter. In some embodiments, the tissue-specific promoter is not an eye-specific promoter. In some embodiments, a tissue-specific promoter does not comprise a sequence that is at least 70% identical (e.g., at least 75%, 80%, 85%, 90%, 95%, 98%, 99%, or 100% identical) to a sequence selected from SEQ ID NOs: 101-104.

In some embodiments, a promoter that is operably linked to an engineered nucleic acid, including e.g., a nucleic acid encoding a rtTA, tTA, OCT4, KLF4, SOX2, or any combination thereof does not comprise an eye-specific promoter. In some embodiments, a promoter that is operably linked to an engineered nucleic acid, including e.g., a nucleic acid encoding a rtTA, tTA, OCT4, KLF4, SOX2, or any combination thereof does not comprise a Synapsin-I promoter, does not comprise a CaMKII-gamma promoter, or a combination thereof. In some embodiments, a promoter that is operably linked to a sequence encoding a transcription factor (e.g., OCT4, SOX2, KLF4, or any combination thereof) and/or encoding an inducing agent does not comprise a sequence that is at least 70% (e.g., at least 75%, 80%, 85%, 90%, 95%, 98%, 99%, or 100%) identical to SEQ ID NO: 157.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid (e.g., engineered nucleic acid) in such a way as to be active in inducing transcriptional activity from the inducible promoter. In certain embodiments, an inducing agent is a tetracycline-sensitive protein (e.g., (TA or rtTA, TetR family regulators).

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, e.g., SEQ ID NO: 26, or TetRKRAB, e.g., SEQ ID NO: 27), a tetracycline operator sequence (tetO) and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al., Nat. Methods. 2012 Feb. 12; 9 (3): 266-9.

In certain embodiments, an inducible promoter comprises a tetracycline (Tet)-responsive element. For example, an inducible promoter may be a TRE3G promoter (e.g., a TRE3G promoter that comprises a sequence that is at least 70% (e.g., at least 75%, 80%, 85%, 90%, 95%, 98%, 99%, or 100%) identical to SEQ ID NO: 7). As an example, a TRE (e.g., TRE2) promoter may comprise a nucleic acid (e.g., engineered nucleic acid) sequence that is at least 70% (e.g., at least 75%, 80%, 85%, 90%, 95%, 98%, 99%, or 100%) identical to SEQ ID NO: 23. As an example, a TRE (e.g., P tight) promoter may comprise a nucleic acid (e.g., engineered nucleic acid) sequence that is at least 70% (e.g., at least 75%, 80%, 85%, 90%, 95%, 98%, 99%, or 100%) identical to SEQ ID NO: 24.

Additional non-limiting examples of inducible promoters include mifepristone-responsive promoters (e.g., GAL4-E1b promoter) and coumermycin-responsive promoters. See, e.g., Zhao et al., Hum Gene Ther. 2003 Nov. 20; 14 (17): 1619-29.

A “reverse tetracycline transactivator” (“rtTA”), as used herein, is an inducing agent that binds to a TRE promoter (e.g., a TRE3G, a TRE2 promoter, or a P tight promoter) in the presence of tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al., Science. 1995 Jun. 23; 268 (5218): 1766-9 and any of the transactivation domains listed herein). The mutant TetR domain is capable of binding to a TRE promoter when bound to tetracycline. See, e.g., International Publication Number WO 2020/069339, entitled Mutant Reverse Tetracycline Transactivators for Expression of Genes, which published on Apr. 2, 2020, which is herein incorporated by reference in its entirety

“SRY-box 2” or “SOX2” is a member of the SRY-related HMG-box (SOX) family of transcription factors. SOX2 has been implicated in promoting embryonic development. Members of the SOX (SRY-related HMG-box) family of transcription factors are characterized by a high mobility group 5 (HMG)-box DNA sequence. This HMG box is a DNA binding domain that is highly conserved throughout eukaryotic species. A SOX2 transcription factor, homolog or variant thereof, as used herein, may be derived from any species, including humans. In certain embodiments, the nucleic acid (e.g., engineered nucleic acid) encoding SOX2 comprises a sequence that is at least 70% (e.g., at least 75%, 80%, 85%, 90%, 95%, 98%, 99%, or 100%) identical to a nucleic acid (e.g., engineered nucleic acid) described in the NCBI RefSeq under accession number NM_011443.4. In certain embodiments, the nucleic acid (e.g., engineered nucleic acid) encoding a human SOX2 comprises a sequence that is at least 70% (e.g., at least 75%, 80%, 85%, 90%, 95%, 98%, 99%, or 100%) identical to a nucleic acid (e.g., engineered nucleic acid) described in the NCBI RefSeq under accession number NM_003106.4. In certain embodiments, SOX2 comprises a nucleic acid (e.g., engineered nucleic acid) sequence that is at least 70% (e.g., at least 75%, 80%, 85%, 90%, 95%, 98%, 99%, or 100%) identical to SEQ ID NO: 3 or SEQ ID NO: 42. SEQ ID NO: 3 is a non-limiting example of a nucleotide sequence encoding SOX2 from Mus musculus. SEQ ID NO: 42 is a non-limiting example of a nucleotide sequence encoding human SOX2. In certain embodiments, the nucleic acid (e.g., engineered nucleic acid) encoding human SOX2 comprises a sequence that is at least 70% (e.g., at least 75%, 80%, 85%, 90%, 95%, 98%, 99%, or 100%) identical to the amino acid sequence described in the NCBI RefSeq under accession number NP_003097.1. In some instances, SOX2 comprises an amino acid sequence that is at least 70% (e.g., at least 75%, 80%, 85%, 90%, 95%, 98%, 99%, or 100%) identical to SEQ ID NO: 4. In some instances, SOX2 comprises an amino acid sequence that is at least 70% (e.g., at least 75%, 80%, 85%, 90%, 95%, 98%, 99%, or 100%) identical to SEQ ID NO: 43. SEQ ID NO: 4 is a non-limiting example of an amino acid sequence encoding SOX2 from Mus musculus. SEQ ID NO: 43 is a non-limiting example of an amino acid sequence encoding human SOX2. Other SOX2 transcription factors (e.g., from other species) are known and nucleic acids (e.g., engineered nucleic acids) encoding SOX2 transcription factors can be found in publically available databases, including GenBank

A “multicistronic vector” is a vector that encodes more than one amino acid sequence (e.g., a vector encoding OCT4 and KLF4, OCT4 and SOX2, KLF4 and SOX2, or OCT4, SOX2, and KL4 (OSK)). A multicistronic vector allows for expression of multiple amino acid sequences from a nucleic acid (e.g., engineered nucleic acid) sequence. Nucleic acid (e.g., engineered nucleic acid) sequences encoding each transcription factor (e.g., OCT4, KLF4, or SOX2) may be connected or separated such that they produce unconnected proteins. For example, internal ribosome entry sites (IRES) or polypeptide cleavage signals may be placed between nucleic acid (e.g., engineered nucleic acid) sequences encoding each transcription factor in a vector. Exemplary polypeptide cleavage signals include 2A peptides (e.g., T2A, P2A, E2A, and F2A). A 2A peptide may comprise a sequence that is at least 70% (e.g., at least 75%, 80%, 85%, 90%, 95%, 98%, 99%, or 100%) identical to SEQ ID NO: 9 or 118. In some embodiments, an expression vector of the present disclosure is a multicistronic expression vector.

“Reversing aging” or “reversing ageing” as used herein refers to modifying the physical characteristics associated with aging. All animals typically go through a period of growth and maturation followed by a period of progressive and irreversible physiological decline ending in death. The length of time from birth to death is known as the life span of an organism, and each organism has a characteristic average life span. Aging is a physical manifestation of the changes underlying the passage of time as measured by percent of average life span.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). In certain embodiments, the animal is a mammal. The animal may be a male or female and at any stage of development. A non-human animal may be a transgenic animal.

One of ordinary skill in the art would recognize that the biological age of a pediatric subject or adult subject may vary depending on the type of animal. As a non-limiting example, an adult mouse may be 1 year of age, while an adult human may be more than 21 years of age. In some embodiments, a pediatric subject is less than 21 years of age, less than 20 years of age, less than 15 years of age, less than 10 years of age, less than 9 years of age, less than 8 years of age, less than 7 years of age, less than 6 years of age, less than 5 years of age, less than 4 years of age, less than 3 years of age, less than 2 years of age, less than 1 year of age, less than 10 months of age, less than 9 months of age, less than 8 months of age, less than 7 months of age, less than 6 months of age, less than 5 months of age, less than 4 months of age, less than 2 months of age, or less than 1 month of age. In some embodiments, an adult subject is at least 3 weeks of age, 1 month of age, at least 2 months of age, at least 3 months of age, at least 4 months of age, at least 5 months of age, at least 6 months of age, at least 7 months of age, at least 8 months of age, at least 9 months of age, at least 10 months of age, at least 11 months of age, at least 1 year of age, at least 2 years of age, at least 3 years of age, at least 5 years of age, at least 10 years of age, at least 15 years of age, at least 20 years of age, at least 25 years of age, at least 30 years of age, at least 40 years of age, at least 50 years of age, at least 55 years of age, at least 60 years of age, at least 65 years of age, at least 70 years of age, at least 75 years of age, at least 80 years of age, at least 90 years of age, or at least 100 years of age. In some embodiments, a middle-aged adult subject is between 1 and 6 months of age, between 6 and 12 months of age, between 1 year and 5 years of age, between 5 years and 10 years of age, between 10 and 20 years of age, between 20 and 30 years of age, between 30 and 50 years of age, between 50 and 60 years of age, between 40 and 60 years of age, between 40 and 50 years of age, or between 45 and 65 years of age. In some embodiments, a senior adult subject is at least 1 month of age, at least 2 months of age, at least 3 months of age, at least 4 months of age, at least 5 months of age, at least 6 months of age, at least 7 months of age, at least 8 months of age, at least 9 months of age, at least 10 months of age, at least 11 months of age, at least 1 year of age, at least 2 years of age, at least 3 years of age, at least 5 years of age, at least 10 years of age, at least 15 years of age, at least 20 years of age, at least 25 years of age, at least 30 years of age, at least 40 years of age, at least 50 years of age, at least 55 years of age, at least 60 years of age, at least 65 years of age, at least 70 years of age, at least 75 years of age, at least 80 years of age, at least 90 years of age, or at least 100 years of age.

A “terminator” or “terminator sequence.” as used herein, is a nucleic acid (e.g., engineered nucleic acid) sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid (e.g., engineered nucleic acid) sequences by upstream promoters. Thus, in certain embodiments, a terminator that ends the production of an RNA transcript is contemplated.

The most commonly used type of terminator is a forward terminator. When placed downstream of a nucleic acid (e.g., engineered nucleic acid) sequence that is usually transcribed, a forward transcriptional terminator will cause transcription to abort. In some embodiments, bidirectional transcriptional terminators may be used, which usually cause transcription to terminate on both the forward and reverse strand. In some embodiments, reverse transcriptional terminators may be used, which usually terminate transcription on the reverse strand only.

Non-limiting examples of mammalian terminator sequences include bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA, and arcA terminator. In certain embodiments, the terminator sequence is SV40 and comprises a sequence that is at least 70% (e.g., at least 75%, 80%, 85%, 90%, 95%, 98%, 99%, or 100%) identical to SEQ ID NO: 8 or 143. In certain embodiments, the terminator sequence is hGH pA and comprises a sequence that is at least 70% (e.g., at least 75%, 80%, 85%, 90%, 95%, 98%, 99%, or 100%) identical to SEQ ID NO: 139, 148, 153, 156, or 161

A “Tet-Off” system, as used herein, is a type of inducible system that is capable of repressing expression of a particular transgene in the presence of tetracycline (e.g., doxycycline (DOX)). Conversely, a Tet-Off system is capable of inducing expression of a particular transgene in the absence of tetracycline (e.g., doxycycline or DOX). In certain embodiments, a Tet-Off system comprises a tetracycline-responsive promoter operably linked to a transgene (e.g., encoding OCT4; KLF4; SOX2; or any combination thereof) and a tetracycline-controlled transactivator (tTA). In some embodiments, an inducing agent is a tTA. The transgene with the tetracycline-responsive promoter (e.g., TRE3G, P tight, or TRE2) and the tetracycline-controlled transactivator may be encoded on the same vector or be encoded on separate vectors. See, e.g., International Publication Number WO 2020/069339, entitled “Mutant Reverse Tetracycline Transactivators for Expression of Genes,” which was published on Apr. 2, 2020, and which is herein incorporated by reference in its entirety. In certain embodiments, an inducing agent is tetracycline-controlled transactivator (tTA) (e.g., any tTA disclosed herein). In certain embodiments, the inducing agent is a tTA and comprises a sequence that is at least 70% (e.g., at least 75%, 80%, 85%, 90%, 95%, 98%, 99%, or 100%) identical to any tTA disclosed herein. In certain embodiments, the inducing agent is a tTA and comprises a sequence that is at least 70% (e.g., at least 75%, 80%, 85%, 90%, 95%, 98%, 99%, or 100%) identical to SEQ ID NO: 138 or 159. In certain embodiments, the inducing agent is a tTA and is encoded by a sequence that comprises a sequence that is at least 70% (e.g., at least 75%, 80%, 85%, 90%, 95%, 98%, 99%, or 100%) identical to SEQ ID NO: 137 or 158. Without being bound by a particular theory, tetracycline (e.g., doxycycline) may bind to the tTA and prevent tTA from binding its cognate promoter (e.g., a promoter comprising a tetracycline response element (TRE)) and driving expression of an operably linked nucleic acid. Without being bound by a particular theory, a nucleic acid (e.g., engineered nucleic acid) encoding an inducing agent may not be on the same vector as any of the nucleic acids (e.g., engineered nucleic acids) encoding OCT4, KLF4, and SOX2 to reduce the size of a viral vector and improve viral titer.

A “Tet-On” system, as used herein, is a type of inducible system that is capable of inducing expression of a particular transgene in the presence of tetracycline (e.g., doxycycline (DOX)). In certain embodiments, a Tet-On system comprises a tetracycline-responsive promoter operably linked to a transgene (e.g., encoding OCT4; KLF4; SOX2; or any combination thereof) and a reverse tetracycline-controlled transactivator (rtTA). For example, the rtTA may be rtTA3, rtTA4, rtTA Advanced, rtTA2S-M2, or variants thereof. In certain embodiments, a nucleic acid (e.g., engineered nucleic acid) encoding rtTA3 comprises a sequence that is at least 70% (e.g., at least 75%, at least 80%, at least 85%, at least 90%, at least 95%, or at least 100%) identical to SEQ ID NO: 10. In certain embodiments, an amino acid sequence encoding rtTA3 comprises a sequence that is at least 70% (e.g., at least 75%, at least 80%, at least 85%, at least 90%, at least 95%, at least 98%, at least 99%, or at least 100%) identical to (SEQ ID NO: 11). In certain embodiments, a nucleic acid (e.g., engineered nucleic acid) encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 12. In certain embodiments, an amino acid sequence encoding rtTA4 comprises a sequence that is at least 70% (e.g., at least 75%, at least 80%, at least 85%, at least 90%, at least 95%, at least 98%, at least 99%, or at least 100%) identical to (SEQ ID NO: 13). In certain embodiments, a nucleic acid (e.g., engineered nucleic acid) encoding rtTA Advanced comprises a sequence that is at least 70% (e.g., at least 75%, at least 80%, at least 85%, at least 90%, at least 95%, at least 98%, at least 99%, or at least 100%) identical to SEQ ID NO: 128. In certain embodiments, an amino acid sequence encoding rtTA Advanced comprises a sequence that is at least 70% (e.g., at least 75%, at least 80%, at least 85%, at least 90%, at least 95%, at least 98%, at least 99%, or at least 100%) identical to (SEQ ID NO: 129). In certain embodiments, a nucleic acid (e.g., engineered nucleic acid) encoding rtTA2S-M2 comprises a sequence that is at least 70% (e.g., at least 75%, at least 80%, at least 85%, at least 90%, at least 95%, at least 98%, at least 99%, or at least 100%) identical to SEQ ID NO: 14. In certain embodiments, an amino acid sequence encoding rtTA2S-M2 comprises a sequence that is at least 70% (e.g., at least 75%, at least 80%, at least 85%, at least 90%, at least 95%, at least 98%, at least 99%, or at least 100%) identical to (SEQ ID NO: 15). The expression cassette encoding a tetracycline-responsive promoter (e.g., a promoter comprising a TRE, including TRE3G, P tight, and TRE2) and a reverse tetracycline-controlled transactivator may be encoded on the same vector or be encoded on separate vectors. See, e.g., International Publication Number WO 2020/069339, entitled “Mutant Reverse Tetracycline Transactivators for Expression of Genes,” which was published on Apr. 2, 2020, and which is herein incorporated by reference in its entirety.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal, damaged, or unhealthy tissue, which may need to be treated. A tissue may also be a normal or healthy tissue that is under a higher than normal risk of becoming abnormal or unhealthy, which may need to be prevented. In certain embodiments, the tissue is considered healthy but suboptimal for performance or survival in current or future conditions. For example, in agricultural practice, environmental conditions including weather and growing conditions (e.g., nutrition) may benefit from any of the methods described herein. In certain embodiments, the tissue is the central nervous system, e.g., the brain. In some embodiments, the cell or tissue is a neuronal cell or nervous tissue, In some embodiments, the cell is a neuron. In some embodiments, the neuron is an excitatory neuron. In some embodiments, the tissue is not eye tissue.

The term “tetracycline repressor” or “TetR” refers to a protein that is capable of binding to a Tet-O sequence (e.g., a Tet-O sequence in a TRE, e.g., a Tet-O sequence may comprise SEQ ID NO: 19) in the absence of tetracycline (e.g., doxycycline) and prevents binding of rtTA (e.g., rtTA3, rtTA4, or variants thereof) in the absence of tetracycline (e.g., doxycycline). TetRs prevent gene expression from promoters comprising a TRE in the absence of tetracycline (e.g., doxycycline). In the presence of tetracycline, TetRs cannot bind promoters comprising a TRE, and TetR cannot prevent transcription. Non-limiting examples of TetRs include tetR (e.g., SEQ ID NO: 26), tetRKRAB (e.g., SEQ ID NO: 28). In some embodiments, a TetR is a TetR fusion (e.g., TRSID, which may be created by fusing TetR to a mSIN30interacting domain (SID) of Mad1). See, e.g., Zhang et al., J Biol Chem. 2001 Nov. 30; 276 (48): 45168-74.

As used herein, a “TRE promoter” is a promoter comprising a tetracycline-responsive element (TRE). As used herein, a TRE comprises at least one (e.g., at least 2, 3, 4, 5, 6, 7, 8, 9, 10, 11, 12, 13, 14, 15, 16, 17, 18, 19, or 20) Tet-O sequences. A non-limiting example of a Tet-O sequence is sequence that is at least 70% (e.g., 75%, 80%, 85%, 90%, 95%, 98%, 99%, or 100%) identical to SEQ ID NO: 19. In some embodiments, a TRE promoter further comprises a minimal promoter located downstream of a tet-O sequence. A minimal promoter is a promoter that comprises the minimal elements of a promoter (e.g., TATA box and transcription initiation site), but is inactive in the absence of an upstream enhancer (e.g., sequences comprising Tet-O). As an example, a minimal promoter may be a minimal CMV promoter that comprises a sequence that is at least 70% (e.g., at least 75%, 80%, 85%, 90%, 95%, 98%, 99%, or 100%) identical to SEQ ID NO: 20. For example, a TRE promoter may be a TRE3G promoter (e.g., a TRE3G promoter that comprises a sequence that is at least 70% (e.g., at least 75%, 80%, 85%, 90%, 95%, 98%, 99%, or 100%) identical to SEQ ID NO: 7. In some embodiments, a TRE promoter is a TRE2 promoter comprising a sequence that is at least 70% (e.g., at least 75%, 80%, 85%, 90%, 95%, 98%, 99%, or 100%) identical to SEQ ID NO: 23. In some embodiments, a TRE promoter is a P tight promoter comprising a sequence that is at least 70% (e.g., at least 75%, 80%, 85%, 90%, 95%, 98%, 99%, or 100%) identical to SEQ ID NO: 24.

The term “tissue repair” in the context of damaged tissue refers to restoration of tissue architecture, function following tissue damage, or a combination thereof. Tissue repair includes tissue regeneration, cell growth, tissue replacement, and/or rewiring of existing tissue (reprogramming).

The term “tissue regeneration” refers to production of new tissue or cells within a tissue that are the same type as the tissue of interest (e.g., same type as the damaged tissue or cell). In some embodiments, the methods provided herein promote organ regeneration.

The term “tissue replacement” refers to production of a different type of tissue compared to the tissue of interest (e.g., connective tissue to replace damaged tissue).

As used herein, the terms “treatment,” “treat,” and “treating” refer to reversing, alleviating, delaying the onset of, or inhibiting the progress of a disease or disorder, or one or more symptoms thereof, as described herein. In certain embodiments, treatment may be administered after one or more symptoms have developed. In other embodiments, treatment may be administered in the absence of symptoms. For example, treatment may be administered to a susceptible individual prior to the onset of symptoms or may be treated with another damaging agent (e.g., in light of a history of symptoms, in light of genetic or other susceptibility factors, a disease therapy, or any combination thereof). Treatment may also be continued after symptoms have resolved, for example, to prevent or delay their recurrence.

The term “variant” refers to a sequence that comprises a modification relative to a wild-type sequence. Non-limiting modifications in an amino acid sequence include insertions, deletions, and point mutations. Non-limiting modifications to nucleic acid (e.g., engineered nucleic acid) sequences include frameshift mutations, nucleotide insertions, and nucleotide deletions.

The term “WPRE” refers to a Woodchuck Hepatitis Virus (WHP) Posttranscriptional Regulatory Element (WPRE). WPREs create tertiary structures in nucleic acids (e.g., expression vectors) and are capable of enhancing transgene expression (e.g., from a viral vector). In certain embodiments, a WPRE sequence is at least 70% (e.g., at least 75%, at least 80%, at least 85%, at least 90%, at least 95%, at least 98%, at least 99%, or at least 100%) identical to SEQ ID NO: 21, 135, 147, 152, 155, or 160.

These and other exemplary substituents are described in more detail in the Detailed Description, Examples, and claims. The invention is not intended to be limited in any manner by the above exemplary listing of substituents.

### BRIEF DESCRIPTION OF THE DRAWINGS

FIGS. 1A-1F show OCT4, SOX2, and KLF4 (OSK) reprogramming increases electric firing activity in iPSC-derived neurons using a tet-inducible all-in-one OSK lentiviral vector. FIG. 1A is a schematic diagram of the all-in-one OSK lentiviral vector. FIG. 1B is a schematic diagram of the I-PpoI lentiviral vector. FIG. 1C shows an experimental outline of reprogramming iPSC-derived neurons. FIG. 1D shows a western blot showing expression of OCT4, SOX2, and KLF4 after doxycycline treatment. FIG. 1E shows electrophysiology measurement using the multi-electrode array. FIG. 1F shows quantification of electric firing spikes in control and reprogrammed neurons. TAM, 4-Hydroxytamoxifen. Dox, doxycycline.

FIGS. 2A-2F show OSK reprogramming improves cognitive performance in middle- and old-aged mice. FIG. 2A shows an experimental outline of OSK delivery to the brain and behavioral tests. FIGS. 2B-2C show results of novel object recognition assay in middle-aged experimental mice at the age of 11 months (FIG. 2B). FIGS. 2C-2D show results of Morris water maze at the age of 12 months (FIG. 2C) and 30 months (FIG. 2D). FIGS. 2E-2F show results of expressing OSK for two months. FIG. 2E shows two months of OSK treatment failed to improve improve cognitive performance in 12-month-old mice. FIG. 2F shows two months of OSK treatment failed to improve improve cognitive performance in 30-month-old mice.

FIGS. 3A-3C show CaMKIIα-tTA and CaMKIIα-rtTA AAV vectors to restrict gene expression in excitatory neurons. FIG. 3A is a schematic of the CaMKIIα-tTA construct. FIG. 3B is a schematic of the CaMKIIα-rtTA construct. FIG. 3C shows body weight of the mice injected with AAVs.

FIGS. 4A-4D show reprogramming using OCT4, KLF4, and SOX2 (OKS) improves cognitive performance in Alzheimer's mice. FIG. 4A shows a design of the inducible neuron-specific OKS (iNOKS) transgenic mice. FIG. 4B shows a schematic showing the experimental design for OKS induction in 5×FAD mice and behavioral assays for cognitive performance. FIG. 4C shows immunofluorescence showing the expression of OCT4, KLF4, and SOX2 in the hippocampus of iNOKS mice after 4 weeks of doxycycline treatment. FIG. 4D shows water T maze results indicating that the reprogrammed 5×FAD mice (FAD-iNOKS) tend to require fewer days to reach the preset learning criterion than the FAD control mice.

FIG. 5 shows RNA-seq analysis of hippocampus samples. Gene Set Enrichment Analysis (GSEA) indicated that the genes involved in learning and memory functions are upregulated in hippocampal samples one month after doxycycline withdrawal (Post OSK), but not immediately after 4 weeks of doxycycline treatment (OSK). Most of these gene sets are downregulated during aging or induction of p25 for 2 weeks or 6 weeks. The p25 induction reference data is from Gjoneska et al. Nature. 2015 Feb. 19; 518 (7539): 365-9. The gene sets for GSEA analysis in FIG. 5 is from the Molecular Signatures Database (MSigDB).

FIGS. 6A-6C show single nucleus multiomics indicating that development-related genomic loci are the hotspots of hypo- and hyper-methylation induced by OSK reprogramming. FIG. 6A shows hierarchical clustering based on single nucleus transcriptomic data. FIG. 6B shows a heatmap showing the expression of OSK in each cluster. FIG. 6C shows enrichment of hypo- and hyper-methylation induced by OSK reprogramming at development-related genomic loci, which are underlined

### DETAILED DESCRIPTION OF CERTAIN EMBODIMENTS OF THE INVENTION

The present disclosure is based, at least in part, on the unexpected results demonstrating that expression of OCT4, SOX2, and KLF4 in the absence of exogenous c-Myc expression can be used to promote partial reprogramming and tissue regeneration in vivo. Surprisingly, using nerve cells as a model tissue, as described herein, in some embodiments, it was determined that the combination of OCT4, SOX2, and KLF4 (OSK) could be used to reset the youthful gene expression patterns and epigenetic age of cells of the central nervous system to improve the cognitive function of animals.

The methods, compositions, uses, and kits of the present disclosure are in part informed by the surprising and unexpected discovery that the spatially and temporally specific induction of OCT4, SOX2, and KLF4 expression in the absence of the induction of c-Myc expression can rejuvenate a cell in the central nervous system that is not in the retina (e.g., brain, spinal cord) without reprogramming the cell to a pluripotent state.

Aspects of the present disclosure provide several systems, compositions, uses, kits, and methods that may be useful for efficient rejuvenation of a cell, tissue, and/or organ in the central nervous system. In some embodiments, the central nervous system does not include the eye (e.g., retina, uvea, pupil, lens, cornea, and/or sclera). In some embodiments, the cell, tissue, and/or organ in the central nervous system is a brain cell, brain tissue, and/or the brain. For example, aspects of the present disclosure provide nucleic acids that may be useful for efficient rejuvenation of the central nervous system by promoting expression of OCT4, SOX2, and/or KLF4 without inducing c-Myc expression. In some embodiments, such a nucleic acid encodes an inducing agent (e.g., tetracycline transactivor or reverse tetracycline transactivator) operably linked to a CaMKIIα promoter. In some embodiments, the CaMKIIα promoter is at least 70% (e.g., at least 75%, 80%, 85%, 90%, 95%, 98%, 99%, or 100%) identical to SEQ ID NO: 149. In some embodiments, the nucleic acid encoding an inducing agent does not comprise a Synapsin-I promoter or a CaMKII-gamma promoter. In some embodiments, the Synapsin-I promoter is at least 70% (e.g., at least 75%, 80%, 85%, 90%, 95%, 98%, 99%, or 100%) identical to SEQ ID NO: 157. In some embodiments, a nucleic acid encoding an inducing agent is a viral vector. In some embodiments, the viral vector is packaged in AAV.PHP.eB virus. In some embodiments, a nucleic acid encoding an inducing agent comprises a sequence that is at least 70% (e.g., at least 75%, 80%, 85%, 90%, 95%, 98%, 99%, or 100%) identical to rtTA2S-M2 (SEQ ID NO: 14), pLVX-rtTA-hOSK-all-in-one(human) (SEQ ID NO: 123), pAAV-CaMKIIα-tTA2 (SEQ ID NO: 124), pAAV-CaMKIIα-rtTA2S-M2 (SEQ ID NO: 125), pAAV-CaMKIIα-rtTA3 (SEQ ID NO: 126), CaMKIIα promoter (SEQ ID NO: 146, 149, or 154); rtTA Advanced in reverse complement (SEQ ID NO: 128), ITA Advanced (SEQ ID NO: 137), and/or tTA (SEQ ID NO: 158). In some embodiments, a nucleic acid encoding an inducing agent does not comprise a sequence that is at least 70% (e.g., at least 75%, 80%, 85%, 90%, 95%, 98%, 99%, or 100%) identical to pAAV-ihSyn1-tTA (SEQ ID NO: 127). In some embodiments, an inducing agent comprises a sequence that is at least 70% (e.g., at least 75%, 80%, 85%, 90%, 95%, 98%, 99%, or 100%) identical to rtTA Advanced (SEQ ID NO: 129), tTA Advanced (SEQ ID NO: 138), rtTA2S-M2 (SEQ ID NO: 15), and/or (TA (SEQ ID NO: 159).

Aspects of the present disclosure provide nucleic acids, which may be useful in inducing expression of OCT4, KLF4, and/or SOX2 in the absence of inducing c-Myc expression in a cell, tissue, or organ of the central nervous system. These nucleic acids may be useful in rejuvenating the cell, tissue, or organ of the central nervous system. In some embodiments, the nucleic acid is a nucleic acid with (a) a nucleic acid sequence that encodes OCT4, KLF4, and SOX2 operably linked to a TRE promoter; and (b) a nucleic acid sequence that encodes rtTA operably linked to a UbC promoter. In some embodiments, the nucleic acid sequence that encodes OCT4, KLF4, and SOX2 further encodes a 2A peptide. In some embodiments, the nucleic acid with (a) and (b) further encodes a neomycin resistance gene and/or comprises a WPRE sequence. In some embodiments, the neomycin resistance gene is operably linked to a PGK promoter. In some embodiments, the nucleic acid with (a) and (b) encodes at least one protein sequence that is at least 70% identical (e.g., at least 75%, 80%, 85%, 90%, 95%, 98%, 99%, or 100% identical) to a sequence selected from: rtTA Advanced (SEQ ID NO: 129); human OCT4 (SEQ ID NO: 41); P2A (SEQ ID NO: 118); human SOX2 (SEQ ID NO: 43); T2A (SEQ ID NO: 9); human KLF4 (SEQ ID NO: 45); and neomycin resistance gene (SEQ ID NO: 134). In some embodiments, the nucleic acid with (a) and (b) comprises at least one sequence that is at least 70% identical (e.g., at least 75%, 80%, 85%, 90%, 95%, 98%, 99%, or 100% identical) to a sequence selected from: rtTA Advanced in reverse complement (SEQ ID NO: 128); UbC promoter in reverse complement (SEQ ID NO: 130); P tight TRE promoter (SEQ ID NO: 24); human OCT4 (SEQ ID NO: 40); P2A (SEQ ID NO: 119); human SOX2 (SEQ ID NO: 42); T2A (SEQ ID NO: 120); human KLF4 (SEQ ID NO: 131); PGK promoter (SEQ ID NO: 132); Neomycin resistance gene (SEQ ID NO: 133); and WPRE (SEQ ID NO: 135). In some embodiments, the nucleic acid with (a) and (b) comprises a sequence that is at least 70% identical (e.g., at least 75%, 80%, 85%, 90%, 95%, 98%, 99%, or 100% identical) to pLVX-rtTA-hOSK-all-in-one (human) (SEQ ID NO: 123).

In some embodiments, a nucleic acid encodes a tTA, wherein the tTA comprises a sequence that is at least 70% identical (e.g., at least 75%, 80%, 85%, 90%, 95%, 98%, 99%, or 100% identical) to SEQ ID NO: 138. In some embodiments, the nucleic acid encoding the (TA is at least 70% identical (e.g., at least 75%, 80%, 85%, 90%, 95%, 98%, 99%, or 100% identical) to SEQ ID NO: 137. In some embodiments, the nucleic acid encoding the tTA comprises a hGH pA sequence and/or a CMV promoter. In some embodiments, the hGH pA sequence is at least 70% identical (e.g., at least 75%, 80%, 85%, 90%, 95%, 98%, 99%, or 100% identical) to SEQ ID NO: 139. In some embodiments, the CMV promoter is at least 70% identical (e.g., at least 75%, 80%, 85%, 90%, 95%, 98%, 99%, or 100% identical) to SEQ ID NO: 136. In some embodiments, the nucleic acid encoding a tTA comprises a sequence that is at least 70% identical (e.g., at least 75%, 80%, 85%, 90%, 95%, 98%, 99%, or 100% identical) to pAAV-CMV-ITA (Advanced) (SEQ ID NO: 32).

In some embodiments, the nucleic comprises a nucleic acid encoding OCT4, SOX2, and KLF4 operably linked to a TRE promoter. In some embodiments, the nucleic acid sequence encoding OCT4, SOX2, and KLF4 operably linked to a TRE promoter further encodes a 2A peptide and/or SV40 pA. In some embodiments, the 2A peptide comprises a sequence that is at least 70% identical (e.g., at least 75%, 80%, 85%, 90%, 95%, 98%, 99%, or 100% identical) to T2A (SEQ ID NO: 9) or P2A (SEQ ID NO: 118). In some embodiments, the nucleic acid sequence encoding OCT4, SOX2, and KLF4 encodes at least one sequence that is 70% identical to a sequence selected from: mouse OCT4 (SEQ ID NO: 2); human OCT4 (SEQ ID NO: 40); mouse SOX2 (SEQ ID NO: 4); human SOX2 (SEQ ID NO: 42); human KLF4 (SEQ ID NO: 131); and mouse KLF4 (SEQ ID NO: 6). In some embodiments, the TRE promoter operably linked to the nucleic acid sequence encoding OCT4, SOX2, and KLF4 is at least 70% identical (e.g., at least 75%, 80%, 85%, 90%, 95%, 98%, 99%, or 100% identical) to SEQ ID NO: 7. In some embodiments, the nucleic acid sequence encoding OCT4, SOX2, and KLF4 operably linked to the TRE promoter comprises at least one sequence that is 70% identical to a sequence selected from: TRE3G (SEQ ID NO: 7): mouse Oct4 (SEQ ID NO: 1): P2A (SEQ ID NO: 144); mouse Klf4 (SEQ ID NO: 145); SV40 pA (SEQ ID NO: 143); mouse Sox2 (SEQ ID NO: 3); and T2A (SEQ ID NO: 120), optionally wherein the sequence is at least 70% identical (e.g., at least 75%, 80%, 85%, 90%, 95%, 98%, 99%, or 100% identical) to pAAV-TRE3G-OSK (mouse) SEQ ID NO: 16.

In some embodiments, the nucleic acid comprises a nucleic acid that encodes an inducing agent and the nucleic acid encoding the inducing agent is operably linked to a CaMKIIα promoter. In some embodiments, the inducing agent is a tTA or rtTA. In some embodiments, the inducing agent comprises a sequence that is at least 70% identical (e.g., at least 75%, 80%, 85%, 90%, 95%, 98%, 99%, or 100% identical) to tTA Advanced (SEQ ID NO: 138), rtTA2S-M2 (SEQ ID NO: 15), or rtTA3 (SEQ ID NO: 11). In some embodiments, the CaMKIIα promoter comprises a sequence that is at least 70% identical (e.g., at least 75%, 80%, 85%, 90%, 95%, 98%, 99%, or 100% identical) to SEQ ID NO: 146, SEQ ID NO: 149, or SEQ ID NO: 154. In some embodiments, the nucleic acid encoding the inducing agent further comprises a WPRE and/or hGH pA sequence. In some embodiments, the WPRE sequence is at least 70% identical (e.g., at least 75%, 80%, 85%, 90%, 95%, 98%, 99%, or 100% identical) to SEQ ID NO: 147, 152, or 155. In some embodiments, the hGH pA sequence is at least 70% identical (e.g., at least 75%, 80%, 85%, 90%, 95%, 98%, 99%, or 100% identical) to SEQ ID NO: 148, 153, or 156.

In some embodiments, the nucleic acid encoding the inducing agent comprises a sequence that is at least 70% identical (e.g., at least 75%, 80%, 85%, 90%, 95%, 98%, 99%, or 100% identical) to a sequence selected from tTA-Advanced (SEQ ID NO: 137), rtTA2S-M2 (SEQ ID NO: 14), or rtTA3 (SEQ ID NO: 10). In some embodiments, the nucleic acid encoding the inducing agent comprises a sequence that is at least 70% identical (e.g., at least 75%, 80%, 85%, 90%, 95%, 98%, 99%, or 100% identical) to a sequence selected from pAAV-CaMKIIα-tTA2 (SEQ ID NO: 124), pAAV-CaMKIIα-rtTA2S-M2 (SEQ ID NO: 125), or pAAV-CaMKIIα-rtTA3 (SEQ ID NO: 126).

In some embodiments, the nucleic acid does not comprise a Synapsin-I promoter operably linked to a nucleic acid sequence encoding an inducing agent. In some embodiments, the nucleic acid does not comprise a sequence that is at least 70% identical (e.g., at least 75%, 80%, 85%, 90%, 95%, 98%, 99%, or 100% identical) to SEQ ID NO: 157. In some embodiments, the nucleic acid does not comprise a sequence that is at least 70% identical (e.g., at least 75%, 80%, 85%, 90%, 95%, 98%, 99%, or 100% identical) to pAAV-ihSyn1-tTA (SEQ ID NO: 127).

In certain embodiments, the expression of one of more of the genes is transient (e.g., using an inducible promoter to regulate gene expression). Expression of one or more of the genes (e.g., OCT4, SOX2, KLF4, or a combination thereof) may be modulated by altering the activity of an inducing agent. As a non-limiting example, tetracycline transactivator (tTA) is capable of inducing expression from a tetracycline-responsive promoter in the absence of tetracycline. When tetracycline is added, tTA can no longer bind to the promoter and cannot induce expression. As another non-limiting example, reverse tetracycline transactivator (rtTA) is capable of inducing expression from a tetracycline-responsive promoter in the presence of tetracycline. When tetracycline is removed, rtTA can no longer bind to the promoter and cannot induce expression. As described herein, vectors encoding OCT4, SOX2, and KLF4 (OSK) increased electric firing of nerve cells and improved cognitive function in vivo. Therefore, the expression of these three genes may be useful in central nervous system tissue and organ regeneration, central nervous system tissue and organ repair, reversing aging of the central nervous system, treating neurodegenerative diseases and conditions, and/or cellular reprogramming of the central nervous system.

In some embodiments, OCT4, SOX2, and/or KLF4 is expressed in the central nervous system for at most one month. Without being bound by a particular theory, expression of OCT4, SOX2, and/or KLF4 for two months may fail to rejuvenate the central nervous system.

In some embodiments, the results disclosed herein suggest that expression of OCT4, SOX2, and KLF4 can allow diseased cells in the central nervous system (e.g., brain) to revert to a healthier state without inducing complete reprogramming. Without being bound by a particular theory, the results disclosed herein suggest that cells maintain a backup epigenome that can be restored using the methods described herein.

Thus, in various embodiments the methods and uses of the invention rejuvenate a cell by restoring the cellular identity of the cell by reversing the effects of or by preventing the effects of one or more dysregulated developmental pathways. For example, in certain embodiments, the methods:
(i) increase the abundance of at least one of histone H2A, histone H2B, histone H3, histone H4, or any combination thereof in the cell;
(ii) increase the abundance of at least one of CHAF1a, CHAF1b, HP1α, NuRD, or any combination thereof in the cell;
(iii) increase at least one heterochromatin mark in the cell, such as, for example, H3K9me3, H3K27me3, or any combination thereof; or decrease one heterochromatin mark, such as H4K20me3 or euchromatin mark H3K4me3;
(iv) increase/decrease DNA methylation of at least one age-related CpG site in the cell towards a younger level;
(v) increase the abundance of lamin B1 in the cell;
(vi) increase acetylation of histone H3 at lysine 27 (H3K27ac), increase acetylation of histone H3 at lysine 56 (H3K56ac), or any combination thereof in the cell;
(vii) decrease acetylation of histone H3 at lysine 122 (H3K122Ac) or histone H4 at lysine 16 (H4K16ac), or any combination thereof in the cell
(viii) decrease the abundance of IL6, Ccl2, Ccl20, Apob, p16, LINE-1 repeats, Sat III repeats, Alu elements, IAP, or any combination thereof;
(ix) restores the balance between euchromatin epigenetic marks, such as H3K4me3, and heterochromatin epigenetic marks, such as H3K9me3 or H3K27me3;
(x) induces the formation of euchromatin;
(xi) restores youthful levels of at least one repressive heterochromatin epigenetic mark; and/or
(xii) restores the expression of at least one gene to youthful levels. In some embodiments, a youth level is the level of expression of the gene in a subject who is between 1-5, 1-10, 1-20, 1-30, or 1-40 years old.

In some embodiments, the at least one gene that is restored to youthful levels is at least one gene selected from the group consisting of RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

In some embodiments, the methods disclosed herein alleviate one or more hallmarks of aging, including genomic instability, telomere attrition, epigenetic alterations, loss of proteostasis, deregulated nutrient sensing, mitochondrial dysfunction, cellular senescence, stem cell exhaustion, and altered intercellular communication. See, e.g., Kennedy et al., Cell. 2013 Jun. 6; 153 (6): 1194-1217.

In some embodiments, methods disclosed herein increase the expression of one or more of the following gene sets: associative learning, excitatory synapse assembly, central nervous system neuron axonogenesis, central nervous system neuron development, memory, regulation of synaptic transmission GABAergic, regulation of postsynapse organization, learning, regulation of neurogenesis, central nervous system neuron differentiation, and/or central nervous system synapse maturation. See, e.g., Molecular Signatures Database (MSigDB) for gene sets. In some embodiments, a gene set is determined using the Biological Process terms of the Gene Ontology (GOBP). In some embodiments, the methods disclosed herein increase expression of one or more of these gene sets by at least 5%, at least 10%, at least 15%, at least 20%, at least 30%, at least 40%, at least 50%, at least 75%, at least 100%, at least 200%, at least 300%, at least 400%, at least 500%, at least 600%, at least 700%, at least 800%, at least 900%, or at least 1000% relative to a control. In some embodiments, a control is the level of expression of one or more gene sets in the absence of inducing OCT4, KLF4, and SOX2 expression. In some embodiments, gene expression is measured at least 4 weeks after induction of OCT4, KLF4, and SOX2 expression has stopped. In some embodiments, gene expression is measured at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 20 weeks, at least 30 weeks, at least 40 weeks, or at least 50 weeks after induction of OCT4, KLF4, and SOX2 expression has stopped.

In some embodiments, a method disclosed herein increases hypo-methylated CpG sites and/or hyper-methylated CpG sites at development-related loci relative to a control. A hypo-methylated CpG site is a CpG site that has decreased methylation relative to a control. A hyper-methylated CpG site is a CpG site that has increased methylation relative to a control. In some embodiments, hypo-methylated CpG sites are increased at development-related loci associated with positive regulation of epidermis development, midgut development, negative regulation of chromatin organization, negative regulation of histone methylation, lens development in camera-type eye, negative regulation of fat cell differentiation, negative regulation of histone modification, lung morphogenesis, DNA methylation on cytosine, and/or neural precursor cell proliferation. In some embodiments, a control is the level of hypo-methylation and/or hyper-methylation at one or more development-related loci in the absence of inducing OCT4, KLF4, and SOX2 expression. In some embodiments, hyper-methylated CpG sites are increased at development-related loci associated with skeletal muscle cell differentiation, heart looping, determination of heart left/right asymmetry, uterus morphogenesis, regulation of Notch signaling pathway, embryonic cranial skeleton morphogenesis, animal organ formation, tricuspid valve morphogenesis, lens fiber cell differentiation, and/or positive regulation of BMP signaling pathway. In some embodiments, a control is the level of hypo-methylation and/or hyper-methylation at one or more development-related loci in the absence of inducing OCT4, KLF4, and SOX2 expression.

In some embodiments, the methods disclosed herein increases hypo-methylated CpG sites and/or hyper-methylated CpG sites at development-related loci relative to a control by at least 5%, at least 10%, at least 15%, at least 20%, at least 30%, at least 40%, at least 50%, at least 75%, at least 100%, at least 200%, at least 300%, at least 400%, at least 500%, at least 600%, at least 700%, at least 800%, at least 900%, or at least 1000% relative to a control. In some embodiments, a control is the level of hypo-methylation and/or hyper-methylation at one or more development-related loci in the absence of inducing OCT4, KLF4, and SOX2 expression.

Without being bound by a particular theory, more specifically, the methods, compositions, uses, and kits of the present disclosure rejuvenate cells in the central nervous system (e.g., brain) by restoring epigenetic information that has been lost due to the aging process, injury, or disease. The methods, compositions, uses, and kits of the present disclosure comprise the transcription factors OCT4, SOX2, and KLF4. OCT4, SOX2, and KLF4 are three of the four “Yamanaka factors”, with the fourth being c-Myc. The Yamanaka factors have traditionally been used to reprogram cells to a pluripotent state. However, the induction of expression of the four transcription factors in transgenic mice resulted in the formation of teratomas in vivo, along with other acute toxicitys like dysplasia in the intestinal epithelium, which can kill the animal in a few days. Moreover, the fact that the four Yamanaka factors are typically used to reprogram cells to a completely pluripotent state, wherein the cell loses its pre-established cellular identity, can be dangerous for in vivo applications where the cellular identity of target cells must be maintained for tissue and/or organ integrity. In contrast, in some embodiments, the methods described herein, allow controlled reprogramming and do not result in global changes in demethylation. In some embodiments, the methods described herein do not require complete de-differentiation of cells.

Cellular reprograming allows for the production of numerous cell types from existing somatic cells. Although the Yamanaka factors (OCT4, SOX2, KLF4 and c-Myc, also known collectively as OSKM) have been shown to induce pluripotency in differentiated cells, administration of these factors may induce teratomas or other cancers in vivo (Takahashi et al., Cell 2006 Aug. 25; 126 (4): 663-76; Abad et al., Nature 2013 Oct. 17; 502 (7471): 340-5). As a result of these safety concerns, use of the Yamanaka factors has largely been limited to in vitro applications. Furthermore, existing methods of gene therapy are plagued by inefficient and inconsistent gene transduction of target cells. The engineered nucleic acids, recombinant viruses comprising the same, pharmaceutical compositions thereof and kits provided herein overcome many of these limitations.

### Engineered Nucleic Acids

The engineered nucleic acids of the present disclosure may encode OCT4, SOX2, KLF4, and homologs or variants (e.g., functional variants) thereof, each alone or in combination and/or comprise a nucleic acid encoding an inducing agent, which may be useful in rejuvenating a cell, tissue, and/or organ in the central nervous system. For example, the cell, tissue, and/or organ may be in a subject in need thereof. In some instances, the central nervous system excludes the retina. In some embodiments, the central nervous system excludes the eye (e.g., the retina, uvea, pupil, lens, cornea, and/or sclera). In some embodiments, the cell, tissue, and/or organ is a brain cell, brain tissue, and/or brain. In some embodiments, the subject in need thereof has a neurological disease. In certain embodiments, an engineered nucleic acid (e.g., engineered nucleic acid) does not encode c-Myc. In certain embodiments, an engineered nucleic acid (e.g., engineered nucleic acid) does not encode a functional c-Myc because it lacks a c-Myc sequence. Assays to determine transcription factor (e.g., OCT4, SOX2, KLF4, or any combination thereof) activity are known in the art and include cell-based transcription assays and in vitro transcription assays. Transcription factor expression may also be determined using other methods including enzyme-linked immunosorbent assays (ELISAs), western blots, and quantification of RNA (e.g., using reverse transcription polymerase chain reaction).

A transcription factor (e.g., OCT4, SOX2, KLF4, or homologs or variants thereof, including mammalian OCT4, mammalian SOX2, and mammalian KLF4) may be encoded by a single nucleic acid, or a single nucleic acid (e.g., engineered nucleic acid) may encode two or more transcription factors (e.g., each operably linked to a different promoter, or both operably linked to the same promoter). For example, in certain embodiments, a nucleic acid (e.g., engineered nucleic acid) may encode OCT4; SOX2; KLF4; OCT4 and SOX2; OCT4 and KLF4; SOX2 and KLF4; or OCT4, SOX2, and KLF4, in any order.

In certain embodiments, an engineered nucleic acid (e.g., engineered nucleic acid) encodes an inducing agent (e.g., tTA or rtTA). In certain embodiments, a nucleic acid (e.g., engineered nucleic acid) may encode one or more transcription factors (e.g., one, two or three transcription factors) and an inducing agent. In certain embodiments, an inducing agent is encoded by a separate nucleic acid (e.g., engineered nucleic acid) that does not also encode a transcription factor (e.g., OCT4, SOX2, or KLF4). In certain embodiments, an inducing agent is encoded by a nucleic acid (e.g., engineered nucleic acid) that also encodes a transcription factor (e.g., OCT4, SOX2, and/or KLF4). In certain embodiments, an inducing agent is encoded by a nucleic acid (e.g., engineered nucleic acid) that also encodes one or more transcription factors selected from the group consisting of OCT4; SOX2; KLF4; and any combinations thereof (e.g., OCT4; SOX2; KLF4; OCT4 and SOX2; OCT4 and KLF4; SOX2 and KLF4; or OCT4, SOX2, and KLF4).

The transcription factors described herein (e.g., OCT4, SOX2, KLF4, or any combination thereof) or inducing agents may comprise one or more amino acid substitutions. Variants can be prepared according to methods for altering polypeptide sequences known to one of ordinary skill in the art such as those found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (c) S, T; (f) Q, N; and (g) E, D.

In certain embodiments, the engineered nucleic acids of the present disclosure comprise RNA (e.g., mRNA) and/or DNA. In some embodiments, the RNA and/or DNA is further modified. As a non-limiting example, a nucleic acid (e.g., engineered nucleic acid) of the present disclosure, may be modified RNA (e.g., mRNA) encoding OCT4, KLF4, SOX2, an inducing, or any combination thereof. See, e.g., Warren et al., Cell Stem Cell. 2010 Nov. 5; 7 (5): 618-30. As a non-limiting example, the engineered nucleic acids (e.g., RNA, including mRNA, or DNA) of the present disclosure may be formulated in a nanoparticle for delivery. See, e.g., Dong et al., Nano Lett. 2016 Feb. 10; 16 (2): 842-8. In some embodiments, the nanoparticle comprises acetylated galactose. See, e.g., Lozano-Torres et al., J Am Chem Soc. 2017 Jul. 5; 139 (26): 8808-8811. In some embodiments, the engineered nucleic acids (e.g., RNA, including mRNA, or DNA) is electroporated or transfected into a cell. In certain embodiments, the engineered nucleic acids are delivered as a naked nucleic acid (e.g., naked DNA or naked RNA).

In some embodiments, an engineered nucleic acid that is formulated in a nanoparticle for delivery is not an AAV vector. Suitable vector backbones for formulation in a nanoparticle include, but are not limited to, NANOPLASMID™ vectors and NTC ‘8’ Series Mammalian Expression Vectors. Non-limiting examples of vector backbones for formulation in a nanoparticle include NTC9385R and NTC8685. Without being bound by a particular theory, NTC ‘8’ Series Mammalian Expression Vectors may be useful as they are generally cleared by cells within weeks. The NTC ‘8’ Series Mammalian Expression Vector comprises a CMV promoter, which can be operably linked to a sequence encoding OCT4, KLF4, SOX2, or a combination thereof. Without being bound by a particular theory, the NANOPLASMID™ vector may be less immunogenic than other vectors and express at a higher level and may express for a long time, which could be useful in long-term expression of an operably linked nucleic acid. In some embodiments, the NANOPLASMID™ vector may be useful in long term expression of OCT4, KLF4, SOX2, or a combination thereof.

Without being bound by a particular theory, modified RNA (e.g., mRNA) may have an advantage of minimal activation of innate immune responses and limited cytotoxicity, thereby allowing robust and sustained protein expression. In some embodiments, the RNA (e.g., mRNA) comprises modifications including complete substitution of either 5-methylcytidine (5mC) for cytidine or pseudouridine (psi) for uridine.

In some embodiments, OCT4, KLF4, and/or SOX2 expression may be activated using a CRISPR-activating system. In some embodiments, expression of one or more transcription factors selected from the group consisting of OCT4, KLF4, SOX2, and combinations thereof may be activated using a CRISPR-activating system. See, e.g., Liao et al., Cell. 2017 Dec. 14; 171 (7): 1495-1507.e15; Liu et al., 2018, Cell Stem Cell 22, 1-10 Feb. 1, 2018. In general, a CRISPR-activating system comprises an enzymatically dead Cas9 nuclease (or nuclease-deficient Cas9 (dCas9)) fused to a transcription activation complex (e.g., comprising VP64, P65, Rta, and/or MPH). Non-limiting examples of sequences encoding VP64, P65, Rta, and/or MPH are provided below. A VP64, P65, Rta, or MPH may be encoded by a sequence that comprises a sequence that is at least 70% (e.g., 75%, 80%, 85%, 90%, 95%, 98%, 99%, or 100%) identical to any of the VP64, P65, Rta, and/or MPH sequences described herein. This Cas9 fusion protein may be referred to as a CRISPR activator. A guide RNA targeting the promoter and/or enhancer region of a gene of interest is used in a CRISPR-activating system to target the dCas9-transcription activation complex and drive expression of the endogenous gene.

In some embodiments, expression of OCT4; KLF4; SOX2; or any combination thereof may be activated using a transcription activator-like effector nucleases (TALEN) or a Zinc-finger nuclease (ZFN) system.

The engineered nucleic acids of the present disclosure may encode sgRNA to target and the promoter and/or enhancer region of the endogenous locus of OCT4, SOX2, and/or KLF4 in a cell. The engineered nucleic acids of the present disclosure may encode sgRNA to target and the promoter and/or enhancer region of the endogenous locus of one or more transcription factors selected from OCT4; SOX2; KLF4; and any combinations thereof in a cell. In some embodiments, the engineered nucleic acid (e.g., expression vector) further encodes a dCas9 (dead Cas9) and a transcriptional activation complex (e.g., VP64, P65, Rta, MPH). In some embodiments, the dCas9 (dead Cas9) and a transcriptional activation complex (e.g., VP64, P65, Rta, MPH) is administered to a cell on a engineered nucleic acid (e.g. expression vector). In some embodiments, the vector encoding the sgRNA and/or a dCas9 (dead Cas9) and a transcriptional activation complex (e.g., VP64, P65, Rta, MPH) is a viral vector (e.g., AAV vector). In some embodiments, dCas9 (dead Cas9) and a transcriptional activation complex (e.g., VP64, P65, Rta, MPH) is introduced into a cell as protein.

In some embodiments, guide RNA targeting the enhancer and/or promoter region of OCT4, SOX2, and/or KLF4 is formulated in a nanoparticle and injected with dCas9-VP64 protein. In some embodiments, guide RNA targeting the enhancer and/or promoter region of OCT4, SOX2, KLF4, or any combination thereof is formulated in a nanoparticle and injected with dCas9-VP64 protein. In some embodiments, the guide RNA and/or nucleic acid encoding dCas9 (dead Cas9) and a transcriptional activation complex (e.g., VP64, P65, Rta, MPH) is administered as naked nucleic acid (e.g., naked DNA formulated in a nanoparticle). In some embodiments, the guide RNA and/or nucleic acid encoding dCas9 (dead Cas9) and a transcriptional activation complex (e.g., VP64, P65, Rta, MPH) is delivered via a recombinant virus (e.g., lentivirus, adenovirus, retrovirus, herpes virus, human papillomavirus, alphavirus, vaccinia virus or adeno-associated virus (AAV)).

Non-limiting example, sequences of guide RNAs targeting the endogenous OCT4 locus or SOX2 locus are provided in Liu et al., Cell Stem Cell. 2018 Feb. 1; 22 (2): 252-261.e4. Non-limiting examples of guide RNAs targeting OCT4, SOX2, and/or KLF4 are also provided in Weltner et al., Nat Commun. 2018 Jul. 6; 9 (1): 2643.

Without being bound by a particular theory, use of a CRISPR-CAS9 system to activation endogenous expression of OCT4, KLF4, and/or SOX2 in the absence of c-Myc expression may obviate potential toxicity associated with exogenous gene expression and/or superphysiological gene expression.

Nucleic acids (e.g., engineered nucleic acids) encoding a transcription factor (OCT4, SOX2, KLF4, or any combination thereof) or encoding an inducing agent may be introduced into an expression vector using conventional cloning techniques. Suitable expression vectors include vectors with a promoter (e.g., a constitutive or inducible promoter, including a TRE promoter) operably-linked to a nucleic acid (e.g., engineered nucleic acid) encoding OCT4, SOX2, KLF4, or any combination thereof, and a terminator sequence (e.g., a SV40 sequence as described herein). In some embodiments, a nucleic acid (e.g., engineered nucleic acid) encodes a promoter operably linked to a nucleic acid encoding an inducing agent. In some embodiments, a vector comprises a WPRE sequence. Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

Vectors of the invention may further comprise a marker sequence for use in the identification of cells that have or have not been transformed or transfected with the vector, or have been reprogrammed. Markers include, for example, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds, genes encoding enzymes with activities detectable by standard assays known in the art (e.g., β-galactosidase, senescence-associated beta-galactosidase, luciferase or alkaline phosphatase), and genes that visibly affect the phenotype of transformed or transfected cells, hosts, colonies or plaques (e.g., green fluorescent protein). In some embodiments, the vectors used herein are capable of autonomous replication and expression of the structural gene products present in the DNA segments to which they are operably linked. In some embodiments, a vector encoding a neomycin resistance gene comprises a sequence that is at least 70% (e.g., at least 75%, 80%, 85%, 90%, 95%, 98%, 99%, or 100%) identical to SEQ ID NO: 133. In some embodiments, a neomycin resistance gene comprises a sequence that is at least 70% (e.g., at least 75%, 80%, 85%, 90%, 95%, 98%, 99%, or 100%) identical to SEQ ID NO: 134.

In some embodiments, an expression vector comprises a sequence that is at least 70% identical (e.g., at least 75%, 80%, 85%, 90%, 95%, 98%, 99%, or 100% identical) to a sequence selected from SEQ ID NOs: 123-127.

In certain embodiments, the expression vector comprises an inducible promoter (e.g., a tetracycline-responsive promoter) operably linked to a sequence encoding a transcription factor (e.g., OCT4, SOX2, KLF4, or any combination thereof).

In certain embodiments, the promoter operably linked to a sequence encoding a transcription factor (e.g., OCT4, SOX2, KLF4, or any combination thereof) and/or encoding an inducing agent is a tissue-specific or cell type-specific promoter (e.g., brain-specific, liver-specific, muscle-specific, nerve cell-specific, glial cell-specific, endothelial cell-specific, lung-specific, heart-specific, bone-specific, intestine-specific, skin-specific promoters, or eye-specific promoter). As an example, the muscle-specific promoter may be a desmin promoter (e.g., a sequence that is at least 70% (e.g., at least 75%, 80%, 85%, 90%, 95%, 98%, 99%, or 100%) identical to SEQ ID NO: 29). In some embodiments, an eye-specific promoter may be a promoter that is at least 70% (e.g., at least 75%, 80%, 85%, 90%, 95%, 98%, 99%, or 100%) identical to a sequence selected from SEQ ID NOs: 101-104. In some embodiments, the tissue-specific promoter is not an eye-specific promoter.

In some embodiments, is the tissue-specific promoter is a central nervous system-specific promoter. In some embodiments, the tissue-specific promoter is a neuron-specific promoter. In some embodiments, a neuron-specific promoter is a CaMKIIα promoter In some embodiments, a brain-specific promoter is a CaMKIIα promoter. In some embodiments, a CaMKIIα promoter comprises a sequence that is at least 70% (e.g., at least 75%, 80%, 85%, 90%, 95%, 98%, 99%, or 100%) identical to SEQ ID NO: 146, 149, or 154.

In some embodiments, a promoter that is operably linked to a sequence encoding a transcription factor (e.g., OCT4, SOX2, KLF4, or any combination thereof) and/or encoding an inducing agent does not comprise an eye-specific promoter. In some embodiments, a promoter that is operably linked to a sequence encoding a transcription factor (e.g., OCT4, SOX2, KLF4, or any combination thereof) and/or encoding an inducing agent does not comprise Synapsin-I promoter, does not comprise a CaMKII-gamma promoter, or a combination thereof. In some embodiments, a promoter that is operably linked to a sequence encoding a transcription factor (e.g., OCT4, SOX2, KLF4, or any combination thereof) does not comprise a sequence that is at least 70% (e.g., at least 75%, 80%, 85%, 90%, 95%, 98%, 99%, or 100%) identical to SEQ ID NO: 157.

In certain embodiments, the promoter operably linked to a sequence encoding a transcription factor (e.g., OCT4, SOX2, KLF4, or any combination thereof) and/or encoding an inducing agent is an age- or senescence-specific (e.g. the age- or senescence-specific promoter, which may be a p16 promoter or a Cas9-directed transcription factor that binds to methylated DNA, which accumulates with age).

In certain embodiments, an expression vector comprises a constitutive promoter operably linked to a nucleic acid (e.g., engineered nucleic acid) encoding OCT4, SOX2, KLF4, or any combination thereof. In some embodiments, such a vector may be inactivated using a Clustered Regularly Interspaced Short Palindromic Repeats (CRISPR)/guide RNA system. For example, a guide RNA may be complementary to the vector and is capable of targeting a Cas9 nuclease to the vector. In some embodiments, the guide RNA is complementary to a transgene (e.g. transgene encoding OCT4, KLF4, SOX2, or a combination thereof) in any of the expression vectors described herein. Cas9 may then generate double-stranded breaks in the vector and/or mutate the vector, rendering the vector inactive.

In certain embodiments, the promoter operably linked to a sequence encoding an inducing agent is a constitutive promoter (e.g., CMV, EF1 alpha, a SV40 promoter, PGK1, UBC, CAG, human beta actin gene promoter, or UAS). In certain embodiments, the promoter operably linked to a sequence encoding an inducing agent is a tissue-specific promoter (e.g., brain-specific, liver-specific, muscle-specific, nerve cell-specific, lung-specific, heart-specific, bone-specific, intestine-specific, skin-specific promoters, or eye-specific promoter). As an example, the muscle-specific promoter may be a desmin promoter (e.g., a sequence that is at least 70% (e.g., at least 75%, 80%, 85%, 90%, 95%, 98%, 99%, or 100%) identical to SEQ ID NO: 29).

A nucleic acid (e.g., engineered nucleic acid) (e.g., an expression vector) may further comprise a separator sequence (e.g., an IRES or a polypeptide cleavage signal). Exemplary polypeptide cleavage signals include 2A peptides (e.g., T2A, P2A, E2A, and F2A). A 2A peptide may comprise a sequence that is at least 70% (e.g., at least 75%, 80%, 85%, 90%, 95%, 98%, 99%, or 100%) identical to SEQ ID NO: 9 or 118. For nucleic acids (e.g., engineered nucleic acids) (e.g., expression vectors) encoding more than one transcription factor (e.g., OCT4, SOX2, KLF4, or any combination thereof), each transcription factor may be operably linked to a different promoter or to the same promoter. The transcription factors may be separated (e.g., by peptide separator sequence) on the nucleic acid. Expression of the nucleic acid (e.g., engineered nucleic acid) results in separate amino acid sequences encoding each transcription factor.

In certain embodiments, an expression vector (e.g., an expression vector encoding OCT4, KLF4, SOX2, or a combination thereof) of the present disclosure may further comprise a selection agent (e.g., an antibiotic, including blasticidin, geneticin, hygromycin B, mycophenolic acid, puromycin, zeocin, actinomycin D, ampicillin, carbenicillin, kanamycin, and neomycin) and/or detectable marker (e.g., GFP, RFP, luciferase, CFP, mCherry, DsRed2FP, mKate, biotin, FLAG-tag, HA-tag, His-tag, Myc-tag, V5-tag, etc.).

In certain embodiments, an expression vector encoding an inducing agent of the present disclosure may further comprise a selection agent (e.g., an antibiotic, including blasticidin, geneticin, hygromycin B, mycophenolic acid, puromycin, zeocin, actinomycin D, ampicillin, carbenicillin, kanamycin, and neomycin) and/or detectable marker (e.g., GFP, RFP, luciferase, CFP, mCherry, DsRed2FP, mKate, biotin, FLAG-tag, HA-tag, His-tag, Myc-tag, V5-tag, etc.).

In some embodiments, an expression vector encoding a neomycin resistance gene comprises a sequence that is at least 70% (e.g., at least 75%, 80%, 85%, 90%, 95%, 98%, 99%, or 100%) identical to SEQ ID NO: 133. In some embodiments, a neomycin resistance gene comprises a sequence that is at least 70% (e.g., at least 75%, 80%, 85%, 90%, 95%, 98%, 99%, or 100%) identical to SEQ ID NO: 134.

In certain embodiments, an expression vector (e.g., encoding OCT4, SOX2, KLF4, or any combination thereof) is present on a viral vector (e.g., AAV vector). In certain embodiments, an expression vector encoding an inducing agent is present on a viral vector (e.g., AAV vector). An AAV vector, as used herein, generally comprises ITRs flanking an expression cassette (e.g., a nucleic acid (e.g., engineered nucleic acid) comprising a promoter sequence operably linked to a sequence encoding OCT4, SOX2, KLF4, or any combination thereof and a terminator sequence, a nucleic acid (e.g., engineered nucleic acid) comprising a promoter sequence operably linked to a sequence encoding an inducing agent, or a combination thereof).

In certain embodiments, the number of base pairs between two ITRs in an AAV vector of the present disclosure is less than 5 kilobases (kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb). In certain embodiments, an AAV vector with a distance of less than 4.7 kb between two ITRs is capable of being packaged into virus at a titer of at least 0.5×10\hat{ }10 particle forming units per ml (pfu/ml), at least 1×10\hat{ }10 pfu/ml, at least 5×10\hat{ }10 pfu/ml, at least 1×10\hat{ }11 pfu/ml, at least 5×10\hat{ }11 pfu/ml, at least 1×10\hat{ }12 pfu/ml, at least 2×10\hat{ }12 pfu/ml, at least 3×10\hat{ }12 pfu/ml, at least 4×10\hat{ }12 pfu/ml, at least 5×10\hat{ }12 pfu/ml, at least 6×10\hat{ }12 pfu/ml, at least 7×10\hat{ }12 pfu/ml, at least 8×10\hat{ }12 pfu/ml, at least 9×10\hat{ }12 pfu/ml, or at least 1×10\hat{ }13 pfu/ml.

In certain embodiments, an expression vector of the present disclosure is at least 1 kilobase (kb) (e.g., at least 1 kb, 2 kb, 3 kb, 4 kb, 5 kb, 6 kb, 7 kb, 8 kb, 9 kb, 10 kb, 50 kb, or 100 kb). In certain embodiments, an expression vector of the present disclosure is less than 10 kb (e.g., less than 9 kb, less 8 kb, less than 7 kb, less than 6 kb, less than 5 kb, less than 4 kb, less than 3 kb, less than 2 kb, or less than 1 kb).

Without being bound by a particular theory, an expression vector (e.g., an AAV vector) that encodes OCT4, SOX2, and KLF4 under one promoter results in more efficient transduction of all three transcription factors in vivo compared to separate nucleic acids (e.g., engineered nucleic acids) encoding one or two of the transcription factors. In certain embodiments, the infection efficiency of a recombinant virus (e.g., lentivirus, alphavirus, vaccinia virus, retrovirus, adenovirus, herpes virus, human papillomavirus, or AAV) harboring a vector of the present disclosure in cells (e.g., animal cells, including mammalian cells) is at least 20% (e.g., at least 30%, at least 40%, at least 50%, at least 60%, at least 70%, at least 80%, or at least 90%, or 100%).

In some embodiments, an engineered nucleic acid described herein comprises a sequence that is at least 70% (e.g., at least 75%, 80%, 85%, 90%, 95%, 98%, 99%, or 100%) identical to a sequence disclosed herein.

In some embodiments, an engineered nucleic acid comprises a sequence that is at least 70% (e.g., at least 75%, 80%, 85%, 90%, 95%, 98%, 99%, or 100%) identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. In some embodiments, an engineered nucleic acid comprises SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. In some embodiments, an engineered nucleic acid (e.g., expression vector) consists of SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. See also, e.g., International Publication Number WO 2020/069373, entitled “Cellular Reprogramming to Reverse Aging and Promote Organ and Tissue regeneration,” which was published on Apr. 2, 2020, and which is herein incorporated by reference in its entirety.

In some embodiments, an engineered nucleic acid encoding an inducing agent comprises a sequence that is at least 70% (e.g., at least 75%, 80%, 85%, 90%, or 100%) identical to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126. In some embodiments, an engineered nucleic acid encoding an inducing agent does not comprise a sequence that is at least 70% (e.g., at least 75%, 80%, 85%, 90%, 95%, 98%, 99%, or 100%) identical to SEQ ID NO: 127. In some embodiments, an engineered nucleic acid encoding an inducing agent comprises SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126. In some embodiments, an engineered nucleic acid encoding an inducing agent does not comprise SEQ ID NO: 127. In some embodiments, the expression vector encoding an inducing agent consists of SEQ ID NO: 10, SEQ ID NO: 12, SEQ ID NO: 14, SEQ ID NO: 17, SEQ ID NO: 31, SEQ ID NO: 32, SEQ ID NO: 123, SEQ ID NO: 124, SEQ ID NO: 125, or SEQ ID NO: 126. See also, e.g., International Publication Number WO 2020/069339, entitled “Mutant Reverse Tetracycline Transactivators for Expression of Genes,” which was published on Apr. 2, 2020, and which is herein incorporated by reference in its entirety.

One aspect of the present disclosure provides vectors (e.g., expression vectors) comprising a first nucleic acid (e.g., engineered nucleic acid) encoding OCT4, a second nucleic acid (e.g., engineered nucleic acid) encoding SOX2, a third nucleic acid (e.g., engineered nucleic acid) encoding KLF4, alone or in combination, and in the absence of an exogenous nucleic acid (e.g., engineered nucleic acid) capable of expressing c-Myc for use in the central nervous system. In certain embodiments, a vector (e.g., expression vector) comprising a first nucleic acid (e.g., engineered nucleic acid) encoding OCT4, a second nucleic acid (e.g., engineered nucleic acid) encoding SOX2, a third nucleic acid (e.g., engineered nucleic acid) encoding KLF4, or any combination thereof. In certain embodiments, the first, second, and third nucleic acids (e.g., engineered nucleic acids) are present on separate expression vectors. In certain embodiments, two of the first, second, and third nucleic acids (e.g., engineered nucleic acids) are present on the same expression vector. In some embodiments, all three nucleic acids (e.g., engineered nucleic acids) are present on the same expression vector. In certain embodiments, the sequence encoding OCT4 is at least 70% (e.g., at least 75%, 80%, 85%, 90%, 95%, 98%, 99%, or 100%) identical to SEQ ID NO: 2 or 41. In certain embodiments, the sequence encoding SOX2 is at least 70% (e.g., at least 75%, 80%, 85%, 90%, 95%, 98%, 99%, or 100%) identical to SEQ ID NO: 4 or 43. In certain embodiments, the sequence encoding KLF4 is at least 70% (e.g., at least 75%, 80%, 85%, 90%, 95%, 98%, 99%, or 100%) identical to SEQ ID NO: 6 or 45. In certain embodiments, one or more of OCT4, SOX2, and KLF4 are human proteins. In certain embodiments, one or more of OCT4, SOX2, and KLF4 are non-human proteins (for example, from other mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). If two or more of OCT4, SOX2, and KLF4 are on one vector, they may be in any order. The words “first,” “second,” and “third” are not meant to imply an order of the genes on the vector. In some embodiments, a vector encodes OCT4, followed by SOX2, and then KLF4. In some embodiments, a vector encodes OCT4, followed by KLF4, and then SOX2. In some embodiments, a vector encodes KLF4, followed by OCT4, and then SOX2. In some embodiments, a vector encodes KLF4, followed by SOX2, and then OCT4. In some embodiments, a vector encodes SOX2, followed by KLF4, and then OCT4. In some embodiments, a vector encodes SOX2, followed by OCT4, and then KLF4.

An expression vector of the present disclosure may further comprise an inducible promoter. An expression vector may only have one inducible promoter. In such instances, the expression of OCT4, SOX2, and KLF4 are under the control of the same inducible promoter. In some instances, an expression vector comprises more than one inducible promoter. The inducible promoter may comprise a tetracycline-responsive element (TRE) (e.g., a TRE3G promoter, a TRE2 promoter, or a P tight promoter), mifepristone-responsive promoters (e.g., GAL4-E1b promoter), or a coumermycin-responsive promoter. As an example, a TRE (e.g., TRE3G) promoter may comprise a nucleic acid (e.g., engineered nucleic acid) sequence that is at least 70% (e.g., at least 75%, 80%, 85%, 90%, 95%, 98%, 99%, or 100%) identical to SEQ ID NO: 7. As an example, a TRE (e.g., TRE2) promoter may comprise a nucleic acid (e.g., engineered nucleic acid) sequence that is at least 70% (e.g., at least 75%, 80%, 85%, 90%, 95%, 98%, 99%, or 100%) identical to SEQ ID NO: 23. As an example, a TRE (e.g., P tight) promoter may comprise a nucleic acid (e.g., engineered nucleic acid) sequence that is at least 70% (e.g., at least 75%, 80%, 85%, 90%, 95%, 98%, 99%, or 100%) identical to SEQ ID NO: 24. See, e.g., International Publication Number WO 2020/069339, entitled “Mutant Reverse Tetracycline Transactivators for Expression of Genes,” which was published on Apr. 2, 2020, and which is herein incorporated by reference in its entirety.

In certain embodiments, an inducing agent is capable of inducing expression of the first (e.g., OCT4), second (e.g., SOX2), third (e.g., KLF4) nucleic acids (e.g., engineered nucleic acids), or any combination thereof from the inducible promoter in the presence of a tetracycline (e.g., doxycycline). In certain embodiments, the inducing agent is a reverse tetracycline-controlled transactivator (rtTA) (e.g., rtTA3, rtTA4, rtTA Advanced, rtTA2S-M2, or any rtTA disclosed herein). In certain embodiments, the inducing agent is a rtTA and comprises a sequence that is at least 70% (e.g., at least 75%, 80%, 85%, 90%, 95%, 98%, 99%, or 100%) identical to any rtTA disclosed herein. In certain embodiments, the rtTA is rtTA3 comprising an amino acid sequence that is at least 70% (e.g., at least 75%, 80%, 85%, 90%, 95%, 98%, 99%, or 100%) identical to SEQ ID NO: 11. In certain embodiments, the rtTA is rtTA Advanced comprising an amino acid sequence that is at least 70% (e.g., at least 75%, 80%, 85%, 90%, 95%, 98%, 99%, or 100%) identical to SEQ ID NO: 129. In certain embodiments, the rtTA is rtTA4 and comprises a sequence that is at least 70% (e.g., at least 75%, 80%, 85%, 90%, 95%, 98%, 99%, or 100%) identical to SEQ ID NO: 13. In certain embodiments, the rtTA is rtTA2S-M2 and comprises a sequence that is at least 70% (e.g., at least 75%, 80%, 85%, 90%, 95%, 98%, 99%, or 100%) identical to SEQ ID NO: 15. In certain embodiments, the inducing agent is tetracycline-controlled transactivator (tTA) (e.g., any tTA disclosed herein). In certain embodiments, the inducing agent is a tTA and comprises a sequence that is at least 70% (e.g., at least 75%, 80%, 85%, 90%, 95%, 98%, 99%, or 100%) identical to any tTA disclosed herein. In certain embodiments, the inducing agent is a tTA and comprises a sequence that is at least 70% (e.g., at least 75%, 80%, 85%, 90%, 95%, 98%, 99%, or 100%) identical to SEQ ID NO: 138 or 159. In certain embodiments, the inducing agent is a tTA and is encoded by a sequence that comprises a sequence that is at least 70% (e.g., at least 75%, 80%, 85%, 90%, 95%, 98%, 99%, or 100%) identical to SEQ ID NO: 137 or 158.

In certain embodiments, an inducing agent is capable of inducing expression of the first nucleic acid (e.g., engineered nucleic acid) (e.g., OCT4), second nucleic acid (e.g., engineered nucleic acid) (e.g., SOX2), third nucleic (e.g., KLF4), or any combination thereof from the inducible promoter in the absence of tetracycline (e.g., doxycycline). In certain embodiments, the inducing agent is tetracycline-controlled transactivator (ITA).

In certain embodiments, an expression vector of the present disclosure comprises a promoter that is specific for the central nervous system. In some embodiments, an expression vector of the present disclosure comprises a neuron-specific promoter. In some embodiments, an expression vector of the present disclosure comprises a brain-specific promoter. In some embodiments, an expression vector comprises a CaMKIIα promoter. In some embodiments, a CaMKIIα promoter comprises a sequence that is at least 70% (e.g., at least 75%, 80%, 85%, 90%, 95%, 98%, 99%, or 100%) identical to SEQ ID NO: 146, 149, or 154.

In some embodiments, an expression vector of the present disclosure does not comprise an eye-specific promoter. In some embodiments, an expression vector does not comprise a Synapsin-I promoter, does not comprise a CaMKII-gamma promoter, or a combination thereof. In some embodiments, an expression vector does not comprise a sequence that is at least 70% (e.g., at least 75%, 80%, 85%, 90%, 95%, 98%, 99%, or 100%) identical to SEQ ID NO: 157.

In certain embodiments, an expression vector of the present disclosure comprises an SV40-derived terminator sequence. In certain embodiments, the SV40-derived sequence is at least 70% (e.g., at least 75%, 80%, 85%, 90%, 95%, 98%, 99%, or 100%) identical to SEQ ID NO: 8 or 143.

In certain embodiments, an expression vector of the present disclosure comprises a separator sequence, which may be useful in producing two separate amino acid sequences from one transcript. The separator sequence may encode a self-cleaving peptide (e.g., 2A peptide, including a 2A peptide sequence that is at least 70% (e.g., at least 75%, 80%, 85%, 90%, 95%, 98%, 99%, or 100%) identical to SEQ ID NO: 9 or 118). In certain embodiments, the separator sequence is an Internal Ribosome Entry Site (IRES).

In certain embodiments, the expression vector is a viral vector (e.g., a lentiviral, a retroviral, or an adeno-associated virus (AAV) vector). An AAV vector of the present disclosure generally comprises inverted terminal repeats (ITRs) flanking a transgene of interest (e.g., a nucleic acid (e.g., engineered nucleic acid) encoding OCT4, SOX2, KLF4, an inducing agent, or a combination thereof). In some embodiments, the distance between two inverted terminal repeats is less than 5.0 kilobases (kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb).

In some embodiments, the expression vector (e.g., viral vector) encoding OCT4, KLF4, and SOX2 comprises a sequence that is at least 70% (e.g., at least 75%, 80%, 85%, 90%, 95%, 98%, 99%, or 100%) identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. Viral vectors include adeno-associated virus (AAV) vectors, retroviral vectors, lentiviral vectors, and herpes viral vectors. In another aspect, the present disclosure provides recombinant viruses (e.g., lentivirus, adenovirus, retrovirus, herpes virus, human papillomavirus, alphavirus, vaccinia virus or adeno-associated virus (AAV)) comprising any of the expression vectors described herein. In certain embodiments, a recombinant virus encodes a transcription factor selected from OCT4; KLF4; SOX2; and any combinations thereof. In certain embodiments, a recombinant virus encodes two or more transcription factors selected from the group consisting of OCT4, KLF4, and SOX2. In certain embodiments, a recombinant virus encodes OCT4 and SOX2, OCT4 and KLF4, OCT4, KLF4, and SOX2, or SOX2 and KLF4. In certain embodiments, a recombinant virus encodes OCT4, KLF4, and SOX2. In certain embodiments, a four or more transcription factors encodes four or more transcription factors (e.g., OCT4, SOX2, KLF4, and another transcription factor).

The expression vector comprising one or more of the first, second, and third nucleic acids (e.g., engineered nucleic acids) may be any of the expression vectors described above and herein. In some embodiments, the first nucleic acid, the second nucleic acid, the third nucleic acid, or any combination thereof are present on separate expression vectors. In certain embodiments, two of the first nucleic acid, the second nucleic acid, the third nucleic acid, or any combination thereof are present on the same expression vector. In certain embodiments, all three nucleic acids (e.g., engineered nucleic acids) are present on the same expression vector. In certain embodiments, at least two of the first, second, or third nucleic acids (e.g., engineered nucleic acids) are operably linked to the same promoter. In certain embodiments, all three of the first, second, and third nucleic acids (e.g., engineered nucleic acids) are operably linked to the same promoter.

In some embodiments, the expression vector (e.g., viral expression vector, including lentiviral, retroviral, adeno-associated viral vectors) comprises an inducible promoter (e.g., a promoter comprising a tetracycline-responsive element (TRE) including a TRE3G sequence, a TRE2 sequence, or a P tight sequence), and the method further comprises administering an inducing agent (e.g., a chemical agent, a nucleic acid (e.g., engineered nucleic acid) (e.g., nucleic acid (e.g., engineered nucleic acid) encoding an inducing agent), a protein, light, or temperature). In some embodiments, a pH is used to induce expression of a nucleic acid operably linked to a promoter. In certain embodiments, a chemical agent capable of modulating the activity of an inducing agent is tetracycline (e.g., doxycycline). As a non-limiting example, tetracycline-controlled transactivator (tTA) is an inducing agent whose activity is inhibited by tetracycline. As a non-limiting example, reverse tetracycline-controlled transactivator (rtTA) is an inducing agent whose activity is activated by tetracycline. The inducing agent (e.g., rtTA or (TA) may be encoded by a fourth nucleic acid (e.g., engineered nucleic acid) that is administered nucleic acid. In certain embodiments, the inducing agent (e.g., a chemical agent, a nucleic acid (e.g., engineered nucleic acid) (e.g., a nucleic acid comprising RNA and/or DNA encoding an inducing agent), a protein, light, a particular pH, or temperature) is introduced simultaneously with the nucleic acids (e.g., engineered nucleic acids) encoding OCT4, SOX2, and KLF4. In certain embodiments, the inducing agent (e.g., a chemical agent, a nucleic acid (e.g., engineered nucleic acid) (e.g., a nucleic acid comprising RNA and/or DNA encoding an inducing agent), a protein, light, a particular pH, or temperature) is introduced simultaneously with the nucleic acids (e.g., engineered nucleic acids) encoding one or more (e.g., two or more or three or more) transcription factors selected from OCT4; SOX2; KLF4; and any combinations thereof. A promoter (e.g., constitutive promoter, including CAG and Ubc, or an inducible promoter) may be operably linked to the nucleic acid (e.g., engineered nucleic acid) encoding the inducing agent. In certain embodiments, the promoter operably linked to the nucleic acid (e.g., engineered nucleic acid) encoding the inducing agent is a tissue-specific promoter.

In certain embodiments, the nucleic acid (e.g., engineered nucleic acid) encoding the inducing agent is present on the same expression vector as at least one of the nucleic acids (e.g., engineered nucleic acids) encoding OCT4, SOX2, KLF4, or a combination thereof. In certain embodiments, the nucleic acid (e.g., engineered nucleic acid) encoding the inducing agent is present on a separate expression vector from the nucleic acid (e.g., engineered nucleic acid) encoding OCT4, the nucleic acid (e.g., engineered nucleic acid) SOX2, and the nucleic acid (e.g., engineered nucleic acid) encoding KLF4. In certain embodiments, the nucleic acids (e.g., engineered nucleic acids) encoding OCT4, SOX2, and KLF4 are present on a first expression vector, and the fourth nucleic acid (e.g., engineered nucleic acid) is present on a second expression vector. In some embodiments, a vector comprises a WPRE sequence and/or a hGH pA terminator sequence.

In some embodiments, a nucleic acid encoding OCT4, SOX2, KLF4, and/or an inducing agent is not present on a viral vector. In some embodiments, a nucleic acid encoding one or more (e.g., two or more or three or more) transcription factors selected from the group consisting of OCT4; SOX2; KLF4; and any combinations thereof is not present on a viral vector. In some embodiments, the nucleic acid is delivered without a viral vector. In some embodiments, delivery of the nucleic acid that is not on a viral vector comprises administration of a naked nucleic acid, electroporation, use of a nanoparticle, and/or use of a liposome.

The expression vectors may be viral vectors (e.g., lentivirus vectors, adenovirus vectors, retrovirus vectors, herpes virus vectors, alphavirus, vaccinia virus, or AAV vectors). For example, the first expression vector encoding OCT4, SOX2, and KLF4 may comprise a sequence that is at least 70% (e.g., at least 75%, 80%, 85%, 90%, 95%, 98%, 99%, or 100%) identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. In certain embodiments, the fourth nucleic acid (e.g., engineered nucleic acid) encoding the inducing agent further comprises an SV-40-derived terminator sequence, including a sequence that is at least 70% (e.g., at least 75%, 80%, 85%, 90%, 95%. 98%, 99%, or 100%) identical to SEQ ID NO: 8 or 143.

In certain embodiments, the inducing agent is capable of inducing expression from the inducible promoter in the presence of tetracycline (e.g., doxycycline). In certain embodiments, the inducing agent is rtTA (e.g., rtTA3, including rtTA3 with a sequence that is at least 70% (e.g., at least 75%, 80%, 85%, 90%, 95%, 98%, 99%, or 100%) identical to SEQ ID NO: 11, rtTA Advanced, including rtTA Advanced with a sequence that is at least 70% (e.g., at least 75%, 80%, 85%, 90%, 95%, 98%, 99%, or 100%) identical to SEQ ID NO: 129, rtTA4, including rtTA4 with a sequence that is at least 70% (e.g., at least 75%, 80%, 85%, 90%, 95%, 98%, 99%, or 100%) identical to SEQ ID NO: 13, and rtTA2S-M2, including rtTA2S-M2 with a sequence that is at least 70% (e.g., at least 75%, 80%, 85%, 90%, 95%, 98%, 99%, or 100%) identical to SEQ ID NO: 15). In certain embodiments, the method further comprises administering tetracycline (e.g., doxycycline) to the cell, tissue, or subject. In certain embodiments, the method comprises removing tetracycline (e.g., doxycycline) from the cell, tissue, or subject.

### Recombinant Viruses

Aspects of the present disclosure provide recombinant viruses (e.g., lentiviruses, alphaviruses, vaccinia viruses, adenoviruses, herpes viruses, retroviruses, or AAVs), which may be useful in delivering a transcription factor and/or inducing agent to a cell, tissue, or organ of the central nervous system. The recombinant viruses (e.g., lentiviruses, alphaviruses, vaccinia viruses, adenoviruses, herpes viruses, retroviruses, or AAVs) may harbor a nucleic acid (e.g., engineered nucleic acid) (e.g., expression vector) encoding a transcription factor (e.g., OCT4, SOX2, KLF4, or any combination thereof), and/or encoding an inducing agent. In some embodiments, a recombinant virus harbors a nucleic acid encoding at least two transcription factors selected from OCT4, SOX2, and KLF4 (e.g., OCT4 and SOX2; KLF4 and SOX2; OCT4, KLF4, and SOX2; or OCT4 and KLF4). In some embodiments, a recombinant virus harbors a nucleic acid encoding at least three transcription factors selected from OCT4, SOX2, and KLF4 (e.g., OCT4, SOX2, and KLF4). In some instances, a recombinant virus of the present disclosure comprises a nucleic acid encoding an inducing agent.

In certain embodiments, recombinant virus is a recombinant AAV. In some embodiments, a recombinant AAV has tissue-specific targeting capabilities, such that a transgene of the AAV will be delivered specifically to one or more predetermined tissue(s). In some embodiments, a recombinant AAV is capable of targeting the central nervous system. Generally, the AAV capsid is a relevant factor in determining the tissue-specific targeting capabilities of an AAV. An AAV capsid may comprise an amino acid sequence derived from AAV1, AAV2, AAV3, AAV4, AAV5, AAV6, AAV7, AAV8, AAV9, AAV10, AAV11, AAV PHP.b and variants thereof. In certain embodiments, the AAV serotype is a variant of AAV9 (e.g., AAV PHP.b). In certain embodiments, the AAV serotype is AAV PHP.eB.Non-limiting examples of the tissue-specificity of AAV serotypes are provided in Table 1. An “x” indicates that the indicated AAV serotype is capable of delivering a transgene to a specific tissue.

In some embodiments, the AAV serotype delivers an engineered nucleic acid to the central nervous system. In some embodiments, the AAV serotype delivers an engineered nucleic acid to the brain. Non-limiting examples of AAV serotypes that deliver an engineered nucleic acid to the brain include AAV9, AAV10, AAV. PHP.b, AAV.PHP.eB, AAV.CAP-B10, and AAV.CAP-B22. In some embodiments, the AAV serotype used is not AAV2, is not AAV9, or a combination thereof. Without being bound by a particular theory, AAV2 and/or AAV9 may have poor delivery to the brain. See also, e.g., Goertsen et al., Nat Neurosci. 2022 January; 25 (1): 106-115, which is herein incorporated by reference in its entirety.

TABLE 1
Non-limiting examples of AAV serotypes and their utility in specific tissues.

| | Relevant Tissue | | | | | | | | |
|---|---|---|---|---|---|---|---|---|---|
| | | | | | | | | | Immune |
| | | | | | | Central | | | System |
| | | | | | | Nervous | | | (T-cells, |
| | | | Muscle | | Central | System | | | B-cells |
| | | | (e.g., | | Nervous | (Blood- | | | and |
| AAV | | | Skeletal | | System | brain | | | Dendritic |
| serotype | Liver | Heart | Muscle) | Eye | (CNS) | barrier) | Pancreas | Lung | Cells) |
| AAV1 | | x | x | | x | | | | |
| AAV2 | x | | x | x | x | | | | |
| AAV3 | x | | x | x | | | | x | |
| AAV4 | | | x | x | x | | | | |
| AAV5 | | | | x | x | | x | x | |
| AAV6 (e.g., | | x | x | | | | | x | x |
| AAV6.2) | | | | | | | | | |
| AAV7 | x | | x | | | | | | |
| AAV8 | x | | x | | x | | x | | |
| AAV9 | x | x | x | x | x | x | x | x | |
| AAV10 (e.g., | x | x | x | x | x | x | x | x | |
| AAVrh10) | | | | | | | | | |
| AAVDJ | x | | x | | x | | | | |
| AAV.PHP.b | | | | | x | x | | | |
| AAV.PHP.eB | | | | | x | x | | | |
| AAV.CAP-B10 | | | | | x | x | | | |
| AAV.CAP-B22 | | | | | x | x | | | |
| AAV-BI30 | | | | | x | x | | | |

Recombinant AAVs comprising a particular capsid protein may be produced using any suitable method. See, e.g., U.S. Patent Application Publication, US 2003/0138772, which is incorporated herein by reference. AAV capsid protein sequences also known in the art. See, e.g., Published PCT Application, WO 2010/138263, which is incorporated herein by reference. Generally, recombinant AAV is produced in a host cell with the following components: (1) a nucleic acid (e.g., engineered nucleic acid) sequence encoding an AAV capsid protein or a fragment thereof, (2) a nucleic acid (e.g., engineered nucleic acid) encoding a functional rep gene, (3) a recombinant AAV vector comprising AAV inverted terminal repeats flanking a transgene (e.g., nucleic acids (e.g., engineered nucleic acids) encoding OCT4, KLF4, SOX2, or a combination thereof), and (4) helper functions that allow for packaging of the recombinant AAV vector into AAV capsid proteins. In some instances, a recombinant AAV vector comprises a nucleic acid encoding an inducing agent. In certain embodiments, the helper functions are introduced via a helper vector that is known in the art. See, e.g., Chan et al. Nat Neurosci. 2017 August; 20 (8): 1172-1179.

In some embodiments, an AAV capsid comprises a targeting moiety for one or more cells of the central nervous system. See, e.g., WO2023004367 and WO2022232327. In some embodiments, the AAV capsid is AAV-B130. See, e.g., Krolak et al., Nat Cardiovasc Res. 2022 April; 1 (4): 389-400.

In some instances, a suitable host cell line (e.g., HEK293T cells) may be used for producing a recombinant AAV disclosed herein following routine practice. One or more expression vectors encoding one or more of the components described above may be introduced into a host cell by exogenous nucleic acids (e.g., engineered nucleic acids), which can be cultured under suitable conditions allowing for production of AAV particles. When needed, a helper vector can be used to facilitate replication, to facilitate assembly of the AAV particles, or any combination thereof. In certain embodiments, the recombinant AAV vector is present on a separate nucleic acid (e.g., engineered nucleic acid) from the other components (e.g., a nucleic acid (e.g., engineered nucleic acid) sequence encoding an AAV capsid protein or a fragment thereof, a nucleic acid (e.g., engineered nucleic acid) encoding a functional rep gene, and helper functions that allow for packaging of the recombinant AAV vector into AAV capsid proteins. In certain embodiments, a host cell may stably express one or more components needed to produce AAV virus. In that case, the remaining components may be introduced into the host cell. The supernatant of the cell culture may be collected, and the viral particles contained therein can be collected via routine methodology.

In some embodiments, the methods described herein include incorporation of one or more nucleic acids into viruses (e.g., adeno-associated virus (AAV) at a high viral titer (e.g., more than 2×10¹² particles per preparation, 1×10¹³ particles per mL), which may be useful in reversing aging, and treating neurological diseases. In some embodiments, a preparation comprises use of ten T150 flasks of HEK293T cells.

### Methods of Activating OCT4, SOX2, and KLF4, Each Alone or in Combination, and Replacements Thereof

Aspects of the present disclosure, in some embodiments, relate to activating OCT4, SOX2, and KLF4, each alone or in combination, in a cell, tissue and/or organ of the central nervous system. In some embodiments, OCT4, SOX2, and KLF4, each alone or in combination, is activated in the absence of c-Myc activation. The cell, tissue, and/or organ may be in vivo (e.g., in a subject) or be ex vivo. As used herein, activation includes any nucleic acid (e.g., nucleic acid comprising RNA, comprising DNA, or any combination thereof), protein, antibody, chemical agent, or any combination thereof that is capable of increasing the biological activity of a protein of interest (e.g., OCT4, SOX2, and/or KLF4). Biological activity (e.g., gene expression, reprogramming ability, transcription factor activity, etc.) may be measured using any routine method known in the art. In some embodiments, any nucleic acid (e.g., nucleic acid comprising RNA, comprising DNA, or any combination thereof), protein, antibody, chemical agent, or any combination thereof described herein replaces OCT4, SOX2 and/or KLF4. In some embodiments, any nucleic acid (e.g., nucleic acid comprising RNA, comprising DNA, or any combination thereof), protein, antibody, chemical agent, or any combination thereof described herein replaces OCT4, SOX2, KLF4, or any combination thereof. In some embodiments, any of the nucleic acids (e.g., engineered nucleic acid) encoding an inducing agent, engineered proteins encoding an inducing agent, chemical agents capable of modulating (e.g., activating or inhibiting) the activity of an inducing agent, and/or recombinant viruses encoding an inducing agent described herein is used to activate an inducing agent.

Activation of OCT4, SOX2, and KLF4, each alone or in combination includes increasing expression (e.g., RNA and/or protein expression) of OCT4, SOX2, and KLF4, each alone or in combination. In some embodiments, the expression of OCT4, SOX2, and KLF4, each alone or in combination is increased by at least 1%, 5%, 10%, 20%, 30%, 40%, 50%, 60%, 70%, 80%, 90%, 100%, 200%, 300%, 400%, 500%, 600%, 700%, 800%, 900%, or 1000% after administration of a nucleic acid (e.g., nucleic acid comprising RNA, comprising DNA, or any combination thereof) encoding OCT4, SOX2, and/or KLF4, protein encoding OCT4, SOX2, and/or KLF4, antibody capable of activating encoding OCT4, SOX2, and/or KLF4, chemical agent capable of activating encoding OCT4, SOX2, and/or KLF4, or any combination thereof to a cell, tissue, organ, and/or subject compared to before administration. In some embodiments, the expression of OCT4, SOX2, and KLF4, each alone or in combination is increased by at least 1%, 5%, 10%, 20%, 30%, 40%, 50%, 60%, 70%, 80%, 90%, 100%, 200%, 300%, 400%, 500%, 600%, 700%, 800%, 900%, or 1000% after administration of a nucleic acid (e.g., nucleic acid comprising RNA, comprising DNA, or any combination thereof) encoding OCT4, SOX2, KLF4, or any combination thereof, protein encoding OCT4, SOX2, KLF4, or any combination thereof, antibody capable of activating encoding OCT4, SOX2, KLF4, or any combination thereof, chemical agent capable of activating encoding OCT4, SOX2, KLF4, or any combination thereof, or any combination thereof to a cell, tissue, organ, and/or subject compared to before administration.

Activation of an inducing agent includes increasing expression (e.g., RNA and/or protein expression) of an inducing agent. In some embodiments, the expression of an inducing agent, is increased by at least 1%, 5%, 10%, 20%, 30%, 40%, 50%, 60%, 70%, 80%, 90%, 100%, 200%, 300%, 400%, 500%, 600%, 700%, 800%, 900%, or 1000% after administration of a nucleic acid (e.g., nucleic acid comprising RNA, comprising DNA, or any combination thereof) encoding the inducing agent, protein encoding the inducing agent, chemical agent capable of modulating the activity of the inducing agent, or any combination thereof to a cell, tissue, organ, and/or subject compared to before administration.

Expression may be measured by any routine method known in the art, including quantification of the level of a protein of interest (e.g., using an ELISA, and/or western blot analysis with antibodies that recognize a protein of interest) or quantification of RNA (e.g., mRNA) levels for a gene of interest (e.g., using reverse transcription polymerase chain reaction).

In addition to the engineered nucleic acids discussed herein, OCT4, SOX2, KLF4, alone or in combination may be activated in a cell, tissue, organ, and/or subject through the use of engineered proteins. For example, protein encoding OCT4, SOX2, and/or KLF4 may be generated (e.g., recombinantly or synthetically) and administered to a cell, tissue, organ, and/or subject through any suitable route. For example, protein encoding one or more transcription factors selected from the group consisting of OCT4; SOX2; KLF4; and any combinations thereof may be generated (e.g., recombinantly or synthetically) and administered to a cell, tissue, organ, and/or subject through any suitable route.

In some embodiments, activating expression of OCT4; SOX2; KLF4; a replacement thereof; or any combination thereof from a tetracycline-inducible expression vector comprises administering a tetracycline (e.g., doxycycline) to a cell, organ, tissue, or a subject. As one of ordinary skill in the art would appreciate, the route of tetracycline administration may be dependent on the type of cell, organ, tissue, and/or characteristics of a subject. In some embodiments, tetracycline is administered directly to a cell, organ, and/or tissue. As a non-limiting example, tetracycline may be administered to the eye of a subject through any suitable method, including eye drops comprising tetracycline, sustained release devices (e.g., micropumps, particles, and/or drug depots), and medicated contact lenses comprising a tetracycline). In some embodiments, tetracycline is administered systemically (e.g., through drinking water or intravenous injection) to a subject. Tetracycline may be administered topically (e.g., in a cream) or through a subcutaneous pump (e.g., to deliver tetracycline to a particular tissue). Tetracycline may be administered intravenously, intradermally, intraarterially, intralesionally, intratumorally, intracranially, intraarticularly, intraprostatically, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in particles (e.g., nanoparticles, microparticles), in lipid compositions (e.g., liposomes), or by other method or any combination of the forgoing as would be known to one of ordinary skill in the art (see, for example, Remington's Pharmaceutical Sciences (1990), incorporated herein by reference).

As a non-limiting example, an engineered protein may be further modified or formulated for delivery to a cell, tissue, organ, and/or subject. For example, protein transduction domains (i.e., PTD or cell-penetrating peptides) may be attached to an engineered protein (e.g., OCT4, SOX2, and/or KLF4). As a non-limiting example, a protein transduction domain (i.e., PTD or cell-penetrating peptide) may be attached to an engineered protein encoding an inducing agent. Without being bound by a particular theory, a protein transduction domain facilitate delivery of a cargo (e.g., a protein, nucleic acids, nanoparticles, viral particles, etc.) across cellular membranes. Protein transduction domains include cationic peptides, hydrophobic peptides, and/or a cell specific peptides. See, e.g., Zhou et al., Cell Stem Cell. 2009 May 8; 4 (5): 381-4; Zahid et al., Curr Gene Ther. 2012 October; 12 (5): 374-80.

In some embodiments, a protein encoding OCT4, SOX2, and/or KLF4, and/or a protein encoding an inducing agent is formulated in a nanoparticle (e.g., for nuclear delivery). In some embodiments, a protein encoding OCT4, SOX2, KLF4, or any combination thereof (e.g., OCT4 and SOX2; KLF4 and SOX2; OCT4 and KLF4; or KLF4, SOX2, and OCT4) is formulated in a nanoparticle (e.g., for nuclear delivery). In certain embodiments, a nanoparticle further comprises a protein encoding an inducing agent. For example, chitosan [poly(N-acetyl glucosamine)] is a biodegradable polysaccharide and may be used to formulate nanoparticles by several methods. In some embodiments, a chitosan polymeric nanoparticle is loaded with protein encoding OCT4, SOX2, and/or KLF4, and/or an inducing agent and is delivered to the nucleus of a cell. See, e.g., Tammam et al., Oncotarget. 2016 Jun. 21; 7 (25): 37728-37739.

In some embodiments, a chemical agent, antibody and/or protein replaces OCT4, SOX2, and/or KLF4. In some embodiments, a chemical agent, antibody, a protein, or any combination thereof replaces OCT4, SOX2, KLF4, or any combination thereof (e.g., OCT4 and SOX2; OCT4 and KLF4; KLF4 and SOX2; or KLF4, SOX2, and OCT4). For example, a chemical agent, antibody and/or protein may promote expression of OCT4, SOX2, and/or KLF4. In certain instances, a chemical agent, antibody and/or protein may promote expression of one or more transcription factors selected from OCT4; SOX2; KLF4; and any combinations thereof. In some embodiments, a chemical agent, antibody and/or protein may activate target genes downstream of OCT4, SOX2, and/or KLF4. In some embodiments, a chemical agent, antibody, a protein, or any combination thereof may activate target genes downstream of one or more transcription factors selected from the group consisting of OCT4; SOX2; KLF4; and any combinations thereof. In some embodiments, a chemical agent, antibody and/or protein is said to replace OCT4, SOX2, and/or KLF4 if the chemical agent, antibody and/or protein may be used together with the other two transcription factors and promote cellular reprogramming. In some embodiments, a chemical agent, antibody, protein, or any combination thereof is said to replace OCT4, SOX2, KLF4, or any combination thereof if the chemical agent, antibody, protein or any combination thereof may be used together with the other two transcription factors and promote cellular reprogramming. For example, cellular reprogramming may be determined by measuring gene expression (e.g., expression of embryonic markers and/or pluripotency markers). In some embodiments, pluripotency markers include AP, SSEA1, and/or Nanog.

In some embodiments, an antibody is used to activate OCT4, SOX2, and/or KLF4. In some embodiments, an antibody is used to activate one or more transcription factors selected from OCT4, SOX2, KLF4, or any combination thereof. In some embodiments, the antibody does not target OCT4, SOX2, and/or KLF4. In some embodiments, the antibody does not target OCT4, SOX2, KLF4, or any combination thereof. In some embodiments, the antibody increases expression of OCT4, SOX2, and/or KLF4. In some embodiments, the antibody increases expression of OCT4, SOX2, KLF4, or any combination thereof. In some embodiments, the antibody does not increase expression of OCT4, SOX2, and/or KLF4. In some embodiments, an antibody replaces OCT4, SOX2, and/or KLF4. In some embodiments, the antibody does not increase expression of OCT4, SOX2, KLF4, or any combination thereof. In some embodiments, an antibody replaces OCT4, SOX2, KLF4, or any combination thereof. Any suitable method of identifying antibodies that can replace a transcription factor (e.g., OCT4, SOX2, and/or KLF4) may be used. Any suitable method of identifying antibodies that can replace a transcription factor (e.g., OCT4, SOX2, KLF4, or any combination thereof) may be used. See, e.g., Blanchard et al., Nat Biotechnol. 2017 October; 35 (10): 960-968.

In some embodiments, another protein (e.g., a nucleic acid encoding the protein or a polypeptide encoding the protein) may be used to replace OCT4, SOX2, and/or KLF4. In some embodiments, another protein (e.g., a nucleic acid encoding the protein or a polypeptide encoding the protein) may be used to replace OCT4, SOX2, KLF4, or a combination thereof. For example, OCT4 may be replaced by Tet1, NR5A-2, Sall4, E-cadherin, NKX3-1, or any combination thereof. In some embodiments, OCT4, SOX2, and/or KLF4 may be replaced by NANOG and/or TET2. In some embodiments, OCT4, SOX2, KLF4, or any combination thereof may be replaced by NANOG and/or TET2. See, e.g., Nat Cell Biol. 2018 August; 20 (8): 900-908; Gao et al., Cell Stem Cell. 2013 Apr. 4; 12 (4): 453-69. Nanog and Lin28 can replace Klf4. See, e.g., Yu et al, Science. 318, 1917-1920, 2007). In some embodiments, OCT4, SOX2, and/or KLF4 is replaced by Tet3 (tet methylcytosine dioxygenase 3). In some embodiments, OCT4, SOX2, KLF4, or any combination thereof is replaced by Tet3 (tet methylcytosine dioxygenase 3). In some embodiments, a nucleic acid encoding a Tet1 DNA demethylase comprises a sequence that is at least 70% (e.g., at least 75%, 80%, 85%, 90%, 95%, 98%, 99%, or 100%) identical to NM_030625.3 or NM_001253857.2. In some embodiments, an amino acid encoding a Tet1 DNA demethylase comprises a sequence that is at least 70% (e.g., at least 75%, 80%, 85%, 90%, 95%, 98%, 99%, or 100%) identical to NP_085128.2 or NP_001240786.1. In some embodiments, a nucleic acid encoding a Tet2 DNA demethylase comprises a sequence that is at least 70% (e.g., at least 75%, 80%, 85%, 90%, 95%, 98%, 99%, or 100%) identical to NM_001127208.2, NM_001040400.2, NM_001346736.1, or NM_017628.4. In some embodiments, an amino acid encoding a Tet2 DNA demethylase comprises a sequence that is at least 70% (e.g., at least 75%, 80%, 85%, 90%, 95%, 98%, 99%, or 100%) identical to NP_060098.3, NP_001035490.2, NP_001333665.1, or NP_001120680.1. In some embodiments, a nucleic acid encoding a Tet3 DNA demethylase comprises a sequence that is at least 70% (e.g., at least 75%, 80%, 85%, 90%, 95%, 98%, 99%, or 100%) identical to NM_001287491.2, NM_001347313.1, NM_183138.2, or NM_001366022.1. In some embodiments, an amino acid encoding a Tet3 DNA demethylase comprises a sequence that is at least 70% (e.g., at least 75%, 80%, 85%, 90%, 95%, 98%, 99%, or 100%) identical to NP_001274420.1, NP_001334242.1, NP_898961.2, or NP_001352951.1. Tet1. Tet2, and/or Tet3 may be derived from any species. In some embodiments, Tet1, Tet2, and/or Tet3 is a truncated form of a wild-type counterpart. As a non-limiting example, Tet1, Tet2, and/or Tet3 is N-terminally truncated compared to a wild-type Tet1, Tet2, and/or Tet3 counterpart and is catalytically active. In some embodiments, Tet1, Tet2, and/or Tet3 only comprises the catalytic domain of Tet1, Tet2, and/or Tet3. In some embodiments, Tet1, Tet2, and/or Tet3 comprises the catalytic domain of Tet1, Tet2, Tet3, or any combination thereof. Non-limiting examples of functional truncated Tet1 may be found in Hrit et al., Elife. 2018 Oct. 16; 7. Pii: e34870.

Additional methods of replacing OCT4, SOX2, and/or KLF4 to promote cellular reprogramming are known in the art. See, e.g., Heng et al., Cell Stem Cell 6, 167-174 (2010); Eguchi et al., Proc. Natl Acad. Sci. USA 113, E8257-E8266 (2016); Gao et al., Cell Stem Cell 12, 453-469 (2013); Long et al., Cell Res. 25, 1171-1174 (2015); Hou et al., Science 341, 651-654 (2013); Redmer et al., EMBO Rep. 12, 720-726 (2011); Tan et al., J. Biol. Chem. 290, 4500-4511 (2014); Anokye-Danso et al., Cell Stem Cell 8, 376-388 (2011); Miyoshi et al., Cell Stem Cell 8, 633-638 (2011); Shu et al., Cell 153, 963-975 (2013); Yu, J. et al., Science 318, 1917-1920 (2007).

In some embodiments, a chemical agent replaces OCT4, SOX2, and/or KLF4 (e.g., can be used in place of OCT4, SOX2, and/or KLF4 along with the other two transcription factors to promote cellular reprogramming). In some embodiments, a chemical agent replaces OCT4, SOX2, KLF4, or any combination thereof (e.g., can be used in place of OCT4, SOX2, KLF4, or any combination thereof, along with the other two transcription factors to promote cellular reprogramming). For example, SOX2 may be replaced by CHIR, FSK, or 616452. OCT4 may be replaced by DZNep. Since Sall4 may be used to replace OCT4 as mentioned above, any compound that replaces Sall4 may also be used to replace OCT4. For example, CHIR, FSK, and 616452 may be used to replace Sall4. Nanog may be replaced with 2i medium. See, e.g., Hou et al., Science. 2013 Aug. 9; 341 (6146): 651-4. See, also, e.g., Zhao et al., Cell. 2015 Dec. 17; 163 (7): 1678-91.

In some embodiments, chemical reprogramming comprises using chemicals that reduce the toxicity of chemical agents that induce reprogramming. Non-limiting examples of chemicals that reduce the toxicity of chemical reprogramming include ROCK inhibitors (e.g., Y27632 and Fasudil) and P38 MAPK inhibitors (e.g., SB203580 and BIRB796). See, e.g., Li et al., Cell Stem Cell. 2015 Aug. 6; 17 (2): 195-203.

OCT4, KLF4, SOX2, replacements, or any combination thereof may be activated (e.g., expression may be induced) in combination with activating an enhancer of reprogramming and/or inhibiting a barrier of reprogramming. An enhancer of reprogramming may be activated using any suitable method known in the art, including overexpression of the enhancer, increasing expression of an endogenous gene encoding the enhancer (e.g., using CRISPR technology), use of a chemical agent and/or antibody to increase the biological activity of the enhancer, and use a chemical agent and/or antibody to promote expression of the enhancer. A barrier of reprogramming may be inhibited using any suitable method known in the art, including knocking down expression of the inhibitor (e.g., with siRNAs, miRNAs, shRNAs), knocking out an endogenous copy of the inhibitor (e.g., using CRISPR technology, TALENs, zinc finger nucleases, etc.), using a chemical agent and/or antibody to decrease the biological activity of the inhibitor, and using a chemical agent and/or antibody to decrease expression of the inhibitor.

Non-limiting examples of enhancers and barriers of reprogramming are provided in Table 2. See also, e.g., Ebrahimi, Cell Regen (Lond). 2015 Nov. 11; 4:10, which is herein incorporated by reference in its entirety.

TABLE 2
Non-limiting examples of strategies to enhance reprogramming.

| Reprogramming | |
|---|---|
| Enhancing Strategy | Enhancers |
| Activation of | C/EBPα; UTF1; Mef2c; Tdgf1; FOXH1; GLIS1; mutated |
| Enhancers | reprogramming factors, MDM2; Bcl-2; CCL2; Kdm3a, Kdm3b, Kdm4c, |
| | and Kdm4b/2b; Jhdm1a/1b; MOF; Mbd1-4 (or their small molecule |
| | activators); Wnt/β-catenin signaling; small molecule Pitstops 1 and 2; |
| | vitamin C, palbiociclib; cytokines, e.g. IL-6; CDK4, CDK8, CDK19; lincU |
| Inhibition of | Barriers |
| Barriers | p53, p57, p38, p16^{Ink4a}/p19^{Arf}, p21^{Cip1}, Rb |
| | TGF-β, MAP kinase, Aurora A kinase, MEK/ERK, Gsk3, Wnt/β-catenin |
| | signaling pathways, LATS2, PKC, IP3K, CDK8, CDK19. |
| | Native/somatic gene or transcriptional regulatory network (GRN/TRN). |
| | Specific members of ADAM family (e.g., ADAM7, ADAM21, |
| | ADAM29), endocytosis: (e.g., DRAM1, SLC17A5, ARSD), |
| | phosphatase: (e.g., PTPRJ, PTPRK, PTPN11). |
| | Chromatin regulators: (e.g., ATF7IP, MacroH2A, Mbd1-4, Setdb1a. |
| | Transcription factors: (e.g., TTF1, TTF2, TMF1, T), Bright. |
| | Fbxw7 (a member of ubiquitin-proteasome system (UPS)) |
| | Lzts1, Ssbp3, Arx, Tfdp1, Nfe2, Ankrd22, Msx3, Dbx1, Lasp1, and Hspa8. |
| | Cytokines e.g., TNFα |
| | Cells (e.g., senescent cells and NK cells) (e.g., navitoclax, BAY117082) |
| | NuRD, Mbd1-4, Gatad2a, Chd4 (see, e.g., Mor et al., Cell Stem Cell. |
| | 2018 Sep. 6; 23(3): 412-425. e10) |
| | KDM1a |
| | Kaiso (see, e.g., Kaplun et al., Biochemistry (Mosc). 2019 |
| | Mar; 84(3): 283-290) |

Additional reprogramming enhancers that may be activated in combination with activation of OCT4, KLF4, SOX2, replacements thereof, or any combination thereof, include histone lysine demethylases (e.g., KDM2, KDM3, and KDM4). Histone lysine demethylases may be activated by being overexpressed in a cell, tissue, organ, and/or a subject. Chemical activators of histone lysine demethylases are also encompassed by the present disclosure. For example, vitamin C may be used to activate KDM3 and/or KDM4.

In some embodiments, OCT4, SOX2, KLF4, replacements thereof, or any combination thereof, is activated along with activation of C/EBPa and Tfcp211. Without being bound by a particular theory, C/EBPa, and Tfcp211 together with Klf4 may drive Tet2-mediated enhancer demethylation and activation during reprogramming.

In some embodiments, OCT4, SOX2, KLF4, replacements thereof, or any combination thereof are activated in a cell, tissue, organ and/or a subject in combination with a cytokine that facilitates reprogramming. IL6 is a non-limiting example of a cytokine. See, e.g., Mosteiro et al, Science. 2016 Nov. 25; 354 (6315), which is herein incorporated by reference in its entirety.

In some embodiments, OCT4, SOX2, KLF4, replacements thereof, or any combination thereof are activated in a cell, tissue, organ and/or a subject in combination with activation of a miRNA (e.g., administration of a miRNA and/or expression of a miRNA). For example, a miRNA that promotes cell cycle progression may be introduced to a cell, tissue, organ, and/or subject. Non-limiting examples of miRNAs that promote cell cycle progression include miR 302-367, miR 371-373, miR-200b, miR-200c, miR-205, miR 290-295, miR-93, miR-106, and miR 135b.

As a non-limiting example, nerve regeneration may be enhanced by combining activation of OCT4, SOX2, KLF4, replacements thereof, or any combination thereof with activation of an enhancer. Non-limiting activation of enhancers include overexpression of a member of the KLF family (e.g., KLF7), overexpression of c-Myc, STAT3 activation, SOX11 overexpression, overexpression of Lin28, overexpression of or delivery of soluble protein encoding insulin-like growth factor 1 (IGF1) and osteopontin (OPN), and activation of B-RAF (e.g., introduction of a gain of function mutation). See also, e.g., Blackmore et al., Proc Natl Acad Sci USA. 2012 May 8; 109 (19): 7517-22; Belin et al., Neuron. 2015 May 20; 86 (4): 1000-1014; Bareyre et al., Proc Natl Acad Sci USA. 2011 Apr. 12; 108 (15): 6282-7; Norsworthy et al., Neuron. 2017 Jun. 21; 94 (6): 1112-1120.e4; Wang et al., Cell Rep. 2018 Sep. 4; 24 (10): 2540-2552.e6; Liu et al., Neuron. 2017 Aug. 16; 95 (4): 817-833; O'Donovan et al., J Exp Med, 2014. 211 (5): p. 801-14, which is herein incorporated by reference in its entirety.

In some embodiments, OCT4, SOX2, KLF4, replacements thereof, or any combination thereof, are activated in a cell, tissue, organ, and/or a subject in combination with suppression or knockdown of reprogramming barriers. Non-limiting examples of reprogramming barriers include Chaf1a, Chaf1b, Ube2i, sumo2, and/or Nudt21. See, e.g., Brumbaugh et al., Cell. 2018 Jan. 11; 172 (1-2): 106-120.e21; Cheloufi et al., Nature. 2015 Dec. 10; 528 (7581): 218-24; and Borkent et al., Stem Cell Reports, 2016. 6 (5): p. 704-716, which is herein incorporated by reference in its entirety.

As a non-limiting example, a reprogramming barrier may be a DNA methyltransferase (DNMT) may be and a DNMT may be inhibited to promote reprogramming of a tissue, cell, and/or organ. Most DNA methyltransferases use S-adenosyl-L-methionine as a methyl donor. DNMT may be from any species. There are at least three different types of methyltransferases. M6A methyltransferases are capable of methylating the amino group at the c-6 position of adenines in DNA (e.g., Enzyme Commission (EC) No. 2.1.1.72). m4C methyltransferases are capable of generating N4-methylcytosine (e.g., Enzyme Commission (EC) No. 2.1.1.113). M5C methyltransferases are capable of generating C5-methylcytosine (e.g., Enzyme Commission (EC) No. 2.1.1.37).

Non-limiting examples of mammalian DNA methyltransferases (DNMTs) include DNMT1 and its isoforms DNMT1b and DNMT10 (oocytes-specific), DNMT3a, DNMT3b, DNMT3L. GenBank Accession Nos. NM_001130823.3 (isoform a), NM_001318730.1 (isoform c), NM_001318731.1 (isoform d), and NM_001379.3 (isoform b) are non-limiting examples of nucleotide sequences encoding human DNMT1. A nucleic acid encoding a DNMT1 may comprise a sequence that is at least 70% (e.g., at least 75%, 80%, 85%, 90%, 95%, 98%, 99%, or 100%) identical to a sequence set forth in GenBank Accession Nos. NM_001130823.3 (isoform a), NM_001318730.1 (isoform c), NM_001318731.1 (isoform d), and/or NM_001379.3 (isoform b). GenBank Accession Nos. NP_001124295.1 (isoform a), NP_001305659.1 (isoform c), NP_001305660.1 (isoform d), and NP_001370.1 (isoform b) are non-limiting examples of amino acid sequences encoding human DNMT1. An amino acid sequence encoding a DNMT1 may comprise a sequence that is at least 70% (e.g., at least 75%, 80%, 85%, 90%, 95%, 98%, 99%, or 100%) identical to a sequence set forth in GenBank Accession Nos. NP_001124295.1 (isoform a), NP_001305659.1 (isoform c), NP_001305660.1 (isoform d), and/or NP_001370.1 (isoform b). A nucleic acid encoding human DNMT3A includes GenBank Accession No. NM_001320892.1, NM_001320893.1, NM_022552.4, NM_153759.3, NM_175629.2, and NM_175630.1. A nucleic acid encoding a DNMT3A may be at least 70% (e.g., at least 75%, 80%, 85%, 90%, 95%, 98%, 99%, or 100%) identical to a sequence set forth in GenBank Accession Nos. NM_001320892.1, NM_001320893.1, NM_022552.4, NM_153759.3, NM_175629.2, and/or NM_175630.1. An amino acid sequence encoding human DNMT3A includes GenBank Accession Nos. NP_001307821.1, NP_001307822.1, NP_072046.2, NP_715640.2, NP_783328.1, and NP_783329.1. An amino acid sequence encoding a DNMT3A may be at least 70% (e.g., at least 75%, 80%, 85%, 90%, 95%, 98%, 99%, or 100%) identical to a sequence set forth in GenBank Accession Nos. NP_001307821.1, NP_001307822.1, NP_072046.2, NP_715640.2, NP_783328.1, and/or NP_783329.1. A nucleic acid encoding human DNMT3B includes GenBank Accession No. NM_001207055.1, NM_001207056.1, NM_006892.3, NM_175848.1, NM_175849.1, and NM_175850.2. A nucleic acid encoding a DNMT3B may be at least 70% (e.g., at least 75%, 80%, 85%, 90%, 95%, 98%, 99%, or 100%) identical to a sequence set forth in GenBank Accession Nos. NM_001207055.1, NM_001207056.1, NM_006892.3, NM_175848.1, NM_175849.1, and/or NM_175850.2. An amino acid sequence encoding human DNMT3B includes GenBank Accession Nos. NP_001193984.1, NP_001193985.1, NP_008823.1, NP_787044.1, NP_787045.1, and NP_787046.1. An amino acid sequence encoding a DNMT3B may be at least 70% (e.g., at least 75%, 80%, 85%, 90%, 95%, 98%, 99%, or 100%) identical to a sequence set forth in GenBank Accession Nos. NP_001193984.1, NP_001193985.1, NP_008823.1, NP_787044.1, NP_787045.1, and/or NP_787046.1. A nucleic acid encoding human DNMT3L includes GenBank Accession No. NM_013369.3 and NM_175867.2. A nucleic acid encoding a DNMT3L may be at least 70% (e.g., at least 75%, 80%, 85%, 90%, 95%, 98%, 99%, or 100%) identical to a sequence set forth in GenBank Accession Nos. NM_013369.3 and/or NM_175867.2. An amino acid sequence encoding human DNMT3L includes GenBank Accession Nos. NP_037501.2 and NP_787063.1. An amino acid sequence encoding a DNMT3L may be at least 70% (e.g., at least 75%, 80%, 85%, 90%, 95%, 98%, 99%, or 100%) identical to a sequence set forth in GenBank Accession Nos. NP_037501.2 and/or NP_787063.1.

A DNMT may be inhibited using any suitable method known in the art. Suitable methods include knockdown of a DNMT mRNA, genetically knocking out a DNMT, and use of a DNMT inhibitor (e.g., chemical inhibitors). DNMT inhibitors are being investigated in clinical trials (e.g., phase III clinical trials) in the United States of America and beyond. Non-limiting examples of DNMT inhibitors include VIDAZA™ (azacitidine) (e.g., for the treatment of Myelodysplastic Syndromes and treatment of acute myeloid leukemia (AML)), DACOGEN™ (decitabine) (e.g., for treatment of AML and treatment of Chronic myeloid leukemia (CML)), and Guadecitabine (SGI-110) (e.g., for treatment of AML). In 2012, the European Union approved DACOGEN™ (decitabine) for use in patients with AML.

A DNMT may be inhibited by inhibiting a DNMT stabilizer. Suitable methods of inhibiting a DNMT stabilizer include knockdown of the mRNA encoding the stabilizer, genetically knocking out the gene that encodes the stabilizer and use of an inhibitor (e.g., chemical inhibitors). As a non-limiting example, KDM1a, which is also referred to as Lsd1 or Aof2, is a stabilizer of DNMT1. See, e.g., Wang et al., Nat Genet. 2009 January; 41 (1): 125-9. In some embodiments, KDM1a expression is knocked down using a shRNA disclosed herein or known in the art. In some embodiments, KDM1a is inhibited to prevent injury induced by hypermethylation from DNMTs, which could be useful in promoting reprogramming.

In some embodiments, a histone methyltransferase is a reprogramming barrier and is inhibited to facilitate reprogramming of a cell, tissue and/or organ. Histone methyltransferases may be inhibited by any suitable method, including use of chemical inhibitors. For example, 3-deazaneplanocin A (Dznep), epz004777, and BIX-01294 are examples of histone methyltransferase inhibitors.

In some embodiments, a reprogramming barrier is a histone deacetylase (HDAC) and a HDAC is inhibited to facilitate reprogramming of a cell, tissue, and/or organ. Non-limiting examples of HDAC inhibitors include valproic acid (VPA), trichostatin A (TSA), suberoylanilide hydroxamic Acid (SAHA), sodium butyrate (SB), Belinostat (PXD101), Panobinostat (LBH589), Quisinostat (JNJ-26481585), Abexinostat (PCI-24781), Givinostat (ITF2357), Resminostat (4SC-201), Phenylbutyrate (PBA), Depsipeptide (romidepsin), Entinostat (MS-275), Mocetinostat (MGCD0103), and Tubastatin A (TBA).

In some embodiments, a reprogramming barrier is a NF-κB, and it is inhibited to facilitate reprogramming of a cell, tissue, and/or organ. Non-limiting examples of NF-κB inhibitor includes BAY 11-7082, TPCA 1, and p65 siRNA. See, e.g., the NF-κB small molecule guide compiled by Abcam, which is available on the Abcam website (www.abcam.com/reagents/nf-kb-small-molecule-guide).

In some embodiments, a reprogramming barrier is a cytokine secreted from senescent cells in which a cytokine is inhibited to facilitate reprogramming of a cell, tissue, and/or organ. None limiting examples of cytokines inhibitors include Anti-TNFα (Mahmoudi et al, Biorxiv, 2018) and drugs, including Navitoclax, that kill senescence cells.

In some embodiments, a reprogramming barrier is a microRNA (miRNA) and a microRNA is inhibited to facilitate reprogramming of a cell, tissue, and/or organ. Non-limiting examples of microRNAs that are reprogramming barriers include miR Let-7 and miR-34. Without being bound by a particular theory, inhibition of miR Let-7 may increase the efficiency of reprogramming because miR Let-7 inhibits the cell cycle and inhibition of miR-34 may facilitate reprogramming because miR-34 inhibits the translation of p53.

In some embodiments, OCT4, SOX2, KLF4, replacements thereof, or any combination thereof is activated in a cell, tissue, organ and/or a subject in combination with inhibition of PTEN, SOCS3, RhoA, and/or ROCK to enhance nerve regeneration. In some embodiments, PTEN is deleted, SOCS3 is deleted, RhoA is knocked down, and/or ROCK is knocked down in a cell, tissue, organ and/or subject. See, e.g., Park et al., Science. 2008 Nov. 7; 322 (5903): 963-6; Smith et al., Neuron. 2009 Dec. 10; 64 (5): 617-23; Koch et al., Front Cell Neurosci. 2014 Sep. 5; 8:273; Koch et al., Cell Death Dis. 2014 May 15; 5:e1225 for descriptions of inhibition of PTEN, SOCS3, RhoA, and/or ROCK. Each reference is incorporated herein by reference in its entirety.

In some embodiments, OCT4, SOX2, KLF4, replacements thereof, or any combination thereof is activated in a cell, tissue, organ and/or a subject in combination with neuronal electrical stimulation (e.g., high-contrast visual stimulation) to promote nerve regeneration. See, e.g., Lim et al., Nat Neurosci. 2016 August; 19 (8): 1073-84 for a description of high-contrast visual stimulation. This reference is incorporated herein by reference in its entirety

In some embodiments, OCT4, SOX2, KLF4, replacements thereof, or any combination thereof is activated in a cell, tissue, organ and/or a subject in combination with gamma band light stimulation to promote nerve regeneration. See, e.g., McDermott et al., J Alzheimers Dis. 2018; 65 (2): 363-392 for a description of gamma band light stimulation. This reference is incorporated herein by reference in its entirety.

In some embodiments, OCT4, SOX2, and/or KLF4 are activated in the central nervous system. In some embodiments, the central nervous system does not include the retina. In some embodiments, the central nervous system does not include the eye (e.g., the retina, uvea, pupil, lens, cornea, and/or sclera). In some embodiments, OCT4, SOX2, and/or KLF4 are activated in the brain. In some embodiments, OCT4, SOX2, and/or KLF4 is activated (e.g., expression of one or more of these transcription factors is induced) for no more than 1 year (e.g., less than 11 months, less than 10 months, less than 9 months, less than 8 months, less than 7 months, less than 6 months, less than 5 months, less than 4 months, less than 3 months, less than 2 months, or less than 1 month). Non-limiting methods of controlling induction of gene expression include use of a Tet-On or Tet-Off gene expression system. For example, gene expression in a Tet-On system can be repressed by removing tetracycline and gene expression can be repressed in a Tet-Off system with the addition of tetracycline.

### Engineered Cells, Tissues, Organs, and Animals

Further aspects of the present disclosure relate to engineered cells, tissues, or organs of a central nervous system comprising any of the compositions disclosed herein, any of the expression vectors disclosed herein, or any of the recombinant viruses disclosed herein. Methods of producing engineered cells, tissues, and organs of the central nervous system are also encompassed by the present disclosure. The engineered cells, for example, may be useful in cell-based therapies (e.g., stem cell therapies). Although stem cell therapy is currently in clinical trials (see, e.g., David Cyranoski, Nature 557, 619-620 (2018), toxicity (e.g., off-target toxicity) is a concern, Without being bound by a particular theory, the engineered cells of the present disclosure (e.g., cells engineered using AAV vectors encoding OCT4, KLF4, and/or SOX2, and/or an inducing agent) may have a lower toxicity because AAV is does not integrate into the genome of host cells and use of the inducible systems described herein to control expression of OCT4, KLF4, and/or SOX2 may allow for precise control (e.g., amount and timing) of gene expression.

Any of the nucleic acids (e.g., engineered nucleic acid) capable of inducing OCT4, KLF4, and/or SOX2 expression (e.g., expression vector), engineered proteins described herein, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein, alone, or in combination may be introduced into a host cell, host tissue, or organ to produce an engineered cell, an engineered tissue, or an engineered organ. Any of the nucleic acids (e.g., engineered nucleic acid) (e.g., expression vector) capable of inducing expression of OCT4; KLF4; SOX2; or any combination thereof, engineered proteins described herein, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein, alone, or in combination may be introduced into a host cell, host tissue, or organ to produce an engineered cell, an engineered tissue, or an engineered organ. In some embodiments, a nucleic acid (e.g., engineered nucleic acid) encoding an inducing agent, an engineered protein encoding an inducing agent, a chemical agent capable of modulating (e.g., activating or inhibiting) the activity of an inducing agent, and/or a recombinant virus encoding an inducing agent is also introduced into a host cell, host tissue, or organ to produce an engineered cell, an engineered tissue, or an engineered organ.

In some embodiments, a viral vector (e.g., an AAV vector, including a vector with a TRE promoter operably linked to a nucleic acid encoding OCT4, KLF4, and SOX2) is packaged into a virus with an AAV-DJ capsid. In some embodiments, the AAV-DJ capsid increases the transduction efficiency into cultured cells compared to cells without the AAV-DJ capsid. In some embodiments, the AAV virus encoding OSK is administered to a cell. In some embodiments, an AAV virus (e.g., AAV-DJ virus) encoding the inducing agent or a protein encoding the inducing agent is administered to the same cells. In some embodiments, the nerve cell is a brain cell.

In some embodiments, the engineered cell is a neuron. Neurons for use in the methods described herein can be of any neuron type. The neurons used in the methods may be from the central nervous system (CNS) (e.g., neurons from the brain or spinal cord) or the peripheral nervous system (PNS) (e.g., neurons from the somatic, autonomic, or enteric nervous systems). The neurons can be afferent neurons, efferent neurons, or interneurons. In some embodiments, the neuron used is a type of neuron found in the brain, such as a cortical neuron, cerebellar neuron, thalamic neuron, hippocampal neuron, hypothalamic neuron, collicular neuron, neuron from the basal ganglia (e.g., a striatal neuron, a neuron of the substantia nigra, or a pallidal neuron), an amygdala neuron, an interneuron, or a brainstem neuron. In some embodiments, the neuron is a cortical neuron. In some embodiments, the neuron is not a neuron from the eye (e.g., a retinal ganglion cell). In some embodiments, the neuron is defined by neurotransmitter (e.g., the neuron is a dopaminergic neuron, a cholinergic neuron, a GABAergic neuron, a glycinergic neuron, a glutamatergic neuron, or a serotonergic neuron). In some embodiments the neuron is a motor neuron. In some embodiments, the neuron is a spinal motor neuron or a spinal interneuron. In some embodiments, the neuron is a cranial nerve or a cranial nerve ganglion. In some embodiments, the neuron is a sensory neuron from the PNS (e.g., a dorsal root ganglion (DRG) neuron, an autonomic ganglion neuron, such as a superior cervical ganglion (SCG) neuron), or an enteric neuron, such as a myenteric or submucosal neuron or ganglion). The neurons may be myelinated or unmyelinated.

Neurons for use in the methods described herein can be derived from any source. In some embodiments, the neuron is a primary neuron isolated from a human subject (e.g., a neuron isolated from resected brain tissue or from a tissue or organ removed during a surgical procedure). In some embodiments, the neuron is a primary neuron isolated from a model organism, such as a mouse, rat, guinea pig, hamster, cat, dog, cow, horse, deer, elk, bison, oxen, camel, llama, rabbit, sheep, goat, or non-human primate. For example, the neuron can be a cortical neuron, dorsal root ganglion neuron, or superior cervical ganglion neuron isolated from a rat or mouse. Neurons can be isolated from organisms that have been genetically modified (e.g., a transgenic mouse in which neurons have been genetically modified to express a fluorophore, knockout or overexpress a gene of interest, or express a construct that allows for inducible control of neuronal activity or gene expression). In some embodiments, the neuron is generated from a stem cell, such as an induced pluripotent stem cell (iPSC, e.g., a human iPSC), embryonic stem cell (ESC), a neural stem cell (NSC), an adult stem cell (e.g., a somatic stem cell or a tissue-specific stem cell), a hematopoietic stem cell, a mesenchymal stem cell, an endothelial stem cell, an epithelial stem cell, or a germline stem cell. For example, the neuron may be a human iPSC-derived motor neuron, a human iPSC-derived cortical neuron, a human iPSC-derived hippocampal neuron, or an iPSC-derived dopaminergic neuron. In some embodiments, the neuron is from a neuronal cell line. In some embodiments, the cell is an excitatory neuron. In some embodiments, the differentiated cell is used for transplantation purposes. In some embodiments, the engineered cell is cultured to create an engineered tissue. In some embodiments, the engineered cell is cultured to create an engineered organ.

Any of the engineered cells, tissues, or organs of the present disclosure may be present in an animal. For example, aspects of the present disclosure provide an animal comprising any of the nucleic acids (e.g., engineered nucleic acid) capable of inducing OCT4, KLF4, and/or SOX2 expression (e.g., expression vector), engineered proteins described herein, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein, alone, or in combination. In some embodiments, the animal is a mouse, rat, guinea pig, hamster, cat, dog, cow, horse, deer, elk, bison, oxen, camel, llama, rabbit, sheep, goat, or non-human primate. In some embodiments, the animal is a transgenic animal.

In some embodiments, an animal comprises a nucleic acid comprising a first nucleic acid (e.g., engineered nucleic acid) encoding OCT4, a second nucleic acid (e.g., engineered nucleic acid) encoding SOX2, a third nucleic acid (e.g., engineered nucleic acid) encoding KLF4, alone or in combination, and in the absence of an exogenous nucleic acid (e.g., engineered nucleic acid) capable of expressing c-Myc for use in the central nervous system. In some embodiments, the nucleic acid encoding OCT4, SOX2, and/or KLF4 comprises an inducible promoter operably linked to any one of the first, second, or third nucleic acids or a combination thereof. In some embodiments, the inducible promoter is responsive to tetracycline. In some embodiments, the animal further comprise a nucleic acid encoding an inducing agent. In some embodiments, the nucleic acid encoding the inducing agent comprises a CAMKIIα promoter operably linked to the nucleic acid sequence encoding the inducing agent. In some embodiments, the inducing agent comprises a tetracycline transactivator (tTA), and/or a reverse tetracycline-controlled transactivator (rtTA). In some embodiments, the animal is a mouse and further has overexpression of APP and PSEN1 with five familial AD mutations (APP KM670/671NL, APP I716V, APP V717I, PSEN1 M146L, PSEN1 L286V), under the control of a Thy1 mini-gene. See, e.g., description of 5×FAD transgenic mice from Oakley et al., J Neurosci. 2006; 26:10129-10140 and Forner et al., Sci Data. 2021.

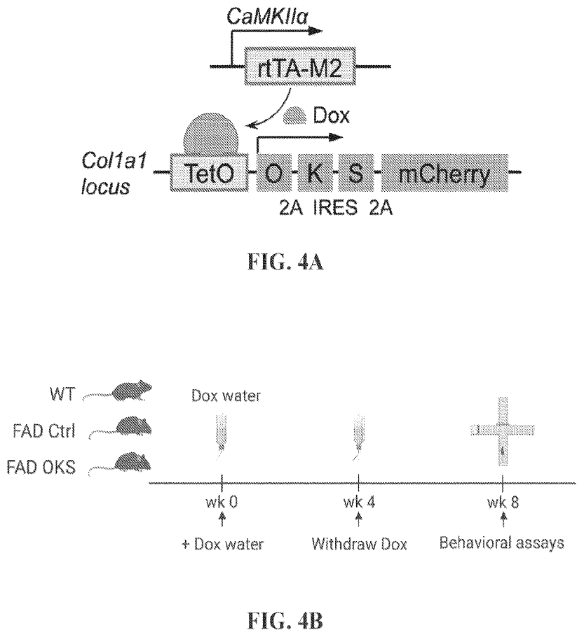

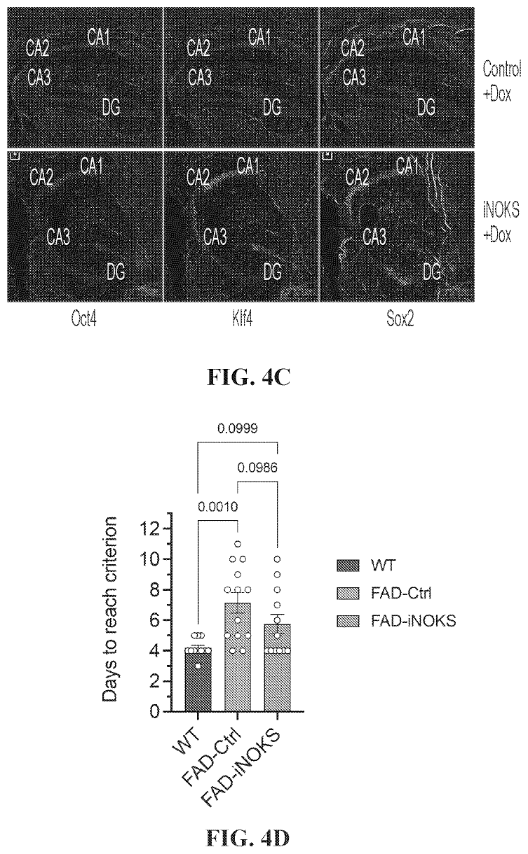

In some embodiments, an animal is a transgenic mouse comprising a nucleic acid at the Col1a1 locus that encodes OCT4, KLF4, and SOX2 in which the nucleic acid sequence encoding OCT4, KLF4, and SOX2 is operably linked to a TetO promoter. In some embodiments, the animal further comprises a nucleic acid encoding rtTA-M2 in which the nucleic acid encoding rtTA-M2 is operably linked to a CAMKIIα promoter. See, e.g., FIG. 4A.

### Compositions

Aspects of the present disclosure provide compositions for use in rejuvenating a cell, tissue, or organ comprising: (a) an agent that induces OCT4 expression; (b) an agent that induce SOX2 expression; and (c) an agent that induces KLF4 expression, wherein the composition does not comprise an agent that induces c-MYC expression, wherein the cell, tissue, or organ is a central nervous system cell, central nervous system tissue, or central nervous system organ, optionally wherein the central nervous system does not include the eye, optionally wherein central nervous system does not include the retina, uvea, pupil, lens, cornea, and/or sclera, optionally wherein the cell, tissue, or organ is a brain cell, brain tissue, or brain.

In some embodiments, the brain cell is a neuron.

In some embodiments, the neuron is an excitatory neuron.

In some embodiments, the brain tissue is nervous tissue.

In some embodiments, the cell, tissue, or organ is in a subject, optionally wherein the composition is administered to a subject in need thereof, optionally wherein the subject has a neurological disorder.

In some embodiments, the composition does not induce OCT4, SOX2, or KLF4 expression in the retina.

In some embodiments, the composition does not induce OCT4, SOX2, or KLF4 expression in the eye (e.g., retina, uvea, pupil, lens, cornea, and/or sclera).

In some embodiments, each agent is independently a nucleic acid, a small molecule, or a polypeptide, optionally wherein the polypeptide is an antibody.

In some embodiments, at least one agent comprises a nanoparticle.

In some embodiments, at least one agent is encapsulated in at least one nanoparticle.

In some embodiments, the nucleic acid is DNA or RNA.

In some embodiments, the DNA is plasmid DNA.

In some embodiments, the RNA is mRNA.

In some embodiments, the agent that induces OCT4 expression is an engineered nucleic acid encoding OCT4.

In some embodiments, the agent that induces SOX2 expression is an engineered nucleic acid encoding SOX2.

In some embodiments, the agent that induces KLF4 expression is an engineered nucleic acid encoding KLF4.

In some embodiments, the agent that induces OCT4 expression is an engineered nucleic acid encoding OCT4, the agent that induces SOX2 expression is an engineered nucleic acid encoding SOX2, and the agent that induces KLF4 expression is an engineered nucleic acid encoding KLF4.

In some embodiments, the engineered nucleic acids are present on one or more expression vectors.

In some embodiments, the engineered nucleic acids are present on the same expression vector.

In some embodiments, the one or more expression vectors include an inducible promoter operably linked to any one of the engineered nucleic acids or a combination thereof.

In some embodiments, the promoter is a TRE3G, a TRE2 promoter, or a P tight promoter.

In some embodiments, said promoter comprises a tetracycline response element (TRE).

In some embodiments, the expression vector comprises a hGH pA terminator sequence, optionally wherein the hGH pA terminator sequence comprises a sequence that is at least 70% identical (e.g., at least 75%, 80%, 85%, 90%, 95%, 98%, 99%, or 100% identical) to SEQ ID NO: 139, 148, 153, 156, or 161.

In some embodiments, the expression vector comprises a WPRE sequence.

In some embodiments, the expression vector comprises a self-cleaving peptide.

In some embodiments, the self-cleaving peptide is a 2A peptide, optionally wherein the 2A peptide sequence comprises a sequence that is at least 70% identical (e.g., at least 75%, 80%, 85%, 90%, 95%, 98%, 99%, or 100% identical) to SEQ ID NO: 118 and/or is encoded by a nucleic acid comprising a sequence that is at least 70% identical (e.g., at least 75%, 80%, 85%, 90%, 95%, 98%, 99%, or 100% identical) to SEQ ID NO: 144.

In some embodiments, the expression vector comprises inverted terminal repeats (ITRs) flanking the first nucleic acid, the second nucleic acid, the third nucleic acid, or a combination thereof, and wherein the distance between the ITRs is 4.7 kb or less.

In some embodiments, the composition further comprises an inducing agent, or wherein the method further comprises administering to said subject an inducing agent.

In some embodiments, the inducing agent comprises a tetracycline, a tetracycline transactivator (tTA), and/or a reverse tetracycline-controlled transactivator (rtTA), optionally wherein the tTA comprises a sequence that is at least 70% identical (e.g., at least 75%, 80%, 85%, 90%, 95%, 98%, 99%, or 100% identical) to 138 or 159, optionally wherein the tTA is encoded by a sequence that is at least 70% identical (e.g., at least 75%, 80%, 85%, 90%, 95%, 98%, 99%, or 100% identical) to 137 or 158, optionally wherein the rtTA comprises a sequence that is at least 70% identical (e.g., at least 75%, 80%, 85%, 90%, 95%, 98%, 99%, or 100% identical) to SEQ ID NO: 11, 129, 13, or 15, optionally wherein the rtTA is encoded by a sequence that is at least 70% identical (e.g., at least 75%, 80%, 85%, 90%, 95%, 98%, 99%, or 100% identical) to SEQ ID NO: 10, 12, 14, or 128.

In some embodiments, the tetracycline is doxycycline.

In some embodiments, the composition comprises an expression vector with an engineered nucleic acid that encodes the tTA and/or rtTA, optionally wherein the engineered nucleic acid that encodes the tTA and/or rtTA comprises a WPRE sequence and/or an hGH pA sequence, optionally wherein the WPRE sequence is at least 70% identical (e.g., at least 75%, 80%, 85%, 90%, 95%, 98%, 99%, or 100% identical) to SEQ ID NO: 21, 135, 147, 152, 155, or 160 and/or the hGH pA sequence is at least 70% identical (e.g., at least 75%, 80%, 85%, 90%, 95%, 98%, 99%, or 100% identical) to SEQ ID NO: 139, 148, 153, 156, or 161.

In some embodiments, the expression vector encoding the tTA and/or rtTA is the same expression vector or is a different expression vector as the engineered nucleic acids encoding OCT4, SOX2, and/or KLF4.

In some embodiments, the rtTA is rtTA3, rtTA Advanced, rtTA2S-M2, or rtTA4, optionally wherein the rtTA comprises a sequence that is at least 70% identical (e.g., at least 75%, 80%, 85%, 90%, 95%, 98%, 99%, or 100% identical) to a sequence selected from the group consisting of SEQ ID NOs: 11, 13, 15, and 129.

In some embodiments, at least one expression vector is a viral vector, optionally wherein at least one expression vector is packaged in a recombinant virus.

In some embodiments, the viral vector is a lentivirus, a retrovirus, an adenovirus, alphavirus, vaccinia virus, human papillomavirus, or an adeno-associated virus (AAV) vector.

In some embodiments, the AAV vector is packaged in AAV-PHP.eB, AAV-PHP.b, AAV.CAP-B10, or AAV.CAP-B22 virus.

In some embodiments, the AAV vector is not AAV2 or AAV9.

In some embodiments, the subject is a human or non-human mammal.

In some embodiments, the expression vector with the engineered nucleic acid that encodes the tTA and/or rtTA comprises a promoter operably linked to the nucleic acid that encodes the tTA and/or rtTA.

In some embodiments, the promoter operably linked to the engineered nucleic acid that encodes the tTa or rtTA is a ubiquitous promoter.

In some embodiments, the ubiquitous promoter is UBC, CMV, PGK1, CAG, optionally wherein the ubiquitous promoter comprises a sequence that is at least 70% identical (e.g., at least 75%, 80%, 85%, 90%, 95%, 98%, 99%, or 100% identical) to 48, 132, 130, 136, or 162.

In some embodiments, the promoter operably linked to the engineered nucleic acid that encodes the tTa or rtTA is a neuron-specific promoter.

In some embodiments, the neuron-specific promoter is CaMKIIα, optionally wherein the CaMKIIα promoter comprises a sequence that is at least 70% identical (e.g., at least 75%, 80%, 85%, 90%, 95%, 98%, 99%, or 100% identical) to SEQ ID NO: 146, 149, or 154.

In some embodiments, the promoter operably linked to the engineered nucleic acid that encodes tTA and/or rtTA is not a Synapsin-I promoter, is not a CaMKII-gamma promoter, or a combination thereof, optionally wherein the Synapsin-I promoter comprises a sequence that is at least 70% identical (e.g., at least 75%, 80%, 85%, 90%, 95%, 98%, 99%, or 100% identical) to SEQ ID NO: 157.

In some embodiments, the composition does not comprise a nucleic acid with a Synapsin-I promoter, does not comprise a CaMKII-gamma promoter, or a combination thereof, optionally wherein the Synapsin-I promoter comprises a sequence that is at least 70% identical (e.g., at least 75%, 80%, 85%, 90%, 95%, 98%, 99%, or 100% identical) to SEQ ID NO: 157.

In some embodiments, the composition is a pharmaceutical composition.

In some embodiments, the composition comprises: (a) a viral vector comprising a nucleic acid encoding a tetracycline-controlled transactivator (ITA) or reverse tetracycline-controlled transactivator (rtTA), wherein the nucleic acid encoding the tTA or rtTA is operably linked to a CaMKIIα promoter, optionally wherein the viral vector is an AAV vector, optionally wherein the AAV vector is packaged in AAV-PHP.eB virus; and (b) an viral vector comprising a first nucleic acid encoding OCT4, a second nucleic acid encoding SOX2, and a third nucleic acid encoding KLF4, wherein the first, second, and third nucleic acids are operably linked to a promoter comprising a tetracycline response element (TRE), optionally wherein the viral vector is an AAV vector, optionally wherein the AAV vector is packaged in AAV-PHP.eB virus.

In some embodiments, the vector in (a) comprises a WPRE sequence and/or a hGH pA terminator sequence, optionally wherein the WPRE sequence comprises a sequence that is at least 70% identical (e.g., at least 75%, 80%, 85%, 90%, 95%, 98%, 99%, or 100% identical) to SEQ ID NO: 21, 135, 147, 152, 155, or 160 and/or wherein the hGH pA terminator sequence comprises a sequence that is at least 70% identical (e.g., at least 75%, 80%, 85%, 90%, 95%, 98%, 99%, or 100% identical) to SEQ ID NO: 139, 148, 153, 156, or 161.

In some embodiments, the viral vector in (a) is the same viral vector as in (b).

In some embodiments, the composition comprises a sequence that is at least 70% identical (e.g., at least 75%, 80%, 85%, 90%, 95%, 98%, 99%, or 100% identical) to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163.

In some embodiments, the composition comprises a sequence that is at least 70% identical (e.g., at least 75%, 80%, 85%, 90%, 95%, 98%, 99%, or 100% identical) to any sequence disclosed in this application.

In some embodiments, the composition comprises a sequence that is at least 70% identical (e.g., at least 75%, 80%, 85%, 90%, 95%, 98%, 99%, or 100% identical) to SEQ ID NO: 123.

In some embodiments, the composition comprises a sequence that is at least 70% identical (e.g., at least 75%, 80%, 85%, 90%, 95%, 98%, 99%, or 100% identical) to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126, optionally wherein the composition does not comprise a sequence that is at least 70% identical (e.g., at least 75%, 80%, 85%, 90%, 95%, 98%, 99%, or 100% identical) to SEQ ID NO: 127.

In some embodiments, the viral vector in part (a) comprises a sequence that is at least 70% identical (e.g., at least 75%, 80%, 85%, 90%, 95%, 98%, 99%, or 100% identical) to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126.

In some embodiments, the viral vector in part (b) comprises a sequence that is at least 70% identical (e.g., at least 75%, 80%, 85%, 90%, 95%, 98%, 99%, or 100% identical) to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163.

In some embodiments, the composition further comprises a pharmaceutically acceptable carrier.

The compositions of the disclosure may comprise at least one of any of the nucleic acids (e.g., engineered nucleic acid) capable of inducing OCT4, KLF4, and/or SOX2 expression (e.g., expression vector) and/or nucleic acids encoding an inducing agent, engineered proteins, engineered cells, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein alone, or in combination. In some embodiments, a composition comprises an inducing agent (e.g., a nucleic acid encoding an inducing agent). In certain embodiments, the compositions of the disclosure comprise at least one of any of the nucleic acids (e.g., engineered nucleic acid) (e.g., expression vector) capable of inducing expression of OCT4; KLF4; SOX2; or any combination thereof, engineered proteins, engineered cells, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein alone, or in combination. In some embodiments, a composition comprises 1, 2, 3, 4, 5, 6, 7, 8, 9, 10, or more different nucleic acids (e.g., engineered nucleic acids) capable of inducing OCT4. KLF4, and/or SOX2 expression (e.g., expression vectors encoding OCT4, KLF4, and/or SOX2). In some embodiments, a composition comprises 1, 2, 3, 4, 5, 6, 7, 8, 9, 10, or more different nucleic acids (e.g., engineered nucleic acids) capable of inducing expression of OCT4; KLF4; SOX2; or any combination thereof (e.g., expression vectors encoding OCT4; KLF4; SOX2; or any combination thereof). In some embodiments, a composition comprises 1, 2, 3, 4, 5, 6, 7, 8, 9, 10, or more different viruses (e.g., lentivirus, alphavirus, vaccinia virus, adenovirus, retrovirus, herpes virus, human papillomavirus, or AAV) each having one or more different transgenes. In some embodiments, a composition comprises 1, 2, 3, 4, 5, 6, 7, 8, 9, 10, or more different chemical agents activating (e.g., inducing expression of) OCT4, KLF4, and/or SOX2. In some embodiments, a composition comprises 1, 2, 3, 4, 5, 6, 7, 8, 9, 10, or more different chemical agents activating (e.g., inducing expression of) OCT4; KLF4; SOX2; or any combination thereof. In some embodiments, a composition further comprises one or more nucleic acids (e.g., engineered nucleic acids) encoding an inducing agent, one or more engineered proteins encoding an inducing agent, one or more chemical agents capable of modulating (e.g., activating or inhibiting) the activity of an inducing agent, and/or one or more recombinant viruses encoding an inducing agent. In some embodiments, a composition comprises engineered cells (e.g., differentiated cells). In some embodiments, a composition comprises an engineered protein encoding OCT4, SOX2, and/or KLF4. In some embodiments, a composition comprises an engineered protein encoding OCT4, SOX2, KLF4, or any combination thereof. In some embodiments, a composition further comprises an engineered protein encoding an inducing agent.

In some embodiments, a composition comprises a vector comprising a nucleic acid encoding a tetracycline-controlled transactivator (tTA) or reverse tetracycline-controlled transactivator (rtTA), wherein the nucleic acid encoding the tTA or rtTA is operably linked to a CaMKIIα promoter; and/or a vector comprising a first nucleic acid encoding OCT4, a second nucleic acid encoding SOX2, and a third nucleic acid encoding KLF4, wherein the first, second, and third nucleic acids are operably linked to a promoter comprising a tetracycline response element (TRE). In some embodiments, one or more vectors are viral vectors. In some embodiments, one or more viral vectors are packaged into AAV-PHP.eB virus.

In some embodiments, a composition further comprises a pharmaceutically acceptable carrier. Suitable carriers may be readily selected by one of skill in the art in view of the indication for which any of the nucleic acids (e.g., engineered nucleic acid) chemical agents capable activating (e.g., inducing expression of) OCT4, KLF4, and/or SOX2, antibodies activating (e.g., inducing expression of) OCT4, KLF4, and/or SOX2, engineered proteins, engineered cells, and/or recombinant viruses (e.g., lentivirus, adenovirus, alphavirus, vaccinia virus, retrovirus, herpes virus, human papillomavirus, or AAV) e.g. is directed. Suitable carriers may be readily selected by one of skill in the art in view of the indication for which the nucleic acids (e.g., engineered nucleic acid) (e.g., expression vectors) capable of inducing expression of OCT4; KLF4; SOX2; or any combination thereof, chemical agents activating (e.g., inducing expression of) OCT4; KLF4; SOX2; or any combination thereof, antibodies activating (e.g., inducing expression of) OCT4; KLF4; SOX2; or any combination thereof, engineered proteins, engineered cells, and/or recombinant viruses (e.g., lentivirus, adenovirus, alphavirus, vaccinia virus, retrovirus, herpes virus, human papillomavirus, or AAV) e.g. is directed. Suitable carriers may also be readily selected by one of skill in the art in view of the indication for which the nucleic acids (e.g., engineered nucleic acids) encoding an inducing agent, engineered proteins encoding an inducing agent, chemical agents capable of modulating (e.g., activating or inhibiting) the activity of an inducing agent, and/or recombinant viruses (e.g., lentivirus, adenovirus, alphavirus, vaccinia virus, retrovirus, herpes virus, human papillomavirus, or AAV) comprising an inducing agent e.g. is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may comprise, in addition to any of the nucleic acids (e.g., engineered nucleic acid) disclosed herein, engineered cells comprising OCT4, KLF4, and/or SOX2, and/or an inducing agent, engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) e.g. and carrier(s), other pharmaceutical ingredients, such as preservatives, or chemical stabilizers. Optionally, the compositions of the disclosure may comprise, in addition to any of the nucleic acids (e.g., engineered nucleic acid) (e.g., expression vector) disclosed herein, engineered cells comprising OCT4; KLF4; SOX2; or any combination thereof; and/or comprising an inducing agent, engineered proteins, chemical agents activating (e.g., inducing expression of) OCT4; KLF4; SOX2; or any combination thereof, antibodies activating (e.g., inducing expression of) OCT4; KLF4; SOX2; or any combination thereof, and/or any of the recombinant viruses described herein (e.g., lentivirus, adenovirus, alphavirus, vaccinia virus, retrovirus, herpes virus, human papillomavirus, or AAV) e.g. and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. The compositions of the present disclosure may further comprise a nucleic acid (e.g., engineered nucleic acids) encoding an inducing agent, an engineered protein encoding an inducing agent, chemical agents capable of modulating (e.g., activating or inhibiting) the activity of an inducing agent, and/or recombinant viruses encoding an inducing agent.

The nucleic acid (e.g., engineered nucleic acid) (e.g., expression vector) capable of inducing expression of OCT4; KLF4; SOX2; or any combination thereof, engineered cells, chemical agents activating (e.g., inducing expression of) OCT4; KLF4; SOX2; or any combination thereof, engineered proteins encoding OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) encoding the same described herein are administered in sufficient amounts to transfect the cells of a desired tissue (e.g., from the central nervous system, e.g., including a brain) and to provide sufficient levels of gene transfer and expression without undue adverse effects. Any of the nucleic acids (e.g., engineered nucleic acids) encoding an inducing agent, an engineered protein encoding an inducing agent, chemical agents capable of modulating (e.g., activating or inhibiting) the activity of an inducing agent, and/or recombinant viruses encoding an inducing agent are administered in sufficient amounts to transfect the cells of a desired tissue (e.g., tissue from the central nervous system, e.g., including the brain) and to provide sufficient levels of gene transfer and expression without undue adverse effects. Examples of pharmaceutically acceptable routes of administration include, but are not limited to, direct delivery to the selected organ (e.g., direct delivery to the central nervous system, e.g., including the brain). Any of the nucleic acids (e.g., engineered nucleic acids) capable of inducing OCT4, KLF4, and/or SOX2 expression (e.g., expression vector), engineered cells, chemical agents activating (e.g., inducing expression of) OCT4, KLF4, and/or SOX2, engineered proteins, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein may be delivered intravenously, intradermally, intraarterially, intralesionally, intratumorally, intracranially, intraarticularly, intraprostatically, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes), or by other method or any combination of the forgoing as would be known to one of ordinary skill in the art. Any of the nucleic acids (e.g., engineered nucleic acids) (e.g., expression vectors) capable of inducing expression of OCT4; KLF4; SOX2; or any combination thereof, engineered cells, chemical agents activating (e.g., inducing expression of) OCT4; KLF4; SOX2; or any combination thereof, engineered proteins, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein may be delivered intravenously, intradermally, intraarterially, intralesionally, intratumorally, intracranially, intraarticularly, intraprostatically, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes), or by other method or any combination of the forgoing as would be known to one of ordinary skill in the art. Any of the nucleic acids encoding an inducing agent, chemical agents capable of modulating the activity of an inducing agent, engineered proteins encoding an inducing agent, and/or recombinant viruses encoding an inducing agent may be may be delivered intravenously, intradermally, intraarterially, intralesionally, intratumorally, intracranially, intraarticularly, intraprostatically, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes), or by other method or any combination of the forgoing as would be known to one of ordinary skill in the art. In some embodiments, any of the compositions described herein is administered to the central nervous system. In some embodiments, any of the compostions described herein is not administered to the eye (e.g., not administered to the retina). In some embodiments, any of the compositions described herein is administered to the brain. Routes of administration may be combined, if desired.

In some embodiments, a nucleic acid is delivered non-virally (e.g., not on a viral vector and/or not in a virus). In some embodiments, a nucleic acid (e.g., RNA or DNA) encoding OCT4, SOX2, and/or KLF4 and/or an inducing agent is administered in a liposome. In some embodiments, a nucleic acid (e.g., RNA or DNA) encoding OCT4, SOX2, KLF4, or any combination thereof, and/or an inducing agent is administered in a liposome. In some embodiments, a nucleic acid (e.g., RNA or DNA) encoding OCT4, SOX2, and/or KLF4 and/or an inducing agent is administered in a particle. In some embodiments, a nucleic acid (e.g., RNA or DNA) encoding OCT4, SOX2, KLF4, or any combination thereof, and/or an inducing agent is administered in a particle. In some embodiments, the nucleic acid is RNA (e.g., mRNA).

In some embodiments, a pharmaceutical composition comprising an expression vector encoding OCT4, KLF4, and/or SOX2 and/or an inducing agent or a pharmaceutical composition comprising a virus harboring the expression vector is administered to a cell, tissue, organ or a subject. In some embodiments, a pharmaceutical composition comprising an expression vector encoding an inducing agent or a pharmaceutical composition comprising a virus harboring the expression vector is administered to a cell, tissue, organ or a subject. For example, the cell, tissue, or organ may be in the central nervous system. The subject may have a neurological disorder. In some embodiments, the virus and/or expression vector encoding OCT4, KLF4, and/or SOX2 is administered systemically. In some embodiments, the virus and/or expression vector encoding an inducing agent is administered systemically. In some embodiments, the virus and/or expression vector encoding OCT4, KLF4, and/or SOX2, and/or encoding an inducing agent is administered locally (e.g., directly to a tissue or organ of interest, including, e.g., a tissue or organ from the central nervous system). In some embodiments, the tissue or organ is brain tissue or is the brain. In some embodiments, a virus and/or expression vector encoding OCT4, KLF4, and/or SOX2 and/or encoding an inducing agent is administered through retro-orbital venous injection. In some embodiments, a virus and/or expression vector encoding OCT4, KLF4, and/or SOX2 and/or encoding an inducing agent is administered by Intrathecal administration. In some embodiments, the inducing agent (e.g., a nucleic acid encoding the inducing agent, a protein encoding the inducing agent, or a virus encoding the inducing agent) and/or chemical agent capable of modulating (e.g., activating or inhibiting) the activity of the inducing agent is administered using the same route of administration as the OCT4, KLF4, and/or SOX2 (e.g., nucleic acid encoding OCT4, KLF4, and/or SOX2). In some embodiments, the inducing agent (e.g., a nucleic acid encoding the inducing agent, a protein encoding the inducing agent, or a virus encoding the inducing agent) and/or chemical agent capable of modulating (e.g., activating or inhibiting) the activity of the inducing agent is administered via a different route of administration as the OCT4, KLF4, and/or SOX2 (e.g., nucleic acid encoding OCT4, KLF4, and/or SOX2).

In some embodiments, a pharmaceutical composition comprising an expression vector encoding OCT4; KLF4; SOX2; or any combination thereof, or a pharmaceutical composition comprising a virus harboring the expression vector is administered to a cell, tissue, organ, or subject. In some embodiments, a pharmaceutical composition comprising an expression vector encoding an inducing agent or a pharmaceutical composition comprising a virus harboring the expression vector is administered to a cell, tissue, organ, or subject. In some embodiments, the virus and/or expression vector encoding OCT4; KLF4; SOX2; or any combination thereof is administered systemically. In some embodiments, the virus and/or expression vector encoding an inducing agent is administered systemically. In some embodiments, the virus and/or expression vector encoding OCT4; KLF4; SOX2; or any combination thereof is administered locally (e.g., directly to a tissue or organ of interest, including the central nervous system). In some embodiments, a virus and/or expression vector encoding an inducing agent is administered locally (e.g., directly to a tissue or organ of interest, including the central nervous system). In some embodiments, the inducing agent (e.g., a nucleic acid encoding the inducing agent, a protein encoding the inducing agent, or a virus encoding the inducing agent) and/or chemical agent capable of modulating (e.g., activating or inhibiting) the activity of the inducing agent is administered using the same route of administration as the OCT4; KLF4; SOX2; or any combination thereof (e.g., nucleic acid encoding OCT4; KLF4; SOX2; OCT4 and SOX2; OCT4 and KLF4; KLF4 and SOX2; or KLF4, OCT4, and SOX2). In some embodiments, the inducing agent (e.g., a nucleic acid encoding the inducing agent, a protein encoding the inducing agent, or a virus encoding the inducing agent) and/or chemical agent capable of modulating (e.g., activating or inhibiting) the activity of the inducing agent is administered via a different route of administration as the OCT4; KLF4; SOX2; or any combination thereof (e.g., nucleic acid encoding nucleic acid encoding OCT4; KLF4; SOX2; OCT4 and SOX2; OCT4 and KLF4; KLF4 and SOX2; or KLF4, OCT4, and SOX2).

In some embodiments, the expression vector is an inducible vector in which a nucleic acid encoding OCT4, KLF4, and/or SOX2 and/or inducing agent, is operably linked to an inducible TRE promoter (e.g., TRE3G, TRE2, or P tight). In some embodiments, the expression vector is an inducible vector in which a nucleic acid encoding OCT4; KLF4; SOX2; or any combination thereof, and/or inducing agent, is operably linked to an inducible TRE promoter (e.g., TRE3G, TRE2, or P tight). In some embodiments, the virus and/or inducible vector is administered with tetracycline (e.g., doxycycline). In some embodiments, the virus and/or expression vector comprising a TRE promoter is administered separately from tetracycline (e.g., doxycycline). For example, any of the viruses and/or expression vectors comprising a TRE promoter described herein may be administered systemically and the tetracycline may be administered locally (e.g., to an organ or tissue of interest). In some embodiments, any of the viruses and/or expression vectors comprising a TRE promoter described herein may be administered locally (e.g., to directly to a tissue or organ of interest, including the central nervous system) and the tetracycline may be administered systemically. As a non-limiting example, a virus and/or expression vector comprising a TRE promoter is administered directly (e.g., injected) into the eye of a subject and the tetracycline (e.g., doxycycline) is administered systemically (e.g., orally as a pill).

In some embodiments, tetracycline is administered intravenously, intradermally, intraarterially, intralesionally, intratumorally, intracranially, intraarticularly, intraprostatically, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, or in lipid compositions. In some embodiments, tetracycline is administered directly to a cell, organ, and/or tissue. As a non-limiting example, tetracycline may be administered to the eye of a subject through any suitable method, including eye drops comprising tetracycline, sustained release devices (e.g., micropumps, particles, and/or drug depots), and medicated contact lenses comprising tetracycline. In some embodiments, tetracycline is administered systemically (e.g., through drinking water or intravenous injection) to a subject. Tetracycline may be administered topically (e.g., in a cream) or through a subcutaneous pump (e.g., to deliver tetracycline to a particular tissue).

As an example, the dose of recombinant virus (e.g., lentivirus, alphaviruses, vaccinia viruses, adenovirus, retrovirus, herpes virus, human papillomavirus, or AAV) virions required to achieve a particular therapeutic effect, e.g., the units of dose in genome copies/per kilogram of body weight (GC/kg), will vary based on several factors including, but not limited to: the route of recombinant virus (e.g., lentivirus, alphaviruses, vaccinia viruses, adenovirus, retrovirus, herpes virus, human papillomavirus, or AAV) virion administration, the level of gene or RNA expression required to achieve a therapeutic effect, the specific disease or disorder being treated, and the stability of the gene or RNA product. One of skill in the art can readily determine a recombinant virus (e.g., lentivirus, alphaviruses, vaccinia viruses, adenovirus, retrovirus, herpes virus, human papillomavirus, or AAV virion) dose range to treat a patient having a particular disease or disorder based on the aforementioned factors, as well as other factors.

An effective amount of a recombinant virus (e.g., lentivirus, alphaviruses, vaccinia viruses, adenovirus, retrovirus, herpes virus, human papillomavirus, or AAV) is an amount sufficient to target infect an animal, target a desired tissue. In some embodiments, an effective amount of an recombinant virus (e.g., lentivirus, alphavirus, vaccinia virus, adenovirus, retrovirus, herpes virus, human papillomavirus, or AAV) is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the recombinant virus (e.g., lentivirus, alphavirus, vaccinia virus, adenovirus, retrovirus, herpes virus, human papillomavirus, or AAV) is generally in the range of from about 1 ml to about 100 ml of solution containing from about 10⁹ to 10¹⁶ genome copies. In some cases, a dosage between about 10¹¹ to 10¹³ recombinant virus (e.g., lentivirus, adenovirus, retrovirus, alphavirus, vaccinia virus, herpes virus, human papillomavirus, or AAV) genome copies is appropriate. In certain embodiments, 10¹⁰ or 10¹¹ recombinant virus (e.g., lentivirus, adenovirus, retrovirus, alphavirus, vaccinia virus, herpes virus, human papillomavirus, or AAV) genome copies is effective to target ocular tissue (e.g., retinal tissue). In some cases, stable transgenic animals are produced by multiple doses of a recombinant virus (e.g., lentivirus, adenovirus, retrovirus, herpes virus, human papillomavirus, alphavirus, vaccinia virus, or AAV).

In some embodiments, a dose of recombinant virus (e.g., lentivirus, adenovirus, retrovirus, herpes virus, human papillomavirus, alphavirus, vaccinia virus, or AAV) is administered to a subject no more than once per calendar day (e.g., a 24-hour period). In some embodiments, a dose of recombinant virus (e.g., lentivirus, alphavirus, vaccinia virus, adenovirus, retrovirus, herpes virus, human papillomavirus, or AAV) is administered to a subject no more than once per 2, 3, 4, 5, 6, or 7 calendar days. In some embodiments, a dose of recombinant virus (e.g., lentivirus, alphavirus, vaccinia virus, adenovirus, retrovirus, herpes virus, human papillomavirus, or AAV) is administered to a subject no more than once per calendar week (e.g., 7 calendar days). In some embodiments, a dose of recombinant virus (e.g., lentivirus, alphavirus, vaccinia virus, adenovirus, retrovirus, herpes virus, human papillomavirus, or AAV) is administered to a subject no more than bi-weekly (e.g., once in a two calendar week period). In some embodiments, a dose of recombinant virus (e.g., lentivirus, alphavirus, vaccinia virus, adenovirus, retrovirus, herpes virus, human papillomavirus, or AAV) is administered to a subject no more than once per calendar month (e.g., once in 30 calendar days). In some embodiments, a dose of recombinant virus (e.g., lentivirus, alphavirus, vaccinia virus, adenovirus, retrovirus, herpes virus, human papillomavirus, or AAV) is administered to a subject no more than once per six calendar months. In some embodiments, a dose of recombinant virus (e.g., lentivirus, alphavirus, vaccinia virus, adenovirus, retrovirus, herpes virus, human papillomavirus, or AAV) is administered to a subject no more than once per calendar year (e.g., 365 days or 366 days in a leap year).

In some embodiments, recombinant virus (e.g., lentivirus, alphavirus, vaccinia virus, adenovirus, retrovirus, herpes virus, human papillomavirus, or AAV) compositions are formulated to reduce aggregation of AAV particles in the composition, particularly where high recombinant virus (e.g., lentivirus, alphavirus, vaccinia virus, adenovirus, retrovirus, herpes virus, human papillomavirus, or AAV) concentrations are present (e.g., ˜10¹³ GC/ml or more). Appropriate methods for reducing aggregation of may be used, including, for example, addition of surfactants, pH adjustment, salt concentration adjustment, etc. (See, e.g., Wright F R, et al., Molecular Therapy (2005) 12, 171-178, the contents of which are incorporated herein by reference.)

As a non-limiting example, delivery of transgenes via AAV have been shown to be feasible and non-toxic in humans. For example, AAV may be delivered to the eye. See, e.g., Smalley, Nat. Biotechnol. 2017 Nov. 9; 35 (11): 998-999.

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the nucleic acids (e.g., engineered nucleic acid) capable of inducing OCT4, KLF4, and/or SOX2 expression (e.g., expression vector), engineered cells comprising OCT4, KLF4, and/or SOX2, engineered proteins encoding Oct4, KLF4, and/or SOX2, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) e.g. in suitably formulated pharmaceutical compositions disclosed herein are delivered directly to target tissue, e.g., direct to a tissue of interest (e.g., nerve tissue).

In some embodiments, the nucleic acids (e.g., engineered nucleic acid) (e.g., expression vector) capable of inducing expression of OCT4; KLF4; SOX2; or any combination thereof, engineered cells comprising OCT4; KLF4; SOX2; or any combination thereof, engineered proteins encoding Oct4, KLF4, SOX2, or a combination thereof, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) e.g. in suitably formulated pharmaceutical compositions disclosed herein are delivered directly to target tissue, e.g., direct to a tissue of interest (e.g., nerve tissue).

In some embodiments, the nucleic acids (e.g., engineered nucleic acid) encoding an inducing agent (e.g., an expression vector), engineered cells comprising an inducing agent, engineered proteins encoding a inducing agent, chemical agents capable of modulating the activity of an inducing agent, and/or recombinant viruses (e.g., lentiviruses, adenoviruses, alphaviruses, vaccinia viruses, retroviruses, herpes viruses, or AAVs) encoding an inducing agent e.g. in suitably formulated pharmaceutical compositions disclosed herein are delivered directly to target tissue, e.g., direct to a tissue of interest (e.g., nerve tissue).

However, in certain circumstances it may be desirable to separately or in addition deliver any of the nucleic acids (e.g., engineered nucleic acid) capable of inducing OCT4. KLF4, and/or SOX2 expression (e.g., expression vector) and/or nucleic acid encoding an inducing agent, nucleic acids (e.g., engineered nucleic acid) capable of inducing expression of a combination of transcription factors selected from OCT4, KLF4, and/or nucleic acid encoding an inducing agent, engineered cells, engineered proteins, chemical agents activating (e.g., inducing expression of) OCT4, KLF4, and/or SOX2, chemical agents activating (e.g., inducing expression of) a combination of transcription factors selected from OCT4, KLF4, and SOX2, chemical agents capable of modulating (e.g., inhibiting or activating) the activity of an inducing agent, antibodies activating (e.g., inducing expression of) OCT4, KLF4, and/or SOX2, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) via another route, e.g., subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver recombinant virus (e.g., lentivirus, alphavirus, vaccinia virus, adenovirus, retrovirus, herpes virus, human papillomavirus, or AAVs). In some embodiments, the mode of administration is by intrastromal injection. In some embodiments, the mode of administration is by retro-orbital venous injection. In some embodiments, the mode of administration is by intrathecal administration.

In some embodiments, the mode of administration does not comprise administration to the eye (e.g., retina, uvea, pupil, lens, cornea, and/or sclera) of the subject. In some embodiments, the mode of administration does not comprise administration to the retina of the subject. In some embodiments, OCT4, KLF4, SOX2, and/or the inducing agent is not expressed in the retina. In some embodiments, OCT4, KLF4, SOX2, and/or the inducing agent is not activated in the retina. In some embodiments, OCT4, KLF4, SOX2, and/or the inducing agent is not expressed in the eye. In some embodiments, OCT4, KLF4, SOX2, and/or the inducing agent is not activated in the eye.

In some embodiments, a nucleic acid (e.g., mRNA) encoding OCT4, SOX2, KLF4, or any combination thereof is nanoformulated into a polyplex, which may be useful, for example, for noninvasive aerosol inhalation and delivery of the nucleic acid to the lung (e.g., lung epithelium). See, e.g., Patel et al., Adv Mater. 2019 Jan. 4:e1805116. doi: 10.1002/adma.201805116 for description of nanoformulated mRNA polyplexes, which is herein incorporated by reference in its entirety.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a suitable sterile aqueous medium may be employed. For example, one dosage may be dissolved in 1 ml of isotonic NaCl solution and either added to 1000 ml of hypodermoclysis fluid or injected at the proposed site of infusion, (see for example, ““Remingto”s Pharmaceutical Science”” 1^{5t}h Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the nucleic acid (e.g., engineered nucleic acid) capable of inducing OCT4, KLF4, and/or SOX2 expression (e.g., expression vector), engineered cells, engineered proteins, chemical agents activating (e.g., inducing expression of) OCT4, KLF4, an and/or SOX2, antibodies activating (e.g., inducing expression of) OCT4, KLF4, and/or SOX2, and/or active recombinant viruses (e.g., lentivirus, adenovirus, alphavirus, vaccinia virus, retrovirus, herpes virus, human papillomavirus, or AAV) e.g. in the required amount in the appropriate solvent with various of the other ingredients enumerated herein, as required, followed by filtered sterilization. In certain embodiments, the sterile injectable solutions are prepared by incorporating a nucleic acid (e.g., engineered nucleic acid) encoding an inducing agent, engineered protein encoding an inducing agent, chemical agents capable of modulating the activity of an inducing agent and/or active recombinant viruses (e.g., lentivirus, adenovirus, alphavirus, vaccinia virus, retrovirus, herpes virus, human papillomavirus, or AAV) encoding an inducing agent e.g. in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions comprising nucleic acids (e.g., engineered nucleic acids) encoding OCT4, KLF4, and/or SOX2 (e.g., expression vector), engineered cells, engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) disclosed herein may also be formulated in a neutral or salt form. The compositions comprising nucleic acids (e.g., engineered nucleic acids) (e.g., expression vector) encoding OCT4; KLF4; SOX2; or any combination thereof, engineered cells, engineered proteins, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) disclosed herein may also be formulated in a neutral or salt form. The compositions may comprise an inducing agent (e.g., a nucleic acid encoding an inducing agent or a protein encoding an inducing agent and/or a recombinant virus encoding an inducing agent) and/or a chemical agent capable of modulating the activity of an inducing agent. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

A carrier includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

Delivery vehicles such as liposomes, nanocapsules, microparticles, microspheres, lipid particles, vesicles, and the like, may be used for the introduction of the compositions of the present disclosure into suitable host cells. In particular, any of the nucleic acids (e.g., engineered nucleic acids) capable of inducing OCT4, KLF4, and/or SOX2 expression (e.g., expression vector), any of the engineered proteins, any of the chemical agents activating (e.g., inducing expression of) OCT4, KLF4, and/or SOX2, any of the antibodies activating (e.g., inducing expression of) OCT4, KLF4, and/or SOX2, engineered cells, and/or any of the recombinant viruses (e.g., lentivirus, adenovirus, alphavirus, vaccinia virus, retrovirus, herpes virus, human papillomavirus, or AAV) may be encapsulated in a lipid particle, a liposome, a vesicle, a nanosphere, or a nanoparticle or the like. In some embodiments, any of the nucleic acids (e.g., engineered nucleic acids) (e.g., expression vector) capable of inducing expression of OCT4; KLF4; SOX2; or any combination thereof, any of the engineered proteins, any of the chemical agents activating (e.g., inducing expression of) OCT4; KLF4; SOX2; or any combination thereof, any of the antibodies activating (e.g., inducing expression of) OCT4; KLF4; SOX2; or any combination thereof, engineered cells, and/or any of the recombinant viruses (e.g., lentivirus, adenovirus, alphavirus, vaccinia virus, retrovirus, herpes virus, human papillomavirus, or AAV) may be encapsulated in a lipid particle, a liposome, a vesicle, a nanosphere, or a nanoparticle or the like. An inducing agent (e.g., a nucleic acid encoding an inducing agent or a protein encoding an inducing agent and/or a recombinant virus encoding an inducing agent) and/or a chemical agent capable of modulating the activity of an inducing agent may be encapsulated in a lipid particle, a liposome, a vesicle, a nanosphere, or a nanoparticle or the like.

In some embodiments, the delivery vehicle targets the cargo. For example, any of the nucleic acids, engineered proteins, chemical agents, antibodies, and/or recombinant viruses (e.g., lentivirus, adenovirus, alphavirus, vaccinia virus, retrovirus, herpes virus, human papillomavirus, or AAV) described herein may be delivered via a nanoparticle that delivers the cargo to a certain tissue or cell type. Nanoparticles coated in galactose polymers, for example, are known to release their cargo within senescent cells as a result of their endogenous beta-galactosidase activity. See, e.g., Lozano-Torres et al., J Am Chem Soc. 2017 Jul. 5; 139 (26): 8808-8811.

In some embodiments, any of the nucleic acids, engineered proteins, chemical agents, antibodies, and/or recombinant viruses (e.g., lentivirus, adenovirus, alphavirus, vaccinia virus, retrovirus, herpes virus, human papillomavirus, or AAV) is formulated in a poly(glycoamidoamine) brush nanoparticles. See, e.g., Dong et al., Nano Lett. 2016 Feb. 10; 16 (2): 842-8.

In some embodiments, any of the nucleic acids, engineered proteins, chemical agents, antibodies, and/or recombinant viruses (e.g., lentivirus, adenovirus, alphavirus, vaccinia virus, retrovirus, herpes virus, human papillomavirus, or AAV) is formulated in a lipid nanoparticle. See, e.g., Cullis and Hope Mol Ther. 2017 Jul. 5; 25 (7): 1467-1475. In some embodiments, the lipid nanoparticle comprises one or more membrane fusion proteins, which deliver plasmids directly into the cytoplasm or the factors OCT4; KLF4; SOX2; or any combination thereof may be fused directly to the targeting protein with or without nanoparticle encapsulation. In some embodiments, the lipid nanoparticle is a Fusogenix lipid nanoparticle. In some embodiments, the lipid nanoparticle is a “Wrapped Liposomes” (WL). See, e.g., Yamauchi et al., Biochim Biophys Acta. 2006 January; 1758 (1): 90-7. In some embodiments, the lipid nanoparticle is a PEGylated liposome (e.g., DOXIL™) (e.g., Allen & Hansen, Biochim Biophys Acta. 1991 Jul. 1; 1066 (1): 29-36.), 1, 2-dioleoyl-sn-glycerol-3 phosphatidylethanolamine (DOPE), a neutral helper lipid phosphatidylethanolamine (PE), or combinations thereof (e.g., Farhood et al., Biochim Biophys Acta. 1995 May 4; 1235 (2): 289-95; Zhou & Huang, Biochim Biophys Acta. 1994 Jan. 19; 1189 (2): 195-203.). In some embodiments, the lipid nanoparticle or fusion protein comprises employs a molecule or protein to mimic methods employed by viruses for intracellular delivery of macromolecules (e.g., Kobayashi et al., Bioconjug Chem. 2009 May 20; 20 (5): 953-9), e.g., using a variety of pH sensitive peptides such as vesicular stomatitis virus proteins (VSV G), phage coat proteins and/or shGALA, and/or Fusion associated small transmembrane (FAST) proteins, e.g., avian reovirus (ARV), nelson bay reovirus (NBV), and baboon reovirus (BBV), aquarcovirus reovirus (AQV) and reptilian reovirus (RRV), and/or Bombesin targeting peptide. See, e.g., Peisajovich et al., Eur J Biochem. 2002 September; 269 (17): 4342-50; Sakurai et al., 2011. See also Nesbitt, Targeted Intracellular Therapeutic Delivery Using Liposomes Formulated with Multifunctional FAST proteins, Western University Thesis, 2012.

In some embodiments, a nucleic acid (e.g., RNA or DNA, including a plasmid) encoding OCT4, KLF4, SOX2, or a combination thereof and/or a nucleic acid encoding an inducing agent is encapsulated in a Fusogenix lipid nanoparticle. In some embodiments, a nucleic acid encoding an inducing agent (e.g., rtTA or (TA) is encapsulated in a Fusogenix lipid nanoparticle. In some embodiments, a lipid nanoparticle comprises a viral membrane protein. Without being bound by a particular theory, a lipid nanoparticle may be non-toxic because it comprises a membrane fusion protein that is not a viral membrane fusion protein. Non-limiting examples of membrane fusion proteins include membrane fusion proteins disclosed in U.S. Pat. Nos. 7,851,595, 8,252,901, International Application Publication No. WO 2012/040825, and International Application Publication No. WO 2002/044206.

In some embodiments, a composition of the present disclosure (e.g., comprising a nucleic acid encoding OCT4, KLF4, SOX2, or a combination thereof and/or a nucleic acid encoding an inducing agent) is delivered non-virally. Methods of non-viral delivery of nucleic acids include lipofection, nucleofection, microinjection, biolistics, virosomes, liposomes, immunoliposomes, polycation or lipid: nucleic acid conjugates, naked nucleic acid (e.g., RNA or DNA), artificial virions, and agent-enhanced uptake of a nucleic acid (e.g., RNA or DNA).

In some embodiments, a cationic lipid is used to deliver a nucleic acid. A cationic lipid is a lipid which has a cationic, or positive, charge at physiologic pH. Cationic lipids can take a variety of forms including, but not limited to, liposomes or micelles. Cationic lipids useful for certain aspects of the present disclosure are known in the art, and, generally comprise both polar and non-polar domains, bind to polyanions, such as nucleic acid molecules or negatively supercharged proteins, and are typically known to facilitate the delivery of nucleic acids into cells. Examples of useful cationic lipids include polyethylenimine, polyamidoamine (PAMAM) starburst dendrimers, Lipofectin (a combination of DOTMA and DOPE, see, e.g., U.S. Pat. Nos. 5,049,386, 4,946,787; and 4,897,355), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.). Exemplary cationic liposomes can be made from N-[1-(2,3-diolcoloxy)-propyl]-N,N,N-trimethylammonium chloride (DOTMA), N-[1-(2,3-diolcoloxy)-propyl]-N,N,N-trimethylammonium methylsulfate (DOTAP), 3β-[N—(N′,N′-dimethylaminocthane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Cationic lipids have been used in the art to deliver nucleic acid molecules to cells (see, e.g., U.S. Pat. Nos. 5,855,910; 5,851,548; 5,830,430; 5,780,053; 5,767,099; 8,569,256; 8,691,750; 8,748,667; 8,758,810; 8,759,104; 8,771,728; Lewis et al. 1996. Proc. Natl. Acad. Sci. USA 93:3176; Hope et al. 1998. Molecular Membrane Biology 15:1).

In addition, other lipid compositions are also known in the art and include, e.g., those taught in U.S. Pat. Nos. 4,235,871; 4,501,728; 4,837,028; 4,737,323. Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Polymer-based delivery systems may also be used to deliver a nucleic acid. Polymers including polyethylenimine (PEI), chitosan, Poly (DL-Lactide) (PLA) and Poly (DL-Lactide-co-glycoside) (PLGA), dedrimers, and Polymethacrylate may be used. See, e.g., Yang et al., Macromol Biosci. 2012 December; 12 (12): 1600-14; Ramamoorth et al., J Clin Diagn Res. 2015 January; 9 (1): GE01-GE06. As a non-limiting example, a cationic polymer may be used. A cationic polymer is a polymer having a net positive charge. Cationic polymers are well known in the art, and include those described in Samal et al., Cationic polymers and their therapeutic potential. Chem Soc Rev. 2012 Nov. 7; 41 (21): 7147-94; in published U.S. patent applications U.S. 2014/0141487 A1, U.S. 2014/0141094 A1, U.S. 2014/0044793 A1, U.S. 2014/0018404 A1, U.S. 2014/0005269 A1, and U.S. 2013/0344117 A1; and in U.S. Pat. Nos. 8,709,466; 8,728,526; 8,759,103; and 8,790,664; the entire contents of each are incorporated herein by reference. Exemplary cationic polymers include, but are not limited to, polyallylamine (PAH); polyethyleneimine (PEI); poly(L-lysine) (PLL); poly(L-arginine) (PLA); polyvinylamine homo- or copolymer; a poly(vinylbenzyl-tri-C1-C4-alkylammonium salt); a polymer of an aliphatic or araliphatic dihalide and an aliphatic N,N,N′,N′-tetra-C1-C4-alkyl-alkylenediamine; a poly(vinylpyridin) or poly(vinylpyridinium salt); a poly(N,N-diallyl-N,N-di-C1-C4-alkyl-ammoniumhalide); a homo- or copolymer of a quaternized di-C1-C4-alkyl-aminoethyl acrylate or methacrylate; POLYQUAD™; a polyaminoamide; and the like.

Such formulations may be preferred for the introduction of pharmaceutically acceptable formulations of any of the nucleic acids, engineered proteins, chemical agents, antibodies, and/or recombinant viruses (e.g., lentivirus, adenovirus, alphavirus, vaccinia virus, retrovirus, herpes virus, human papillomavirus, or AAV) disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868; and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500.ANG., containing an aqueous solution in the core.

Alternatively, nanocapsule formulations of the recombinant virus (e.g., lentivirus, alphavirus, vaccinia virus, adenovirus, retrovirus, herpes virus, human papillomavirus, or AAV) may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Kits and Related Compositions

Any of the nucleic acids, engineered proteins, chemical agents, antibodies, and/or recombinant viruses described herein may, in some embodiments, be assembled into pharmaceutical or diagnostic or research kits to facilitate their use in therapeutic, diagnostic or research applications. For example, a kit may be used in treating a neurological disease. In some embodiments, a kit may be used to rejuvenate a cell, tissue, or organ from the central nervous system. A kit may include one or more containers housing one or more components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, a kit comprises instructions for instructions for rejuvenating a cell, tissue, or organ of the central nervous system (e.g., instructions for rejuvenating the cell, tissue, or organ of the central nervous system of a subject in need thereof). In some embodiments, the subject has or is suspected of having a neurological disorder. In some embodiments the cell, tissue, or organ from the central nervous system is a brain cell, brain tissue, or brain. In some embodiments, the kit comprises instructions for treating neurological disorder.

In some embodiments, a kit comprises: (a) a container housing any of the compositions disclosed herein, any of the expression vectors disclosed herein, or any of the recombinant viruses disclosed herein, and (b) instructions for rejuvenating a cell, a tissue, or an organ of a central nervous system, optionally instructions for rejuvenating the cell, tissue, or organ of a subject in need thereof, optionally wherein the cell, tissue, or organ from the central nervous system is a brain cell, brain tissue, or brain.

In some embodiments, a kits comprises (a) a first container housing a viral vector comprising a nucleic acid encoding a tetracycline-controlled transactivator (tTA) or a reverse tetracycline-controlled transactivator (rtTA), wherein the nucleic acid encoding the tTA or rtTA is operably linked to a CaMKIIα promoter; (b) a second container housing a viral vector comprising a first nucleic acid encoding OCT4, a second nucleic acid encoding SOX2, and a third nucleic acid encoding KLF4, wherein the first, second, and third nucleic acids are operably linked to a promoter comprising a tetracycline response element (TRE), and (c) instructions for rejuvenating a cell, tissue, or organ, optionally instructions for rejuvenating the cell, tissue, or organ of a subject in need thereof, optionally wherein the viral vector in (a) and/or (b) is an AAV vector, optionally wherein the AAV vector is packaged in AAV-PHP.b virus, optionally wherein the AAV-PHP.b virus is AAV.PHP.eB virus, optionally wherein the cell, tissue, or organ from the central nervous system is a brain cell, brain tissue, or brain.

In some embodiments, the viral vector in part (a) comprises a sequence that is at least 70% identical (e.g., at least 75%, 80%, 85%, 90%, 95%, 98%, 99%, or 100% identical) to 90% identical to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126, optionally wherein the AAV-PHP.b vector is an AAV.PHP.eB vector.

In some embodiments, the viral vector in part (b) comprises a sequence that is at least 70% identical (e.g., at least 75%, 80%, 85%, 90%, 95%, 98%, 99%, or 100% identical) to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163, optionally wherein the AAV-PHP.b vector is an AAV.PHP.eB vector.

In some embodiments, the instant disclosure relates to a kit for producing a recombinant virus (e.g., lentivirus, alphavirus, vaccinia virus, adenovirus, retrovirus, herpes virus, human papillomavirus, or AAV) and/or engineered cells, the kit comprising a container housing an engineered nucleic acid (e.g., engineered nucleic acid) encoding OCT4, KLF4, SOX2, or a combination thereof and/or an engineered nucleic acid encoding an inducing agent and/or host cells. In some embodiments, the kit further comprises instructions for producing the recombinant virus (e.g., lentivirus, alphavirus, vaccinia virus, adenovirus, retrovirus, herpes virus, human papillomavirus, or AAV) and/or instructions for producing engineered cells. In some embodiments, the kit further comprises at least one container housing a recombinant AAV vector, wherein the recombinant AAV vector comprises a transgene (e.g., a gene associated with a neurological disease, such as a neurodegenerative disease).

In some embodiments, the instant disclosure relates to a kit comprising a container housing any of the engineered nucleic acids (e.g., expression vectors), chemical agents, antibodies, engineered cells, or recombinant viruses described herein. For example, an engineered nucleic acid (e.g., an expression vector) or recombinant virus encoding KLF4, SOX2, OCT4, or a combination thereof may comprise a sequence that is at least 70% (e.g., at least 75%, 80%, 85%, 90%, 95%, 98%, 99%, or 100%) identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. In some embodiments, an engineered nucleic acid (e.g., expression vector) or recombinant virus encoding KLF4, SOX2, OCT4, or a combination thereof comprises SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. In some embodiments, an engineered nucleic acid (e.g., expression vector) encoding these three transcription factors consists of SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163. The kit may further comprise an expression vector or recombinant virus encoding an inducing agent. In some embodiments, an expression vector encoding an inducing agent comprises a sequence that is at least 70% (e.g., at least 75%, 80%, 85%, 90%, or 100%) identical to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126. In some embodiments, an expression vector encoding an inducing agent does not comprise a sequence that is at least 70% (e.g., at least 75%, 80%, 85%, 90%, or 100%) identical to SEQ ID NO: 127. In some embodiments, an expression vector encoding an inducing agent comprises SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126. In some embodiments, an expression vector does not comprise SEQ ID NO: 127. In some embodiments, the expression vector encoding an inducing agent consists of SEQ ID NO: 10, SEQ ID NO: 12, SEQ ID NO: 14, SEQ ID NO: 17, SEQ ID NO: 31, SEQ ID NO: 32, SEQ ID NO: 123, SEQ ID NO: 124, SEQ ID NO: 125, or SEQ ID NO: 126. See also, e.g., International Publication Number WO 2020/069339, entitled “Mutant Reverse Tetracycline Transactivators for Expression of Genes,” which published on Apr. 2, 2020, and which is herein incorporated by reference in its entirety.

In yet another aspect, the present disclosure provides kits comprising any of the nucleic acids (e.g., engineered nucleic acid) capable of inducing OCT4, KLF4, inducing agent, and/or SOX2 expression (e.g., expression vector), any of the engineered proteins described herein, any of the chemical agents activating (e.g., inducing expression of) OCT4, KLF4, an inducing agent, and/or SOX2, any of the antibodies activating (e.g., inducing expression of) OCT4, KLF4, an inducing agent, and/or SOX2, and/or any of the recombinant viruses (e.g., lentivirus, adenovirus, alphavirus, vaccinia virus, retrovirus, herpes virus, human papillomavirus, or AAV) described herein. In yet another aspect, the present disclosure provides kits comprising any of the nucleic acids (e.g., engineered nucleic acid) acids capable of inducing OCT4, KLF4, and/or SOX2 expression (e.g., expression vector), any of the engineered proteins described herein, any of the chemical agents activating (e.g., inducing expression of) OCT4, KLF4, and/or SOX2, any of the antibodies activating (e.g., inducing expression of) OCT4, KLF4, and/or SOX2, and/or any of the recombinant viruses (e.g., lentivirus, adenovirus, alphavirus, vaccinia virus, retrovirus, herpes virus, human papillomavirus, or AAV) described herein. In some embodiments, the kit further comprises a nucleic acid (e.g., engineered nucleic acid) encoding an inducing agent, an engineered protein encoding an inducing agent, a chemical agent capable of modulating (e.g., activating or inhibiting) the activity of an inducing agent, and/or a recombinant virus encoding an inducing agent. In yet another aspect, the present disclosure provides kits comprising any of the nucleic acids (e.g., engineered nucleic acid) (e.g., expression vector) capable of inducing expression of one or more transcription factors selected from the group consisting of OCT4; SOX2; KLF4; and any combinations thereof, any of the engineered proteins described herein, any of the chemical agents activating (e.g., inducing expression of) one or more transcription factors selected from the group consisting of OCT4; SOX2; KLF4; and any combinations thereof, any of the antibodies activating (e.g., inducing expression of) OCT4; SOX2; KLF4; and any combinations thereof, and/or any of the recombinant viruses (e.g., lentivirus, adenovirus, alphavirus, vaccinia virus, retrovirus, herpes virus, human papillomavirus, or AAV) described herein. In certain embodiments, a kit further comprises a nucleic acid (e.g., engineered nucleic acid) (e.g., expression vector) encoding an inducing agent (e.g., rtTA or (TA), any of the engineered proteins encoding an inducing agent, any of the chemical agents capable of activating (e.g., inducing expression of) an inducing agent, and/or any of the recombinant viruses (e.g., lentivirus, adenovirus, alphavirus, vaccinia virus, retrovirus, herpes virus, human papillomavirus, or AAV) encoding an inducing agent.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

The kit may have a variety of forms, such as a blister pouch, a shrink-wrapped pouch, a vacuum scalable pouch, a scalable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

### Therapeutic Applications

Aspects of the present disclosure relate to methods of rejuvenating a cell, tissue, and/or organ, comprising administering to the cell, tissue, or organ any of the compositions disclosed herein. In some embodiments, the composition comprises: (a) an agent that induces OCT4 expression; (b) an agent that induce SOX2 expression; and (c) an agent that induces KLF4 expression, wherein the composition does not comprise an agent that induces c-MYC expression, wherein the cell, tissue, or organ is a central nervous system cell, central nervous system tissue, or central nervous system organ, optionally wherein the central nervous system does not include the eye (e.g., retina, uvea, pupil, lens, cornea, and/or sclera), optionally wherein the cell, tissue, or organ is a brain cell, brain tissue, or brain.

In some embodiments, the composition induces the expression of OCT4, SOX2, and KLF4 for a time period that is sufficient to rejuvenate the cell, tissue, and/or organ.

In some embodiments, the time period sufficient to rejuvenate the cell, tissue, or organ is approximately one month.

In some embodiments, the expression of OCT4, SOX2, and KLF4 is induced for less than two months (e.g., less than 60 days, less than 50 days, less than 40 days, less than 30 days, less than 20 days, less than 10 days, less than 5 days, or less than 1 day).

In some embodiments, the expression of OCT4, SOX2, and KLF4 is induced for at most one month (e.g., at most 1 week, at most 2 weeks, at most 3 weeks, at most 1 day, at most 3 days, at most 5 days, at most 10 days, at most 15 days, at most 20 days, or at most 25 days).

In some embodiments, rejuvenating the cell, tissue, or organ comprises restoring epigenetic information in the cell, tissue, and/or organ.

In some embodiments, rejuvenating the cell, tissue, or organ comprises restoring epigenetic information lost due to aging, injury, disease, or any combination thereof in the cell, tissue, or organ.

In some embodiments, rejuvenating the cell, tissue, or organ comprises reestablishing the epigenetic status of the cell, tissue, or organ an epigenetic status that is similar to the status formed soon after fertilization or final differentiation.

In some embodiments, the composition is administered through retro-orbital venous injection.

In some embodiments, said administration is intrathecal administration.

In some embodiments, the composition is systemically administered, optionally wherein the systematic injection is intravenous injection.

In some embodiments, the composition is not administered to the eye (retina, uvea, pupil, lens, cornea, and/or sclera) of the subject.

In some embodiments, the composition is not administered to the retina of the subject.

In some embodiments, the composition is used to improve cognitive function of the subject.

In some embodiments, the composition is used to improve the memory of the subject.

In some embodiments, the subject has or is suspected of having a neurological disorder.

In some embodiments, the neurological disorder is a neurodegenerative disorder.

In some embodiments, the neurodegenerative disorder is Alzheimer's Disease, Parkinson's Disease, dementia, Friedreich ataxia, amyotrophic lateral sclerosis, or vascular dementia.

Any of the nucleic acids (e.g., engineered nucleic acids) capable of inducing OCT4, KLF4, and/or SOX2 expression (e.g., expression vector), engineered cells, engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein may be used for regulating (e.g., inducing or inducing and stopping) cellular reprogramming, tissue repair, tissue regeneration, organ regeneration, reversing aging, treating a disease, or any combination thereof. In some embodiments, the disease is a neurological disease. In some embodiments, a composition described herein is used to improve the cognitive ability of a subject. In some embodiments, a composition described herein is used to improve memory of a subject. Any of the nucleic acids (e.g., engineered nucleic acids) capable of inducing expression of a combination of transcription factors selected from OCT4, KLF4, and SOX2 (e.g., OCT4 and KLF4, OCT4 and SOX2, SOX2 and KLF4, or KLF4, OCT4, and SOX2), engineered cells, engineered proteins, chemical agents activating (e.g., inducing expression of) a combination of transcription factors selected from OCT4, KLF4, and SOX2 (e.g., OCT4 and KLF4, OCT4 and SOX2, SOX2 and KLF4, or KLF4, OCT4, and SOX2), antibodies activating (e.g., inducing expression of) combination of transcription factors selected from OCT4, KLF4, and SOX2 (e.g., OCT4 and KLF4, OCT4 and SOX2, SOX2 and KLF4, or KLF4, OCT4, and SOX2), and/or recombinant viruses (e.g., lentivirus, adenovirus, alphavirus, vaccinia virus, retrovirus, herpes virus, human papillomavirus, or AAV) described herein may be used for regulating (e.g., inducing or inducing and stopping) cellular reprogramming, tissue repair, tissue regeneration, organ regeneration, reversing aging, treating a disease, or any combination thereof. In some embodiments, any of the nucleic acid (e.g., engineered nucleic acid) capable of inducing OCT4, KLF4, and/or SOX2 expression (e.g., expression vector), any of the engineered cells, any of the engineered proteins, any of the chemical agents activating (e.g., inducing expression of) OCT4, KLF4, and/or SOX2, any of the antibodies activating (e.g., inducing expression of) OCT4, KLF4, and/or SOX2, and/or any of the recombinant viruses (e.g., lentivirus, adenovirus, alphavirus, vaccinia virus, retrovirus, herpes virus, human papillomavirus, or AAV) may be useful in regulating cellular reprogramming, tissue repair, tissue survival, tissue regeneration, tissue growth, tissue function, organ regeneration, organ survival, organ function, or any combination thereof, optionally wherein regulating comprises inducing cellular reprogramming, reversing aging, improving tissue function, improving organ function, tissue repair, tissue survival, tissue regeneration, tissue growth, angiogenesis, scar formation, the appearance of aging, organ regeneration, organ survival, altering the taste and quality of agricultural products derived from animals, treating a disease, or any combination thereof, in vivo or in vitro may be administered to a cell, tissue, or organ that is in vivo (e.g., part of a subject), or may be administered to a cell, tissue, or organ ex vivo. In some embodiments, any of the nucleic acid (e.g., engineered nucleic acid) (e.g., expression vector) capable of inducing expression of OCT4; KLF4; SOX2; or any combination thereof, any of the engineered cells, any of the engineered proteins, any of the chemical agents activating (e.g., inducing expression of) OCT4; KLF4; SOX2; or any combination thereof, any of the antibodies activating (e.g., inducing expression of) OCT4, KLF4, SOX2, or a combination thereof, and/or any of the recombinant viruses (e.g., lentivirus, adenovirus, alphavirus, vaccinia virus, retrovirus, herpes virus, human papillomavirus, or AAV) may be useful in regulating cellular reprogramming, tissue repair, tissue survival, tissue regeneration, tissue growth, tissue function, organ regeneration, organ survival, organ function, or any combination thereof, optionally wherein regulating comprises inducing cellular reprogramming, reversing aging, improving tissue function, improving organ function, tissue repair, tissue survival, tissue regeneration, tissue growth, angiogenesis, scar formation, the appearance of aging, organ regeneration, organ survival, altering the taste and quality of agricultural products derived from animals, treating a disease, or any combination thereof, in vivo or in vitro may be administered to a cell, tissue, or organ that is in vivo (e.g., part of a subject), or may be administered to a cell, tissue, or organ ex vivo. As used herein, regulating may refer to any type of modulation, including inducing or promoting, inhibiting, and/or stopping. Angiogenesis refers to growth of new blood vessels, including capillaries. In some embodiments, the cell, tissue, or organ is a cell, tissue, or organ of the central nervous system. In some embodiments, the cell is a brain cell. In some embodiments, the tissue is brain tissue. In some embodiments, the organ is a brain.

Aspects of the present disclosure also provide methods of regulating cellular reprogramming, promoting tissue repair, promoting tissue survival, promoting tissue regeneration, promoting tissue growth, regulating tissue function, promoting organ regeneration, promoting organ survival, regulating organ function, treating and/or preventing disease, or any combination thereof in the central nervous system (e.g., brain). Regulating may comprise inducing cellular reprogramming, reversing aging, improving tissue function, improving organ function, tissue repair, tissue survival, tissue regeneration, tissue growth, promoting angiogenesis, reducing scar formation, promoting organ regeneration, promoting organ survival, treating a disease, or any combination thereof, in vivo or in vitro. The methods may comprise administering any of the nucleic acids described herein (e.g., DNA and/or RNA), any of the engineered proteins encoding KLF4, OCT4, SOX2, and/or an inducing agent, any of the chemical agents activating (e.g., inducing expression of) OCT4, KLF4, and/or SOX2, antibodies activating (e.g., inducing expression of) OCT4, KLF4, and/or SOX2, any of the recombinant viruses, and/or any of the inducing agents described herein. The methods may comprise administering any of the nucleic acids described herein (e.g., DNA and/or RNA), any of the engineered proteins encoding KLF4, SOX2, OCT4, or any combination thereof, any of the chemical agents activating (e.g., inducing expression of) OCT4; KLF4; SOX2; or any combination thereof, antibodies activating (e.g., inducing expression of) OCT4; KLF4; SOX2; or any combination thereof and/or any of the recombinant viruses described herein. In certain embodiments, the engineered nucleic acids comprise DNA and/or RNA. The engineered nucleic acid may be an expression vector or not an expression vector. For example, the engineered nucleic acid may be mRNA or plasmid DNA. In certain embodiments, the method further comprises administering a nucleic acid (e.g., engineered nucleic acid) encoding an inducing agent, an engineered protein encoding an inducing agent, a chemical agent capable of modulating (e.g., activating or inhibiting) the activity of an inducing agent, and/or a recombinant virus encoding an inducing agent. For example, the engineered nucleic acid may be mRNA or plasmid DNA.

In some instances, a viral vector (e.g., lentivirus vector, alphavirus vector, vaccinia virus vector, adenovirus vector, herpes virus vector, retrovirus vector, or AAV vector) is administered in a recombinant virus (e.g., lentivirus, alphavirus, vaccinia virus, adenovirus, herpes virus, human papillomavirus, retrovirus, or AAV). Without being bound by a particular theory, transient expression of OCT4, SOX2, and KLF4 may result in partial reprogramming of a cell. For example, partial reprogramming may induce a fully differentiated cell to rejuvenate and gain pluripotency. In some embodiments, transient expression of OCT4, SOX2, and/or KLF4 does not induce expression of stem cell markers (e.g., Nanog).

In some embodiments, transient expression of OCT4, SOX2, KLF4, or a combination thereof does not induce expression of stem cell markers (e.g., Nanog). Without being bound by any particular theory, Nanog activation may induce teratomas and cause death of the host. In some embodiments, the method does not induce teratoma formation. In some embodiments, the method does not induce unwanted cell proliferation. In some embodiments, the method does not induce malignant cell growth. In some embodiments, the method does not induce cancer. In some embodiments, the method does not induce glaucoma or macular degeneration. In some embodiments, transient expression is at most 1 hour, 5 hours, 24 hours, 2 days, 3 days, 4 days, 5, days, or 1 week. In some instances, prolonged expression (e.g., continued expression for at least 5 days, at least 1 week, or at least 1 month) of OCT4, SOX2, and KLF4, results in full reprogramming of a cell. In some instances, prolonged expression (e.g., continued expression for at least 5 days, at least 1 week, or at least 1 month) of OCT4, SOX2, KLF4, or a combination thereof, results in full reprogramming of a cell. For example, prolonged expression fully reprograms a cell into a pluripotent cell (e.g., induced pluripotent cell).

Without being bound by a particular theory, expression of OCT4, SOX2, and KLF4 may promote cellular reprogramming, promote tissue regeneration, promote organ regeneration, reverse aging, treat a disease, or any combination thereof because OCT4, SOX2, and KLF4 induce partial reprogramming. As used herein, partial or incomplete reprogramming of a cell refers to a cell that are not stem cells, but have youthful characteristics. In some embodiments, a youthful characteristic is an epigenome that is similar to a young cell. In some embodiments, a stem cell shows higher levels of Nanog expression compared to a cell that is not a stem cell. In some embodiments, youthful characteristics refers to rejuvenation of a cell without changing cell identity. See, e.g., shown in FIG. 16, in which the expression of histone and Chaf (Chromatin assembly factor) genes decline during aging in ear fibroblasts from aged mice (12 months or 15 months) compared to those from young mice, short term of OSKM (3 days) or OSK expression (5 days) induction can reset their gene expression level to young state, without making the cells into a stem cell (e.g., Nanog expression is not induced in these cells).

To practice this embodiment, an effective amount of any of the nucleic acids (e.g., engineered nucleic acids) capable of inducing OCT4, KLF4, and/or SOX2 expression (e.g., expression vector), engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) are administered to a cell, a tissue, organ, and/or subject. In some embodiments, an effective amount of any of the nucleic acids (e.g., engineered nucleic acids) (e.g., expression vector) capable of inducing expression of OCT4, KLF4, SOX2, or a combination thereof, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) are administered to a cell, a tissue, organ, and/or subject. Engineered cells may be administered to any tissue, organ, and/or subject. When the expression vector comprises an inducible promoter (e.g., a TRE promoter, including a TRE3G, TRE2, or P tight), the inducing agent may also be introduced into the cell (e.g., simultaneously or sequentially with one or more nucleic acids (e.g., engineered nucleic acids) encoding OCT4, SOX2, KLF4, or any combination thereof). In one embodiment, OCT4, SOX2, and KLF4 are encoded by one expression vector that is separate from an expression vector encoding the inducing agent. In some instances, the inducing agent is encoded by the same expression vector that encodes OCT4, SOX2, KLF4, or any combination thereof.

In some instances, an inducing agent (e.g., a nucleic acid encoding an inducing agent, an engineered protein encoding an inducing agent, or a virus encoding an inducing agent) and/or a chemical agent (e.g., tetracycline) that is capable of modulating (e.g., activating or inhibiting) activity of the inducing agent is also introduced into a cell, tissue, organ, and/or subject. In certain embodiments, a cell, tissue, subject, and/or organ is further cultured in the presence or absence of a chemical agent that is capable of modulating the activity of an inducing agent (e.g., tetracycline, which includes doxycycline). For a Tet-On system, the inducing agent may be rtTA (e.g., rtTA3 or rtTA4), and the inducing agent promotes expression of OCT4, SOX2, KLF4, or any combination thereof in the presence of tetracycline. For a Tet-Off system, the inducing agent may be tTA, and the inducing agent promotes expression of OCT4, SOX2, KLF4, or any combination thereof in the absence of tetracycline.

Administration of an expression vector encoding a transcription factor described herein and in some cases the inducing agent (e.g., a nucleic acid (e.g., engineered nucleic acid) encoding an inducing agent or the inducing agent as protein) and/or chemical agent that is capable of modulating the activity of the inducing agent under suitable conditions for expression may increase expression of the transgene by at least 10%, at least 20%, at least 30%, at least 40%, at least 50%, at least 60%, at least 70%, at least 80%, at least 90%, at least 100%, at least 200%, at least 300%, at least 400%, at least 500%, at least 500%, at least 600%, at least 700%, at least 800%, at least 900%, or at least 1,000% in a cell. Gene expression may be determined by routine methods including enzyme-linked immunosorbent assays (ELISAs), western blots, and quantification of RNA (e.g., reverse transcription polymerase chain reaction).

In some embodiments, OCT4, SOX2, and/or KLF4 is activated (e.g., expression of one or more of these transcription factors is induced) for a time period sufficient to rejuvenate a cell, tissue, or organ. In some embodiments, the cell or tissue is from the central nervous system. In some embodiments, the organ is the brain. In some embodiments, OCT4, SOX2, and/or KLF4 is activated (e.g., expression of one or more of these transcription factors is induced) for no more than 1 year (e.g., less than 11 months, less than 10 months, less than 9 months, less than 8 months, less than 7 months, less than 6 months, less than 5 months, less than 4 months, less than 3 months, less than 2 months, or less than 1 month). In some embodiments, OCT4, SOX2, and/or KLF4 is activated (e.g., expression of one or more of these transcription factors is induced) for approximately 1 month. In some embodiments, OCT4, SOX2, and/or KLF4 is activated (e.g., expression of one or more of these transcription factors is induced) for less than 2 months.

In some embodiments, any of the nucleic acids (e.g., engineered nucleic acid) capable of inducing OCT4, KLF4, and/or SOX2 expression (e.g., expression vector), engineered proteins described herein, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein, alone, or in combination may be introduced to a tissue, cell, or organ ex vivo (e.g., not in a subject) and the tissue, cell, and/or organ may be further cultured ex vivo. In some embodiments, any of the nucleic acids (e.g., engineered nucleic acid) (e.g., expression vector) capable of inducing expression of OCT4; KLF4; SOX2; or any combination thereof, engineered proteins described herein, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein, alone, or in combination may be introduced to a tissue, cell, or organ ex vivo (e.g., not in a subject) and the tissue, cell, and/or organ may be further cultured ex vivo. In some instances, an inducing agent and/or a chemical agent capable of modulating the activity of the inducing agent is introduced to a tissue, cell, and/or organ ex vivo and the tissue, cell, and/or organ may be further cultured ex vivo. In some embodiments, engineered cells are cultured to produce an engineered tissue. In some embodiments, engineered cells are cultured to produce an engineered organ. In some embodiments, an engineered tissue is cultured to produce an engineered organ. These methods may be useful in producing an engineered (e.g., nerve) cell, engineered (e.g., nerve) tissue or organ (e.g., brain) for transplantation into a subject. In some embodiments, the engineered cell, tissue, and/or organ is transplanted into a subject.

In some embodiments, cells, tissues, organs, or any combination thereof to be engineered are autologous to the subject, e.g., obtained from a subject in need thereof. Administration of autologous cells, autologous tissues, autologous organs, or any combination thereof may result in reduced rejection of the cells, tissues, organs, or any combination thereof compared to administration of non-autologous cells, non-autologous tissue and/or non-autologous organs. Alternatively, the cells, tissues, or organs to be engineered may be allogenic cells, allogenic tissues, or allogenic organs. For example, allogenic cells, allogenic tissue, allogenic organs, or any combination thereof may be derived from a donor (e.g., from a particular species) and administered to a recipient (e.g., from the same species) who is different from the donor. In some embodiments, allogenic cells, allogenic tissue, allogenic organs, or any combination thereof may be derived from a donor subject from a particular species and administered to a recipient subject from a different species from the donor.

In some embodiments, engineered cells comprise more than one cell type (e.g., more than one type of nerve cell).

As a non-limiting example, the methods described herein may be used to produce engineered cell, tissue, or organ of the central nervous system. The engineered organ, engineered tissue, engineered organ, or any combination thereof may be administered to a subject. In some embodiments, administration of an engineered cell, engineered tissue, engineered organ, or a combination thereof improves survival of a subject (e.g., increases the lifespan of a subject relative to not receiving the engineered cell, tissue, or organ).

A pharmaceutical composition described herein may be administered to a subject in need thereof. Non-limiting examples of subjects include any animal (e.g., mammals, including humans). A subject may be suspected of having, be at risk for or have a condition. For example, the condition may be an injury or a disease and the condition may affect any tissue (e.g., nerve tissue). Non-limiting examples of conditions, diseases, and disorders include acute injuries, neurodegenerative disease, chronic diseases, proliferative diseases, cardiovascular diseases, genetic diseases, inflammatory diseases, autoimmune diseases, neurological diseases, hematological diseases, painful conditions, psychiatric disorders, metabolic disorders, cancers, aging, age-related diseases, and diseases affecting any tissue in a subject. In some embodiments, the disease is an ocular disease.

In certain embodiments, any of the nucleic acids (e.g., engineered nucleic acid) capable of inducing OCT4, KLF4, and/or SOX2 expression (e.g., expression vector), engineered proteins described herein, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein, alone, or in combination may be introduced to a subject prior to the onset of a disease (e.g., to prevent a disease or to prevent damage to a cell, tissue, or organ). In certain embodiments, any of the nucleic acids (e.g., engineered nucleic acid) (e.g., expression vector) capable of inducing expression of OCT4; KLF4; SOX2; or any combination thereof, engineered proteins described herein, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein, alone, or in combination may be introduced to a subject prior to the onset of a disease (e.g., to prevent a disease or to prevent damage to a cell, tissue, or organ). In certain embodiments, an inducing agent and/or a chemical agent capable of modulating activity of the inducing agent may be introduced to a subject prior to the onset of a disease. In some embodiments, the subject may be a healthy subject. In certain embodiments, any of the nucleic acids (e.g., engineered nucleic acid) capable of inducing OCT4, KLF4, and/or SOX2 expression (e.g., expression vector), engineered proteins described herein, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein, alone, or in combination may be introduced to a subject following the onset of disease (e.g., to alleviate the damage or symptoms associated with a disease). In certain embodiments, any of the nucleic acids (e.g., engineered nucleic acid) capable of inducing expression of OCT4; KLF4; SOX2; or any combination thereof expression, engineered proteins described herein, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein, alone or in combination, may be introduced to a subject following the onset of disease (e.g., to alleviate the damage or symptoms associated with a disease). In some embodiments, OCT4, KLF4, and/or SOX2 expression is induced prior to the onset of a disease. In some embodiments, expression of OCT4; KLF4; SOX2; or any combination thereof is induced prior to the onset of a disease. In some embodiments, OCT4, KLF4, and/or SOX2 expression is induced after the onset of a disease. In some embodiments, expression of OCT4; KLF4; SOX2; or any combination thereof is induced after the onset of a disease. In some embodiments, OCT4, KLF4, and/or SOX2 expression is induced in a young subject, young cell, young tissue, and/or young organ. In some embodiments, OCT4, KLF4, and/or SOX2 expression is induced in an aged subject, aged cell, aged tissue, and/or aged organ. In some embodiments, expression of OCT4; KLF4; SOX2; or any combination thereof is induced in a young subject, young cell, young tissue, and/or young organ. In some embodiments expression of, OCT4; KLF4; SOX2; or any combination thereof is induced in an aged subject, aged cell, aged tissue, and/or aged organ. In certain embodiments, an inducing agent and/or a chemical agent capable of modulating activity of the inducing agent may be introduced to a subject following the onset of a disease.

In certain embodiments, the tissue may be considered healthy but suboptimal for performance or survival in current or future conditions (e.g., in agriculture, or adverse conditions including disease treatments, toxic therapies, sun exposure, or space travel outside the earth's atmosphere).

In certain embodiments, the condition is aging. All animals typically go through a period of growth and maturation followed by a period of progressive and irreversible physiological decline ending in death. The length of time from birth to death is known as the life span of an organism, and each organism has a characteristic average life span. Aging is a physical manifestation of the changes underlying the passage of time as measured by percent of average life span.

In some embodiments, the condition is a neurological disorder. In some embodiments, the condition affects the central nervous system. In some embodiments, the condition is a condition affecting the brain. In some embodiments, the condition does not affect the eye.

In some cases, characteristics of aging can be quite obvious. For example, characteristics of older humans include skin wrinkling, graying of the hair, baldness, and cataracts, as well as hypermelanosis, osteoporosis, cerebral cortical atrophy, lymphoid depletion, thymic atrophy, increased incidence of diabetes type II, atherosclerosis, cancer, and heart disease. Nehlin et al. (2000), Annals NY Acad Sci 980:176-79. Other aspects of mammalian aging include weight loss, lordokyphosis (hunchback spine), absence of vigor, lymphoid atrophy, decreased bone density, dermal thickening and subcutaneous adipose tissue, decreased ability to tolerate stress (including heat or cold, wounding, anesthesia, and hematopoietic precursor cell ablation), liver pathology, atrophy of intestinal villi, skin ulceration, amyloid deposits, and joint diseases. Tyner et al. (2002), Nature 415:45-53. In some embodiments, aging is determined by a decrease in cognitive ability.

Those skilled in the art will recognize that the aging process is also manifested at the cellular level, as well as in mitochondria. Cellular aging is manifested in loss of doubling capacity, increased levels of apoptosis, changes in differentiated phenotype, and changes in metabolism, e.g., decreased levels of protein synthesis and turnover.

Given the programmed nature of cellular and organismal aging, it is possible to evaluate the “biological age” of a cell or organism by means of phenotypic characteristics that are correlated with aging. For example, biological age can be deduced from patterns of gene expression, resistance to stress (e.g., oxidative or genotoxic stress), rate of cellular proliferation, and the metabolic characteristics of cells (e.g., rates of protein synthesis and turnover, mitochondrial function, ubiquinone biosynthesis, cholesterol biosynthesis, ATP levels within the cell, levels of a Krebs cycle intermediate in the cell, glucose metabolism, nucleic acid (e.g., engineered nucleic acid) metabolism, ribosomal translation rates, etc.). As used herein, “biological age” is a measure of the age of a cell or organism based upon the molecular characteristics of the cell or organism. Biological age is distinct from “temporal age,” which refers to the age of a cell or organism as measured by days, months, and years.

The rate of aging of an organism, e.g., an invertebrate (e.g., a worm or a fly) or a vertebrate (e.g., a rodent, e.g., a mouse) can be determined by a variety of methods, e.g., by one or more of: a) assessing the life span of the cell or the organism; (b) assessing the presence or abundance of a gene transcript or gene product in the cell or organism that has a biological age-dependent expression pattern; (c) evaluating resistance of the cell or organism to stress, e.g., genotoxic stress (e.g., etoposide, UV irradiation, exposure to a mutagen, and so forth) or oxidative stress; (d) evaluating one or more metabolic parameters of the cell or organism; (c) evaluating the proliferative capacity of the cell or a set of cells present in the organism; and (f) evaluating physical appearance or behavior of the cell or organism. In one example, evaluating the rate of aging includes directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined by measuring an age-related parameter. Examples of age-related parameters include: appearance, e.g., visible signs of age; the expression of one or more genes or proteins (e.g., genes or proteins that have an age-related expression pattern); resistance to oxidative stress; metabolic parameters (e.g., protein synthesis or degradation, ubiquinone biosynthesis, cholesterol biosynthesis, ATP levels, glucose metabolism, nucleic acid (e.g., engineered nucleic acid) metabolism, ribosomal translation rates, etc.); and cellular proliferation (e.g., of retinal cells, bone cells, white blood cells, etc.).

Aging can also be determined by the rate of change of biomarkers (e.g., epigenetic marks including DNA methylation level of CpG island in the genome (known as the “Horvath Clock”) beta-galactosidase-positive cells in cells, gene expression changes, or certain changes to the abundance of molecules in the bloodstream). An example is an algorithm from Segterra Inc. that determines “InnerAge” based on blood biomarkers (see InsideTracker.com).

As shown in the Examples herein, recombinant viruses (e.g., AAVs) encoding OCT4, KLF4, and SOX2 promoted regeneration of axons, which may be used to prevent or alleviate neurodegeneration that is often associated with aging. The methods may be used to prevent or alleviate neurodegeneration and peripheral neuropathies associated. Neurodegenerative diseases include Parkinson's disease, Alzheimer's disease, multiple sclerosis, aminotropic lateral sclerosis (ALS), Huntington's disease, and muscular dystrophy. Neurodegeneration may be quantified using any method known in the art. For example, the executive function of an individual may be determined (Moreira et al., Front Aging Neurosci. 2017 Nov. 9; 9:369).

In some embodiments, expression, induction, or activation of OCT4, SOX2, KLF4, or a combination thereof as described herein increases the number of axons per nerve in a tissue, organ, or a subject relative to a control. In some embodiments, a method described herein increases the number of axons per nerve by at least 1.5 fold, by at least 2 fold, by at least 3 fold, by at least 5 fold, by at least 6 fold, by at least 7 fold, by at least 8 fold, by at least 9 fold, by at least 10 fold, by at least 20 fold, by at least 30 fold, by at least 40 fold, by at least 50 fold, by at least 60 fold, by at least 70 fold, by at least 80 fold, by at least 90 fold, or by at least 100 fold relative to a control. In some embodiments, the control is the number of axons per nerve in the tissue, organ, or subject prior to expression, induction, or activation of OCT4, SOX2, KLF4, or a combination thereof.

In some embodiments, the age-related condition is a neurodegenerative disease.

In some embodiments, expression, induction, or activation of OCT4, SOX2, KLF4, or a combination thereof in a neuron increases neurite area of the neuron by at least 1.5 fold, by at least 2 fold, by at least 3 fold, by at least 5 fold, by at least 6 fold, by at least 7 fold, by at least 8 fold, by at least 9 fold, by at least 10 fold, by at least 20 fold, by at least 30 fold, by at least 40 fold, by at least 50 fold, by at least 60 fold, by at least 70 fold, by at least 80 fold, by at least 90 fold, or by at least 100 fold relative to the neuron without expression, induction, or activation of OCT4, SOX2, KLF4, or a combination thereof.

In some embodiments, expression, induction, or activation of OCT4, SOX2, KLF4, or a combination thereof as described herein increases the axon density in a tissue, organ, or a subject relative to a control. In some embodiments, a method described herein increases axon density at least 1.5 fold, by at least 2 fold, by at least 3 fold, by at least 5 fold, by at least 6 fold, by at least 7 fold, by at least 8 fold, by at least 9 fold, by at least 10 fold, by at least 20 fold, by at least 30 fold, by at least 40 fold, by at least 50 fold, by at least 60 fold, by at least 70 fold, by at least 80 fold, by at least 90 fold, or by at least 100 fold relative to a control. In some embodiments, the control is the axon density in the tissue, organ, or subject prior to expression, induction, or activation of OCT4, SOX2, KLF4, or a combination thereof.

In some embodiments, expression, induction, or activation of OCT4, SOX2, KLF4, or a combination thereof in a subject increases the neuronal activity and/or cognitive function of the subject relative to a control. In some embodiments, a method described herein increases neuronal activity and/or cognitive function of a subject by at least 1.5 fold, by at least 2 fold, by at least 3 fold, by at least 5 fold, by at least 6 fold, by at least 7 fold, by at least 8 fold, by at least 9 fold, by at least 10 fold, by at least 20 fold, by at least 30 fold, by at least 40 fold, by at least 50 fold, by at least 60 fold, by at least 70 fold, by at least 80 fold, by at least 90 fold, or by at least 100 fold relative to a control. In some embodiments, the control is the neuronal activity and/or cognitive function of the subject prior to expression, induction, or activation of OCT4, SOX2, KLF4, or a combination thereof. In some embodiments, cognitive function is measured by memory of a subject. In some embodiments, memory refers to long-term memory, short-term memory, or a combination thereof. In some embodiments, cognitive function is measured using one or more of the following tests: Montreal Cognitive Assessment (MoCA), Mini-Mental State Exam (MMSE), Mini-Cog, and/or a test described herein. See, e.g., Arevalo-Rodriguez et al., Cochrane Database Syst Rev. 2015 March; 2015 (3): CD010783, Breton et al., Int J Geriatr Psychiatry. 2019 February; 34 (2): 233-242 and Example 1 below.

In some embodiments, expression, induction, or activation of OCT4, SOX2, KLF4, or a combination thereof increases electric firing of one or more neurons relative to a control. In some embodiments, the neuron is an excitatory neuron. In some embodiments, a method described herein increases electric firing of a neuron by at least 1.5 fold, by at least 2 fold, by at least 3 fold, by at least 5 fold, by at least 6 fold, by at least 7 fold, by at least 8 fold, by at least 9 fold, by at least 10 fold, by at least 20 fold, by at least 30 fold, by at least 40 fold, by at least 50 fold, by at least 60 fold, by at least 70 fold, by at least 80 fold, by at least 90 fold, or by at least 100 fold relative to a control. In some embodiments, the control is the electric firing of a neuron prior to expression, induction, or activation of OCT4, SOX2, KLF4, or a combination thereof. In some embodiments, the electric firing of a neuron is measured over a period of time. In some embodiments, the period of time is at least 1 minutes, at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 20 minutes, at least 25 minutes, at least 30 minutes, at least 1 hour, at least 2 hours, at least 3 hours, at least 4 hours, or at least 5 hours. See, e.g., Example 1 below.

In some embodiments, any of the nucleic acids (e.g., engineered nucleic acids) capable of inducing OCT4, KLF4, and/or SOX2 expression (e.g., expression vector), engineered cells, engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein may be used to treat and/or prevent any of the diseases described herein. In some embodiments, an inducing agent and/or a chemical agent capable of modulating activity of the inducing agent is also used.

As a non-limiting example, an engineered cell of the present disclosure may be used to replace a dysfunctional cell in a subject in need thereof. As another non-limiting example, any of the nucleic acids (e.g., engineered nucleic acids) capable of inducing OCT4, KLF4, and/or SOX2 expression (e.g., expression vector), nucleic acids (e.g., engineered nucleic acids) (e.g., expression vector) capable of inducing expression of a combination of at least two (e.g., at least three) transcription factors selected from OCT4, KLF4, and SOX2, engineered proteins, chemical agents activating (e.g., inducing expression of) OCT4, KLF4, and/or SOX2, chemical agents activating (e.g., inducing expression of) a combination of at least two (e.g., at least three) transcription factors selected from OCT4, KLF4, and SOX2, antibodies activating (e.g., inducing expression of) OCT4, KLF4, and/or SOX2, antibodies activating (e.g., inducing expression of) a combination of at least two (e.g., at least three) transcription factors selected from OCT4, KLF4, and SOX2, and/or recombinant viruses (e.g., lentivirus, adenovirus, alphavirus, vaccinia virus, retrovirus, herpes virus, human papillomavirus, or AAV) may be used to (e.g., incompletely or fully) reprogram a cell in vivo or in vitro. In some embodiments, an inducing agent and/or a chemical agent capable of modulating activity of the inducing agent is also used. For example, any of the any of the nucleic acids (e.g., engineered nucleic acids) capable of inducing OCT4, KLF4, and/or SOX2 expression (e.g., expression vector), engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) may be used to produce an engineered cell. For example, any of the nucleic acids (e.g., engineered nucleic acids) (e.g., expression vector) capable of inducing expression of OCT4, KLF4, SOX2, or a combination thereof, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) may be used to produce an engineered cell. The engineered cell may then be administered to a subject in need thereof. In some embodiments, the engineered cell is cultured in the presence of an inducing agent and/or a chemical agent capable of modulating activity of the inducing agent. In some embodiments, an inducing agent and/or a chemical agent capable of modulating activity of the inducing agent is also administered to the subject.

Non-limiting uses of the nucleic acids (e.g., engineered nucleic acids) capable of inducing OCT4, KLF4, and/or SOX2 expression (e.g., expression vector), nucleic acids (e.g., engineered nucleic acids) (e.g., expression vector) capable of inducing expression of a combination of at least two (e.g., at least three) transcription factors selected from OCT4, KLF4, and SOX2, engineered proteins, chemical agents activating (e.g., inducing expression of) OCT4, KLF4, and/or SOX2, chemical agents activating (e.g., inducing expression of) a combination of at least two (e.g., at least three) transcription factors selected from OCT4, KLF4, and SOX2, engineered cells, antibodies activating (e.g., inducing expression of) OCT4, KLF4, and/or SOX2, antibodies activating (e.g., inducing expression of) a combination of at least two (e.g., at least three) transcription factors selected from OCT4, KLF4, and SOX2, and/or recombinant viruses (e.g., lentivirus, adenovirus, alphavirus, vaccinia virus, retrovirus, herpes virus, human papillomavirus, or AAV) include wound healing, bleed out, injuries, broken bones, gunshot wounds, cuts, scarring during surgery (e.g., cesarean). In some embodiments, an inducing agent and/or a chemical agent capable of modulating activity of the inducing agent is also used.

In some embodiments, any of the of the nucleic acids (e.g., engineered nucleic acids) capable of inducing OCT4, KLF4, and/or SOX2 expression (e.g., expression vector), nucleic acids (e.g., engineered nucleic acids) (e.g., expression vector) capable of inducing expression of a combination of at least two (e.g., at least three) transcription factors selected from OCT4, KLF4, and SOX2, engineered cells, engineered proteins, chemical agents activating (e.g., inducing expression of) OCT4, an KLF4, and/or SOX2, chemical agents activating (e.g., inducing expression of) a combination of at least two (e.g., at least three) transcription factors selected from OCT4, KLF4, and SOX2, antibodies activating (e.g., inducing expression of) OCT4, KLF4, and/or SOX2, antibodies activating (e.g., inducing expression of) a combination of at least two (e.g., at least three) transcription factors selected from OCT4, KLF4, and SOX2, and/or recombinant viruses (e.g., lentivirus, adenovirus, alphavirus, vaccinia virus, retrovirus, herpes virus, human papillomavirus, or AAV) are used to treat disease that affects a non-human subject (e.g., a disease affecting livestock, domesticated pets, and/or other non-human animals). In some embodiments, an inducing agent and/or a chemical agent capable of modulating activity of the inducing agent is also used. For example, the disease may be a cattle disease, a primate (e.g., cynomolgus monkeys, rhesus monkeys) disease, a disease affecting a commercially relevant animal, such as cattle, pigs, horses, sheep, goats, cats, and/or dogs) and/or a disease affecting birds (e.g., commercially relevant birds, such as chickens, ducks, geese, and/or turkeys). The source organism of OCT4, KLF4, and/or SOX2 may be chosen to match the subject being treated. As a non-limiting example, a non-human animal may be treated using a method disclosed herein for veterinary or research purposes and the OCT4, KLF4, and/or SOX2 may be the same species as the non-human animal being used.

In some embodiments, any of the nucleic acids (e.g., engineered nucleic acids) capable of inducing OCT4, KLF4, and/or SOX2 expression (e.g., expression vector), nucleic acids (e.g., engineered nucleic acids) (e.g., expression vector) capable of inducing expression of a combination of at least two (e.g., at least three) transcription factors selected from OCT4, KLF4, and SOX2, engineered cells, engineered proteins, chemical agents activating (e.g., inducing expression of) OCT4, KLF4, and/or SOX2, chemical agents activating (e.g., inducing expression of) a combination of at least two (e.g., at least three) transcription factors selected from OCT4, KLF4, and SOX2, antibodies activating (e.g., inducing expression of) OCT4, KLF4, and/or SOX2, antibodies activating (e.g., inducing expression of) a combination of at least two (e.g., at least three) transcription factors selected from OCT4, KLF4, and SOX2, and/or recombinant viruses (e.g., lentivirus, adenovirus, alphavirus, vaccinia virus, retrovirus, herpes virus, human papillomavirus, or AAV) described herein are used to promote wound healing (e.g., for a cut), treat an injury (e.g., broken bones, bleeding out, gun shot injury, and/or reduce scarring during surgery). In some embodiments, surgery includes cesarean. In some embodiments, an inducing agent and/or a chemical agent capable of modulating activity of the inducing agent is also used.

In some embodiments, any of the nucleic acids (e.g., engineered nucleic acids) capable of inducing OCT4, KLF4, and/or SOX2 expression (e.g., expression vector), nucleic acids (e.g., engineered nucleic acids) (e.g., expression vector) capable of inducing expression of a combination of at least two (e.g., at least three) transcription factors selected from OCT4, KLF4, and SOX2, engineered cells, engineered proteins, chemical agents activating (e.g., inducing expression of) OCT4, KLF4, and/or SOX2, chemical agents activating (e.g., inducing expression of) a combination of at least two (e.g., at least three) transcription factors selected from OCT4, KLF4, and SOX2, antibodies activating (e.g., inducing expression of) OCT4, KLF4, and/or SOX2, antibodies activating (e.g., inducing expression of) a combination of at least two (e.g., at least three) transcription factors selected from OCT4, KLF4, and SOX2, and/or recombinant viruses (e.g., lentivirus, adenovirus, alphavirus, vaccinia virus, retrovirus, herpes virus, human papillomavirus, or AAV) described herein are useful in healing an injury and/or inflammation. In some embodiments, an inducing agent and/or a chemical agent capable of modulating activity of the inducing agent is also used. In some embodiments, the inflammation is hyperinflammation, which may be a side effect of aging. In some embodiments, the hyperinflammation is inflammaging.

In some embodiments, any of the nucleic acids (e.g., engineered nucleic acids) capable of inducing OCT4, KLF4, and/or SOX2 expression (e.g., expression vectors), engineered cells, engineered proteins, chemical agents activating (e.g., inducing expression of) OCT4, KLF4, and/or SOX2, antibodies activating (e.g., inducing expression of) OCT4, KLF4, and/or SOX2, and/or recombinant viruses (e.g., lentivirus, adenovirus, alphavirus, vaccinia virus, retrovirus, herpes virus, human papillomavirus, or AAV) described herein provide a healing capacity.

In some embodiments, any of the nucleic acids (e.g., engineered nucleic acids), engineered cells, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) described herein provide a healing capacity.

In some embodiments, any of the nucleic acids (e.g., engineered nucleic acids), engineered cells, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) described herein are useful in enhancing or rejuvenating optimal or sub-optimal organs. As a non-limiting example, any of the compositions described herein (e.g., recombinant viruses including recombinant AAV viruses) encoding OCT4, KLF4, SOX2, or a combination thereof and/or encoding an inducing agent may be useful in enhancing or rejuvenating suboptimal organs (e.g., from older individuals) that are used for transplantation or to promote organ survival during transport or to promote organ survival after reimplantation of the organ into a subject.

Any of the nucleic acids (e.g., engineered nucleic acids) (e.g., expression vectors) capable of inducing expression of OCT4, KLF4, SOX2, or a combination thereof, engineered cells, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) described herein may be used to rejuvenate or increase the survival and longevity of cells (e.g., hematopoietic stem cells, T-cells, etc.) that are used for transplantation. In some embodiments, recombinant viruses (e.g., AAV viruses) encoding OCT4, KLF4, SOX2, or a combination thereof are useful in rejuvenating or increasing the survival and longevity of cells (e.g., hematopoietic stem cells, T-cells, etc.) that are used for transplantation.

In some embodiments, any of the nucleic acids (e.g., engineered nucleic acids) (e.g., expression vectors) capable of inducing expression of OCT4, KLF4, SOX2, or a combination thereof, engineered cells, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) described herein is used to prevent or relieve the side effects of a toxin and/or a drug (e.g., a chemotherapy) in a subject. Non-limiting examples of side effects include hair loss and peripheral neuropathy. Chemotherapies include vincristine (VCS). In certain embodiments, a composition comprising a recombinant virus (e.g., AAV virus) encoding SOX2, KLF4, OCT4, or a combination thereof, is administered to treat (e.g., recover from) or prevent the side effects induced by a toxin and/or damaging drug therapy (e.g., a chemotherapy drug including VCS).

In some embodiments, any of the nucleic acids (e.g., engineered nucleic acids) (e.g., expression vectors) capable of inducing expression of OCT4, KLF4, SOX2, or a combination thereof, engineered cells, engineered proteins, chemical agents activating (e.g., inducing expression of) OCT4, KLF4, SOX2, or a combination thereof, antibodies activating (e.g., inducing expression of) OCT4, KLF4, SOX2, or a combination thereof, and/or recombinant viruses (e.g., lentivirus, adenovirus, alphavirus, vaccinia virus, retrovirus, herpes virus, human papillomavirus, or AAV) described herein is administered to a subject to prevent or relieve the side effects of a toxin and/or a drug (e.g., a chemotherapy).

In some embodiments, any of the nucleic acids (e.g., engineered nucleic acids) (e.g., expression vectors) capable of inducing expression of OCT4, KLF4, SOX2, or a combination thereof, engineered cells, engineered proteins, chemical agents activating (e.g., inducing expression of) OCT4; KLF4; SOX2; or any combination thereof, antibodies activating (e.g., inducing expression of) OCT4; KLF4; SOX2; or any combination thereof, and/or recombinant viruses (e.g., lentivirus, adenovirus, alphavirus, vaccinia virus, retrovirus, herpes virus, human papillomavirus, or AAV) described herein is administered to a subject to protect a tissue, organ, and/or entire body of the subject from radiation (e.g., prevent the damaging effects of radiation). In certain embodiments, AAV encoding OCT4, SOX2, KLF4, or any combination thereof, is administered to a subject to protect a tissue, organ, and/or entire body of the subject from radiation protect (e.g., prevent the damaging effects of radiation).

Methods for identifying subjects suspected of having a condition may include physical examination, subject's family medical history, subject's medical history, biopsy, genetic testing, DNA sequencing of pathogens or the microbiome, proteomics, or a number of imaging technologies such as ultrasonography, computed tomography, magnetic resonance imaging, magnetic resonance spectroscopy, or positron emission tomography.

Effective amounts of the engineered nucleic acids (e.g., expression vectors, including viral vectors), viruses (e.g., lentiviruses, retroviruses, adenoviruses, retroviruses, alphaviruses, vaccinia viruses, or AAVs) or compositions thereof vary, as recognized by those skilled in the art, depending on route of administration, excipient usage, and co-usage with other active agents. The quantity to be administered depends on the subject to be treated, including, for example, the age of the subject, the gravity of the condition, the weight of the subject, the genetics of the subject, the cells, tissue, or organ to be targeted, or any combination thereof.

Expression of one or more transcription factors of the present disclosure (e.g., OCT4; KLF4; SOX2; or any combination thereof) may result in reprogramming of a cell, tissue repair, tissue regeneration, increase blood flow, organ regeneration, improved immunity, reversal of aging, counter senescence, or any combination thereof. Cellular reprogramming may be determined by determining the extent of differentiation of a cell (e.g., by determining the expression of one or more lineage markers or pluripotency markers, including OCT4, KLF4, SOX2, NANOG, ESRRB, NR4A2, and C/EBPa). The differentiation potential of a cell may also be determined using routine differentiation assays or gene expression patterns. Tissue repair may be determined by tissue replacement and tissue regeneration assays. For example, tissue replacement assays include wound healing assays in cell culture or in mice. Tissue regeneration may be determined by quantifying a particular cell type following expression of one or more transcription factors compared to before expression of OCT4, KLF4, and SOX2. Tissue regeneration may be determined by quantifying a particular cell type following expression of one or more transcription factors compared to before expression of OCT4; KLF4; SOX2; or any combination thereof. In some instances, the methods described herein promote organ regeneration (e.g. liver regeneration or reversal of liver fibrosis and regrowth). In some instances, the methods described herein promote tissue and cell survival. Cell survival in the face of adversity and damage may be determined using assays for cell viability that are standard in the art (e.g., testing neuronal survival with the nano-glo live cell assay from Promega corp.). In some instances, the methods described herein may prevent axonal or Wallerian degeneration, which may be determined by quantifying the rate of axonal degeneration after nerve crush in vitro using nerve cell cultures or in rat and mouse nerve crush models known to those skilled in the art.

In some embodiments, the methods described herein do not induce teratoma formation. In some embodiments, expression of OCT4, SOX2, KLF4, or a combination thereof or activation of OCT4, SOX2, KLF4, or a combination thereof in a subject, tissue, or organ, results in at least 10%, at least 20%, at least 30%, at least 40%, at least 50%, at least 60%, at least 70%, at least 80%, at least 90%, or at least 100% reduction in teratoma formation as compared to expression of OCT4, SOX2, KLF4, or a combination thereof and c-MYC or activation of OCT4, SOX2, KLF4, or a combination thereof and c-MYC in the subject, tissue, or organ. In some embodiments, expression of OCT4, SOX2, and KLF4 or activation of OCT4, SOX2, and KLF4 in a subject, tissue, or organ, results in at least 10%, at least 20%, at least 30%, at least 40%, at least 50%, at least 60%, at least 70%, at least 80%, at least 90%, or at least 100% reduction in teratoma formation as compared to expression of OCT4, SOX2, and KLF4, and c-MYC or activation of OCT4, SOX2, KLF4, and c-MYC in the subject, tissue, or organ. In some embodiments, the number of teratomas or the size of a teratoma in a subject, tissue, or organ is the same or is reduced following expression of OCT4, SOX2, KLF4, or a combination thereof or activation of OCT4, SOX2, KLF4, or a combination thereof in a subject, tissue, or organ as compared to the number of teratomas or the size of a teratoma in the subject, tissue, or organ prior to activation or expression of OCT4, SOX2, KLF4, or a combination thereof.

In some embodiments, the methods described herein do not induce unwanted cell proliferation. In some embodiments, the unwanted cell proliferation is aberrant cell proliferation, which may be benign or cancerous. In some embodiments, expression of OCT4, SOX2, KLF4, or a combination thereof or activation of OCT4, SOX2, KLF4, or a combination thereof in a subject, tissue, or organ reduces unwanted cell proliferation in a subject, tissue, or organ, by at least 10%, at least 20%, at least 30%, at least 40%, at least 50%, at least 60%, at least 70%, at least 80%, at least 90%, or at least 100% as compared to the same method with c-Myc expression or activation. In some embodiments, unwanted cell proliferation in a subject, tissue, or organ is the same or is reduced following expression of OCT4, SOX2, KLF4, or a combination thereof or activation of OCT4, SOX2, KLF4, or a combination thereof in a subject, tissue, or organ as compared to the amount of unwanted cell proliferation in the subject, tissue, or organ prior to activation or expression of OCT4, SOX2, KLF4, or a combination thereof.

In some embodiments, the methods described herein do not induce tumor formation or tumor growth. In some embodiments, expression of OCT4, SOX2, KLF4, or a combination thereof or activation of OCT4, SOX2, KLF4, or a combination thereof in a subject, tissue, or organ reduces the number of tumors or the size of a tumor in a subject, tissue, or organ, by at least 10%, at least 20%, at least 30%, at least 40%, at least 50%, at least 60%, at least 70%, at least 80%, at least 90%, or at least 100% as compared to the same method with c-Myc expression or activation. In some embodiments, the number of tumors or the size of a tumor in a subject, tissue, or organ is the same or is reduced following expression of OCT4, SOX2, KLF4, or a combination thereof or activation of OCT4, SOX2, KLF4, or a combination thereof in a subject, tissue, or organ as compared to the number of tumors or the size of a tumor in the subject, tissue, or organ prior to activation or expression of OCT4, SOX2, KLF4, or a combination thereof. In some embodiments, a method described herein does not induce cancer. In some embodiments, a method described herein does not induce glaucoma.

In yet another aspect, the present disclosure provides methods of regulating (e.g., inducing) cellular reprogramming, tissue repair, tissue regeneration, organ regeneration, reversing aging, or any combination thereof comprising administering to a cell a first nucleic acid (e.g., engineered nucleic acid) encoding OCT4, a second nucleic acid (e.g., engineered nucleic acid) encoding SOX2, and a third nucleic acid (e.g., engineered nucleic acid) encoding KLF4 in the absence of an exogenous nucleic acid (e.g., engineered nucleic acid) capable of expressing c-Myc. In certain embodiments, the first nucleic acid (e.g., engineered nucleic acid) encoding OCT4, the second nucleic acid (e.g., engineered nucleic acid) encoding SOX2, and the third nucleic acid (e.g., engineered nucleic acid) encoding KLF4 is administered to a subject. The subject may be human or non-human. Non-human subjects include, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). In certain embodiments, the three nucleic acids (e.g., engineered nucleic acids) are administered simultaneously. In certain embodiments, the three nucleic acids (e.g., engineered nucleic acids) are administered simultaneously on the same vector.

In yet another aspect, the present disclosure provides methods of regulating (e.g., inducing) cellular reprogramming, tissue repair, tissue regeneration, organ regeneration, reversing aging, or any combination thereof comprising administering to a cell a first nucleic acid (e.g., engineered nucleic acid) encoding OCT4, a second nucleic acid (e.g., engineered nucleic acid) encoding SOX2, a third nucleic acid (e.g., engineered nucleic acid) encoding KLF4, or any combination thereof. In certain embodiments, the first nucleic acid (e.g., engineered nucleic acid) encoding OCT4, the second nucleic acid (e.g., engineered nucleic acid) encoding SOX2, the third nucleic acid (e.g., engineered nucleic acid) encoding KLF4, or any combination thereof is administered to a subject.

In some embodiments, OCT4, SOX2, and/or KLF4 is activated (e.g., expression of one or more of these transcription factors is induced) for a time period sufficient to rejuvenate a cell, tissue, or organ. In some embodiments, the cell or tissue is from the central nervous system. In some embodiments, the organ is the brain. In some embodiments, OCT4, SOX2, and/or KLF4 is activated (e.g., expression of one or more of these transcription factors is induced) for no more than 1 year (e.g., less than 11 months, less than 10 months, less than 9 months, less than 8 months, less than 7 months, less than 6 months, less than 5 months, less than 4 months, less than 3 months, less than 2 months, or less than 1 month). In some embodiments, OCT4, SOX2, and/or KLF4 is activated (e.g., expression of one or more of these transcription factors is induced) for approximately 1 month. In some embodiments, OCT4, SOX2, and/or KLF4 is activated (e.g., expression of one or more of these transcription factors is induced) for less than 2 months.

Further aspects of the disclosure relate to methods of reprogramming comprising rejuvenating the epigenetic clock of a cell, tissue, organ, subject, or any combination thereof. In some embodiments, the cell, tissue, or organ is from the central nervous system. In some embodiments, the subject has a neurological disorder.

Further aspects of the disclosure relate to methods of reprogramming comprising altering the expression of one or more genes associated with ageing.

Further aspects of the disclosure relate to methods comprising resetting the transcriptional profile of an old cell, an old organ, an old tissue, and/or any combination thereof in vitro. In some embodiments, the cell, tissue, or organ is from the central nervous system.

Further aspects of the disclosure relate to methods comprising resetting the transcriptional profile of an old cell, an old organ, an old tissue, an old subject and/or any combination thereof in vivo. In some embodiments, the cell, tissue, or organ is from the central nervous system. In some embodiments, the subject has a neurological disorder. Further aspects of the disclosure relate to methods of transdifferentiating cells, e.g., to produce cells of the central nervous system, including neurons. In some embodiments, a cell of the central nervous system is a brain cell.

Methods of reprogramming are also provided herein. In some embodiments, a method of reprogramming described herein comprises reversing or rejuvenating the epigenetic clock of a cell, tissue, organ, or a subject. In some embodiments, the epigenetic clock may be partially or fully reversed. In some embodiments, the epigenetic clock of a cell, tissue, organ, or a subject is measured using DNA methylation-based age (DNAmAGE or DNAm age). In some embodiments, a method described herein reduces the DNAmAge age of a cell by 1%, 5%, 10%, 20%, 30%, 40%, 50%, 60%, 70%, 80%, 90%, or 100%.

In some embodiments, a method of reprogramming described herein comprises altering the expression of one or more genes associated with ageing. In some embodiments, expression of a gene is increased by at least 1%, at least 5%, at least 10%, at least 20%, at least 30%, at least 40%, at least 50%, at least 60%, at least 70%, at least 80%, at least 90%, or at least 100%. In some embodiments, expression of a gene is reduced by at least 1%, at least 5%, at least 10%, at least 20%, at least 30%, at least 40%, at least 50%, at least 60%, at least 70%, at least 80%, at least 90%, or at least 100%. In some embodiments, expression of one or more genes following performance of a method is determined relative to expression of the one or more genes prior to performance of the method. In some embodiments, expression of one or more genes is determined relative to expression of the one or more genes in a young cell, a young subject, a young tissue, a young organ, or any combination thereof. In some embodiments, expression of one or more genes is determined relative to expression of the one or more genes in an old cell, an old subject, an old tissue, an old organ, or any combination thereof.

A gene associated with ageing may be a gene whose expression is altered in an old, an old tissue, an old organ, an old subject, or any combination thereof as compared to a young counterpart. Non-limiting examples of genes include RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

Aspects of the present disclosure relate to methods comprising resetting the transcriptional profile of an old cell, an old organ, an old tissue, and/or any combination thereof in vitro. Aspects of the present disclosure relate to methods comprising resetting the transcriptional profile of an old cell, an old organ, an old tissue, an old subject and/or any combination thereof in vivo. In some embodiments, resetting the transcriptional profile an old cell, an old organ, an old tissue, an old subject and/or any combination thereof comprises altering the gene expression of one or more genes associated with ageing. In some embodiments, resetting the transcriptional profile an old cell, an old organ, an old tissue, an old subject and/or any combination thereof comprises reversing the epigenetic clock. In some embodiments, the transcription profile of an old cell is reset. In some embodiments, the transcriptional profile of an old cell, an old organ, an old tissue, an old subject, or any combination thereof is reset to that of a young cell, a young tissue, a young organ, a young subject, or any combination thereof. In some embodiments, a method described herein reverses one or more changes in gene expression that are detected between an old cell, an old organ, an old tissue, an old subject, or any combination thereof and a control. In some embodiments, the control is a young cell, a young organ, a young tissue, a young subject, or any combination thereof. In some embodiments, the transcriptional profile of an old cell, an old organ, an old tissue, an old subject, or any combination thereof is changed from a young counterpart. In some embodiments, a method described herein resets at least 10%, at least 20%, at least 30%, at least 40%, at least 50%, at least 60%, at least 70%, at least 80%, at least 90%, or 100% of the gene expression change in an old cell, an old organ, an old tissue, an old subject, or any combination thereof to a young level. In some embodiments, the method alters the expression of one or more genes associated with ageing. In some embodiments, the one or more genes is a central nervous system gene. In some embodiments, the one or more genes is a brain gene. Without being bound by a particular theory, resetting of a central nervous system (e.g., brain) gene expression level in an aged cell to a young level may be indicative of an improvement of nerve cell function. In some embodiments, one or more genes associated with ageing is at least one gene selected from the group consisting of RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

In some embodiments, the method does not induce malignant cell growth. In some embodiments, the method does not induce tumor growth or tumor formation. In some embodiments, the method does not induce glaucoma or macular degeneration.

In some aspects, the cellular reprogramming methods described herein may be used to promote the transdifferentiation of cells, which may be useful in treatment of disease. In some embodiments, the methods described herein may improve the efficiency of existing methods of transdifferentiation. For example, OCT4, SOX2, KLF4, or a combination thereof may be activated (e.g., expressed) in one cell type along with one or more perturbations of genes that affect cell fate to promote lineage reprogramming or conversion to another cell type. In some embodiments, the perturbation is reducing expression of a lineage determining factor. In some embodiments, the perturbation is expression of a lineage determining factor. In some embodiments, the lineage determining factor is a lineage transcription factor.

Additional non-limiting examples of transdifferentiation inducing factors for production of various cell types may be found in Cieslar-Pobuda et al., Biochim Biophys Acta Mol Cell Res. 2017 July; 1864 (7): 1359-1369, which is herein incorporated by reference in its entirety. See e.g., Table 4 of Cieslar-Pobuda et al., Biochim Biophys Acta Mol Cell Res. 2017 July; 1864 (7): 1359-1369.

Induction of OCT4, SOX2, KLF4, or a combination thereof may increase the efficiency of trandifferentiation of cells by at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000%, including all values in between, as compared to a control. The efficiency of transdifferentiation may be measured by any suitable method including comparing the percentage of cells that were transdifferentiated when OCT4, SOX2, KLF4, or a combination thereof was activated as compared to control cells in which OCT4, SOX2, KLF4, or a combination thereof was not activated.

These and other aspects of the present invention will be further appreciated upon consideration of the following Examples, which are intended to illustrate certain particular embodiments of the invention but are not intended to limit its scope, as defined by the claims.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1: Reprogramming of the Brain Using OCT4, SOX2, and KLF4

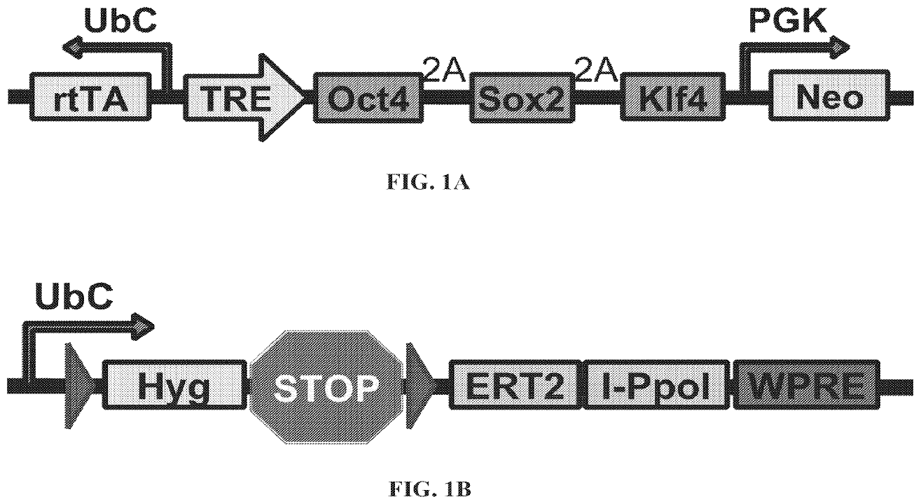

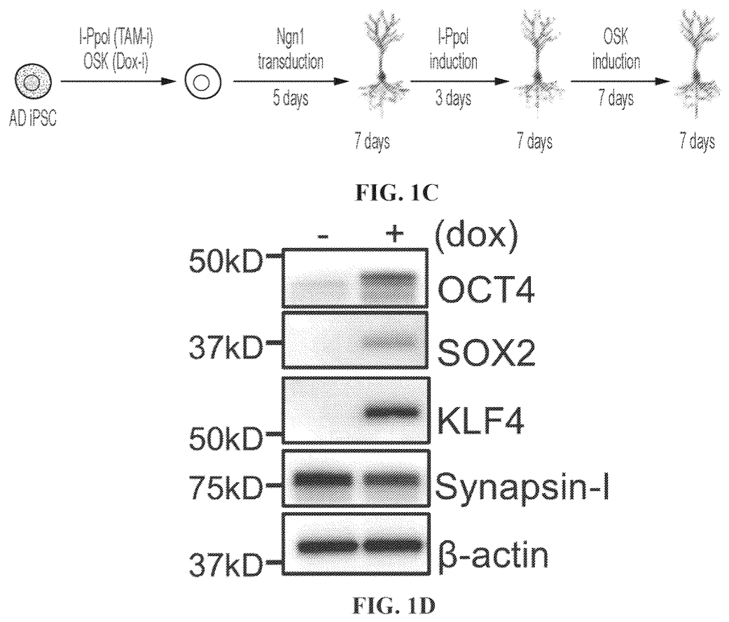

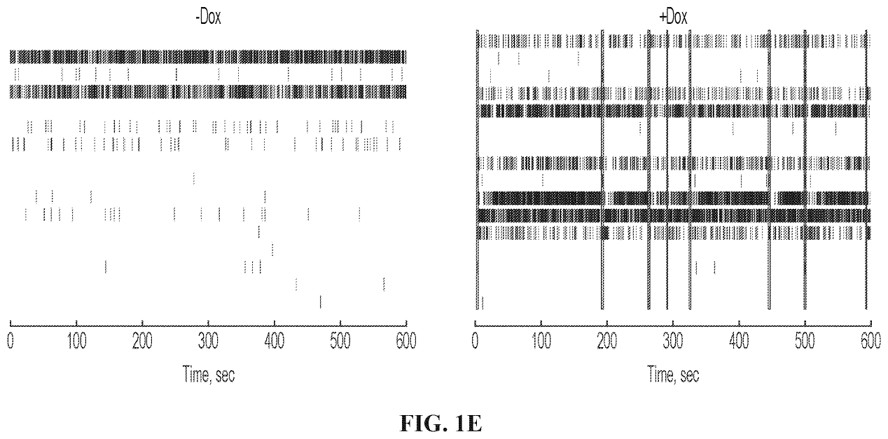

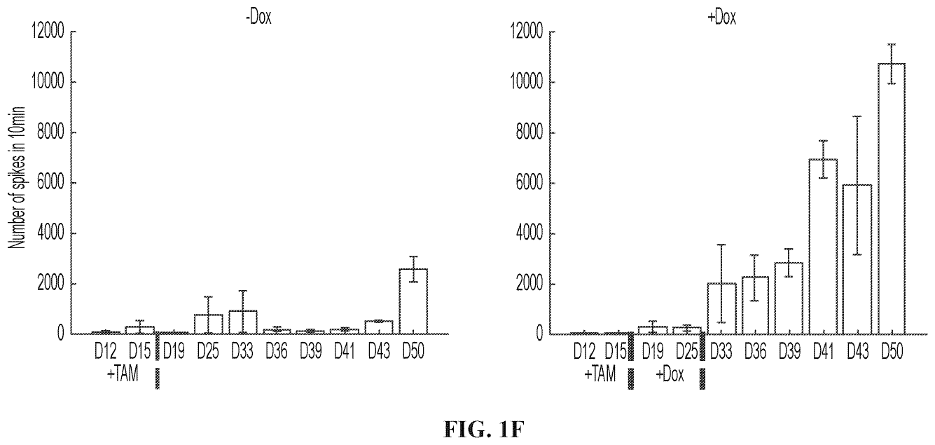

To test whether epigenetic reprogramming improves neuronal function, an all-in-one lentiviral vector was developed to efficiently deliver tetracycline-inducible expression of OCT4, SOX2, and KLF4 genes (OSK) (FIG. 1A). A major obstacle to study aging using iPSC-derived neurons is that the aging signatures in the transcriptome and epigenome are eliminated during the full reprogramming process (Mertens et al., Directly Reprogrammed Human Neurons Retain Aging-Associated Transcriptomic Signatures and Reveal Age-Related Nucleocytoplasmic Defects. Cell Stem Cell, 2015. 17 (6): p. 705-718). To age the iPSC-derived neurons, a tamoxifen-inducible I-PpoI lentiviral vector was developed under hygromycin selection (FIG. 1B). Both lentiviral vectors were integrated into iPSCs harboring two ApoE4 alleles, one of the strongest risk factors for AD (FIG. 1C). iPSC-derived neurons by expressing NGN1 were treated with 1 μM tamoxifen for three days to induce aging, after which, OSK expression was induced by 1 μg/mL doxycycline for 7 days. Western blotting verified the robust expression of OSK (FIG. 1D). To measure the electric firing activity of reprogrammed neurons, a multi-electrode array was used (MEA) (FIG. 1E). OSK expression increased the firing activity and synchrony of I-PpoI-damaged neurons (FIGS. 1E and 1F), indicating that reprogramming improved neuronal function.

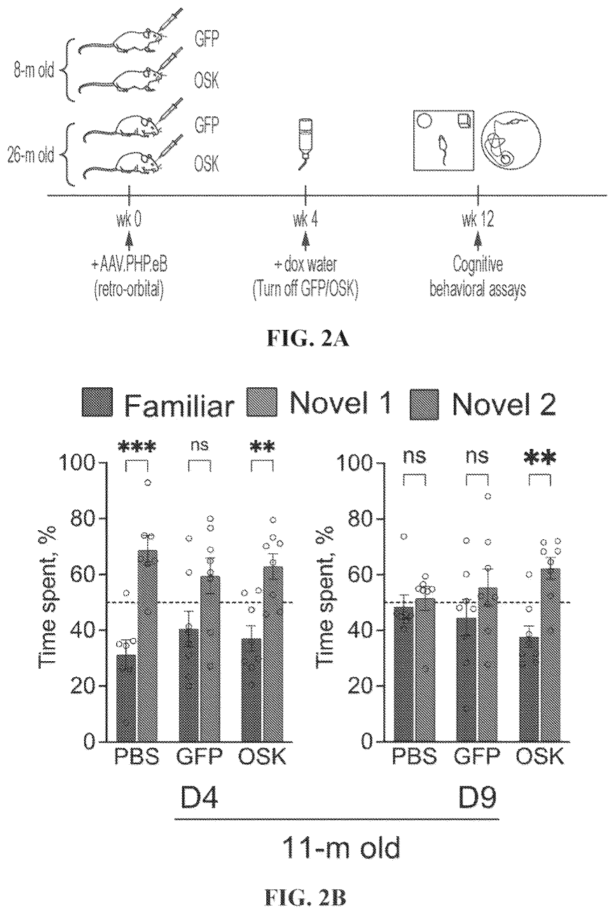

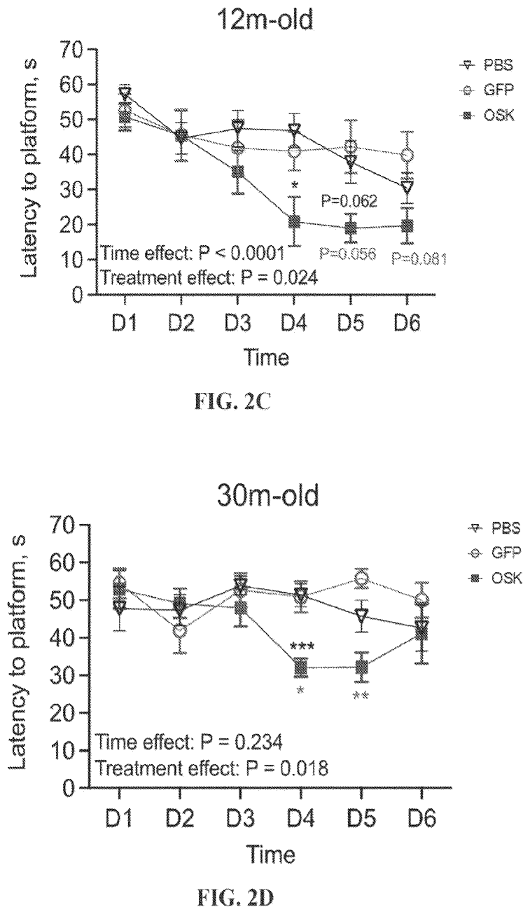

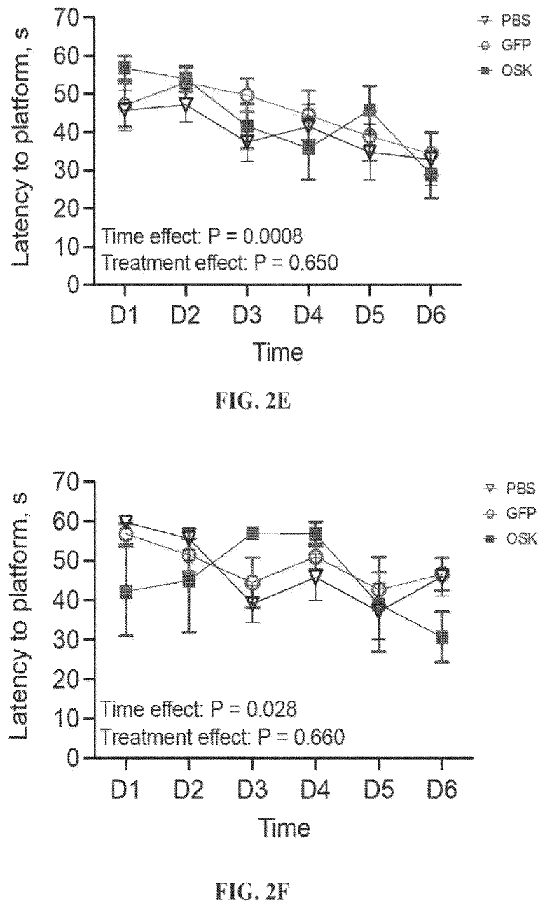

Next, this functional improvement in neurons was tested to determine whether it confers benefits in vivo. Middle- and old-age mice were injected with AAV.PHP.B virus to deliver either GFP or OSK expression under the control of tetracycline transactivator (tTA) into the brain (FIG. 2A). Four weeks after injection, 1 g/L doxycycline was administrated through drinking water to turn off the expression of GFP or OSK. Behavioral assays including novel object recognition (NOR) and Morris water maze (MWM) were used to assess the cognitive performance of the mice. After familiarizing the mice with two identical objects for three days, one of the objects was replaced with a novel object (Novel 1) at day 4 to test short-term memory and with a second novel object (Novel 2) at day 9 to test long-term memory of the mice. Strikingly, OSK-reprogrammed mice were able to distinguish the familiar and novel objects at both day 4 and day 9, but mice from the PBS and GFP groups could only do so at day 4, suggesting that OSK reprogramming improved the long-term memory of the mice in 11-month-old middle-aged mice (FIG. 2B). MWM measures the ability of the mice to learn to escape to a hidden platform under the water. It was found that OSK-treated mice learned this task significantly faster than PBS- and GFP-treated mice at both 12 months of age (FIG. 2C) and 30 months of age (FIG. 2D), indicating that reprogramming improved the cognitive performance of middle-age and old-age mice. Notably, while age-related cognitive decline was observed between 12-month-old mice treated with PBS in FIG. 2C and 30-month-old mice treated with PBS in FIG. 2D, OSK improved the learning efficiency of both sets of mice. Without being bound by a particular theory, expression of OSK in the brain may be useful in reversing cognitive decline in patients with Alzheimer's Disease.

It was found that expression of OCT4, KLF4, and SOX2 for one month delivered by AAV.PHP.eB virus improved cognitive function of mice, but expression of OCT4, KLF4, and SOX2 for two months did not (FIGS. 2E-2F).

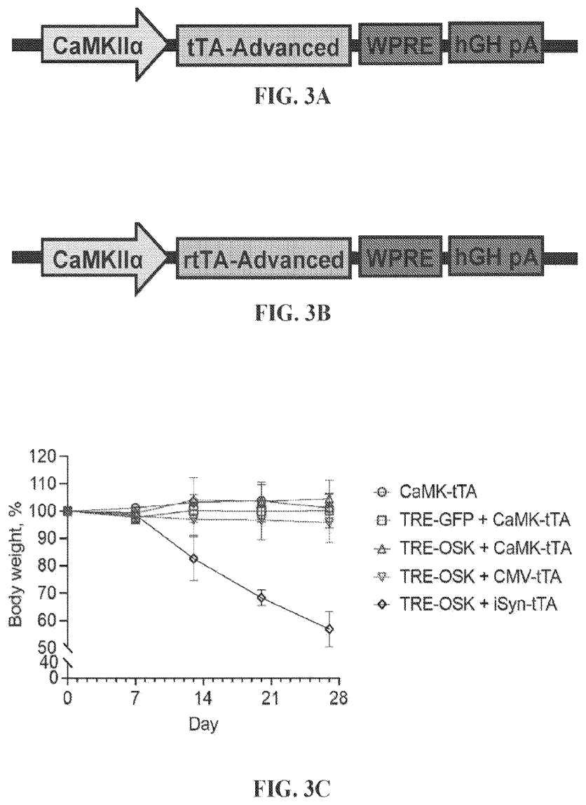

Previous studies suggest that continuous reprogramming in vivo is detrimental, which leads to rapid death of the mice (Ocampo et al., In Vivo Amelioration of Age-Associated Hallmarks by Partial Reprogramming. Cell, 2016. 167 (7): p. 1719-1733 e12). However, the results presented herein showed that continuous reprogramming in the brain for at least 1 month is not detrimental (FIGS. 2A-2D), suggesting that restricting reprogramming to post-mitotic cells may be a much safer strategy to conduct in vivo reprogramming. Therefore, two AAV vectors were developed to express either tTA (Tet-Off) or rtTA (Tet-On) under the control of CaMKIIα promoter in order to reprogram excitatory neurons (FIGS. 3A-3B). In FIGS. 2A-2F and FIGS. 3A-3C, AAV vectors were packaged into AAV-PHP.eB viruses. Excitatory neurons are the leading types of neurons that are damaged in AD patients (Fu et al., A tau homeostasis signature is linked with the cellular and regional vulnerability of excitatory neurons to tau pathology. Nat Neurosci, 2019. 22 (1): p. 47-56). OSK expression under the control of the CaMKIIα promoter does not lead to any body weight loss compared to Synapsin-I promoter, indicating that CaMKIIα promoter is a safe choice to conduct neuronal reprogramming in the brain (FIG. 3C).

The methods presented in this Example provide a promising strategy to conduct neuronal reprogramming to treat neurodegeneration. Without being bound by a particular theory, several of the AAV vectors disclosed herein may be useful in humans as gene therapy for neurodegenerative diseases.

The following sequences are associated with pLVX-rtTA-hOSK-all-in-one (human) (SEQ ID NO: 123) in FIG. 1A: rtTA Advanced in reverse complement (SEQ ID NO: 128); Amino acid sequence of rtTA Advanced (SEQ ID NO: 129); UbC promoter in reverse complement (SEQ ID NO: 130); P tight TRE promoter (SEQ ID NO: 24); human OCT4 (SEQ ID NO: 40); Amino acid sequence of human OCT4 (SEQ ID NO: 41); P2A (SEQ ID NO: 119); Amino acid sequence of P2A (SEQ ID NO: 118); human SOX2 (SEQ ID NO: 42); Amino acid sequence of human SOX2 (SEQ ID NO: 43); T2A (SEQ ID NO: 120); Amino acid sequence of T2A (SEQ ID NO: 9); human KLF4 (SEQ ID NO: 131); Amino acid sequence of human KLF4 (SEQ ID NO: 45); PGK promoter (SEQ ID NO: 132); Neomycin resistance gene (SEQ ID NO: 133); amino acid sequence of Neomycin resistance gene (SEQ ID NO: 134); and WPRE (SEQ ID NO: 135).

The following sequences associated with pAAV-CMV-tTA (Advanced) (SEQ ID NO: 32) in FIG. 2A: CMV promoter (SEQ ID NO: 136); tTA Advanced (SEQ ID NO: 137); Amino acid sequence of tTA Advanced (SEQ ID NO: 138); and hGH pA (SEQ ID NO: 139).

The following sequences associated with pAAV-TRE3G-EGFP (SEQ ID NO: 140) in FIG. 2A: TRE3G (SEQ ID NO: 7); EGFP (SEQ ID NO: 141); amino acid sequence of EGFP (SEQ ID NO: 142); and SV40 pA ((SEQ ID NO: 143).

The following sequences associated with pAAV-TRE3G-OSK (mouse) (Seq ID NO: 16) in FIG. 2A: TRE3G (SEQ ID NO: 7): mouse Oct4 (SEQ ID NO: 1): amino acid sequence of mouse OCT4 (SEQ ID NO: 2); P2A (SEQ ID NO: 144); amino acid sequence of P2A (SEQ ID NO: 118); mouse Sox2 (SEQ ID NO: 3); amino acid sequence of mouse SOX2 (SEQ ID NO: 4); T2A (SEQ ID NO: 120); amino acid sequence of T2A (SEQ ID NO: 9); mouse Klf4 (SEQ ID NO: 145); amino acid sequence of mouse KLF4 (SEQ ID NO: 6); and SV40 pA (SEQ ID NO: 143).

The following sequences associated with pAAV-CaMKIIα-tTA2 (SEQ ID NO: 124) in FIG. 3A: CaMKIIα promoter (SEQ ID NO: 146); tTA-Advanced (SEQ ID NO: 137); amino acid sequence of tTA Advanced (SEQ ID NO: 138); WPRE (SEQ ID NO: 147); and hGH pA (SEQ ID NO: 148).

The following sequences associated with pAAV-CaMKIIα-rtTA2S-M2 (SEQ ID NO: 125) in FIG. 3B: CaMKIIα promoter (SEQ ID NO: 149); rtTA2S-M2 (SEQ ID NO: 14); amino acid sequence of rtTA2S-M2 (SEQ ID NO: 15); WPRE (SEQ ID NO: 152); and hGH pA (SEQ ID NO: 153).

The following sequences associated with pAAV-CaMKIIα-rtTA3 (SEQ ID NO: 126) in FIG. 3B: CaMKIIα promoter (SEQ ID NO: 154); rtTA3 (SEQ ID NO: 10); amino acid sequence of rtTA3 (SEQ ID NO: 11); WPRE (SEQ ID NO: 155); and hGH pA (SEQ ID NO: 156).

The following sequences are associated with pAAV-ihSyn1-tTA (SEQ ID NO: 127) in FIG. 3C: Synapsin-I promoter (SEQ ID NO: 157); tTA (SEQ ID NO: 158); amino acid sequence of tTA (SEQ ID NO: 159); WPRE (SEQ ID NO: 160); and hGH pA (SEQ ID NO: 161).

### Example 2: Reprogramming the Brain in Alzheimer's Disease

To test the effect of OCT4, SOX2, and KLF4 (OSK) reprogramming on the progression of AD, an inducible neuron-specific OCT4, KLF4, and SOX2 (OKS) (INOKS) transgenic mouse was generated (FIG. 4A). In the iNOKS mice, M2-rtTA is under the control of the CaMKIIα promoter, which is mainly active in excitatory neurons. When treated with doxycycline, the iNOKS mice will express OCT4, KLF4, and SOX2 in their excitatory neurons. Next, the iNOKS mice were crossed with the 5×FAD mice to get the 5×FAD-iNOKS mice (5×FAD-rtTA-OKS). The FAD Ctrl mice include both the 5×FAD-rtTA mice and the 5×FAD-OKS mice that do not express OKS upon doxycycline treatment. After 4 weeks of doxycycline treatment, all mice were subjected to behavioral assays at week 8 (FIG. 4B). Doxycycline treatment induced the expression of OCT4, KLF4, and SOX2 in all regions of the hippocampus such as CA1, CA2, CA3, and dentate gyrus (DG) (FIG. 4C). It was found that the reprogrammed 5×FAD mice tend to require fewer days to reach the preset learning criterion (FIG. 4D).

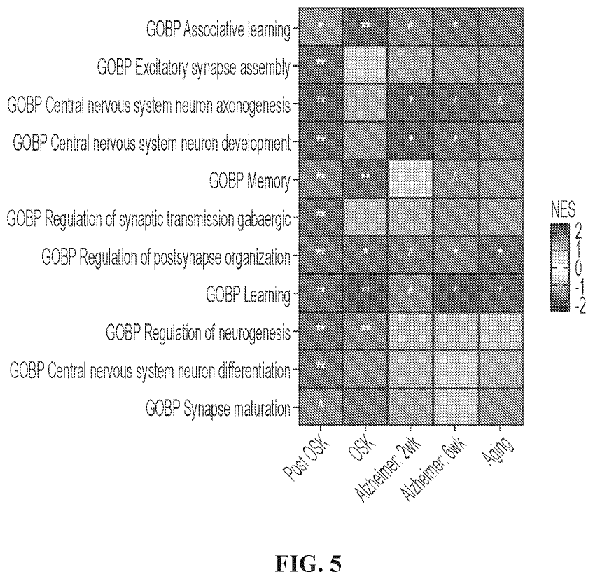

Next, RNA-seq analysis was performed of the hippocampal samples that were collected at one month after doxycycline withdrawal (Post OSK) or immediately after 4 weeks of doxycycline treatment (OSK). RNA-seq analysis was conducted in iNOKS mice. Gene Set Enrichment Analysis (GSEA) indicated that the genes involved in learning and memory functions are upregulated in Post OSK samples. The gene sets for GSEA analysis in FIG. 5 is from the Molecular Signatures Database (MSigDB). Such gene sets are downregulated in OSK samples, implying a transient dedifferentiation process during neuron reprogramming (FIG. 5). Most of these gene sets are downregulated during aging or after induction of p25 for 2 weeks or 6 weeks. The p25 induction reference data is from Gjoneska et al. Nature. 2015 Feb. 19; 518 (7539): 365-9. This result indicates that OSK reprogramming improves cognitive performance by upregulating cognition-related genes.

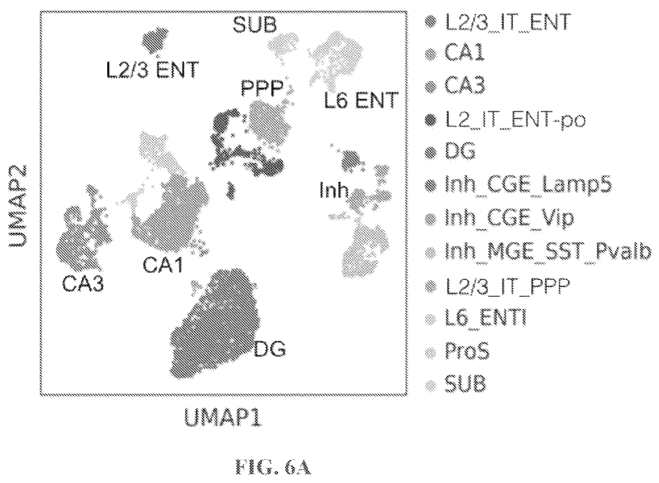

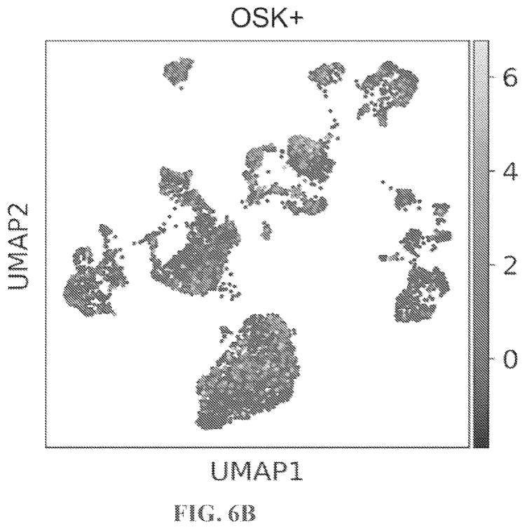

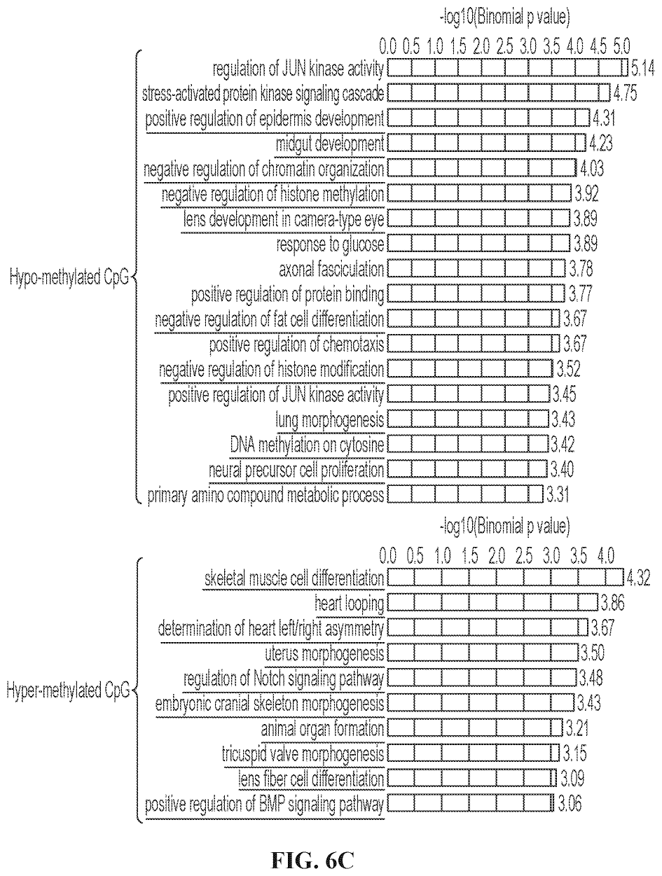

To investigate how OSK interacts with the epigenome in the hippocampal neurons, a single-nucleus multi-omics analysis was performed using iNOKS mice. Most cell types in the hippocampus were successfully identified (FIG. 6A). OSK expression was mainly detected in a subset of cells in the excitatory neuronal clusters, and very few in the inhibitory clusters (FIG. 6B). Hypo- and hyper-methylated CpG sites were identified by comparing OSK+ and OSK− cells within the same mouse. Hypomethylated CpGs were defined by comparing the OSK+ and OSK− cells within each cluster and the CpGs with significantly lower methylation (p<0.05) were selected. Hypermethylated CpGs were defined by comparing the OSK+ and OSK− cells within each cluster and the CpGs with significantly higher methylation (p<0.05) were selected. Gene ontology analysis was performed on the hypo- and hyper-methylated cites. Differentially methylated site (DMS) analysis indicated that OSK-induced hypo- and hyper-methylation were enriched at development-related genomic loci (FIG. 6C), suggesting that developmental network is involved in the epigenomic reprogramming process induced by OSK in neurons.

### Example 3. Non-Limiting Examples of Sequences

Nucleotide sequence encoding Mus Musculus OCT4 (no stop codon) (SEQ ID NO: 1):

ATGGCTGGACACCTGGCTTCAGACTTCGCCTTCTCACCCCCACCAGGTGGGGGTGATGGGTCAGCA
GGGCTGGAGCCGGGCTGGGTGGATCCTCGAACCTGGCTAAGCTTCCAAGGGCCTCCAGGTGGGCC
TGGAATCGGACCAGGCTCAGAGGTATTGGGGATCTCCCCATGTCCGCCCGCATACGAGTTCTGCGG
AGGGATGGCATACTGTGGACCTCAGGTTGGACTGGGCCTAGTCCCCCAAGTTGGCGTGGAGACTTT
GCAGCCTGAGGGCCAGGCAGGAGCACGAGTGGAAAGCAACTCAGAGGGAACCTCCTCTGAGCCCT
GTGCCGACCGCCCCAATGCCGTGAAGTTGGAGAAGGTGGAACCAACTCCCGAGGAGTCCCAGGAC
ATGAAAGCCCTGCAGAAGGAGCTAGAACAGTTTGCCAAGCTGCTGAAGCAGAAGAGGATCACCTT
GGGGTACACCCAGGCCGACGTGGGGCTCACCCTGGGCGTTCTCTTTGGAAAGGTGTTCAGCCAGA
CCACCATCTGTCGCTTCGAGGCCTTGCAGCTCAGCCTTAAGAACATGTGTAAGCTGCGGCCCCTGC
TGGAGAAGTGGGTGGAGGAAGCCGACAACAATGAGAACCTTCAGGAGATATGCAAATCGGAGAC
CCTGGTGCAGGCCCGGAAGAGAAAGCGAACTAGCATTGAGAACCGTGTGAGGTGGAGTCTGGAG
ACCATGTTTCTGAAGTGCCCGAAGCCCTCCCTACAGCAGATCACTCACATCGCCAATCAGCTTGGG
CTAGAGAAGGATGTGGTTCGAGTATGGTTCTGTAACCGGCGCCAGAAGGGCAAAAGATCAAGTAT
TGAGTATTCCCAACGAGAAGAGTATGAGGCTACAGGGACACCTTTCCCAGGGGGGGCTGTATCCT
TTCCTCTGCCCCCAGGTCCCCACTTTGGCACCCCAGGCTATGGAAGCCCCCACTTCACCACACTCTA
CTCAGTCCCTTTTCCTGAGGGCGAGGCCTTTCCCTCTGTTCCCGTCACTGCTCTGGGCTCTCCCATG
CATTCAAAC

Amino acid sequence encoding Mus Musculus OCT4 (SEQ ID NO: 2):

MAGHLASDFAFSPPPGGGDGSAGLEPGWVDPRTWLSFQGPPGGPGIGPGSEVLGISPCPPAYEFCGGMA
YCGPQVGLGLVPQVGVETLQPEGQAGARVESNSEGTSSEPCADRPNAVKLEKVEPTPEESQDMKALQ
KELEQFAKLLKQKRITLGYTQADVGLTLGVLFGKVFSQTTICRFEALQLSLKNMCKLRPLLEKWVEEA
DNNENLQEICKSETLVQARKRKRTSIENRVRWSLETMFLKCPKPSLQQITHIANQLGLEKDVVRVWFC
NRRQKGKRSSIEYSQREEYEATGTPFPGGAVSFPLPPGPHFGTPGYGSPHFTTLYSVPFPEGEAFPSVPVT
ALGSPMHSN

Nucleotide sequence encoding Mus Musculus SOX2 (no stop codon) (SEQ ID NO: 3):

ATGTATAACATGATGGAGACGGAGCTGAAGCCGCCGGGCCCGCAGCAAGCTTCGGGGGGCGGCG
GCGGAGGAGGCAACGCCACGGCGGCGGCGACCGGCGGCAACCAGAAGAACAGCCCGGACCGCGT
CAAGAGGCCCATGAACGCCTTCATGGTATGGTCCCGGGGGCAGCGGCGTAAGATGGCCCAGGAGA
ACCCCAAGATGCACAACTCGGAGATCAGCAAGCGCCTGGGCGCGGAGTGGAAACTTTTGTCCGAG
ACCGAGAAGCGGCCGTTCATCGACGAGGCCAAGCGGCTGCGCGCTCTGCACATGAAGGAGCACCC
GGATTATAAATACCGGCCGCGGCGGAAAACCAAGACGCTCATGAAGAAGGATAAGTACACGCTTC
CCGGAGGCTTGCTGGCCCCCGGCGGGAACAGCATGGCGAGCGGGGTTGGGGTGGGCGCCGGCCTG
GGTGCGGGCGTGAACCAGCGCATGGACAGCTACGCGCACATGAACGGCTGGAGCAACGGCAGCT
ACAGCATGATGCAGGAGCAGCTGGGCTACCCGCAGCACCCGGGCCTCAACGCTCACGGCGCGGCA
CAGATGCAACCGATGCACCGCTACGACGTCAGCGCCCTGCAGTACAACTCCATGACCAGCTCGCA
GACCTACATGAACGGCTCGCCCACCTACAGCATGTCCTACTCGCAGCAGGGCACCCCCGGTATGGC
GCTGGGCTCCATGGGCTCTGTGGTCAAGTCCGAGGCCAGCTCCAGCCCCCCCGTGGTTACCTCTTC
CTCCCACTCCAGGGCGCCCTGCCAGGCCGGGGACCTCCGGGACATGATCAGCATGTACCTCCCCGG
CGCCGAGGTGCCGGAGCCCGCTGCGCCCAGTAGACTGCACATGGCCCAGCACTACCAGAGCGGCC
CGGTGCCCGGCACGGCCATTAACGGCACACTGCCCCTGTCGCACATG

Amino acid sequence encoding Mus Musculus SOX2 (translated) (SEQ ID NO: 4)

MYNMMETELKPPGPQQASGGGGGGGNATAAATGGNQKNSPDRVKRPMNAFMVWSRGQRRKMAQE
NPKMHNSEISKRLGAEWKLLSETEKRPFIDEAKRLRALHMKEHPDYKYRPRRKTKTLMKKDKYTLPG
GLLAPGGNSMASGVGVGAGLGAGVNQRMDSYAHMNGWSNGSYSMMQEQLGYPQHPGLNAHGAAQ
MQPMHRYDVSALQYNSMTSSQTYMNGSPTYSMSYSQQGTPGMALGSMGSVVKSEASSSPPVVTSSSH
SRAPCQAGDLRDMISMYLPGAEVPEPAAPSRLHMAQHYQSGPVPGTAINGTLPLSHM

Nucleotide sequence encoding Mus Musculus KLF4 (no stop codon) (SEQ ID NO: 5):

ATGAGGCAGCCACCTGGCGAGTCTGACATGGCTGTCAGCGACGCTCTGCTCCCGTCCTTCTCCACG
TTCGCGTCCGGCCCGGCGGGAAGGGAGAAGACACTGCGTCCAGCAGGTGCCCCGACTAACCGTTG
GCGTGAGGAACTCTCTCACATGAAGCGACTTCCCCCACTTCCCGGCCGCCCCTACGACCTGGCGGC
GACGGTGGCCACAGACCTGGAGAGTGGCGGAGCTGGTGCAGCTTGCAGCAGTAACAACCCGGCCC
TCCTAGCCCGGAGGGAGACCGAGGAGTTCAACGACCTCCTGGACCTAGACTTTATCCTTTCCAACT
CGCTAACCCACCAGGAATCGGTGGCCGCCACCGTGACCACCTCGGCGTCAGCTTCATCCTCGTCTT
CCCCAGCGAGCAGCGGCCCTGCCAGCGCGCCCTCCACCTGCAGCTTCAGCTATCCGATCCGGGCCG
GGGGTGACCCGGGCGTGGCTGCCAGCAACACAGGTGGAGGGCTCCTCTACAGCCGAGAATCTGCG
CCACCTCCCACGGCCCCCTTCAACCTGGCGGACATCAATGACGTGAGCCCCTCGGGCGGCTTCGTG
GCTGAGCTCCTGCGGCCGGAGTTGGACCCAGTATACATTCCGCCACAGCAGCCTCAGCCGCCAGGT
GGCGGGCTGATGGGCAAGTTTGTGCTGAAGGCGTCTCTGACCACCCCTGGCAGCGAGTACAGCAG
CCCTTCGGTCATCAGTGTTAGCAAAGGAAGCCCAGACGGCAGCCACCCCGTGGTAGTGGCGCCCT
ACAGCGGTGGCCCGCCGCGCATGTGCCCCAAGATTAAGCAAGAGGCGGTCCCGTCCTGCACGGTC
AGCCGGTCCCTAGAGGCCCATTTGAGCGCTGGACCCCAGCTCAGCAACGGCCACCGGCCCAACAC
ACACGACTTCCCCCTGGGGCGGCAGCTCCCCACCAGGACTACCCCTACACTGAGTCCCGAGGAACT
GCTGAACAGCAGGGACTGTCACCCTGGCCTGCCTCTTCCCCCAGGATTCCATCCCCATCCGGGGCC
CAACTACCCTCCTTTCCTGCCAGACCAGATGCAGTCACAAGTCCCCTCTCTCCATTATCAAGAGCTC
ATGCCACCGGGTTCCTGCCTGCCAGAGGAGCCCAAGCCAAAGAGGGGAAGAAGGTCGTGGCCCCG
GAAAAGAACAGCCACCCACACTTGTGACTATGCAGGCTGTGGCAAAACCTATACCAAGAGTTCTC
ATCTCAAGGCACACCTGCGAACTCACACAGGCGAGAAACCTTACCACTGTGACTGGGACGGCTGT
GGGTGGAAATTCGCCCGCTCCGATGAACTGACCAGGCACTACCGCAAACACACAGGGCACCGGCC
CTTTCAGTGCCAGAAGTGCGACAGGGCCTTTTCCAGGTCGGACCACCTTGCCTTACACATGAAGAG
GCAC

Amino acid sequence encoding Mus Musculus KLF4 (translated) (SEQ ID NO: 6):

MRQPPGESDMAVSDALLPSFSTFASGPAGREKTLRPAGAPTNRWREELSHMKRLPPLPGRPYDLAATV
ATDLESGGAGAACSSNNPALLARRETEEFNDLLDLDFILSNSLTHQESVAATVTTSASASSSSSPASSGP
ASAPSTCSFSYPIRAGGDPGVAASNTGGGLLYSRESAPPPTAPFNLADINDVSPSGGFVAELLRPELDPV
YIPPQQPQPPGGGLMGKFVLKASLTTPGSEYSSPSVISVSKGSPDGSHPVVVAPYSGGPPRMCPKIKQEA
VPSCTVSRSLEAHLSAGPQLSNGHRPNTHDFPLGRQLPTRTTPTLSPEELLNSRDCHPGLPLPPGFHPHPG
PNYPPFLPDQMQSQVPSLHYQELMPPGSCLPEEPKPKRGRRSWPRKRTATHTCDYAGCGKTYTKSSHL
KAHLRTHTGEKPYHCDWDGCGWKFARSDELTRHYRKHTGHRPFQCQKCDRAFSRSDHLALHMKRH

TRE3G promoter sequence (non-limiting example of a TRE promoter) (SEQ ID NO: 7):

TTTACTCCCTATCAGTGATAGAGAACGTATGAAGAGTTTACTCCCTATCAGTGATAGAGAACGTAT
GCAGACTTTACTCCCTATCAGTGATAGAGAACGTATAAGGAGTTTACTCCCTATCAGTGATAGAGA
ACGTATGACCAGTTTACTCCCTATCAGTGATAGAGAACGTATCTACAGTTTACTCCCTATCAGTGA
TAGAGAACGTATATCCAGTTTACTCCCTATCAGTGATAGAGAACGTATAAGCTTTAGGCGTGTACG
GTGGGCGCCTATAAAAGCAGAGCTCGTTTAGTGAACCGTCAGATCGCCTGGAGCAATTCCACAAC
ACTTTTGTCTTATACCAACTTTCCGTACCACTTCCTACCCTCGTAAA

SV40-derived terminator sequence (SEQ ID NO: 8):

TGCGCGCAGCGGCCGACCATGGCCCAACTTGTTTATTGCAGCTTATAATGGTTACAAATAAAGCAA
TAGCATCACAAATTTCACAAATAAAGCATTTTTTTCACTGCATTCTAGTTGTGGTTTGTCCAAACTC
ATCAATGTATCTTATCATGTCTGGATCTCGGTACCG

T2A sequence (SEQ ID NO: 9): GSGEGRGSLLTCGDVEENPGP

Nucleotide sequence encoding rtTA3 (with 2 VP16 domain at 3′ end) (SEQ ID NO: 10):

ATGTCTAGGCTGGACAAGAGCAAAGTCATAAACGGAGCTCTGGAATTACTCAATGGTGTCGGTAT
CGAAGGCCTGACGACAAGGAAACTCGCTCAAAAGCTGGGAGTTGAGCAGCCTACCCTGTACTGGC
ACGTGAAGAACAAGCGGGCCCTGCTCGATGCCCTGCCAATCGAGATGCTGGACAGGCATCATACC
CACTTCTGCCCCCTGGAAGGCGAGTCATGGCAAGACTTTCTGCGGAACAACGCCAAGTCATACCGC
TGTGCTCTCCTCTCACATCGCGACGGGGCTAAAGTGCATCTCGGCACCCGCCCAACAGAGAAACA
GTACGAAACCCTGGAAAATCAGCTCGCGTTCCTGTGTCAGCAAGGCTTCTCCCTGGAGAACGCACT
GTACGCTCTGTCCGCCGTGGGCCACTTTACACTGGGCTGCGTATTGGAGGAACAGGAGCATCAAGT
AGCAAAAGAGGAAAGAGAGACACCTACCACCGATTCTATGCCCCCACTTCTGAGACAAGCAATTG
AGCTGTTCGACCGGCAGGGAGCCGAACCTGCCTTCCTTTTCGGCCTGGAACTAATCATATGTGGCC
TGGAGAAACAGCTAAAGTGCGAAAGCGGCGGGCCGACCGACGCCCTTGACGATTTTGACTTAGAC
ATGCTCCCAGCCGATGCCCTTGACGATTTTGACCTTGACATGCTCCCCGGGTAA

Amino acid sequence encoding rtTA3 (SEQ ID NO: 11):

MSRLDKSKVINGALELLNGVGIEGLTTRKLAQKLGVEQPTLYWHVKNKRALLDALPIEMLDRHHTHF
CPLEGESWQDFLRNNAKSYRCALLSHRDGAKVHLGTRPTEKQYETLENQLAFLCQQGFSLENALYALS
AVGHFTLGCVLEEQEHQVAKEERETPTTDSMPPLLRQAIELFDROGAEPAFLFGLELIICGLEKQLKCES
GGPTDALDDFDLDMLPADALDDFDLDMLPG

Nucleotide sequence encoding rtTA4 (with 3 VP16 domain at 3′ end) (SEQ ID NO: 12):

ATGTCCCGCTTGGATAAGAGCAAGGTAATAAATAGCGCACTCGAACTCCTCAACGGCGTGGGCAT
CGAAGGTCTGACTACTCGAAAGCTCGCCCAGAAATTGGGTGTGGAGCAACCTACATTGTATTGGC
ATGTCAAGAACAAAAGAGCCCTGCTGGACGCTCTTCCTATTGAAATGCTTGACAGGCATCACACTC
ATTCCTGCCCCCTTGAGGTCGAGAGTTGGCAAGATTTTCTCCGAAACAATGCAAAGTCCTACCGCT
GCGCACTTTTGTCCCATAGGGATGGAGCAAAAGTGCACCTGGGAACCAGGCCAACAGAGAAACAA
TACGAGACTCTCGAGAACCAGTTGGCTTTCTTGTGCCAACAGGGGTTCTCACTTGAAAATGCCCTT
TACGCACTGTCAGCCGTTGGACATTTTACCCTGGGGTGCGTTCTTGAGGAGCAAGAACATCAGGTT
GCTAAGGAGGAGCGCGAGACTCCAACCACTGATTCTATGCCACCTTTGCTGAAACAGGCCATTGA
ACTTTTCGATAGACAGGGTGCTGAACCTGCCTTTCTCTTCGGGTTGGAGCTGATTATTTGTGGTCTC
GAAAAACAGCTGAAATGTGAAAGTGGTGGCCCTACTGACGCCCTCGATGATTTCGACCTGGATAT
GCTGCCAGCCGATGCACTTGATGATTTCGATTTGGATATGCTTCCAGCCGACGCACTGGACGACTT
CGATTTGGACATGCTTCCCGGTTAA

Amino acid sequence encoding rtTA4 (SEQ ID NO: 13):

MSRLDKSKVINSALELLNGVGIEGLTTRKLAQKLGVEQPTLYWHVKNKRALLDALPIEMLDRHHTHSC
PLEVESWQDFLRNNAKSYRCALLSHRDGAKVHLGTRPTEKQYETLENQLAFLCQQGFSLENALYALSA
VGHFTLGCVLEEQEHQVAKEERETPTTDSMPPLLKQAIELFDRQGAEPAFLFGLELIICGLEKQLKCESG
GPTDALDDFDLDMLPADALDDFDLDMLPADALDDFDLDMLPG

Nucleic acid sequence of pAAV-TRE3G-OSK-SV40 pA, TRE-OSK-SV40, or TRE3G-OSK-SV40 pA vector (SEQ ID NO: 16):

TTATGCAGTGCTGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGTAATGGT
AACAACGTTGCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAGCACTGGGGCCAGATG
GTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCCGAAGGTAACTG
GCTTCAGCAGAGCGCAGATACCAAATACTGTCCTTCTAGTGTAGCCGTAGTTAGGCCACCACTTCA
AGAACTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGTATCT
TTATAGTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCCTTTT
GCTCACATGTTCTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGG
CACCCCAGGCTTTACACTTTATGCTTCCGGCTCGTATGTTGTGTGGAATTGTGAGCGGATAACAATT
TCACACAGGAAACAGCTATGACCATGATTACGCCAGATTTAATTAAGGCCTTAATTAGGCTGCGCG
CTCGCTCGCTCACTGAGGCCGCCCGGGCAAAGCCCGGGCGTCGGGCGACCTTTGGTCGCCCGGCCT
CAGTGAGCGAGCGAGCGCGCAGAGAGGGAGTGGCCAACTCCATCACTAGGGGTTCCTTGTAGTTA
ATGATTAACCCGCCATGCTACTTATCTACGTAGCCATGCTCTAGGAAGATCGGAATTCTTTACTCCC
TATCAGTGATAGAGAACGTATGAAGAGTTTACTCCCTATCAGTGATAGAGAACGTATGCAGACTTT
ACTCCCTATCAGTGATAGAGAACGTATAAGGAGTTTACTCCCTATCAGTGATAGAGAACGTATGAC
CAGTTTACTCCCTATCAGTGATAGAGAACGTATCTACAGTTTACTCCCTATCAGTGATAGAGAACG
TATATCCAGTTTACTCCCTATCAGTGATAGAGAACGTATAAGCTTTAGGCGTGTACGGTGGGCGCC
TATAAAAGCAGAGCTCGTTTAGTGAACCGTCAGATCGCCTGGAGCAATTCCACAACACTTTTGTCT
TATACCAACTTTCCGTACCACTTCCTACCCTCGTAAAGCGGCCGCGCCACCATGGCTGGACACCTG
GCTTCAGACTTCGCCTTCTCACCCCCACCAGGTGGGGGTGATGGGTCAGCAGGGCTGGAGCCGGG
CTGGGTGGATCCTCGAACCTGGCTAAGCTTCCAAGGGCCTCCAGGTGGGCCTGGAATCGGACCAG
GCTCAGAGGTATTGGGGATCTCCCCATGTCCGCCCGCATACGAGTTCTGCGGAGGGATGGCATACT
GTGGACCTCAGGTTGGACTGGGCCTAGTCCCCCAAGTTGGCGTGGAGACTTTGCAGCCTGAGGGCC
AGGCAGGAGCACGAGTGGAAAGCAACTCAGAGGGAACCTCCTCTGAGCCCTGTGCCGACCGCCCC
AATGCCGTGAAGTTGGAGAAGGTGGAACCAACTCCCGAGGAGTCCCAGGACATGAAAGCCCTGCA
GAAGGAGCTAGAACAGTTTGCCAAGCTGCTGAAGCAGAAGAGGATCACCTTGGGGTACACCCAGG
CCGACGTGGGGCTCACCCTGGGCGTTCTCTTTGGAAAGGTGTTCAGCCAGACCACCATCTGTCGCT
TCGAGGCCTTGCAGCTCAGCCTTAAGAACATGTGTAAGCTGCGGCCCCTGCTGGAGAAGTGGGTG
GAGGAAGCCGACAACAATGAGAACCTTCAGGAGATATGCAAATCGGAGACCCTGGTGCAGGCCC
GGAAGAGAAAGCGAACTAGCATTGAGAACCGTGTGAGGTGGAGTCTGGAGACCATGTTTCTGAAG
TGCCCGAAGCCCTCCCTACAGCAGATCACTCACATCGCCAATCAGCTTGGGCTAGAGAAGGATGT
GGTTCGAGTATGGTTCTGTAACCGGCGCCAGAAGGGCAAAAGATCAAGTATTGAGTATTCCCAAC
GAGAAGAGTATGAGGCTACAGGGACACCTTTCCCAGGGGGGGCTGTATCCTTTCCTCTGCCCCCAG
GTCCCCACTTTGGCACCCCAGGCTATGGAAGCCCCCACTTCACCACACTCTACTCAGTCCCTTTTCC
TGAGGGCGAGGCCTTTCCCTCTGTTCCCGTCACTGCTCTGGGCTCTCCCATGCATTCAAACGCTAGC
GGCAGCGGCGCCACGAACTTCTCTCTGTTAAAGCAAGCAGGAGATGTTGAAGAAAACCCCGGGCC
TGCATGCATGTATAACATGATGGAGACGGAGCTGAAGCCGCCGGGCCCGCAGCAAGCTTCGGGGG
GCGGCGGCGGAGGAGGCAACGCCACGGCGGCGGCGACCGGCGGCAACCAGAAGAACAGCCCGGA
CCGCGTCAAGAGGCCCATGAACGCCTTCATGGTATGGTCCCGGGGGCAGCGGCGTAAGATGGCCC
AGGAGAACCCCAAGATGCACAACTCGGAGATCAGCAAGCGCCTGGGCGCGGAGTGGAAACTTTTG
TCCGAGACCGAGAAGCGGCCGTTCATCGACGAGGCCAAGCGGCTGCGCGCTCTGCACATGAAGGA
GCACCCGGATTATAAATACCGGCCGCGGCGGAAAACCAAGACGCTCATGAAGAAGGATAAGTAC
ACGCTTCCCGGAGGCTTGCTGGCCCCCGGCGGGAACAGCATGGCGAGCGGGGTTGGGGTGGGCGC
CGGCCTGGGTGCGGGCGTGAACCAGCGCATGGACAGCTACGCGCACATGAACGGCTGGAGCAACG
GCAGCTACAGCATGATGCAGGAGCAGCTGGGCTACCCGCAGCACCCGGGCCTCAACGCTCACGGC
GCGGCACAGATGCAACCGATGCACCGCTACGACGTCAGCGCCCTGCAGTACAACTCCATGACCAG
CTCGCAGACCTACATGAACGGCTCGCCCACCTACAGCATGTCCTACTCGCAGCAGGGCACCCCCGG
TATGGCGCTGGGCTCCATGGGCTCTGTGGTCAAGTCCGAGGCCAGCTCCAGCCCCCCCGTGGTTAC
CTCTTCCTCCCACTCCAGGGCGCCCTGCCAGGCCGGGGACCTCCGGGACATGATCAGCATGTACCT
CCCCGGCGCCGAGGTGCCGGAGCCCGCTGCGCCCAGTAGACTGCACATGGCCCAGCACTACCAGA
GCGGCCCGGTGCCCGGCACGGCCATTAACGGCACACTGCCCCTGTCGCACATGGCATGCGGCTCC
GGCGAGGGCAGGGGAAGTCTTCTAACATGCGGGGACGTGGAGGAAAATCCCGGCCCACTCGAGAT
GAGGCAGCCACCTGGCGAGTCTGACATGGCTGTCAGCGACGCTCTGCTCCCGTCCTTCTCCACGTT
CGCGTCCGGCCCGGCGGGAAGGGAGAAGACACTGCGTCCAGCAGGTGCCCCGACTAACCGTTGGC
GTGAGGAACTCTCTCACATGAAGCGACTTCCCCCACTTCCCGGCCGCCCCTACGACCTGGCGGCGA
CGGTGGCCACAGACCTGGAGAGTGGCGGAGCTGGTGCAGCTTGCAGCAGTAACAACCCGGCCCTC
CTAGCCCGGAGGGAGACCGAGGAGTTCAACGACCTCCTGGACCTAGACTTTATCCTTTCCAACTCG
CTAACCCACCAGGAATCGGTGGCCGCCACCGTGACCACCTCGGCGTCAGCTTCATCCTCGTCTTCC
CCAGCGAGCAGCGGCCCTGCCAGCGCGCCCTCCACCTGCAGCTTCAGCTATCCGATCCGGGCCGG
GGGTGACCCGGGCGTGGCTGCCAGCAACACAGGTGGAGGGCTCCTCTACAGCCGAGAATCTGCGC
CACCTCCCACGGCCCCCTTCAACCTGGCGGACATCAATGACGTGAGCCCCTCGGGCGGCTTCGTGG
CTGAGCTCCTGCGGCCGGAGTTGGACCCAGTATACATTCCGCCACAGCAGCCTCAGCCGCCAGGTG
GCGGGCTGATGGGCAAGTTTGTGCTGAAGGCGTCTCTGACCACCCCTGGCAGCGAGTACAGCAGC
CCTTCGGTCATCAGTGTTAGCAAAGGAAGCCCAGACGGCAGCCACCCCGTGGTAGTGGCGCCCTA
CAGCGGTGGCCCGCCGCGCATGTGCCCCAAGATTAAGCAAGAGGCGGTCCCGTCCTGCACGGTCA
GCCGGTCCCTAGAGGCCCATTTGAGCGCTGGACCCCAGCTCAGCAACGGCCACCGGCCCAACACA
CACGACTTCCCCCTGGGGCGGCAGCTCCCCACCAGGACTACCCCTACACTGAGTCCCGAGGAACTG
CTGAACAGCAGGGACTGTCACCCTGGCCTGCCTCTTCCCCCAGGATTCCATCCCCATCCGGGGCCC
AACTACCCTCCTTTCCTGCCAGACCAGATGCAGTCACAAGTCCCCTCTCTCCATTATCAAGAGCTC
ATGCCACCGGGTTCCTGCCTGCCAGAGGAGCCCAAGCCAAAGAGGGGAAGAAGGTCGTGGCCCCG
GAAAAGAACAGCCACCCACACTTGTGACTATGCAGGCTGTGGCAAAACCTATACCAAGAGTTCTC
ATCTCAAGGCACACCTGCGAACTCACACAGGCGAGAAACCTTACCACTGTGACTGGGACGGCTGT
GGGTGGAAATTCGCCCGCTCCGATGAACTGACCAGGCACTACCGCAAACACACAGGGCACCGGCC
CTTTCAGTGCCAGAAGTGCGACAGGGCCTTTTCCAGGTCGGACCACCTTGCCTTACACATGAAGAG
GCACTAAATGACTAGTGCGCGCAGCGGCCGACCATGGCCCAACTTGTTTATTGCAGCTTATAATGG
TTACAAATAAAGCAATAGCATCACAAATTTCACAAATAAAGCATTTTTTTCACTGCATTCTAGTTG
TGGTTTGTCCAAACTCATCAATGTATCTTATCATGTCTGGATCTCGGTACCGGATCCAAATTCCCGA
TAAGGATCTTCCTAGAGCATGGCTACGTAGATAAGTAGCATGGCGGGTTAATCATTAACTACAAG
GAACCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGCCT
TAATTAACCTAATTCACTGGCCGTCGTTTTACAACGTCGTGACTGGGAAAACCCTGGCGTTACCCA
ACTTAATCGCCTTGCAGCACATCCCCCTTTCGCCAGCTGGCGTAATAGCGAAGAGGCCCGCACCGA
TCGCCCTTCCCAACAGTTGCGCAGCCTGAATGGCGAATGGGACGCGCCCTGTAGCGGCGCATTAA
GCGCGGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAATCGGGGG
CTCCCTTTAGGGTTCCGATTTAGTGCTTTACGGCACCTCGACCCCAAAAAACTTGATTAGGGTGAT
GGTTCACGTAGTGGGCCATCGCCCTGATAGACGGTTTTTCGCCCTTTGACGTTGGAGTCCACGTTCT
TTAATAGTGGACTCTTGTTCCAAACTGGAACAACACTCAACCCTATCTCGGTCTATTCTTTTGATTT
ATAAGGGATTTTGCCGATTTCGGCCTATTGGTTAAAAAATGAGCTGATTTAACAAAAATTTAACGC
GAATTTTAACAAAATATTAACGTTTATAATTTCAGGTGGCATCTTTCGGGGAAATGTGCGCGGAAC
CCCTATTTGTTTATTTTTCTAAATACATTCAAATATGTATCCGCTCATGAGACAATAACCCTGATAA
ATGCTTCAATAATATTGAAAAAGGAAGAGTATGAGTATTCAACATTTCCGTGTCGCCCTTATTCCC
TTTTTTGCGGCATTTTGCCTTCCTGTTTTTGCTCACCCAGAAACGCTGGTGAAAGTAAAAGATGCTG
AAGATCAGTTGGGTGCACGAGTGGGTTACATCGAACTGGATCTCAATAGTGGTAAGATCCTTGAG
AGTTTTCGCCCCGAAGAACGTTTTCCAATGATGAGCACTTTTAAAGTTCTGCTATGTGGCGCGGTA
TTATCCCGTATTGACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAGAGAA

Nucleic acid sequence of pAAV-UBC-rtTA4-WPRE3-SV40 pA vector (SEQ ID NO: 17):

TTATGCAGTGCTGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGTAATGGT
AACAACGTTGCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAGCACTGGGGCCAGATG
GTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCA
TATATACTTTAGATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCCGAAGGTAACTG
GCTTCAGCAGAGCGCAGATACCAAATACTGTCCTTCTAGTGTAGCCGTAGTTAGGCCACCACTTCA
AGAACTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGTATCT
TTATAGTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCCTTTT
GCTCACATGTTCTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGG
CACCCCAGGCTTTACACTTTATGCTTCCGGCTCGTATGTTGTGTGGAATTGTGAGCGGATAACAATT
TCACACAGGAAACAGCTATGACCATGATTACGCCAGATTTAATTAAGGCCTTAATTAGGCTGCGCG
CTCGCTCGCTCACTGAGGCCGCCCGGGCAAAGCCCGGGCGTCGGGCGACCTTTGGTCGCCCGGCCT
CAGTGAGCGAGCGAGCGCGCAGAGAGGGAGTGGCCAACTCCATCACTAGGGGTTCCTTGTAGTTA
ATGATTAACCCGCCATGCTACTTATCTACGTAGCCATGCTCTAGGAAGATCGGAATTCCTGATCTG
GCCTCCGCGCCGGGTTTTGGCGCCTCCCGCGGGCGCCCCCCTCCTCACGGCGAGCGCTGCCACGTC
AGACGAAGGGCGCAGCGAGCGTCCTGATCCTTCCGCCCGGACGCTCAGGACAGCGGCCCGCTGCT
CATAAGACTCGGCCTTAGAACCCCAGTATCAGCAGAAGGACATTTTAGGACGGGACTTGGGTGAC
TCTAGGGCACTGGTTTTCTTTCCAGAGAGCGGAACAGGCGAGGAAAAGTAGTCCCTTCTCGGCGAT
TCTGCGGAGGGATCTCCGTGGGGCGGTGAACGCCGATGATTATATAAGGACGCGCCGGGTGTGGC
ACAGCTAGTTCCGTCGCAGCCGGGATTTGGGTCGCGGTTCTTGTTTGTGGATCGCTGTGATCGTCA
CTTGGTGAGTAGCGGGCTGCTGGGCTGGCCGGGGCTTTCGTGGCCGCCGGGCCGCTCGGTGGGAC
GGAAGCGTGTGGAGAGACCGCCAAGGGCTGTAGTCTGGGTCCGCGAGCAAGGTTGCCCTGAACTG
GGGGTTGGGGGGAGCGCAGCAAAATGGCGGCTGTTCCCGAGTCTTGAATGGAAGACGCTTGTGAG
GCGGGCTGTGAGGTCGTTGAAACAAGGTGGGGGGCATGGTGGGCGGCAAGAACCCAAGGTCTTGA
GGCCTTCGCTAATGCGGGAAAGCTCTTATTCGGGTGAGATGGGCTGGGGCACCATCTGGGGACCCT
GACGTGAAGTTTGTCACTGACTGGAGAACTCGGTTTGTCGTCTGTTGCGGGGGCGGCAGTTATGCG
GTGCCGTTGGGCAGTGCACCCGTACCTTTGGGAGCGCGCGCCTCGTCGTGTCGTGACGTCACCCGT
TCTGTTGGCTTATAATGCAGGGTGGGGCCACCTGCCGGTAGGTGTGCGGTAGGCTTTTCTCCGTCG
CAGGACGCAGGGTTCGGGCCTAGGGTAGGCTCTCCTGAATCGACAGGCGCCGGACCTCTGGTGAG
GGGAGGGATAAGTGAGGCGTCAGTTTCTTTGGTCGGTTTTATGTACCTATCTTCTTAAGTAGCTGA
AGCTCCGGTTTTGAACTATGCGCTCGGGGTTGGCGAGTGTGTTTTGTGAAGTTTTTTAGGCACCTTT
TGAAATGTAATCATTTGGGTCAATATGTAATTTTCAGTGTTAGACTAGTAAATTGTCCGCTAAATTC
TGGCCGTTTTTGGCTTTTTTGTTAGACGAAGCGGCCGCATTAAACGCCACCATGTCCCGCTTGGATA
AGAGCAAGGTAATAAATAGCGCACTCGAACTCCTCAACGGCGTGGGCATCGAAGGTCTGACTACT
CGAAAGCTCGCCCAGAAATTGGGTGTGGAGCAACCTACATTGTATTGGCATGTCAAGAACAAAAG
AGCCCTGCTGGACGCTCTTCCTATTGAAATGCTTGACAGGCATCACACTCATTCCTGCCCCCTTGAG
GTCGAGAGTTGGCAAGATTTTCTCCGAAACAATGCAAAGTCCTACCGCTGCGCACTTTTGTCCCAT
AGGGATGGAGCAAAAGTGCACCTGGGAACCAGGCCAACAGAGAAACAATACGAGACTCTCGAGA
ACCAGTTGGCTTTCTTGTGCCAACAGGGGTTCTCACTTGAAAATGCCCTTTACGCACTGTCAGCCGT
TGGACATTTTACCCTGGGGTGCGTTCTTGAGGAGCAAGAACATCAGGTTGCTAAGGAGGAGCGCG
AGACTCCAACCACTGATTCTATGCCACCTTTGCTGAAACAGGCCATTGAACTTTTCGATAGACAGG
GTGCTGAACCTGCCTTTCTCTTCGGGTTGGAGCTGATTATTTGTGGTCTCGAAAAACAGCTGAAAT
GTGAAAGTGGTGGCCCTACTGACGCCCTCGATGATTTCGACCTGGATATGCTGCCAGCCGATGCAC
TTGATGATTTCGATTTGGATATGCTTCCAGCCGACGCACTGGACGACTTCGATTTGGACATGCTTCC
CGGTTAAACTAGTCTAGCAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTATTGCTTC
CCGTATGGCTTTCATTTTCTCCTCCTTGTATAAATCCTGGTTAGTTCTTGCCACGGCGGAACTCATC
GCCGCCTGCCTTGCCCGCTGCTGGACAGGGGCTCGGCTGTTGGGCACTGACAATTCCGTGGTGTTT
ATTTGTGAAATTTGTGATGCTATTGCTTTATTTGTAACCATTCTAGCTTTATTTGTGAAATTTGTGAT
GCTATTGCTTTATTTGTAACCATTATAAGCTGCAATAAACAAGTTAACAACAACAATTGCATTCAT
TTTATGTTTCAGGTTCAGGGGGAGATGTGGGAGGTTTTTTAAAGCGGGGGATCCAAATTCCCGATA
AGGATCTTCCTAGAGCATGGCTACGTAGATAAGTAGCATGGCGGGTTAATCATTAACTACAAGGA
ACCCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAGCCTTA
ATTAACCTAATTCACTGGCCGTCGTTTTACAACGTCGTGACTGGGAAAACCCTGGCGTTACCCAAC
TTAATCGCCTTGCAGCACATCCCCCTTTCGCCAGCTGGCGTAATAGCGAAGAGGCCCGCACCGATC
GCCCTTCCCAACAGTTGCGCAGCCTGAATGGCGAATGGGACGCGCCCTGTAGCGGCGCATTAAGC
GCGGCGGGTGTGGTGGTTACGCGCAGCGTGACCGCTACACTTGCCAGCGCCCTAGCGCCCGCTCCT
TTCGCTTTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTAGGGTGATGG
TTCACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACCCTATCTCGGTCTATTCTTTTGATTTAT
AAGGGATTTTGCCGATTTCGGCCTATTGGTTAAAAAATGAGCTGATTTAACAAAAATTTAACGCGA
ATTTTAACAAAATATTAACGTTTATAATTTCAGGTGGCATCTTTCGGGGAAATGTGCGCGGAACCC
CTATTTGTTTATTTTTCTAAATACATTCAAATATGTATCCGCTCATGAGACAATAACCCTGATAAAT
GCTTCAATAATATTGAAAAAGGAAGAGTATGAGTATTCAACATTTCCGTGTCGCCCTTATTCCCTTT
TTTGCGGCATTTTGCCTTCCTGTTTTTGCTCACCCAGAAACGCTGGTGAAAGTAAAAGATGCTGAA
GATCAGTTGGGTGCACGAGTGGGTTACATCGAACTGGATCTCAATAGTGGTAAGATCCTTGAGAGT
TTTCGCCCCGAAGAACGTTTTCCAATGATGAGCACTTTTAAAGTTCTGCTATGTGGCGCGGTATTAT
CCCGTATTGACGCCGGGCAAGAGCAACTCGGTCGCCGCATACACTATTCTCAGAATGACTTGGTTG
AGTACTCACCAGTCACAGAAAAGCATCTTACGGATGGCATGACAGTAAGAGAA

UBC promoter sequence (SEQ ID NO: 18):

GATCTGGCCTCCGCGCCGGGTTTTGGCGCCTCCCGCGGGCGCCCCCCTCCTCACGGCGAGCGCTGC
CACGTCAGACGAAGGGCGCAGCGAGCGTCCTGATCCTTCCGCCCGGACGCTCAGGACAGCGGCCC
GCTGCTCATAAGACTCGGCCTTAGAACCCCAGTATCAGCAGAAGGACATTTTAGGACGGGACTTG
GGTGACTCTAGGGCACTGGTTTTCTTTCCAGAGAGCGGAACAGGCGAGGAAAAGTAGTCCCTTCTC
GGCGATTCTGCGGAGGGATCTCCGTGGGGCGGTGAACGCCGATGATTATATAAGGACGCGCCGGG
TGTGGCACAGCTAGTTCCGTCGCAGCCGGGATTTGGGTCGCGGTTCTTGTTTGTGGATCGCTGTGA
TCGTCACTTGGTGAGTAGCGGGCTGCTGGGCTGGCCGGGGCTTTCGTGGCCGCCGGGCCGCTCGGT
GGGACGGAAGCGTGTGGAGAGACCGCCAAGGGCTGTAGTCTGGGTCCGCGAGCAAGGTTGCCCTG
AACTGGGGGTTGGGGGGAGCGCAGCAAAATGGCGGCTGTTCCCGAGTCTTGAATGGAAGACGCTT
GTGAGGCGGGCTGTGAGGTCGTTGAAACAAGGTGGGGGGCATGGTGGGCGGCAAGAACCCAAGG
TCTTGAGGCCTTCGCTAATGCGGGAAAGCTCTTATTCGGGTGAGATGGGCTGGGGCACCATCTGGG
GACCCTGACGTGAAGTTTGTCACTGACTGGAGAACTCGGTTTGTCGTCTGTTGCGGGGGCGGCAGT
TATGCGGTGCCGTTGGGCAGTGCACCCGTACCTTTGGGAGCGCGCGCCTCGTCGTGTCGTGACGTC
ACCCGTTCTGTTGGCTTATAATGCAGGGTGGGGCCACCTGCCGGTAGGTGTGCGGTAGGCTTTTCT
CCGTCGCAGGACGCAGGGTTCGGGCCTAGGGTAGGCTCTCCTGAATCGACAGGCGCCGGACCTCT
GGTGAGGGGAGGGATAAGTGAGGCGTCAGTTTCTTTGGTCGGTTTTATGTACCTATCTTCTTAAGT
AGCTGAAGCTCCGGTTTTGAACTATGCGCTCGGGGTTGGCGAGTGTGTTTTGTGAAGTTTTTTAGG
CACCTTTTGAAATGTAATCATTTGGGTCAATATGTAATTTTCAGTGTTAGACTAGTAAATTGTCCGC
TAAATTCTGGCCGTTTTTGGCTTTTTTGTTAGAC

Tet-O sequence (SEQ ID NO: 19): TCCCTATCAGTGATAGAGA

Nucleic acid sequence encoding minimal CMV promoter (SEQ ID NO: 20):

GCTTTAGGCGTGTACGGTGGGCGCCTATAAAAGCAGAGCTCGTTTAGT
GAACCGTCAGATCGCCTGGA

Nucleic acid sequence encoding WPRE (SEQ ID NO: 21):

AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTATGGCT
TTCATTTTCTCCTCCTTGTATAAATCCTGGTTAGTTCTTGCCACGGCGGAACTCATCGCCGCCTGCC
TTGCCCGCTGCTGGACAGGGGCTCGGCTGTTGGGCACTGACAATTCCGTGGTGTT

Nucleic acid sequence encoding inverted terminal repeat sequence (SEQ ID NO: 22):

CCTTAATTAGGCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCATCAC
TAGGGGTTCCT

Nucleic acid sequence of a TRE2 promoter (a non-limiting example of a TRE promoter) (SEQ ID NO: 23):

AATTCGTACACGCCTACCTCGACCCATCAAGTGCCACCTGACGTCTCCCTATCAGTGATAGAGAAG
TCGACACGTCTCGAGCTCCCTATCAGTGATAGAGAAGGTACGTCTAGAACGTCTCCCTATCAGTGA
TAGAGAAGTCGACACGTCTCGAGCTCCCTATCAGTGATAGAGAAGGTACGTCTAGAACGTCTCCCT
ATCAGTGATAGAGAAGTCGACACGTCTCGAGCTCCCTATCAGTGATAGAGAAGGTACGTCTAGAA
CGTCTCCCTATCAGTGATAGAGAAGTCGACACGTCTCGAGCTCCCTATCAGTGATAGAGAAGGTAC
CCCCTATATAAGCAGAGCTCGTTTAGTGAACCGTCAGATCGCCTGGAGACGCCATCCACGCTGTTT
TGACCTCCATAGAAGACACCGGGACCGATCCAGCCTGGATCGC

Nucleic acid sequence of P tight promoter (a non-limiting example of a TRE promoter) (SEQ ID NO: 24):

GAGTTTACTCCCTATCAGTGATAGAGAACGTATGTCGAGTTTACTCCCTATCAGTGATAGAGAACG
ATGTCGAGTTTACTCCCTATCAGTGATAGAGAACGTATGTCGAGTTTACTCCCTATCAGTGATAGA
GAACGTATGTCGAGTTTACTCCCTATCAGTGATAGAGAACGTATGTCGAGTTTATCCCTATCAGTG
ATAGAGAACGTATGTCGAGTTTACTCCCTATCAGTGATAGAGAACGTATGTCGAGGTAGGCGTGTA
CGGTGGGAGGCCTATATAAGCAGAGCTCGTTTAGTGAACCGTCAGATCGCC

Nucleic acid sequence encoding TetR (SEQ ID NO: 25):

ATGGCTAGATTAGATAAAAGTAAAGTGATTAACAGCGCATTAGAGCTGCTTAATGAGGTCGGAAT
CGAAGGTTTAACAACCCGTAAACTCGCCCAGAAGCTAGGTGTAGAGCAGCCTACATTGTATTGGC
ATGTAAAAAATAAGCGGGCTTTGCTCGACGCCTTAGCCATTGAGATGTTAGATAGGCACCATACTC
ACTTTTGCCCTTTAGAAGGGGAAAGCTGGCAAGATTTTTTACGTAATAACGCTAAAAGTTTTAGAT
GTGCTTTACTAAGTCATCGCGATGGAGCAAAAGTACATTTAGGTACACGGCCTACAGAAAAACAG
TATGAAACTCTCGAAAATCAATTAGCCTTTTTATGCCAACAAGGTTTTTCACTAGAGAATGCATTA
TATGCACTCAGCGCTGTGGGGCATTTTACTTTAGGTTGCGTATTGGAAGATCAAGAGCATCAAGTC
GCTAAAGAAGAAAGGGAAACACCTACTACTGATAGTATGCCGCCATTATTACGACAAGCTATCGA
ATTATTTGATCACCAAGGTGCAGAGCCAGCCTTCTTATTCGGCCTTGAATTGATCATATGCGGATT
AGAAAAACAACTTAAATGTGAAAGTGGG

Amino acid sequence encoding TetR (SEQ ID NO: 26):

MARLDKSKVINSALELLNEVGIEGLTTRKLAQKLGVEQPTLYWHVKNKRALLDALAIEMLDRHHTHF
CPLEGESWQDFLRNNAKSFRCALLSHRDGAKVHLGTRPTEKQYETLENQLAFLCQQGFSLENALYALS
AVGHFTLGCVLEDQEHQVAKEERETPTTDSMPPLLRQAIELFDHQGAEPAFLFGLELIICGLEKQLKCES
G

Nucleic acid sequence encoding TetR-Krab (SEQ ID NO: 27)

ATGGCTAGATTAGATAAAAGTAAAGTGATTAACAGCGCATTAGAG
CTGCTTAATGAGGTCGGAATCGAAGGTTTAACAACCCGTAAACTC
GCCCAGAAGCTAGGTGTAGAGCAGCCTACATTGTATTGGCATGTA
AAAAATAAGCGGGCTTTGCTCGACGCCTTAGCCATTGAGATGTTA
GATAGGCACCATACTCACTTTTGCCCTTTAGAAGGGGAAAGCTGG
CAAGATTTTTTACGTAATAACGCTAAAAGTTTTAGATGTGCTTTA
CTAAGTCATCGCGATGGAGCAAAAGTACATTTAGGTACACGGCCT
ACAGAAAAACAGTATGAAACTCTCGAAAATCAATTAGCCTTTTTA
TGCCAACAAGGTTTTTCACTAGAGAATGCATTATATGCACTCAGC
GCTGTGGGGCATTTTACTTTAGGTTGCGTATTGGAAGATCAAGAG
CATCAAGTCGCTAAAGAAGAAAGGGAAACACCTACTACTGATAGT
ATGCCGCCATTATTACGACAAGCTATCGAATTATTTGATCACCAA
GGTGCAGAGCCAGCCTTCTTATTCGGCCTTGAATTGATCATATGC
GGATTAGAAAAACAACTTAAATGTGAAAGTGGGTCGCCAAAAAAG
AAGAGAAAGGTCGACGGCGGTGGTGCTTTGTCTCCTCAGCACTCT
GCTGTCACTCAAGGAAGTATCATCAAGAACAAGGAGGGCATGGAT
GCTAAGTCACTAACTGCCTGGTCCCGGACACTGGTGACCTTCAAG
GATGTATTTGTGGACTTCACCAGGGAGGAGTGGAAGCTGCTGGAC
ACTGCTCAGCAGATCGTGTACAGAAATGTGATGCTGGAGAACTAT
AAGAACCTGGTTTCCTTGGGTTATCAGCTTACTAAGCCAGATGTG
ATCCTCCGGTTGGAGAAGGGAGAAGAGCCCTGGCTGGTGGAGAGA
GAAATTCACCAAGAGACCCATCCTGATTCAGAGACTGCATTTGAA
ATCAAATCATCAGTTTAA

Amino acid sequence encoding TetR-KRAB (SEQ ID NO: 28):

MARLDKSKVINSALELLNEVGIEGLTTRKLAQKLGVEQPTLYWHV
KNKRALLDALAIEMLDRHHTHFCPLEGESWQDFLRNNAKSFRCAL
LSHRDGAKVHLGTRPTEKQYETLENQLAFLCQQGFSLENALYALS
AVGHFTLGCVLEDQEHQVAKEERETPTTDSMPPLLRQAIELFDHQ
GAEPAFLFGLELIICGLEKQLKCESGSPKKKRKVDGGGALSPQHS
AVTQGSIIKNKEGMDAKSLTAWSRTLVTFKDVFVDFTREEWKLLD
TAQQIVYRNVMLENYKNLVSLGYQLTKPDVILRLEKGEEPWLVER
EIHQETHPDSETAFEIKSSV

Desmin promoter (SEQ ID NO: 29):

ACCTTGCTTCCTAGCTGGGCCTTTCCTTCTCCTCTATAAATACCA
GCTCTGGTATTTCGCCTTGGCAGCTGTTGCTGCTAGGGAGACGGC
TGGCTTGACATGCATCTCCTGACAAAACACAAACCCGTGGTGTGA
GTGGGTGTGGGCGGTGTGAGTAGGGGGATGAATCAGAGAGGGGGC
GAGGGAGACAGGGGCGCAGGAGTCAGGCAAAGGCGATGCGGGGGT
GCGACTACACGCAGTTGGAAACAGTCGTCAGAAGATTCTGGAAAC
TATCTTGCTGGCTATAAACTTGAGGGAAGCAGAAGGCCAACATTC
CTCCCAAGGGAAACTGAGGCTCAGAGTTAAAACCCAGGTATCAGT
GATATGCATGTGCCCCGGCCAGGGTCACTCTCTGACTAACCGGTA
CCTACCCTACAGGCCTACCTAGAGACTCTTTTGAAAGGATGGTAG
AGACCTGTCCGGGCTTTGCCCACAGTCGTTGGAAACCTCAGCATT
TTCTAGGCAACTTGTGCGAATAAAACACTTCGGGGGTCCTTCTTG
TTCATTCCAATAACCTAAAACCTCTCCTCGGAGAAAATAGGGGGC
CTCAAACAAACGAAATTCTCTAGCCCGCTTTCCCCAGGATAAGGC
AGGCATCCAAATGGAAAAAAAGGGGCCGGCCGGGGGTCTCCTGTC
AGCTCCTTGCCCTGTGAAACCCAGCAGGCCTGCCTGTCTTCTGTC
CTCTTGGGGCTGTCCAGGGGCGCAGGCCTCTTGCGGGGGAGCTGG
CCTCCCCGCCCCCTCGCCTGTGGCCGCCCTTTTCCTGGCAGGACA
GAGGGATCCTGCAGCTGTCAGGGGAGGGGCGCCGGGGGGTGATGT
CAGGAGGGCTACAAATAGTGCAGACAGCTAAGGGGCTCCGTCACC
CATCTTCACATCCACTCCAGCCGGCTGCCCGCCCGCTGCCTCCTC
TGTGCGTCCGCCCAGCCAGCCTCGTCCACGCC

Desmin-rtTA4 vector (SEQ ID NO: 30):

TTATGCAGTGCTGCCATAACCATGAGTGATAACACTGCGGCCAAC
TTACTTCTGACAACGATCGGAGGACCGAAGGAGCTAACCGCTTTT
TTGCACAACATGGGGGATCATGTAACTCGCCTTGATCGTTGGGAA
CCGGAGCTGAATGAAGCCATACCAAACGACGAGCGTGACACCACG
ATGCCTGTAGTAATGGTAACAACGTTGCGCAAACTATTAACTGGC
GAACTACTTACTCTAGCTTCCCGGCAACAATTAATAGACTGGATG
GAGGCGGATAAAGTTGCAGGACCACTTCTGCGCTCGGCCCTTCCG
GCTGGCTGGTTTATTGCTGATAAATCTGGAGCCGGTGAGCGTGGG
TCTCGCGGTATCATTGCAGCACTGGGGCCAGATGGTAAGCCCTCC
CGTATCGTAGTTATCTACACGACGGGGAGTCAGGCAACTATGGAT
GAACGAAATAGACAGATCGCTGAGATAGGTGCCTCACTGATTAAG
CATTGGTAACTGTCAGACCAAGTTTACTCATATATACTTTAGATT
GATTTAAAACTTCATTTTTAATTTAAAAGGATCTAGGTGAAGATC
CTTTTTGATAATCTCATGACCAAAATCCCTTAACGTGAGTTTTCG
TTCCACTGAGCGTCAGACCCCGTAGAAAAGATCAAAGGATCTTCT
TGAGATCCTTTTTTTCTGCGCGTAATCTGCTGCTTGCAAACAAAA
AAACCACCGCTACCAGCGGTGGTTTGTTTGCCGGATCAAGAGCTA
CCAACTCTTTTTCCGAAGGTAACTGGCTTCAGCAGAGCGCAGATA
CCAAATACTGTCCTTCTAGTGTAGCCGTAGTTAGGCCACCACTTC
AAGAACTCTGTAGCACCGCCTACATACCTCGCTCTGCTAATCCTG
TTACCAGTGGCTGCTGCCAGTGGCGATAAGTCGTGTCTTACCGGG
TTGGACTCAAGACGATAGTTACCGGATAAGGCGCAGCGGTCGGGC
TGAACGGGGGGTTCGTGCACACAGCCCAGCTTGGAGCGAACGACC
TACACCGAACTGAGATACCTACAGCGTGAGCTATGAGAAAGCGCC
ACGCTTCCCGAAGGGAGAAAGGCGGACAGGTATCCGGTAAGCGGC
AGGGTCGGAACAGGAGAGCGCACGAGGGAGCTTCCAGGGGGAAAC
GCCTGGTATCTTTATAGTCCTGTCGGGTTTCGCCACCTCTGACTT
GAGCGTCGATTTTTGTGATGCTCGTCAGGGGGGCGGAGCCTATGG
AAAAACGCCAGCAACGCGGCCTTTTTACGGTTCCTGGCCTTTTGC
TGGCCTTTTGCTCACATGTTCTTTCCTGCGTTATCCCCTGATTCT
GTGGATAACCGTATTACCGCCTTTGAGTGAGCTGATACCGCTCGC
CGCAGCCGAACGACCGAGCGCAGCGAGTCAGTGAGCGAGGAAGCG
GAAGAGCGCCCAATACGCAAACCGCCTCTCCCCGCGCGTTGGCCG
ATTCATTAATGCAGCTGGCACGACAGGTTTCCCGACTGGAAAGCG
GGCAGTGAGCGCAACGCAATTAATGTGAGTTAGCTCACTCATTAG
GCACCCCAGGCTTTACACTTTATGCTTCCGGCTCGTATGTTGTGT
GGAATTGTGAGCGGATAACAATTTCACACAGGAAACAGCTATGAC
CATGATTACGCCAGATTTAATTAAGGCCTTAATTAGGCTGCGCGC
TCGCTCGCTCACTGAGGCCGCCCGGGCAAAGCCCGGGCGTCGGGC
GACCTTTGGTCGCCCGGCCTCAGTGAGCGAGCGAGCGCGCAGAGA
GGGAGTGGCCAACTCCATCACTAGGGGTTCCTTGTAGTTAATGAT
TAACCCGCCATGCTACTTATCTACGTAGCCATGCTCTAGGAAGAT
CGGAATTCCTAGATCTACCTTGCTTCCTAGCTGGGCCTTTCCTTC
TCCTCTATAAATACCAGCTCTGGTATTTCGCCTTGGCAGCTGTTG
CTGCTAGGGAGACGGCTGGCTTGACATGCATCTCCTGACAAAACA
CAAACCCGTGGTGTGAGTGGGTGTGGGCGGTGTGAGTAGGGGGAT
GAATCAGAGAGGGGGCGAGGGAGACAGGGGCGCAGGAGTCAGGCA
AAGGCGATGCGGGGGTGCGACTACACGCAGTTGGAAACAGTCGTC
AGAAGATTCTGGAAACTATCTTGCTGGCTATAAACTTGAGGGAAG
CAGAAGGCCAACATTCCTCCCAAGGGAAACTGAGGCTCAGAGTTA
AAACCCAGGTATCAGTGATATGCATGTGCCCCGGCCAGGGTCACT
CTCTGACTAACCGGTACCTACCCTACAGGCCTACCTAGAGACTCT
TTTGAAAGGATGGTAGAGACCTGTCCGGGCTTTGCCCACAGTCGT
TGGAAACCTCAGCATTTTCTAGGCAACTTGTGCGAATAAAACACT
TCGGGGGTCCTTCTTGTTCATTCCAATAACCTAAAACCTCTCCTC
GGAGAAAATAGGGGGCCTCAAACAAACGAAATTCTCTAGCCCGCT
TTCCCCAGGATAAGGCAGGCATCCAAATGGAAAAAAAGGGGCCGG
CCGGGGGTCTCCTGTCAGCTCCTTGCCCTGTGAAACCCAGCAGGC
CTGCCTGTCTTCTGTCCTCTTGGGGCTGTCCAGGGGCGCAGGCCT
CTTGCGGGGGAGCTGGCCTCCCCGCCCCCTCGCCTGTGGCCGCCC
TTTTCCTGGCAGGACAGAGGGATCCTGCAGCTGTCAGGGGAGGGG
CGCCGGGGGGTGATGTCAGGAGGGCTACAAATAGTGCAGACAGCT
AAGGGGCTCCGTCACCCATCTTCACATCCACTCCAGCCGGCTGCC
CGCCCGCTGCCTCCTCTGTGCGTCCGCCCAGCCAGCCTCGTCCAC
GCCAAGCTTGCGGCCGCATTAAACGCCACCATGTCCCGCTTGGAT
AAGAGCAAGGTAATAAATAGCGCACTCGAACTCCTCAACGGCGTG
GGCATCGAAGGTCTGACTACTCGAAAGCTCGCCCAGAAATTGGGT
GTGGAGCAACCTACATTGTATTGGCATGTCAAGAACAAAAGAGCC
CTGCTGGACGCTCTTCCTATTGAAATGCTTGACAGGCATCACACT
CATTCCTGCCCCCTTGAGGTCGAGAGTTGGCAAGATTTTCTCCGA
AACAATGCAAAGTCCTACCGCTGCGCACTTTTGTCCCATAGGGAT
GGAGCAAAAGTGCACCTGGGAACCAGGCCAACAGAGAAACAATAC
GAGACTCTCGAGAACCAGTTGGCTTTCTTGTGCCAACAGGGGTTC
TCACTTGAAAATGCCCTTTACGCACTGTCAGCCGTTGGACATTTT
ACCCTGGGGTGCGTTCTTGAGGAGCAAGAACATCAGGTTGCTAAG
GAGGAGCGCGAGACTCCAACCACTGATTCTATGCCACCTTTGCTG
AAACAGGCCATTGAACTTTTCGATAGACAGGGTGCTGAACCTGCC
TTTCTCTTCGGGTTGGAGCTGATTATTTGTGGTCTCGAAAAACAG
CTGAAATGTGAAAGTGGTGGCCCTACTGACGCCCTCGATGATTTC
GACCTGGATATGCTGCCAGCCGATGCACTTGATGATTTCGATTTG
GATATGCTTCCAGCCGACGCACTGGACGACTTCGATTTGGACATG
CTTCCCGGTTAAACTAGTCTAGCAATCAACCTCTGGATTACAAAA
TTTGTGAAAGATTGACTGGTATTCTTAACTATGTTGCTCCTTTTA
CGCTATGTGGATACGCTGCTTTAATGCCTTTGTATCATGCTATTG
CTTCCCGTATGGCTTTCATTTTCTCCTCCTTGTATAAATCCTGGT
TAGTTCTTGCCACGGCGGAACTCATCGCCGCCTGCCTTGCCCGCT
GCTGGACAGGGGCTCGGCTGTTGGGCACTGACAATTCCGTGGTGT
TTATTTGTGAAATTTGTGATGCTATTGCTTTATTTGTAACCATTC
TAGCTTTATTTGTGAAATTTGTGATGCTATTGCTTTATTTGTAAC
CATTATAAGCTGCAATAAACAAGTTAACAACAACAATTGCATTCA
TTTTATGTTTCAGGTTCAGGGGGAGATGTGGGAGGTTTTTTAAAG
CGGGGGATCCAAATTCCCGATAAGGATCTTCCTAGAGCATGGCTA
CGTAGATAAGTAGCATGGCGGGTTAATCATTAACTACAAGGAACC
CCTAGTGATGGAGTTGGCCACTCCCTCTCTGCGCGCTCGCTCGCT
CACTGAGGCCGGGCGACCAAAGGTCGCCCGACGCCCGGGCTTTGC
CCGGGCGGCCTCAGTGAGCGAGCGAGCGCGCAGCCTTAATTAACC
TAATTCACTGGCCGTCGTTTTACAACGTCGTGACTGGGAAAACCC
TGGCGTTACCCAACTTAATCGCCTTGCAGCACATCCCCCTTTCGC
CAGCTGGCGTAATAGCGAAGAGGCCCGCACCGATCGCCCTTCCCA
ACAGTTGCGCAGCCTGAATGGCGAATGGGACGCGCCCTGTAGCGG
CGCATTAAGCGCGGCGGGTGTGGTGGTTACGCGCAGCGTGACCGC
TACACTTGCCAGCGCCCTAGCGCCCGCTCCTTTCGCTTTCTTCCC
TTCCTTTCTCGCCACGTTCGCCGGCTTTCCCCGTCAAGCTCTAAA
TCGGGGGCTCCCTTTAGGGTTCCGATTTAGTGCTTTACGGCACCT
CGACCCCAAAAAACTTGATTAGGGTGATGGTTCACGTAGTGGGCC
ATCGCCCTGATAGACGGTTTTTCGCCCTTTGACGTTGGAGTCCAC
GTTCTTTAATAGTGGACTCTTGTTCCAAACTGGAACAACACTCAA
CCCTATCTCGGTCTATTCTTTTGATTTATAAGGGATTTTGCCGAT
TTCGGCCTATTGGTTAAAAAATGAGCTGATTTAACAAAAATTTAA
CGCGAATTTTAACAAAATATTAACGTTTATAATTTCAGGTGGCAT
CTTTCGGGGAAATGTGCGCGGAACCCCTATTTGTTTATTTTTCTA
AATACATTCAAATATGTATCCGCTCATGAGACAATAACCCTGATA
AATGCTTCAATAATATTGAAAAAGGAAGAGTATGAGTATTCAACA
TTTCCGTGTCGCCCTTATTCCCTTTTTTGCGGCATTTTGCCTTCC
TGTTTTTGCTCACCCAGAAACGCTGGTGAAAGTAAAAGATGCTGA
AGATCAGTTGGGTGCACGAGTGGGTTACATCGAACTGGATCTCAA
TAGTGGTAAGATCCTTGAGAGTTTTCGCCCCGAAGAACGTTTTCC
AATGATGAGCACTTTTAAAGTTCTGCTATGTGGCGCGGTATTATC
CCGTATTGACGCCGGGCAAGAGCAACTCGGTCGCCGCATACACTA
TTCTCAGAATGACTTGGTTGAGTACTCACCAGTCACAGAAAAGCA
TCTTACGGATGGCATGACAGTAAGAGAA

pAAV2_CMV_rtTA (V16) (SEQ ID NO: 31):

AAATTGTAAACGTTAATATTTTGTTAAAATTCGCGTTAAATTTTT
GTTAAATCAGCTCATTTTTTAACCAATAGGCCGAAATCGGCAAAA
TCCCTTATAAATCAAAAGAATAGCCCGAGATAGGGTTGAGTGTTG
TTCCAGTTTGGAACAAGAGTCCACTATTAAAGAACGTGGACTCCA
ACGTCAAAGGGCGAAAAACCGTCTATCAGGGCGATGGCCCACTAC
GTGAACCATCACCCAAATCAAGTTTTTTGGGGTCGAGGTGCCGTA
AAGCACTAAATCGGAACCCTAAAGGGAGCCCCCGATTTAGAGCTT
GACGGGGAAAGCCGGCGAACGTGGCGAGAAAGGAAGGGAAGAAAG
CGAAAGGAGCGGGCGCTAGGGCGCTGGCAAGTGTAGCGGTCACGC
TGCGCGTAACCACCACACCCGCCGCGCTTAATGCGCCGCTACAGG
GCGCGTACTATGGTTGCTTTGACGTATGCGGTGTGAAATACCGCA
CAGATGCGTAAGGAGAAAATACCGCATCAGGCGCCCCTGCAGGCA
GCTGCGCGCTCGCTCGCTCACTGAGGCCGCCCGGGCAAAGCCCGG
GCGTCGGGCGACCTTTGGTCGCCCGGCCTCAGTGAGCGAGCGAGC
GCGCAGAGAGGGAGTGGCCAACTCCATCACTAGGGGTTCCTGCGG
CCGCTCGGTCCGCACGATCTCAATTCGGCCATTACGGCCGGATCC
GGCTCGAGGAGCTTGGCCCATTGCATACGTTGTATCCATATCATA
ATATGTACATTTATATTGGCTCATGTCCAACATTACCGCCATGTT
GACATTGATTATTGACTAGTTATTAATAGTAATCAATTACGGGGT
CATTAGTTCATAGCCCATATATGGAGTTCCGCGTTACATAACTTA
CGGTAAATGGCCCGCCTGGCTGACCGCCCAACGACCCCCGCCCAT
TGACGTCAATAATGACGTATGTTCCCATAGTAACGCCAATAGGGA
CTTTCCATTGACGTCAATGGGTGGAGTATTTACGCTAAACTGCCC
ACTTGGCAGTACATCAAGTGTATCATATGCCAAGTACGCCCCCTA
TTGACGTCAATGACGGTAAATGGCCCGCCTGGCATTATGCCCAGT
ACATGACCTTATGGGACTTTCCTACTTGGCAGTACATCTACGTAT
TAGTCATCGCTATTACCATGGTGATGCGGTTTTGGCAGTACATCA
ATGGGCGTGGATAGCGGTTTGACTCACGGGGATTTCCAAGTCTCC
ACCCCATTGACGTCAATGGGAGTTTGTTTTGGCACCAAAATCAAC
GGGACTTTCCAAAATGTCGTAACAACTCCGCCCCATTGACGCAAA
TGGGCGGTAGGCGTGTACGGTGGGAGGTCTATATAAGCAGAGCTC
GTTTAGTGAACCGTCAGATCGCCTGGAGACGCCATCCACGCTGTT
TTGACCTCCATAGAAGACACCGGGACCGATCCAGCCTCCGCGGCC
CCGAATTCACCATGTCTAGACTGGACAAGAGCAAAATCATAAACA
GCGCTCTGGAATTACTCAATGGAGTCGGTATCGAAGGCCTGACGA
CAAGGAAACTCGCTCAAAAGCTGGGAGTTGAGCAGCCTACCCTGT
ACTGGCACGTGAAGAACAAGCGGGCCCTGCTCGATGCCCTGCCAA
TCGAGATGCTGGACAGGCATCATACCCACAGCTGCCCCCTGGAAG
GCGAGTCATGGCAAGACTTTCTGCGGAACAACGCCAAGTCATACC
GCTGTGCTCTCCTCTCACATCGCGACGGGGCTAAAGTGCATCTCG
GCACCCGCCCAACAGAGAAACAGTACGAAACCCTGGAAAATCAGC
TCGCGTTCCTGTGTCAGCAAGGCTTCTCCCTGGAGAACGCACTGT
ACGCTCTGTCCGCCGTGGGCCACTTTACACTGGGCTGCGTATTGG
AGGAACAGGAGCATCAAGTAGCAAAAGAGGAAAGAGAGACACCTA
CCACCGATTCTATGCCCCCACTTCTGAAGCAAGCAATTGAGCTGT
TCGACCGGCAGGGAGCCGAACCTGCCTTCCTTTTCGGCCTGGAAC
TAATCATATGTGGCCTGGAGAAACAGCTAAAGTGCGAAAGCGGCG
GGCCGACCGACGCCCTTGACGATTTTGACTTAGACATGCTCCCAG
CCGATGCCCTTGACGACTTTGACCTTGATATGCTGCCTGCTGACG
CTCTTGACGATTTTGACCTTGACATGCTCCCCGGGTAACTAAGTA
AGGATCATCTTAATTAAATCGATAAGGATCTGGCCGCCTCGGCCT
AATCAACCTCTGGATTACAAAATTTGTGAAAGATTGACTGGTATT
CTTAACTATGTTGCTCCTTTTACGCTATGTGGATACGCTGCTTTA
ATGCCTTTGTATCATGCTATTGCTTCCCGTATGGCTTTCATTTTC
TCCTCCTTGTATAAATCCTGGTTGCTGTCTCTTTATGAGGAGTTG
TGGCCCGTTGTCAGGCAACGTGGCGTGGTGTGCACTGTGTTTGCT
GACGCAACCCCCACTGGTTGGGGCATTGCCACCACCTGTCAGCTC
CTTTCCGGGACTTTCGCTTTCCCCCTCCCTATTGCCACGGCGGAA
CTCATCGCCGCCTGCCTTGCCCGCTGCTGGACAGGGGCTCGGCTG
TTGGGCACTGACAATTCCGTGGTGTTGTCGGGGAAATCATCGTCC
TTTCCTTGGCTGCTCGCCTGTGTTGCCACCTGGATTCTGCGCGGG
ACGTCCTTCTGCTACGTCCCTTCGGCCCTCAATCCAGCGGACCTT
CCTTCCCGCGGCCTGCTGCCGGCTCTGCGGCCTCTTCCGCGTCTT
CGCCTTCGCCCTCAGACGAGTCGGATCTCCCTTTGGGCCGCCTCC
CCGCCAGACATGATAAGATACATTGATGAGTTTGGACAAACCACA
ACTAGAATGCAGTGAAAAAAATGCTTTATTTGTGAAATTTGTGAT
GCTATTGCTTTATTTGTAACCATTATAAGCTGCAATAAACAAGTT
AACAACAACAATTGCATTCATTTTATGTTTCAGGTTCAGGGGGAG
ATGTGGGAGGTTTTTTAAAGCAAGTAAAACCTCTACAAATGTGGT
AACTAGCGCGTGCGGCCGCAGGAACCCCTAGTGATGGAGTTGGCC
ACTCCCTCTCTGCGCGCTCGCTCGCTCACTGAGGCCGGGCGACCA
AAGGTCGCCCGACGCCCGGGCTTTGCCCGGGCGGCCTCAGTGAGC
GAGCGAGCGCGCAGCTGCCTGCAGGACATGTGAGCAAAAGGCCAG
CAAAAGGCCAGGAACCGTAAAAAGGCCGCGTTGCTGGCGTTTTTC
CATAGGCTCCGCCCCCCTGACGAGCATCACAAAAATCGACGCTCA
AGTCAGAGGTGGCGAAACCCGACAGGACTATAAAGATACCAGGCG
TTTCCCCCTGGAAGCTCCCTCGTGCGCTCTCCTGTTCCGACCCTG
CCGCTTACCGGATACCTGTCCGCCTTTCTCCCTTCGGGAAGCGTG
GCGCTTTCTCATAGCTCACGCTGTAGGTATCTCAGTTCGGTGTAG
GTCGTTCGCTCCAAGCTGGGCTGTGTGCACGAACCCCCCGTTCAG
CCCGACCGCTGCGCCTTATCCGGTAACTATCGTCTTGAGTCCAAC
CCGGTAAGACACGACTTATCGCCACTGGCAGCAGCCACTGGTAAC
AGGATTAGCAGAGCGAGGTATGTAGGCGGTGCTACAGAGTTCTTG
AAGTGGTGGCCTAACTACGGCTACACTAGAAGGACAGTATTTGGT
ATCTGCGCTCTGCTGAAGCCAGTTACCTTCGGAAAAAGAGTTGGT
AGCTCTTGATCCGGCAAACAAACCACCGCTGGTAGCGGTGGTTTT
TTTGTTTGCAAGCAGCAGATTACGCGCAGAAAAAAAGGATCTCAA
GAAGATCCTTTGATCTTTTCTACGGGGTCTGACGCTCAGTGGAAC
GAAAACTCACGTTAAGGGATTTTGGTCATGAGATTATCAAAAAGG
ATCTTCACCTAGATCCTTTTAAATTAAAAATGAAGTTTTAAATCA
ATCTAAAGTATATATGAGTAAACTTGGTCTGACAGTTACCAATGC
TTAATCAGTGAGGCACCTATCTCAGCGATCTGTCTATTTCGTTCA
TCCATAGTTGCCTGACTCCCCGTCGTGTAGATAACTACGATACGG
GAGGGCTTACCATCTGGCCCCAGTGCTGCAATGATACCGCGAGAC
CCACGCTCACCGGCTCCAGATTTATCAGCAATAAACCAGCCAGCC
GGAAGGGCCGAGCGCAGAAGTGGTCCTGCAACTTTATCCGCCTCC
ATCCAGTCTATTAATTGTTGCCGGGAAGCTAGAGTAAGTAGTTCG
CCAGTTAATAGTTTGCGCAACGTTGTTGCCATTGCTACAGGCATC
GTGGTGTCACGCTCGTCGTTTGGTATGGCTTCATTCAGCTCCGGT
TCCCAACGATCAAGGCGAGTTACATGATCCCCCATGTTGTGCAAA
AAAGCGGTTAGCTCCTTCGGTCCTCCGATCGTTGTCAGAAGTAAG
TTGGCCGCAGTGTTATCACTCATGGTTATGGCAGCACTGCATAAT
TCTCTTACTGTCATGCCATCCGTAAGATGCTTTTCTGTGACTGGT
GAGTACTCAACCAAGTCATTCTGAGAATAGTGTATGCGGCGACCG
AGTTGCTCTTGCCCGGCGTCAATACGGGATAATACCGCGCCACAT
AGCAGAACTTTAAAAGTGCTCATCATTGGAAAACGTTCTTCGGGG
CGAAAACTCTCAAGGATCTTACCGCTGTTGAGATCCAGTTCGATG
TAACCCACTCGTGCACCCAACTGATCTTCAGCATCTTTTACTTTC
ACCAGCGTTTCTGGGTGAGCAAAAACAGGAAGGCAAAATGCCGCA
AAAAAGGGAATAAGGGCGACACGGAAATGTTGAATACTCATACTC
TTCCTTTTTCAATATTATTGAAGCATTTATCAGGGTTATTGTCTC
ATGAGCGGATACATATTTGAATGTATTTAGAAAAATAAACAAATA
GGGGTTCCGCGCACATTTCCCCGAAAAGTGCCACCTGACGTCTAA
GAAACCATTATTATCATGACATTAACCTATAAAAATAGGCGTATC
ACGAGGCCCTTTCGTCTCGCGCGTTTCGGTGATGACGGTGAAAAC
CTCTGACACATGCAGCTCCCGGAGACGGTCACAGCTTGTCTGTAA
GCGGATGCCGGGAGCAGACAAGCCCGTCAGGGCGCGTCAGCGGGT
GTTGGCGGGTGTCGGGGCTGGCTTAACTATGCGGCATCAGAGCAG
ATTGTACTGAGAGTGCACCATA

CMV-tTA (SEQ ID NO: 32):

CCTGCAGGCAGCTGCGCGCTCGCTCGCTCACTGAGGCCGCCCGGG
CAAAGCCCGGGCGTCGGGCGACCTTTGGTCGCCCGGCCTCAGTGA
GCGAGCGAGCGCGCAGAGAGGGAGTGGCCAACTCCATCACTAGGG
GTTCCTGCGGCCGCACGCGTGGAGCTAGTTATTAATAGTAATCAA
TTACGGGGTCATTAGTTCATAGCCCATATATGGAGTTCCGCGTTA
CATAACTTACGGTAAATGGCCCGCCTGGCTGACCGCCCAACGACC
CCCGCCCATTGACGTCAATAATGACGTATGTTCCCATAGTAACGT
CAATAGGGACTTTCCATTGACGTCAATGGGTGGAGTATTTACGGT
AAACTGCCCACTTGGCAGTACATCAAGTGTATCATATGCCAAGTA
CGCCCCCTATTGACGTCAATGACGGTAAATGGCCCGCCTGGCATT
ATGCCCAGTACATGACCTTATGGGACTTTCCTACTTGGCAGTACA
TCTACGTATTAGTCATCGCTATTACCATGGTGATGCGGTTTTGGC
AGTACATCAATGGGCGTGGATAGCGGTTTGACTCACGGGGATTTC
CAAGTCTCCACCCCATTGACGTCAATGGGAGTTTGTTTTGCACCA
AAATCAACGGGACTTTCCAAAATGTCGTAACAACTCCGCCCCATT
GACGCAAATGGGCGGTAGGCGTGTACGGTGGGAGGTCTATATAAG
CAGAGCTCGTTTAGTGAACCGTCAGATCGCCTGGAGACGCCATCC
ACGCTGTTTTGACCTCCATAGAAGACACCGGGACCGATCCAGCCT
CCGCGGATTCGAATCCCGGCCGGGAACGGTGCATTGGAACGCGGA
TTCCCCGTGCCAAGAGTGACGTAAGTACCGCCTATAGAGTCTATA
GGCCCACAAAAAATGCTTTCTTCTTTTAATATACTTTTTTGTTTA
TCTTATTTCTAATACTTTCCCTAATCTCTTTCTTTCAGGGCAATA
ATGATACAATGTATCATGCCTCTTTGCACCATTCTAAAGAATAAC
AGTGATAATTTCTGGGTTAAGGCAATAGCAATATTTCTGCATATA
AATATTTCTGCATATAAATTGTAACTGATGTAAGAGGTTTCATAT
TGCTAATAGCAGCTACAATCCAGCTACCATTCTGCTTTTATTTTA
TGGTTGGGATAAGGCTGGATTATTCTGAGTCCAAGCTAGGCCCTT
TTGCTAATCATGTTCATACCTCTTATCTTCCTCCCACAGCTCCTG
GGCAACGTGCTGGTCTGTGTGCTGGCCCATCACTTTGGCAAAGAA
TTGGGATTCGAACATCGATTGAATTCATGTCTAGACTGGACAAGA
GCAAAGTCATAAACTCTGCTCTGGAATTACTCAATGAAGTCGGTA
TCGAAGGCCTGACGACAAGGAAACTCGCTCAAAAGCTGGGAGTTG
AGCAGCCTACCCTGTACTGGCACGTGAAGAACAAGCGGGCCCTGC
TCGATGCCCTGGCAATCGAGATGCTGGACAGGCATCATACCCACT
TCTGCCCCCTGGAAGGCGAGTCATGGCAAGACTTTCTGCGGAACA
ACGCCAAGTCATTCCGCTGTGCTCTCCTCTCACATCGCGACGGGG
CTAAAGTGCATCTCGGCACCCGCCCAACAGAGAAACAGTACGAAA
CCCTGGAAAATCAGCTCGCGTTCCTGTGTCAGCAAGGCTTCTCCC
TGGAGAACGCACTGTACGCTCTGTCCGCCGTGGGCCACTTTACAC
TGGGCTGCGTATTGGAGGATCAGGAGCATCAAGTAGCAAAAGAGG
AAAGAGAGACACCTACCACCGATTCTATGCCCCCACTTCTGAGAC
AAGCAATTGAGCTGTTCGACCATCAGGGAGCCGAACCTGCCTTCC
TTTTCGGCCTGGAACTAATCATATGTGGCCTGGAGAAACAGCTAA
AGTGCGAAAGCGGCGGGCCGGCCGACGCCCTTGACGATTTTGACT
TAGACATGCTCCCAGCCGATGCCCTTGACGACTTTGACCTTGATA
TGCTGCCTGCTGACGCTCTTGACGATTTTGACCTTGACATGCTCC
CCGGATGAGGATCCTCTAGAGTCGACCTGCAGAAGCTTGCCTCGA
GCAGCGCTGCTCGAGAGATCTACGGGTGGCATCCCTGTGACCCCT
CCCCAGTGCCTCTCCTGGCCCTGGAAGTTGCCACTCCAGTGCCCA
CCAGCCTTGTCCTAATAAAATTAAGTTGCATCATTTTGTCTGACT
AGGTGTCCTTCTATAATATTATGGGGTGGAGGGGGGTGGTATGGA
GCAAGGGGCAAGTTGGGAAGACAACCTGTAGGGCCTGCGGGGTCT
ATTGGGAACCAAGCTGGAGTGCAGTGGCACAATCTTGGCTCACTG
CAATCTCCGCCTCCTGGGTTCAAGCGATTCTCCTGCCTCAGCCTC
CCGAGTTGTTGGGATTCCAGGCATGCATGACCAGGCTCAGCTAAT
TTTTGTTTTTTTGGTAGAGACGGGGTTTCACCATATTGGCCAGGC
TGGTCTCCAACTCCTAATCTCAGGTGATCTACCCACCTTGGCCTC
CCAAATTGCTGGGATTACAGGCGTGAACCACTGCTCCCTTCCCTG
TCCTTCTGATTTTGTAGGTAACCACGTGCGGACCGAGCGGCCGCA
GGAACCCCTAGTGATGGAGTTGGCCACTCCCTCTCTGCGCGCTCG
CTCGCTCACTGAGGCCGGGCGACCAAAGGTCGCCCGACGCCCGGG
CTTTGCCCGGGCGGCCTCAGTGAGCGAGCGAGCGCGCAGCTGCCT
GCAGGGGCGCCTGATGCGGTATTTTCTCCTTACGCATCTGTGCGG
TATTTCACACCGCATACGTCAAAGCAACCATAGTACGCGCCCTGT
AGCGGCGCATTAAGCGCGGCGGGTGTGGTGGTTACGCGCAGCGTG
ACCGCTACACTTGCCAGCGCCCTAGCGCCCGCTCCTTTCGCTTTC
TTCCCTTCCTTTCTCGCCACGTTCGCCGGCTTTCCCCGTCAAGCT
CTAAATCGGGGGCTCCCTTTAGGGTTCCGATTTAGTGCTTTACGG
CACCTCGACCCCAAAAAACTTGATTTGGGTGATGGTTCACGTAGT
GGGCCATCGCCCTGATAGACGGTTTTTCGCCCTTTGACGTTGGAG
TCCACGTTCTTTAATAGTGGACTCTTGTTCCAAACTGGAACAACA
CTCAACCCTATCTCGGGCTATTCTTTTGATTTATAAGGGATTTTG
CCGATTTCGGCCTATTGGTTAAAAAATGAGCTGATTTAACAAAAA
TTTAACGCGAATTTTAACAAAATATTAACGTTTACAATTTTATGG
TGCACTCTCAGTACAATCTGCTCTGATGCCGCATAGTTAAGCCAG
CCCCGACACCCGCCAACACCCGCTGACGCGCCCTGACGGGCTTGT
CTGCTCCCGGCATCCGCTTACAGACAAGCTGTGACCGTCTCCGGG
AGCTGCATGTGTCAGAGGTTTTCACCGTCATCACCGAAACGCGCG
AGACGAAAGGGCCTCGTGATACGCCTATTTTTATAGGTTAATGTC
ATGATAATAATGGTTTCTTAGACGTCAGGTGGCACTTTTCGGGGA
AATGTGCGCGGAACCCCTATTTGTTTATTTTTCTAAATACATTCA
AATATGTATCCGCTCATGAGACAATAACCCTGATAAATGCTTCAA
TAATATTGAAAAAGGAAGAGTATGAGTATTCAACATTTCCGTGTC
GCCCTTATTCCCTTTTTTGCGGCATTTTGCCTTCCTGTTTTTGCT
CACCCAGAAACGCTGGTGAAAGTAAAAGATGCTGAAGATCAGTTG
GGTGCACGAGTGGGTTACATCGAACTGGATCTCAACAGCGGTAAG
ATCCTTGAGAGTTTTCGCCCCGAAGAACGTTTTCCAATGATGAGC
ACTTTTAAAGTTCTGCTATGTGGCGCGGTATTATCCCGTATTGAC
GCCGGGCAAGAGCAACTCGGTCGCCGCATACACTATTCTCAGAAT
GACTTGGTTGAGTACTCACCAGTCACAGAAAAGCATCTTACGGAT
GGCATGACAGTAAGAGAATTATGCAGTGCTGCCATAACCATGAGT
GATAACACTGCGGCCAACTTACTTCTGACAACGATCGGAGGACCG
AAGGAGCTAACCGCTTTTTTGCACAACATGGGGGATCATGTAACT
CGCCTTGATCGTTGGGAACCGGAGCTGAATGAAGCCATACCAAAC
GACGAGCGTGACACCACGATGCCTGTAGCAATGGCAACAACGTTG
CGCAAACTATTAACTGGCGAACTACTTACTCTAGCTTCCCGGCAA
CAATTAATAGACTGGATGGAGGCGGATAAAGTTGCAGGACCACTT
CTGCGCTCGGCCCTTCCGGCTGGCTGGTTTATTGCTGATAAATCT
GGAGCCGGTGAGCGTGGGTCTCGCGGTATCATTGCAGCACTGGGG
CCAGATGGTAAGCCCTCCCGTATCGTAGTTATCTACACGACGGGG
AGTCAGGCAACTATGGATGAACGAAATAGACAGATCGCTGAGATA
GGTGCCTCACTGATTAAGCATTGGTAACTGTCAGACCAAGTTTAC
TCATATATACTTTAGATTGATTTAAAACTTCATTTTTAATTTAAA
AGGATCTAGGTGAAGATCCTTTTTGATAATCTCATGACCAAAATC
CCTTAACGTGAGTTTTCGTTCCACTGAGCGTCAGACCCCGTAGAA
AAGATCAAAGGATCTTCTTGAGATCCTTTTTTTCTGCGCGTAATC
TGCTGCTTGCAAACAAAAAAACCACCGCTACCAGCGGTGGTTTGT
TTGCCGGATCAAGAGCTACCAACTCTTTTTCCGAAGGTAACTGGC
TTCAGCAGAGCGCAGATACCAAATACTGTCCTTCTAGTGTAGCCG
TAGTTAGGCCACCACTTCAAGAACTCTGTAGCACCGCCTACATAC
CTCGCTCTGCTAATCCTGTTACCAGTGGCTGCTGCCAGTGGCGAT
AAGTCGTGTCTTACCGGGTTGGACTCAAGACGATAGTTACCGGAT
AAGGCGCAGCGGTCGGGCTGAACGGGGGGTTCGTGCACACAGCCC
AGCTTGGAGCGAACGACCTACACCGAACTGAGATACCTACAGCGT
GAGCTATGAGAAAGCGCCACGCTTCCCGAAGGGAGAAAGGCGGAC
AGGTATCCGGTAAGCGGCAGGGTCGGAACAGGAGAGCGCACGAGG
GAGCTTCCAGGGGGAAACGCCTGGTATCTTTATAGTCCTGTCGGG
TTTCGCCACCTCTGACTTGAGCGTCGATTTTTGTGATGCTCGTCA
GGGGGGCGGAGCCTATGGAAAAACGCCAGCAACGCGGCCTTTTTA
CGGTTCCTGGCCTTTTGCTGGCCTTTTGCTCACATGT

pAAV-Tet-O-OSK-SV40LpA (or pAAV-TRE2-OSK-SV40LpA) (SEQ ID NO: 33):

TTATGCAGTGCTGCCATAACCATGAGTGATAACACTGCGGCCAAC
TTACTTCTGACAACGATCGGAGGACCGAAGGAGCTAACCGCTTTT
TTGCACAACATGGGGGATCATGTAACTCGCCTTGATCGTTGGGAA
CCGGAGCTGAATGAAGCCATACCAAACGACGAGCGTGACACCACG
ATGCCTGTAGTAATGGTAACAACGTTGCGCAAACTATTAACTGGC
GAACTACTTACTCTAGCTTCCCGGCAACAATTAATAGACTGGATG
GAGGCGGATAAAGTTGCAGGACCACTTCTGCGCTCGGCCCTTCCG
GCTGGCTGGTTTATTGCTGATAAATCTGGAGCCGGTGAGCGTGGG
TCTCGCGGTATCATTGCAGCACTGGGGCCAGATGGTAAGCCCTCC
CGTATCGTAGTTATCTACACGACGGGGAGTCAGGCAACTATGGAT
GAACGAAATAGACAGATCGCTGAGATAGGTGCCTCACTGATTAAG
CATTGGTAACTGTCAGACCAAGTTTACTCATATATACTTTAGATT
GATTTAAAACTTCATTTTTAATTTAAAAGGATCTAGGTGAAGATC
CTTTTTGATAATCTCATGACCAAAATCCCTTAACGTGAGTTTTCG
TTCCACTGAGCGTCAGACCCCGTAGAAAAGATCAAAGGATCTTCT
TGAGATCCTTTTTTTCTGCGCGTAATCTGCTGCTTGCAAACAAAA
AAACCACCGCTACCAGCGGTGGTTTGTTTGCCGGATCAAGAGCTA
CCAACTCTTTTTCCGAAGGTAACTGGCTTCAGCAGAGCGCAGATA
CCAAATACTGTCCTTCTAGTGTAGCCGTAGTTAGGCCACCACTTC
AAGAACTCTGTAGCACCGCCTACATACCTCGCTCTGCTAATCCTG
TTACCAGTGGCTGCTGCCAGTGGCGATAAGTCGTGTCTTACCGGG
TTGGACTCAAGACGATAGTTACCGGATAAGGCGCAGCGGTCGGGC
TGAACGGGGGGTTCGTGCACACAGCCCAGCTTGGAGCGAACGACC
TACACCGAACTGAGATACCTACAGCGTGAGCTATGAGAAAGCGCC
ACGCTTCCCGAAGGGAGAAAGGCGGACAGGTATCCGGTAAGCGGC
AGGGTCGGAACAGGAGAGCGCACGAGGGAGCTTCCAGGGGGAAAC
GCCTGGTATCTTTATAGTCCTGTCGGGTTTCGCCACCTCTGACTT
GAGCGTCGATTTTTGTGATGCTCGTCAGGGGGGCGGAGCCTATGG
AAAAACGCCAGCAACGCGGCCTTTTTACGGTTCCTGGCCTTTTGC
TGGCCTTTTGCTCACATGTTCTTTCCTGCGTTATCCCCTGATTCT
GTGGATAACCGTATTACCGCCTTTGAGTGAGCTGATACCGCTCGC
CGCAGCCGAACGACCGAGCGCAGCGAGTCAGTGAGCGAGGAAGCG
GAAGAGCGCCCAATACGCAAACCGCCTCTCCCCGCGCGTTGGCCG
ATTCATTAATGCAGCTGGCACGACAGGTTTCCCGACTGGAAAGCG
GGCAGTGAGCGCAACGCAATTAATGTGAGTTAGCTCACTCATTAG
GCACCCCAGGCTTTACACTTTATGCTTCCGGCTCGTATGTTGTGT
GGAATTGTGAGCGGATAACAATTTCACACAGGAAACAGCTATGAC
CATGATTACGCCAGATTTAATTAAGGCCTTAATTAGGCTGCGCGC
TCGCTCGCTCACTGAGGCCGCCCGGGCAAAGCCCGGGCGTCGGGC
GACCTTTGGTCGCCCGGCCTCAGTGAGCGAGCGAGCGCGCAGAGA
GGGAGTGGCCAACTCCATCACTAGGGGTTCCTTGTAGTTAATGAT
TAACCCGCCATGCTACTTATCTACGTAGCCATGCTCTAGGAAGAT
CGGAATTCGTACACGCCTACCTCGACCCATCAAGTGCCACCTGAC
GTCTCCCTATCAGTGATAGAGAAGTCGACACGTCTCGAGCTCCCT
ATCAGTGATAGAGAAGGTACGTCTAGAACGTCTCCCTATCAGTGA
TAGAGAAGTCGACACGTCTCGAGCTCCCTATCAGTGATAGAGAAG
GTACGTCTAGAACGTCTCCCTATCAGTGATAGAGAAGTCGACACG
TCTCGAGCTCCCTATCAGTGATAGAGAAGGTACGTCTAGAACGTC
TCCCTATCAGTGATAGAGAAGTCGACACGTCTCGAGCTCCCTATC
AGTGATAGAGAAGGTACCCCCTATATAAGCAGAGCTCGTTTAGTG
AACCGTCAGATCGCCTGGAGACGCCATCCACGCTGTTTTGACCTC
CATAGAAGACACCGGGACCGATCCAGCCTGGATCGCGGCCGCGCC
ACCATGGCTGGACACCTGGCTTCAGACTTCGCCTTCTCACCCCCA
CCAGGTGGGGGTGATGGGTCAGCAGGGCTGGAGCCGGGCTGGGTG
GATCCTCGAACCTGGCTAAGCTTCCAAGGGCCTCCAGGTGGGCCT
GGAATCGGACCAGGCTCAGAGGTATTGGGGATCTCCCCATGTCCG
CCCGCATACGAGTTCTGCGGAGGGATGGCATACTGTGGACCTCAG
GTTGGACTGGGCCTAGTCCCCCAAGTTGGCGTGGAGACTTTGCAG
CCTGAGGGCCAGGCAGGAGCACGAGTGGAAAGCAACTCAGAGGGA
ACCTCCTCTGAGCCCTGTGCCGACCGCCCCAATGCCGTGAAGTTG
GAGAAGGTGGAACCAACTCCCGAGGAGTCCCAGGACATGAAAGCC
CTGCAGAAGGAGCTAGAACAGTTTGCCAAGCTGCTGAAGCAGAAG
AGGATCACCTTGGGGTACACCCAGGCCGACGTGGGGCTCACCCTG
GGCGTTCTCTTTGGAAAGGTGTTCAGCCAGACCACCATCTGTCGC
TTCGAGGCCTTGCAGCTCAGCCTTAAGAACATGTGTAAGCTGCGG
CCCCTGCTGGAGAAGTGGGTGGAGGAAGCCGACAACAATGAGAAC
CTTCAGGAGATATGCAAATCGGAGACCCTGGTGCAGGCCCGGAAG
AGAAAGCGAACTAGCATTGAGAACCGTGTGAGGTGGAGTCTGGAG
ACCATGTTTCTGAAGTGCCCGAAGCCCTCCCTACAGCAGATCACT
CACATCGCCAATCAGCTTGGGCTAGAGAAGGATGTGGTTCGAGTA
TGGTTCTGTAACCGGCGCCAGAAGGGCAAAAGATCAAGTATTGAG
TATTCCCAACGAGAAGAGTATGAGGCTACAGGGACACCTTTCCCA
GGGGGGGCTGTATCCTTTCCTCTGCCCCCAGGTCCCCACTTTGGC
ACCCCAGGCTATGGAAGCCCCCACTTCACCACACTCTACTCAGTC
CCTTTTCCTGAGGGCGAGGCCTTTCCCTCTGTTCCCGTCACTGCT
CTGGGCTCTCCCATGCATTCAAACGCTAGCGGCAGCGGCGCCACG
AACTTCTCTCTGTTAAAGCAAGCAGGAGATGTTGAAGAAAACCCC
GGGCCTGCATGCATGTATAACATGATGGAGACGGAGCTGAAGCCG
CCGGGCCCGCAGCAAGCTTCGGGGGGCGGCGGCGGAGGAGGCAAC
GCCACGGCGGCGGCGACCGGCGGCAACCAGAAGAACAGCCCGGAC
CGCGTCAAGAGGCCCATGAACGCCTTCATGGTATGGTCCCGGGGG
CAGCGGCGTAAGATGGCCCAGGAGAACCCCAAGATGCACAACTCG
GAGATCAGCAAGCGCCTGGGCGCGGAGTGGAAACTTTTGTCCGAG
ACCGAGAAGCGGCCGTTCATCGACGAGGCCAAGCGGCTGCGCGCT
CTGCACATGAAGGAGCACCCGGATTATAAATACCGGCCGCGGCGG
AAAACCAAGACGCTCATGAAGAAGGATAAGTACACGCTTCCCGGA
GGCTTGCTGGCCCCCGGCGGGAACAGCATGGCGAGCGGGGTTGGG
GTGGGCGCCGGCCTGGGTGCGGGCGTGAACCAGCGCATGGACAGC
TACGCGCACATGAACGGCTGGAGCAACGGCAGCTACAGCATGATG
CAGGAGCAGCTGGGCTACCCGCAGCACCCGGGCCTCAACGCTCAC
GGCGCGGCACAGATGCAACCGATGCACCGCTACGACGTCAGCGCC
CTGCAGTACAACTCCATGACCAGCTCGCAGACCTACATGAACGGC
TCGCCCACCTACAGCATGTCCTACTCGCAGCAGGGCACCCCCGGT
ATGGCGCTGGGCTCCATGGGCTCTGTGGTCAAGTCCGAGGCCAGC
TCCAGCCCCCCCGTGGTTACCTCTTCCTCCCACTCCAGGGCGCCC
TGCCAGGCCGGGGACCTCCGGGACATGATCAGCATGTACCTCCCC
GGCGCCGAGGTGCCGGAGCCCGCTGCGCCCAGTAGACTGCACATG
GCCCAGCACTACCAGAGCGGCCCGGTGCCCGGCACGGCCATTAAC
GGCACACTGCCCCTGTCGCACATGGCATGCGGCTCCGGCGAGGGC
AGGGGAAGTCTTCTAACATGCGGGGACGTGGAGGAAAATCCCGGC
CCACTCGAGATGAGGCAGCCACCTGGCGAGTCTGACATGGCTGTC
AGCGACGCTCTGCTCCCGTCCTTCTCCACGTTCGCGTCCGGCCCG
GCGGGAAGGGAGAAGACACTGCGTCCAGCAGGTGCCCCGACTAAC
CGTTGGCGTGAGGAACTCTCTCACATGAAGCGACTTCCCCCACTT
CCCGGCCGCCCCTACGACCTGGCGGCGACGGTGGCCACAGACCTG
GAGAGTGGCGGAGCTGGTGCAGCTTGCAGCAGTAACAACCCGGCC
CTCCTAGCCCGGAGGGAGACCGAGGAGTTCAACGACCTCCTGGAC
CTAGACTTTATCCTTTCCAACTCGCTAACCCACCAGGAATCGGTG
GCCGCCACCGTGACCACCTCGGCGTCAGCTTCATCCTCGTCTTCC
CCAGCGAGCAGCGGCCCTGCCAGCGCGCCCTCCACCTGCAGCTTC
AGCTATCCGATCCGGGCCGGGGGTGACCCGGGCGTGGCTGCCAGC
AACACAGGTGGAGGGCTCCTCTACAGCCGAGAATCTGCGCCACCT
CCCACGGCCCCCTTCAACCTGGCGGACATCAATGACGTGAGCCCC
TCGGGCGGCTTCGTGGCTGAGCTCCTGCGGCCGGAGTTGGACCCA
GTATACATTCCGCCACAGCAGCCTCAGCCGCCAGGTGGCGGGCTG
ATGGGCAAGTTTGTGCTGAAGGCGTCTCTGACCACCCCTGGCAGC
GAGTACAGCAGCCCTTCGGTCATCAGTGTTAGCAAAGGAAGCCCA
GACGGCAGCCACCCCGTGGTAGTGGCGCCCTACAGCGGTGGCCCG
CCGCGCATGTGCCCCAAGATTAAGCAAGAGGCGGTCCCGTCCTGC
ACGGTCAGCCGGTCCCTAGAGGCCCATTTGAGCGCTGGACCCCAG
CTCAGCAACGGCCACCGGCCCAACACACACGACTTCCCCCTGGGG
CGGCAGCTCCCCACCAGGACTACCCCTACACTGAGTCCCGAGGAA
CTGCTGAACAGCAGGGACTGTCACCCTGGCCTGCCTCTTCCCCCA
GGATTCCATCCCCATCCGGGGCCCAACTACCCTCCTTTCCTGCCA
GACCAGATGCAGTCACAAGTCCCCTCTCTCCATTATCAAGAGCTC
ATGCCACCGGGTTCCTGCCTGCCAGAGGAGCCCAAGCCAAAGAGG
GGAAGAAGGTCGTGGCCCCGGAAAAGAACAGCCACCCACACTTGT
GACTATGCAGGCTGTGGCAAAACCTATACCAAGAGTTCTCATCTC
AAGGCACACCTGCGAACTCACACAGGCGAGAAACCTTACCACTGT
GACTGGGACGGCTGTGGGTGGAAATTCGCCCGCTCCGATGAACTG
ACCAGGCACTACCGCAAACACACAGGGCACCGGCCCTTTCAGTGC
CAGAAGTGCGACAGGGCCTTTTCCAGGTCGGACCACCTTGCCTTA
CACATGAAGAGGCACTAAATGACTAGTCTAGCAATCAACCTCTGG
ATTACAAAATTTGTGAAAGATTGACTGGTATTCTTAACTATGTTG
CTCCTTTTACGCTATGTGGATACGCTGCTTTAATGCCTTTGTATC
ATGCTATTGCTTCCCGTATGGCTTTCATTTTCTCCTCCTTGTATA
AATCCTGGTTAGTTCTTGCCACGGCGGAACTCATCGCCGCCTGCC
TTGCCCGCTGCTGGACAGGGGCTCGGCTGTTGGGCACTGACAATT
CCGTGGTGTTTATTTGTGAAATTTGTGATGCTATTGCTTTATTTG
TAACCATTCTAGCTTTATTTGTGAAATTTGTGATGCTATTGCTTT
ATTTGTAACCATTATAAGCTGCAATAAACAAGTTAACAACAACAA
TTGCATTCATTTTATGTTTCAGGTTCAGGGGGAGATGTGGGAGGT
TTTTTAAAGCGGGGGATCCAAATTCCCGATAAGGATCTTCCTAGA
GCATGGCTACGTAGATAAGTAGCATGGCGGGTTAATCATTAACTA
CAAGGAACCCCTAGTGATGGAGTTGGCCACTCCCTCTCTGCGCGC
TCGCTCGCTCACTGAGGCCGGGCGACCAAAGGTCGCCCGACGCCC
GGGCTTTGCCCGGGCGGCCTCAGTGAGCGAGCGAGCGCGCAGCCT
TAATTAACCTAATTCACTGGCCGTCGTTTTACAACGTCGTGACTG
GGAAAACCCTGGCGTTACCCAACTTAATCGCCTTGCAGCACATCC
CCCTTTCGCCAGCTGGCGTAATAGCGAAGAGGCCCGCACCGATCG
CCCTTCCCAACAGTTGCGCAGCCTGAATGGCGAATGGGACGCGCC
CTGTAGCGGCGCATTAAGCGCGGCGGGTGTGGTGGTTACGCGCAG
CGTGACCGCTACACTTGCCAGCGCCCTAGCGCCCGCTCCTTTCGC
TTTCTTCCCTTCCTTTCTCGCCACGTTCGCCGGCTTTCCCCGTCA
AGCTCTAAATCGGGGGCTCCCTTTAGGGTTCCGATTTAGTGCTTT
ACGGCACCTCGACCCCAAAAAACTTGATTAGGGTGATGGTTCACG
TAGTGGGCCATCGCCCTGATAGACGGTTTTTCGCCCTTTGACGTT
GGAGTCCACGTTCTTTAATAGTGGACTCTTGTTCCAAACTGGAAC
AACACTCAACCCTATCTCGGTCTATTCTTTTGATTTATAAGGGAT
TTTGCCGATTTCGGCCTATTGGTTAAAAAATGAGCTGATTTAACA
AAAATTTAACGCGAATTTTAACAAAATATTAACGTTTATAATTTC
AGGTGGCATCTTTCGGGGAAATGTGCGCGGAACCCCTATTTGTTT
ATTTTTCTAAATACATTCAAATATGTATCCGCTCATGAGACAATA
ACCCTGATAAATGCTTCAATAATATTGAAAAAGGAAGAGTATGAG
TATTCAACATTTCCGTGTCGCCCTTATTCCCTTTTTTGCGGCATT
TTGCCTTCCTGTTTTTGCTCACCCAGAAACGCTGGTGAAAGTAAA
AGATGCTGAAGATCAGTTGGGTGCACGAGTGGGTTACATCGAACT
GGATCTCAATAGTGGTAAGATCCTTGAGAGTTTTCGCCCCGAAGA
ACGTTTTCCAATGATGAGCACTTTTAAAGTTCTGCTATGTGGCGC
GGTATTATCCCGTATTGACGCCGGGCAAGAGCAACTCGGTCGCCG
CATACACTATTCTCAGAATGACTTGGTTGAGTACTCACCAGTCAC
AGAAAAGCATCTTACGGATGGCATGACAGTAAGAGAA

VP64, 4 repeats of VP16 (SEQ ID NO: 34) (Non-limiting example of a transactivation domain):

GAGGCCAGCGGTTCCGGACGGGCTGACGCATTGGACGATTTTGAT
CTGGATATGCTGGGAAGTGACGCCCTCGATGATTTTGACCTTGAC
ATGCTTGGTTCGGATGCCCTTGATGACTTTGACCTCGACATGCTC
GGCAGTGACGCCCTTGATGATTTCGACCTGGACATGCTGATTAAC
TCTAGA

P65 (SEQ ID NO: 35) (Non-limiting example of a transactivation domain):

AGCCAGTACCTGCCCGACACCGACGACCGGCACCGGATCGAGGAA
AAGCGGAAGCGGACCTACGAGACATTCAAGAGCATCATGAAGAAG
TCCCCCTTCAGCGGCCCCACCGACCCTAGACCTCCACCTAGAAGA
ATCGCCGTGCCCAGCAGATCCAGCGCCAGCGTGCCAAAACCTGCC
CCCCAGCCTTACCCCTTCACCAGCAGCCTGAGCACCATCAACTAC
GACGAGTTCCCTACCATGGTGTTCCCCAGCGGCCAGATCTCTCAG
GCCTCTGCTCTGGCTCCAGCCCCTCCTCAGGTGCTGCCTCAGGCT
CCTGCTCCTGCACCAGCTCCAGCCATGGTGTCTGCACTGGCTCAG
GCACCAGCACCCGTGCCTGTGCTGGCTCCTGGACCTCCACAGGCT
GTGGCTCCACCAGCCCCTAAACCTACACAGGCCGGCGAGGGCACA
CTGTCTGAAGCTCTGCTGCAGCTGCAGTTCGACGACGAGGATCTG
GGAGCCCTGCTGGGAAACAGCACCGATCCTGCCGTGTTCACCGAC
CTGGCCAGCGTGGACAACAGCGAGTTCCAGCAGCTGCTGAACCAG
GGCATCCCTGTGGCCCCTCACACCACCGAGCCCATGCTGATGGAA
TACCCCGAGGCCATCACCCGGCTCGTGACAGGCGCTCAGAGGCCT
CCTGATCCAGCTCCTGCCCCTCTGGGAGCACCAGGCCTGCCTAAT
GGACTGCTGTCTGGCGACGAGGACTTCAGCTCTATCGCCGATATG
GATTTCTCAGCCTTGCTG

RTA (SEQ ID NO: 36) (Non-limiting example of a transactivation domain):

CGGGATTCCAGGGAAGGGATGTTTTTGCCGAAGCCTGAGGCCGGC
TCCGCTATTAGTGACGTGTTTGAGGGCCGCGAGGTGTGCCAGCCA
AAACGAATCCGGCCATTTCATCCTCCAGGAAGTCCATGGGCCAAC
CGCCCACTCCCCGCCAGCCTCGCACCAACACCAACCGGTCCAGTA
CATGAGCCAGTCGGGTCACTGACCCCGGCACCAGTCCCTCAGCCA
CTGGATCCAGCGCCCGCAGTGACTCCCGAGGCCAGTCACCTGTTG
GAGGATCCCGATGAAGAGACGAGCCAGGCTGTCAAAGCCCTTCGG
GAGATGGCCGATACTGTGATTCCCCAGAAGGAAGAGGCTGCAATC
TGTGGCCAAATGGACCTTTCCCATCCGCCCCCAAGGGGCCATCTG
GATGAGCTGACAACCACACTTGAGTCCATGACCGAGGATCTGAAC
CTGGACTCACCCCTGACCCCGGAATTGAACGAGATTCTGGATACC
TTCCTGAACGACGAGTGCCTCTTGCATGCCATGCATATCAGCACA
GGACTGTCCATCTTCGACACATCTCTGTTT

MPH MS2-P65-HSF1 (SEQ ID NO: 37) (Non-limiting example of a transactivation domain):

GCTTCAAACTTTACTCAGTTCGTGCTCGTGGACAATGGTGGGACA
GGGGATGTGACAGTGGCTCCTTCTAATTTCGCTAATGGGGTGGCA
GAGTGGATCAGCTCCAACTCACGGAGCCAGGCCTACAAGGTGACA
TGCAGCGTCAGGCAGTCTAGTGCCCAGAAGAGAAAGTATACCATC
AAGGTGGAGGTCCCCAAAGTGGCTACCCAGACAGTGGGCGGAGTC
GAACTGCCTGTCGCCGCTTGGAGGTCCTACCTGAACATGGAGCTC
ACTATCCCAATTTTCGCTACCAATTCTGACTGTGAACTCATCGTG
AAGGCAATGCAGGGGCTCCTCAAAGACGGTAATCCTATCCCTTCC
GCCATCGCCGCTAACTCAGGTATCTACAGCGCTGGAGGAGGTGGA
AGCGGAGGAGGAGGAAGCGGAGGAGGAGGTAGCGGACCTAAGAAA
AAGAGGAAGGTGGCGGCCGCTGGATCCCCTTCAGGGCAGATCAGC
AACCAGGCCCTGGCTCTGGCCCCTAGCTCCGCTCCAGTGCTGGCC
CAGACTATGGTGCCCTCTAGTGCTATGGTGCCTCTGGCCCAGCCA
CCTGCTCCAGCCCCTGTGCTGACCCCAGGACCACCCCAGTCACTG
AGCGCTCCAGTGCCCAAGTCTACACAGGCCGGCGAGGGGACTCTG
AGTGAAGCTCTGCTGCACCTGCAGTTCGACGCTGATGAGGACCTG
GGAGCTCTGCTGGGGAACAGCACCGATCCCGGAGTGTTCACAGAT
CTGGCCTCCGTGGACAACTCTGAGTTTCAGCAGCTGCTGAATCAG
GGCGTGTCCATGTCTCATAGTACAGCCGAACCAATGCTGATGGAG
TACCCCGAAGCCATTACCCGGCTGGTGACCGGCAGCCAGCGGCCC
CCCGACCCCGCTCCAACTCCCCTGGGAACCAGCGGCCTGCCTAAT
GGGCTGTCCGGAGATGAAGACTTCTCAAGCATCGCTGATATGGAC
TTTAGTGCCCTGCTGTCACAGATTTCCTCTAGTGGGCAGGGAGGA
GGTGGAAGCGGCTTCAGCGTGGACACCAGTGCCCTGCTGGACCTG
TTCAGCCCCTCGGTGACCGTGCCCGACATGAGCCTGCCTGACCTT
GACAGCAGCCTGGCCAGTATCCAAGAGCTCCTGTCTCCCCAGGAG
CCCCCCAGGCCTCCCGAGGCAGAGAACAGCAGCCCGGATTCAGGG
AAGCAGCTGGTGCACTACACAGCGCAGCCGCTGTTCCTGCTGGAC
CCCGGCTCCGTGGACACCGGGAGCAACGACCTGCCGGTGCTGTTT
GAGCTGGGAGAGGGCTCCTACTTCTCCGAAGGGGACGGCTTCGCC
GAGGACCCCACCATCTCCCTGCTGACAGGCTCGGAGCCTCCCAAA
GCCAAGGACCCCACTGTCTCC

OCT4-2A-SOX2-2A-KLF4 (non-limiting example of nucleic acid sequence encoding human OCT4, human SOX2, and human KLF4, each separated by a 2A peptide) (SEQ ID NO: 38):

ATGGCGGGACACCTGGCTTCGGATTTCGCCTTCTCGCCCCCTCCA
GGTGGTGGAGGTGATGGGCCAGGGGGGCCGGAGCCGGGCTGGGTT
GATCCTCGGACCTGGCTAAGCTTCCAAGGCCCTCCTGGAGGGCCA
GGAATCGGGCCGGGGGTTGGGCCAGGCTCTGAGGTGTGGGGGATT
CCCCCATGCCCCCCGCCGTATGAGTTCTGTGGGGGGATGGCGTAC
TGTGGGCCCCAGGTTGGAGTGGGGCTAGTGCCCCAAGGCGGCTTG
GAGACCTCTCAGCCTGAGGGCGAAGCAGGAGTCGGGGTGGAGAGC
AACTCCGATGGGGCCTCCCCGGAGCCCTGCACCGTCACCCCTGGT
GCCGTGAAGCTGGAGAAGGAGAAGCTGGAGCAAAACCCGGAGGAG
TCCCAGGACATCAAAGCTCTGCAGAAAGAACTCGAGCAATTTGCC
AAGCTCCTGAAGCAGAAGAGGATCACCCTGGGATATACACAGGCC
GATGTGGGGCTCACCCTGGGGGTTCTATTTGGGAAGGTATTCAGC
CAAACGACCATCTGCCGCTTTGAGGCTCTGCAGCTTAGCTTCAAG
AACATGTGTAAGCTGCGGCCCTTGCTGCAGAAGTGGGTGGAGGAA
GCTGACAACAATGAAAATCTTCAGGAGATATGCAAAGCAGAAACC
CTCGTGCAGGCCCGAAAGAGAAAGCGAACCAGTATCGAGAACCGA
GTGAGAGGCAACCTGGAGAATTTGTTCCTGCAGTGCCCGAAACCC
ACACTGCAGCAGATCAGCCACATCGCCCAGCAGCTTGGGCTCGAG
AAGGATGTGGTCCGAGTGTGGTTCTGTAACCGGCGCCAGAAGGGC
AAGCGATCAAGCAGCGACTATGCACAACGAGAGGATTTTGAGGCT
GCTGGGTCTCCTTTCTCAGGGGGACCAGTGTCCTTTCCTCTGGCC
CCAGGGCCCCATTTTGGTACCCCAGGCTATGGGAGCCCTCACTTC
ACTGCACTGTACTCCTCGGTCCCTTTCCCTGAGGGGGAAGCCTTT
CCCCCTGTCTCTGTCACCACTCTGGGCTCTCCCATGCATTCAAAC
GCTAGCGGCAGCGGCGCCACGAACTTCTCTCTGTTAAAGCAAGCA
GGAGATGTTGAAGAAAACCCCGGGCCTGCATGCATGTACAACATG
ATGGAGACGGAGCTGAAGCCGCCGGGCCCGCAGCAAACTTCGGGG
GGCGGCGGCGGCAACTCCACCGCGGCGGCGGCCGGCGGCAACCAG
AAAAACAGCCCGGACCGCGTCAAGCGGCCCATGAATGCCTTCATG
GTGTGGTCCCGCGGGCAGCGGCGCAAGATGGCCCAGGAGAACCCC
AAGATGCACAACTCGGAGATCAGCAAGCGCCTGGGCGCCGAGTGG
AAACTTTTGTCGGAGACGGAGAAGCGGCCGTTCATCGACGAGGCT
AAGCGGCTGCGAGCGCTGCACATGAAGGAGCACCCGGATTATAAA
TACCGGCCCCGGCGGAAAACCAAGACGCTCATGAAGAAGGATAAG
TACACGCTGCCCGGCGGGCTGCTGGCCCCCGGCGGCAATAGCATG
GCGAGCGGGGTCGGGGTGGGCGCCGGCCTGGGCGCGGGCGTGAAC
CAGCGCATGGACAGTTACGCGCACATGAACGGCTGGAGCAACGGC
AGCTACAGCATGATGCAGGACCAGCTGGGCTACCCGCAGCACCCG
GGCCTCAATGCGCACGGCGCAGCGCAGATGCAGCCCATGCACCGC
TACGACGTGAGCGCCCTGCAGTACAACTCCATGACCAGCTCGCAG
ACCTACATGAACGGCTCGCCCACCTACAGCATGTCCTACTCGCAG
CAGGGCACCCCTGGCATGGCTCTTGGCTCCATGGGTTCGGTGGTC
AAGTCCGAGGCCAGCTCCAGCCCCCCTGTGGTTACCTCTTCCTCC
CACTCCAGGGCGCCCTGCCAGGCCGGGGACCTCCGGGACATGATC
AGCATGTATCTCCCCGGCGCCGAGGTGCCGGAACCCGCCGCCCCC
AGCAGACTTCACATGTCCCAGCACTACCAGAGCGGCCCGGTGCCC
GGCACGGCCATTAACGGCACACTGCCCCTCTCACACATGGCATGC
GGCTCCGGCGAGGGCAGGGGAAGTCTTCTAACATGCGGGGACGTG
GAGGAAAATCCCGGCCCACTCGAGATGGCTGTCAGCGACGCGCTG
CTCCCATCTTTCTCCACGTTCGCGTCTGGCCCGGCGGGAAGGGAG
AAGACACTGCGTCAAGCAGGTGCCCCGAATAACCGCTGGCGGGAG
GAGCTCTCCCACATGAAGCGACTTCCCCCAGTGCTTCCCGGCCGC
CCCTATGACCTGGCGGCGGCGACCGTGGCCACAGACCTGGAGAGC
GGCGGAGCCGGTGCGGCTTGCGGCGGTAGCAACCTGGCGCCCCTA
CCTCGGAGAGAGACCGAGGAGTTCAACGATCTCCTGGACCTGGAC
TTTATTCTCTCCAATTCGCTGACCCATCCTCCGGAGTCAGTGGCC
GCCACCGTGTCCTCGTCAGCGTCAGCCTCCTCTTCGTCGTCGCCG
TCGAGCAGCGGCCCTGCCAGCGCGCCCTCCACCTGCAGCTTCACC
TATCCGATCCGGGCCGGGAACGACCCGGGCGTGGCGCCGGGCGGC
ACGGGCGGAGGCCTCCTCTATGGCAGGGAGTCCGCTCCCCCTCCG
ACGGCTCCCTTCAACCTGGCGGACATCAACGACGTGAGCCCCTCG
GGCGGCTTCGTGGCCGAGCTCCTGCGGCCAGAATTGGACCCGGTG
TACATTCCGCCGCAGCAGCCGCAGCCGCCAGGTGGCGGGCTGATG
GGCAAGTTCGTGCTGAAGGCGTCGCTGAGCGCCCCTGGCAGCGAG
TACGGCAGCCCGTCGGTCATCAGCGTCAGCAAAGGCAGCCCTGAC
GGCAGCCACCCGGTGGTGGTGGCGCCCTACAACGGCGGGCCGCCG
CGCACGTGCCCCAAGATCAAGCAGGAGGCGGTCTCTTCGTGCACC
CACTTGGGCGCTGGACCCCCTCTCAGCAATGGCCACCGGCCGGCT
GCACACGACTTCCCCCTGGGGCGGCAGCTCCCCAGCAGGACTACC
CCGACCCTGGGTCTTGAGGAAGTGCTGAGCAGCAGGGACTGTCAC
CCTGCCCTGCCGCTTCCTCCCGGCTTCCATCCCCACCCGGGGCCC
AATTACCCATCCTTCCTGCCCGATCAGATGCAGCCGCAAGTCCCG
CCGCTCCATTACCAAGAGCTCATGCCACCCGGTTCCTGCATGCCA
GAGGAGCCCAAGCCAAAGAGGGGAAGACGATCGTGGCCCCGGAAA
AGGACCGCCACCCACACTTGTGATTACGCGGGCTGCGGCAAAACC
TACACAAAGAGTTCCCATCTCAAGGCACACCTGCGAACCCACACA
GGTGAGAAACCTTACCACTGTGACTGGGACGGCTGTGGATGGAAA
TTCGCCCGCTCAGATGAACTGACCAGGCACTACCGTAAACACACG
GGGCACCGCCCGTTCCAGTGCCAAAAATGCGACCGAGCATTTTCC
AGGTCGGACCACCTCGCCTTACACATGAAGAGGCATTTT

OCT4-2A-SOX2-2A-KLF4 (non-limiting example of an amino acid sequence encoding human OCT4, human SOX2, and human KLF4, each separated by a 2A peptide) (SEQ ID NO: 39):

MAGHLASDFAFSPPPGGGGDGPGGPEPGWVDPRTWLSFQGPPGGP
GIGPGVGPGSEVWGIPPCPPPYEFCGGMAYCGPQVGVGLVPQGGL
ETSQPEGEAGVGVESNSDGASPEPCTVTPGAVKLEKEKLEQNPEE
SQDIKALQKELEQFAKLLKQKRITLGYTQADVGLTLGVLFGKVFS
QTTICRFEALQLSFKNMCKLRPLLQKWVEEADNNENLQEICKAET
LVQARKRKRTSIENRVRGNLENLFLQCPKPTLQQISHIAQQLGLE
KDVVRVWFCNRRQKGKRSSSDYAQREDFEAAGSPFSGGPVSFPLA
PGPHFGTPGYGSPHFTALYSSVPFPEGEAFPPVSVTTLGSPMHSN
ASGSGATNFSLLKQAGDVEENPGPACMYNMMETELKPPGPQQTSG
GGGGNSTAAAAGGNQKNSPDRVKRPMNAFMVWSRGQRRKMAQENP
KMHNSEISKRLGAEWKLLSETEKRPFIDEAKRLRALHMKEHPDYK
YRPRRKTKTLMKKDKYTLPGGLLAPGGNSMASGVGVGAGLGAGVN
QRMDSYAHMNGWSNGSYSMMQDQLGYPQHPGLNAHGAAQMQPMHR
YDVSALQYNSMTSSQTYMNGSPTYSMSYSQQGTPGMALGSMGSVV
KSEASSSPPVVTSSSHSRAPCQAGDLRDMISMYLPGAEVPEPAAP
SRLHMSQHYQSGPVPGTAINGTLPLSHMACGSGEGRGSLLTCGDV
EENPGPLEMAVSDALLPSFSTFASGPAGREKTLRQAGAPNNRWRE
ELSHMKRLPPVLPGRPYDLAAATVATDLESGGAGAACGGSNLAPL
PRRETEEFNDLLDLDFILSNSLTHPPESVAATVSSSASASSSSSP
SSSGPASAPSTCSFTYPIRAGNDPGVAPGGTGGGLLYGRESAPPP
TAPFNLADINDVSPSGGFVAELLRPELDPVYIPPQQPQPPGGGLM
GKFVLKASLSAPGSEYGSPSVISVSKGSPDGSHPVVVAPYNGGPP
RTCPKIKQEAVSSCTHLGAGPPLSNGHRPAAHDFPLGRQLPSRTT
PTLGLEEVLSSRDCHPALPLPPGFHPHPGPNYPSFLPDQMQPQVP
PLHYQELMPPGSCMPEEPKPKRGRRSWPRKRTATHTCDYAGCGKT
YTKSSHLKAHLRTHTGEKPYHCDWDGCGWKFARSDELTRHYRKHT
GHRPFQCQKCDRAFSRSDHLALHMKRHF

Human OCT4 nucleic acid sequence (non-limiting example of a nucleic acid sequence encoding human OCT4) (SEQ ID NO: 40):

ATGGCGGGACACCTGGCTTCGGATTTCGCCTTCTCGCCCCCTCCA
GGTGGTGGAGGTGATGGGCCAGGGGGGCCGGAGCCGGGCTGGGTT
GATCCTCGGACCTGGCTAAGCTTCCAAGGCCCTCCTGGAGGGCCA
GGAATCGGGCCGGGGGTTGGGCCAGGCTCTGAGGTGTGGGGGATT
CCCCCATGCCCCCCGCCGTATGAGTTCTGTGGGGGGATGGCGTAC
TGTGGGCCCCAGGTTGGAGTGGGGCTAGTGCCCCAAGGCGGCTTG
GAGACCTCTCAGCCTGAGGGCGAAGCAGGAGTCGGGGTGGAGAGC
AACTCCGATGGGGCCTCCCCGGAGCCCTGCACCGTCACCCCTGGT
GCCGTGAAGCTGGAGAAGGAGAAGCTGGAGCAAAACCCGGAGGAG
TCCCAGGACATCAAAGCTCTGCAGAAAGAACTCGAGCAATTTGCC
AAGCTCCTGAAGCAGAAGAGGATCACCCTGGGATATACACAGGCC
GATGTGGGGCTCACCCTGGGGGTTCTATTTGGGAAGGTATTCAGC
CAAACGACCATCTGCCGCTTTGAGGCTCTGCAGCTTAGCTTCAAG
AACATGTGTAAGCTGCGGCCCTTGCTGCAGAAGTGGGTGGAGGAA
GCTGACAACAATGAAAATCTTCAGGAGATATGCAAAGCAGAAACC
CTCGTGCAGGCCCGAAAGAGAAAGCGAACCAGTATCGAGAACCGA
GTGAGAGGCAACCTGGAGAATTTGTTCCTGCAGTGCCCGAAACCC
ACACTGCAGCAGATCAGCCACATCGCCCAGCAGCTTGGGCTCGAG
AAGGATGTGGTCCGAGTGTGGTTCTGTAACCGGCGCCAGAAGGGC
AAGCGATCAAGCAGCGACTATGCACAACGAGAGGATTTTGAGGCT
GCTGGGTCTCCTTTCTCAGGGGGACCAGTGTCCTTTCCTCTGGCC
CCAGGGCCCCATTTTGGTACCCCAGGCTATGGGAGCCCTCACTTC
ACTGCACTGTACTCCTCGGTCCCTTTCCCTGAGGGGGAAGCCTTT
CCCCCTGTCTCTGTCACCACTCTGGGCTCTCCCATGCATTCAAAC

Human OCT4 amino acid sequence (non-limiting example of an amino acid sequence encoding human OCT4) (SEQ ID NO: 41):

MAGHLASDFAFSPPPGGGGDGPGGPEPGWVDPRTWLSFQGPPGGP
GIGPGVGPGSEVWGIPPCPPPYEFCGGMAYCGPQVGVGLVPQGGL
ETSQPEGEAGVGVESNSDGASPEPCTVTPGAVKLEKEKLEQNPEE
SQDIKALQKELEQFAKLLKQKRITLGYTQADVGLTLGVLFGKVFS
QTTICRFEALQLSFKNMCKLRPLLQKWVEEADNNENLQEICKAET
LVQARKRKRTSIENRVRGNLENLFLQCPKPTLQQISHIAQQLGLE
KDVVRVWFCNRRQKGKRSSSDYAQREDFEAAGSPFSGGPVSFPLA
PGPHFGTPGYGSPHFTALYSSVPFPEGEAFPPVSVTTLGSPMHSN

Human SOX2 nucleic acid sequence (non-limiting example of a nucleic acid sequence encoding human SOX2) (SEQ ID NO: 42):

ATGTACAACATGATGGAGACGGAGCTGAAGCCGCCGGGCCCGCAG
CAAACTTCGGGGGGCGGCGGCGGCAACTCCACCGCGGCGGCGGCC
GGCGGCAACCAGAAAAACAGCCCGGACCGCGTCAAGCGGCCCATG
AATGCCTTCATGGTGTGGTCCCGCGGGCAGCGGCGCAAGATGGCC
CAGGAGAACCCCAAGATGCACAACTCGGAGATCAGCAAGCGCCTG
GGCGCCGAGTGGAAACTTTTGTCGGAGACGGAGAAGCGGCCGTTC
ATCGACGAGGCTAAGCGGCTGCGAGCGCTGCACATGAAGGAGCAC
CCGGATTATAAATACCGGCCCCGGCGGAAAACCAAGACGCTCATG
AAGAAGGATAAGTACACGCTGCCCGGCGGGCTGCTGGCCCCCGGC
GGCAATAGCATGGCGAGCGGGGTCGGGGTGGGCGCCGGCCTGGGC
GCGGGCGTGAACCAGCGCATGGACAGTTACGCGCACATGAACGGC
TGGAGCAACGGCAGCTACAGCATGATGCAGGACCAGCTGGGCTAC
CCGCAGCACCCGGGCCTCAATGCGCACGGCGCAGCGCAGATGCAG
CCCATGCACCGCTACGACGTGAGCGCCCTGCAGTACAACTCCATG
ACCAGCTCGCAGACCTACATGAACGGCTCGCCCACCTACAGCATG
TCCTACTCGCAGCAGGGCACCCCTGGCATGGCTCTTGGCTCCATG
GGTTCGGTGGTCAAGTCCGAGGCCAGCTCCAGCCCCCCTGTGGTT
ACCTCTTCCTCCCACTCCAGGGCGCCCTGCCAGGCCGGGGACCTC
CGGGACATGATCAGCATGTATCTCCCCGGCGCCGAGGTGCCGGAA
CCCGCCGCCCCCAGCAGACTTCACATGTCCCAGCACTACCAGAGC
GGCCCGGTGCCCGGCACGGCCATTAACGGCACACTGCCCCTCTCA
CACATG

Human SOX2 amino acid sequence (non-limiting example of an amino acid sequence encoding human SOX2) (SEQ ID NO: 43):

MYNMMETELKPPGPQQTSGGGGGNSTAAAAGGNQKNSPDRVKRPM
NAFMVWSRGQRRKMAQENPKMHNSEISKRLGAEWKLLSETEKRPF
IDEAKRLRALHMKEHPDYKYRPRRKTKTLMKKDKYTLPGGLLAPG
GNSMASGVGVGAGLGAGVNQRMDSYAHMNGWSNGSYSMMQDQLGY
PQHPGLNAHGAAQMQPMHRYDVSALQYNSMTSSQTYMNGSPTYSM
SYSQQGTPGMALGSMGSVVKSEASSSPPVVTSSSHSRAPCQAGDL
RDMISMYLPGAEVPEPAAPSRLHMSQHYQSGPVPGTAINGTLPLS
HM

Human KLF4 (non-limiting example of a nucleotide sequence encoding human KLF4) (SEQ ID NO: 44):

ATGGCTGTCAGCGACGCGCTGCTCCCATCTTTCTCCACGTTCGCG
TCTGGCCCGGCGGGAAGGGAGAAGACACTGCGTCAAGCAGGTGCC
CCGAATAACCGCTGGCGGGAGGAGCTCTCCCACATGAAGCGACTT
CCCCCAGTGCTTCCCGGCCGCCCCTATGACCTGGCGGCGGCGACC
GTGGCCACAGACCTGGAGAGCGGCGGAGCCGGTGCGGCTTGCGGC
GGTAGCAACCTGGCGCCCCTACCTCGGAGAGAGACCGAGGAGTTC
AACGATCTCCTGGACCTGGACTTTATTCTCTCCAATTCGCTGACC
CATCCTCCGGAGTCAGTGGCCGCCACCGTGTCCTCGTCAGCGTCA
GCCTCCTCTTCGTCGTCGCCGTCGAGCAGCGGCCCTGCCAGCGCG
CCCTCCACCTGCAGCTTCACCTATCCGATCCGGGCCGGGAACGAC
CCGGGCGTGGCGCCGGGCGGCACGGGCGGAGGCCTCCTCTATGGC
AGGGAGTCCGCTCCCCCTCCGACGGCTCCCTTCAACCTGGCGGAC
ATCAACGACGTGAGCCCCTCGGGCGGCTTCGTGGCCGAGCTCCTG
CGGCCAGAATTGGACCCGGTGTACATTCCGCCGCAGCAGCCGCAG
CCGCCAGGTGGCGGGCTGATGGGCAAGTTCGTGCTGAAGGCGTCG
CTGAGCGCCCCTGGCAGCGAGTACGGCAGCCCGTCGGTCATCAGC
GTCAGCAAAGGCAGCCCTGACGGCAGCCACCCGGTGGTGGTGGCG
CCCTACAACGGCGGGCCGCCGCGCACGTGCCCCAAGATCAAGCAG
GAGGCGGTCTCTTCGTGCACCCACTTGGGCGCTGGACCCCCTCTC
AGCAATGGCCACCGGCCGGCTGCACACGACTTCCCCCTGGGGCGG
CAGCTCCCCAGCAGGACTACCCCGACCCTGGGTCTTGAGGAAGTG
CTGAGCAGCAGGGACTGTCACCCTGCCCTGCCGCTTCCTCCCGGC
TTCCATCCCCACCCGGGGCCCAATTACCCATCCTTCCTGCCCGAT
CAGATGCAGCCGCAAGTCCCGCCGCTCCATTACCAAGAGCTCATG
CCACCCGGTTCCTGCATGCCAGAGGAGCCCAAGCCAAAGAGGGGA
AGACGATCGTGGCCCCGGAAAAGGACCGCCACCCACACTTGTGAT
TACGCGGGCTGCGGCAAAACCTACACAAAGAGTTCCCATCTCAAG
GCACACCTGCGAACCCACACAGGTGAGAAACCTTACCACTGTGAC
TGGGACGGCTGTGGATGGAAATTCGCCCGCTCAGATGAACTGACC
AGGCACTACCGTAAACACACGGGGCACCGCCCGTTCCAGTGCCAA
AAATGCGACCGAGCATTTTCCAGGTCGGACCACCTCGCCTTACAC
ATGAAGAGGCATTTT

Human KLF4 (non-limiting example of an amino acid sequence encoding human KLF4) (SEQ ID NO: 45):

MAVSDALLPSFSTFASGPAGREKTLRQAGAPNNRWREELSHMKRL
PPVLPGRPYDLAAATVATDLESGGAGAACGGSNLAPLPRRETEEF
NDLLDLDFILSNSLTHPPESVAATVSSSASASSSSSPSSSGPASA
PSTCSFTYPIRAGNDPGVAPGGTGGGLLYGRESAPPPTAPFNLAD
INDVSPSGGFVAELLRPELDPVYIPPQQPQPPGGGLMGKFVLKAS
LSAPGSEYGSPSVISVSKGSPDGSHPVVVAPYNGGPPRTCPKIKQ
EAVSSCTHLGAGPPLSNGHRPAAHDFPLGRQLPSRTTPTLGLEEV
LSSRDCHPALPLPPGFHPHPGPNYPSFLPDQMQPQVPPLHYQELM
PPGSCMPEEPKPKRGRRSWPRKRTATHTCDYAGCGKTYTKSSHLK
AHLRTHTGEKPYHCDWDGCGWKFARSDELTRHYRKHTGHRPFQCQ
KCDRAFSRSDHLALHMKRHF

Human RCVRN (recoverin) promoter (non-limiting example of a human RCVRN (recoverin) promoter) (SEQ ID NO: 46):

ATTTTAATCTCACTAGGGTTCTGGGAGCACCCCCCCCCACCGCTC
CCGCCCTCCACAAAGCTCCTGGGCCCCTCCTCCCTTCAAGGATTG
CGAAGAGCTGGTCGCAAATCCTCCTAAGCCACCAGCATCTCGGTC
TTCAGCTCACACCAGCCTTGAGCCCAGCCTGCGGCCAGGGGACCA
CGCACGTCCCACCCACCCAGCGACTCCCCAGCCGCTGCCCACTCT
TCCTCACTCA

RSV promoter (non-limiting example of a RSV promoter) (SEQ ID NO: 47):

AATGTAGTCTTATGCAATACTCTTGTAGTCTTGCAACATGGTAAC
GATGAGTTAGCAACATGCCTTACAAGGAGAGAAAAAGCACCGTGC
ATGCCGATTGGTGGAAGTAAGGTGGTACGATCGTGCCTTATTAGG
AAGGCAACAGACGGGTCTGACATGGATTGGACGAACCACTGAATT
GCCGCATTGCAGAGATATTGTATTTAAGTGCCTAGCTCGATACAT
AAAC

CMV promoter (non-limiting example of a CMV promoter) (SEQ ID NO: 48):

CATTGATTATTGACTAGTTATTAATAGTAATCAATTACGGGGTCA
TTAGTTCATAGCCCATATATGGAGTTCCGCGTTACATAACTTACG
GTAAATGGCCCGCCTGGCTGACCGCCCAACGACCCCCGCCCATTG
ACGTCAATAATGACGTATGTTCCCATAGTAACGCCAATAGGGACT
TTCCATTGACGTCAATGGGTGGACTATTTACGGTAAACTGCCCAC
TTGGCAGTACATCAAGTGTATCATATGCCAAGTACGCCCCCTATT
GACGTCAATGACGGTAAATGGCCCGCCTGGCATTATGCCCAGTAC
ATGACCTTATGGGACTTTCCTACTTGGCAGTACATCTACGTATTA
GTCATCGCTATTACCATGGTGATGCGGTTTTGGCAGTACATCAAT
GGGCGTGGATAGCGGTTTGACTCACGGGGATTTCCAAGTCTCCAC
CCCATTGACGTCAATGGGAGTTTGTTTTGGCACCAAAATCAACGG
GACTTTCCAAAATGTCGTAACAACTCCGCCCCATTGACGCAAATG
GGCGGTAGGCGTGTACGGTGGGAGGTCTATATAAGCAGAGCTGGT
TTAGTGAACCGTCAGATCCGCTAGAGATCCGC

EFS promoter (non-limiting example of an EFS promoter) (SEQ ID NO: 49):

TCGAGTGGCTCCGGTGCCCGTCAGTGGGCAGAGCGCACATCGCCC
ACAGTCCCCGAGAAGTTGGGGGGAGGGGTCGGCAATTGAACCGGT
GCCTAGAGAAGGTGGCGCGGGGTAAACTGGGAAAGTGATGTCGTG
TACTGGCTCCGCCTTTTTCCCGAGGGTGGGGGAGAACCGTATATA
AGTGCAGTAGTCGCCGTGAACGTTCTTTTTCGCAACGGGTTTGCC
GCCAGAACACAGGTGTCGTGACCGCGG

Human GRK1 (rhodopsin kinase) promoter (non-limiting example of a human promoter) (SEQ ID NO: 50):

Gggccccagaagcctggtggttgtttgtccttctcaggggaaaag
tgaggcggccccttggaggaaggggccgggcagaatgatctaatc
ggattccaagcagctcaggggattgtctttttctagcaccttctt
gccactcctaagcgtcctccgtgaccccggctgggatttcgcctg
gtgctgtgtcagccccggtctcccaggggcttcccagtggtcccc
aggaaccctcgacagggcccggtctctctcgtccagcaagggcag
ggacgggccacaggccaagggc

Human CRX (cone rod homeobox transcription factor) promoter (non-limiting example of a human CRX promoter) (SEQ ID NO: 51):

Gcctgtagccttaatctctcctagcagggggtttgggggagggag
gaggagaaagaaagggccccttatggctgagacacaatgacccag
ccacaaggagggattaccgggcg

Human NRL promoter (neural retina leucine zipper transcription factor enhancer upstream of the human TK terminal promoter) (non-limiting example of a human NRL promoter) (SEQ ID NO: 52):

Aggtaggaagtggcctttaactccatagaccctatttaaacagct
tcggacaggtttaaacatctccttggataattcctagtatccctg
ttcccactcctactcagggatgatagctctaagaggtgttagggg
attaggctgaaaatgtaggtcacccctcagccatctgggaactag
aatgagtgagagaggagagaggggcagagacacacacattcgcat
attaaggtgacgcgtgtggcctcgaacaccgagcgaccctgcagc
gacccgcttaa

Human red opsin promoter (hred promoter) (SEQ ID NO: 101):

Gatccggttccaggcctcggccctaaatagtctccctgggctttc
aagagaaccacatgagaaaggaggattcgggctctgagcagtttc
accacccaccccccagtctgcaaatcctgacccgtgggtccacct
gccccaaaggcggacgcaggacagtagaagggaacagagaacaca
taaacacagagagggccacagcggctcccacagtcaccgccacct
tcctggcggggatgggtggggcgtctgagtttggttcccagcaaa
tccctctgagccgcccttgcgggctcgcctcaggagcaggggagc
aagaggtgggaggaggaggtctaagtcccaggcccaattaagaga
tcaggtagtgtagggtttgggagcttttaaggtgaagaggcccgg
gctgatcccacaggccagtataaagcgccgtgaccctcaggtgat
gcgccagggccggctgccgtcggggacagggctttccatagc

Human rhodopsin promoter (rho promoter) (SEQ ID NO: 102):

Agttaatgattaacccgccatgctacttatctacgtagccatgct
ctaggaagatcggaattcgcccttaagctagcagatcttccccac
ctagccacctggcaaactgctccttctctcaaaggcccaaacatg
gcctcccagactgcaacccccaggcagtcaggccctgtctccaca
acctcacagccaccctggacggaatctgcttcttcccacatttga
gtcctcctcagcccctgagctcctctgggcagggctgtttctttc
catctttgtattcccaggggcctgcaaataaatgtttaatgaacg
aacaagagagtgaattccaattccatgcaacaaggattgggctcc
tgggccctaggctatgtgtctggcaccagaaacggaagctgcagg
ttgcagcccctgccctcatggagctcctcctgtcagaggagtgtg
gggactggatgactccagaggtaacttgtgggggaacgaacaggt
aaggggctgtgtgacgagatgagagactgggagaataaaccagaa
agtctctagctgtccagaggacatagcacagaggcccatggtccc
tatttcaaacccaggccaccagactgagctgggaccttgggacag
acaagtcatgcagaagttaggggaccttctcctcccttttcctgg
atggatcctgagtaccttctcctccctgacctcaggcttcctcct
agtgtcaccttggcccctcttagaagccaattaggccctcagttt
ctgcagcggggattaatatgattatgaacacccccaatctcccag
atgctgattcagccaggagcttaggagggggaggtcactttataa
gggtctgggggggtcagaacccagagtcatcccctgaattctgca

Mouse cone arrestin promoter (mcar promoter) (SEQ ID NO: 103):

Ggttcttcccattttggctacatggtctttttttttacctttttg
gttcctttggccttttggcttttggcttccagggcttctggatcc
cccccaacccctcccatacacatacacatgtgcactcgtgcactc
aacccagcacaggataatgttcattcttgacctttccacatacat
ctggctatgttctctctcttatctacaataaatctcctccactat
acttaggagcagttatgttcttcttctttctttcttttttttttt
tttcattcagtaacatcatcagaatcccctagctctggcctacct
cctcagtaacaatcagctgatccctggccactaatctgtactcac
taatctgttttccaaactcttggcccctgagctaattatagcagt
gcttcatgccacccaccccaaccctattcttgttctctgactccc
actaatctacacattcagaggattgtggatataagaggctgggag
gccagcttagcaaccagagctggagg

Human rhodopsin kinase promoter (hrk promoter) (SEQ ID NO: 104):

Gggccccagaagcctggtggttgtttgtccttctcaggggaaaag
tgaggcggccccttggaggaaggggccgggcagaatgatctaatc
ggattccaagcagctcaggggattgtctttttctagcaccttctt
gccactcctaagcgtcctccgtgaccccggctgggatttagcctg
gtgctgtgtcagccccggtctcccaggggcttcccagtggtcccc
aggaaccctcgacagggcccggtctctctcgtccagcaagggcag
ggacgggccacaggccaagggc

TRE-human OSK-SV40 (SEQ ID NO: 105):

TTATGCAGTGCTGCCATAACCATGAGTGATAACACTGCGGCCAACTTACTTCTGACAA
CGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTA
GTAATGGTAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAGATTTAATTAAGGCCTTAATTAGG
CTGCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCTT
GTAGTTAATGATTAACCCGCCATGCTACTTATCTACGTAGCCATGCTCTAGGAAGATCGGAATTCT
TTACTCCCTATCAGTGATAGAGAACGTATGAAGAGTTTACTCCCTATCAGTGATAGAGAACGTATG
CAGACTTTACTCCCTATCAGTGATAGAGAACGTATAAGGAGTTTACTCCCTATCAGTGATAGAGAA
CGTATGACCAGTTTACTCCCTATCAGTGATAGAGAACGTATCTACAGTTTACTCCCTATCAGTGAT
AGAGAACGTATATCCAGTTTACTCCCTATCAGTGATAGAGAACGTATAAGCTTTAGGCGTGTACGG
TGGGCGCCTATAAAAGCAGAGCTCGTTTAGTGAACCGTCAGATCGCCTGGAGCAATTCCACAACA
CTTTTGTCTTATACCAACTTTCCGTACCACTTCCTACCCTCGTAAAGCGGCCGCGCCACCATGGCGG
GACACCTGGCTTCGGATTTCGCCTTCTCGCCCCCTCCAGGTGGTGGAGGTGATGGGCCAGGGGGGC
CGGAGCCGGGCTGGGTTGATCCTCGGACCTGGCTAAGCTTCCAAGGCCCTCCTGGAGGGCCAGGA
ATCGGGCCGGGGGTTGGGCCAGGCTCTGAGGTGTGGGGGATTCCCCCATGCCCCCCGCCGTATGA
GTTCTGTGGGGGGATGGCGTACTGTGGGCCCCAGGTTGGAGTGGGGCTAGTGCCCCAAGGCGGCT
TGGAGACCTCTCAGCCTGAGGGCGAAGCAGGAGTCGGGGTGGAGAGCAACTCCGATGGGGCCTCC
CCGGAGCCCTGCACCGTCACCCCTGGTGCCGTGAAGCTGGAGAAGGAGAAGCTGGAGCAAAACCC
GGAGGAGTCCCAGGACATCAAAGCTCTGCAGAAAGAACTCGAGCAATTTGCCAAGCTCCTGAAGC
AGAAGAGGATCACCCTGGGATATACACAGGCCGATGTGGGGCTCACCCTGGGGGTTCTATTTGGG
AAGGTATTCAGCCAAACGACCATCTGCCGCTTTGAGGCTCTGCAGCTTAGCTTCAAGAACATGTGT
AAGCTGCGGCCCTTGCTGCAGAAGTGGGTGGAGGAAGCTGACAACAATGAAAATCTTCAGGAGAT
ATGCAAAGCAGAAACCCTCGTGCAGGCCCGAAAGAGAAAGCGAACCAGTATCGAGAACCGAGTG
AGAGGCAACCTGGAGAATTTGTTCCTGCAGTGCCCGAAACCCACACTGCAGCAGATCAGCCACAT
CGCCCAGCAGCTTGGGCTCGAGAAGGATGTGGTCCGAGTGTGGTTCTGTAACCGGCGCCAGAAGG
GCAAGCGATCAAGCAGCGACTATGCACAACGAGAGGATTTTGAGGCTGCTGGGTCTCCTTTCTCAG
GGGGACCAGTGTCCTTTCCTCTGGCCCCAGGGCCCCATTTTGGTACCCCAGGCTATGGGAGCCCTC
ACTTCACTGCACTGTACTCCTCGGTCCCTTTCCCTGAGGGGGAAGCCTTTCCCCCTGTCTCTGTCAC
CACTCTGGGCTCTCCCATGCATTCAAACGCTAGCGGCAGCGGCGCCACGAACTTCTCTCTGTTAAA
GCAAGCAGGAGATGTTGAAGAAAACCCCGGGCCTGCATGCATGTACAACATGATGGAGACGGAG
CTGAAGCCGCCGGGCCCGCAGCAAACTTCGGGGGGCGGCGGCGGCAACTCCACCGCGGCGGCGGC
CGGCGGCAACCAGAAAAACAGCCCGGACCGCGTCAAGCGGCCCATGAATGCCTTCATGGTGTGGT
CCCGCGGGCAGCGGCGCAAGATGGCCCAGGAGAACCCCAAGATGCACAACTCGGAGATCAGCAA
GCGCCTGGGCGCCGAGTGGAAACTTTTGTCGGAGACGGAGAAGCGGCCGTTCATCGACGAGGCTA
AGCGGCTGCGAGCGCTGCACATGAAGGAGCACCCGGATTATAAATACCGGCCCCGGCGGAAAACC
AAGACGCTCATGAAGAAGGATAAGTACACGCTGCCCGGCGGGCTGCTGGCCCCCGGCGGCAATAG
CATGGCGAGCGGGGTCGGGGTGGGCGCCGGCCTGGGCGCGGGCGTGAACCAGCGCATGGACAGTT
ACGCGCACATGAACGGCTGGAGCAACGGCAGCTACAGCATGATGCAGGACCAGCTGGGCTACCCG
CAGCACCCGGGCCTCAATGCGCACGGCGCAGCGCAGATGCAGCCCATGCACCGCTACGACGTGAG
CGCCCTGCAGTACAACTCCATGACCAGCTCGCAGACCTACATGAACGGCTCGCCCACCTACAGCAT
GTCCTACTCGCAGCAGGGCACCCCTGGCATGGCTCTTGGCTCCATGGGTTCGGTGGTCAAGTCCGA
GGCCAGCTCCAGCCCCCCTGTGGTTACCTCTTCCTCCCACTCCAGGGCGCCCTGCCAGGCCGGGGA
CCTCCGGGACATGATCAGCATGTATCTCCCCGGCGCCGAGGTGCCGGAACCCGCCGCCCCCAGCA
GACTTCACATGTCCCAGCACTACCAGAGCGGCCCGGTGCCCGGCACGGCCATTAACGGCACACTG
CCCCTCTCACACATGGCATGCGGCTCCGGCGAGGGCAGGGGAAGTCTTCTAACATGCGGGGACGT
GGAGGAAAATCCCGGCCCACTCGAGATGGCTGTCAGCGACGCGCTGCTCCCATCTTTCTCCACGTT
CGCGTCTGGCCCGGCGGGAAGGGAGAAGACACTGCGTCAAGCAGGTGCCCCGAATAACCGCTGGC
GGGAGGAGCTCTCCCACATGAAGCGACTTCCCCCAGTGCTTCCCGGCCGCCCCTATGACCTGGCGG
CGGCGACCGTGGCCACAGACCTGGAGAGCGGCGGAGCCGGTGCGGCTTGCGGCGGTAGCAACCTG
GCGCCCCTACCTCGGAGAGAGACCGAGGAGTTCAACGATCTCCTGGACCTGGACTTTATTCTCTCC
AATTCGCTGACCCATCCTCCGGAGTCAGTGGCCGCCACCGTGTCCTCGTCAGCGTCAGCCTCCTCTT
CGTCGTCGCCGTCGAGCAGCGGCCCTGCCAGCGCGCCCTCCACCTGCAGCTTCACCTATCCGATCC
GGGCCGGGAACGACCCGGGCGTGGCGCCGGGCGGCACGGGCGGAGGCCTCCTCTATGGCAGGGA
GTCCGCTCCCCCTCCGACGGCTCCCTTCAACCTGGCGGACATCAACGACGTGAGCCCCTCGGGCGG
CTTCGTGGCCGAGCTCCTGCGGCCAGAATTGGACCCGGTGTACATTCCGCCGCAGCAGCCGCAGCC
GCCAGGTGGCGGGCTGATGGGCAAGTTCGTGCTGAAGGCGTCGCTGAGCGCCCCTGGCAGCGAGT
ACGGCAGCCCGTCGGTCATCAGCGTCAGCAAAGGCAGCCCTGACGGCAGCCACCCGGTGGTGGTG
GCGCCCTACAACGGCGGGCCGCCGCGCACGTGCCCCAAGATCAAGCAGGAGGCGGTCTCTTCGTG
CACCCACTTGGGCGCTGGACCCCCTCTCAGCAATGGCCACCGGCCGGCTGCACACGACTTCCCCCT
GGGGCGGCAGCTCCCCAGCAGGACTACCCCGACCCTGGGTCTTGAGGAAGTGCTGAGCAGCAGGG
ACTGTCACCCTGCCCTGCCGCTTCCTCCCGGCTTCCATCCCCACCCGGGGCCCAATTACCCATCCTT
CCTGCCCGATCAGATGCAGCCGCAAGTCCCGCCGCTCCATTACCAAGAGCTCATGCCACCCGGTTC
CTGCATGCCAGAGGAGCCCAAGCCAAAGAGGGGAAGACGATCGTGGCCCCGGAAAAGGACCGCC
ACCCACACTTGTGATTACGCGGGCTGCGGCAAAACCTACACAAAGAGTTCCCATCTCAAGGCACA
CCTGCGAACCCACACAGGTGAGAAACCTTACCACTGTGACTGGGACGGCTGTGGATGGAAATTCG
CCCGCTCAGATGAACTGACCAGGCACTACCGTAAACACACGGGGCACCGCCCGTTCCAGTGCCAA
AAATGCGACCGAGCATTTTCCAGGTCGGACCACCTCGCCTTACACATGAAGAGGCATTTTTAAATG
ACTAGTGCGCGCAGCGGCCGACCATGGCCCAACTTGTTTATTGCAGCTTATAATGGTTACAAATAA
AGCAATAGCATCACAAATTTCACAAATAAAGCATTTTTTTCACTGCATTCTAGTTGTGGTTTGTCCA
AACTCATCAATGTATCTTATCATGTCTGGATCTCGGTACCGGATCCAAATTCCCGATAAGGATCTTC
CTAGAGCATGGCTACGTAGATAAGTAGCATGGCGGGTTAATCATTAACTACAAGGAACCCCTAGT
GATGGAGTTGGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGCCTTAATTAACCTA
ATTCACTGGCCGTCGTTTTACAACGTCGTGACTGGGAAAACCCTGGCGTTACCCAACTTAATCGCC
TTGCAGCACATCCCCCTTTCGCCAGCTGGCGTAATAGCGAAGAGGCCCGCACCGATCGCCCTTCCC
AACAGTTGCGCAGCCTGAATGGCGAATGGGACGCGCCCTGTAGCGGCGCATTAAGCGCGGCGGGT
GTGGTGGTTACGCGCAGCGTGACCGCTACACTTGCCAGCGCCCTAGCGCCCGCTCCTTTCGCTTTCT
TCCCTTCCTTTCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGGTTCACGTAG
TGGGCCATCGCCCTGATAGACGGTTTTTCGCCCTTTGACGTTGGAGTCCACGTTCTTTAATAGTGGA
CTCTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAACA
AAATATTAACGTTTATAATTTCAGGTGGCATCTTTCGGGGAAATGTGCGCGGAACCCCTATTTGTTT
ATTTTTCTAAATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTG
GGTGCACGAGTGGGTTACATCGAACTGGATCTCAATAGTGGTAAGATCCTTGAGAGTTTTCGCCCC
GAAGAACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAA

EFS-human OSK-SV40 (SEQ ID NO: 106):

TTATGCAGTGCTGCCATAACCATGAGTGATAACACTGCGGCCAACTTACTTCTGACAA
CGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTA
GTAATGGTAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAGATTTAATTAAGGCCTTAATTAGG
CTGCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCTT
GTAGTTAATGATTAACCCGCCATGCTACTTATCTACGTAGCCATGCTCTAGGAAGATCGGAATTCT
CGAGTGGCTCCGGTGCCCGTCAGTGGGCAGAGCGCACATCGCCCACAGTCCCCGAGAAGTTGGGG
GGAGGGGTCGGCAATTGAACCGGTGCCTAGAGAAGGTGGCGCGGGGTAAACTGGGAAAGTGATG
TCGTGTACTGGCTCCGCCTTTTTCCCGAGGGTGGGGGAGAACCGTATATAAGTGCAGTAGTCGCCG
TGAACGTTCTTTTTCGCAACGGGTTTGCCGCCAGAACACAGGTGTCGTGACGCGGGCGGCCGCGCC
ACCATGGCGGGACACCTGGCTTCGGATTTCGCCTTCTCGCCCCCTCCAGGTGGTGGAGGTGATGGG
CCAGGGGGGCCGGAGCCGGGCTGGGTTGATCCTCGGACCTGGCTAAGCTTCCAAGGCCCTCCTGG
AGGGCCAGGAATCGGGCCGGGGGTTGGGCCAGGCTCTGAGGTGTGGGGGATTCCCCCATGCCCCC
CGCCGTATGAGTTCTGTGGGGGGATGGCGTACTGTGGGCCCCAGGTTGGAGTGGGGCTAGTGCCC
CAAGGCGGCTTGGAGACCTCTCAGCCTGAGGGCGAAGCAGGAGTCGGGGTGGAGAGCAACTCCG
ATGGGGCCTCCCCGGAGCCCTGCACCGTCACCCCTGGTGCCGTGAAGCTGGAGAAGGAGAAGCTG
GAGCAAAACCCGGAGGAGTCCCAGGACATCAAAGCTCTGCAGAAAGAACTCGAGCAATTTGCCAA
GCTCCTGAAGCAGAAGAGGATCACCCTGGGATATACACAGGCCGATGTGGGGCTCACCCTGGGGG
TTCTATTTGGGAAGGTATTCAGCCAAACGACCATCTGCCGCTTTGAGGCTCTGCAGCTTAGCTTCA
AGAACATGTGTAAGCTGCGGCCCTTGCTGCAGAAGTGGGTGGAGGAAGCTGACAACAATGAAAAT
CTTCAGGAGATATGCAAAGCAGAAACCCTCGTGCAGGCCCGAAAGAGAAAGCGAACCAGTATCG
AGAACCGAGTGAGAGGCAACCTGGAGAATTTGTTCCTGCAGTGCCCGAAACCCACACTGCAGCAG
ATCAGCCACATCGCCCAGCAGCTTGGGCTCGAGAAGGATGTGGTCCGAGTGTGGTTCTGTAACCG
GCGCCAGAAGGGCAAGCGATCAAGCAGCGACTATGCACAACGAGAGGATTTTGAGGCTGCTGGGT
CTCCTTTCTCAGGGGGACCAGTGTCCTTTCCTCTGGCCCCAGGGCCCCATTTTGGTACCCCAGGCTA
TGGGAGCCCTCACTTCACTGCACTGTACTCCTCGGTCCCTTTCCCTGAGGGGGAAGCCTTTCCCCCT
GTCTCTGTCACCACTCTGGGCTCTCCCATGCATTCAAACGCTAGCGGCAGCGGCGCCACGAACTTC
TCTCTGTTAAAGCAAGCAGGAGATGTTGAAGAAAACCCCGGGCCTGCATGCATGTACAACATGAT
GGAGACGGAGCTGAAGCCGCCGGGCCCGCAGCAAACTTCGGGGGGCGGCGGCGGCAACTCCACC
GCGGCGGCGGCCGGCGGCAACCAGAAAAACAGCCCGGACCGCGTCAAGCGGCCCATGAATGCCTT
CATGGTGTGGTCCCGCGGGCAGCGGCGCAAGATGGCCCAGGAGAACCCCAAGATGCACAACTCGG
AGATCAGCAAGCGCCTGGGCGCCGAGTGGAAACTTTTGTCGGAGACGGAGAAGCGGCCGTTCATC
GACGAGGCTAAGCGGCTGCGAGCGCTGCACATGAAGGAGCACCCGGATTATAAATACCGGCCCCG
GCGGAAAACCAAGACGCTCATGAAGAAGGATAAGTACACGCTGCCCGGCGGGCTGCTGGCCCCCG
GCGGCAATAGCATGGCGAGCGGGGTCGGGGTGGGCGCCGGCCTGGGCGCGGGCGTGAACCAGCG
CATGGACAGTTACGCGCACATGAACGGCTGGAGCAACGGCAGCTACAGCATGATGCAGGACCAGC
TGGGCTACCCGCAGCACCCGGGCCTCAATGCGCACGGCGCAGCGCAGATGCAGCCCATGCACCGC
TACGACGTGAGCGCCCTGCAGTACAACTCCATGACCAGCTCGCAGACCTACATGAACGGCTCGCC
CACCTACAGCATGTCCTACTCGCAGCAGGGCACCCCTGGCATGGCTCTTGGCTCCATGGGTTCGGT
GGTCAAGTCCGAGGCCAGCTCCAGCCCCCCTGTGGTTACCTCTTCCTCCCACTCCAGGGCGCCCTG
CCAGGCCGGGGACCTCCGGGACATGATCAGCATGTATCTCCCCGGCGCCGAGGTGCCGGAACCCG
CCGCCCCCAGCAGACTTCACATGTCCCAGCACTACCAGAGCGGCCCGGTGCCCGGCACGGCCATT
AACGGCACACTGCCCCTCTCACACATGGCATGCGGCTCCGGCGAGGGCAGGGGAAGTCTTCTAAC
ATGCGGGGACGTGGAGGAAAATCCCGGCCCACTCGAGATGGCTGTCAGCGACGCGCTGCTCCCAT
CTTTCTCCACGTTCGCGTCTGGCCCGGCGGGAAGGGAGAAGACACTGCGTCAAGCAGGTGCCCCG
AATAACCGCTGGCGGGAGGAGCTCTCCCACATGAAGCGACTTCCCCCAGTGCTTCCCGGCCGCCCC
TATGACCTGGCGGCGGCGACCGTGGCCACAGACCTGGAGAGCGGCGGAGCCGGTGCGGCTTGCGG
CGGTAGCAACCTGGCGCCCCTACCTCGGAGAGAGACCGAGGAGTTCAACGATCTCCTGGACCTGG
ACTTTATTCTCTCCAATTCGCTGACCCATCCTCCGGAGTCAGTGGCCGCCACCGTGTCCTCGTCAGC
GTCAGCCTCCTCTTCGTCGTCGCCGTCGAGCAGCGGCCCTGCCAGCGCGCCCTCCACCTGCAGCTT
CACCTATCCGATCCGGGCCGGGAACGACCCGGGCGTGGCGCCGGGCGGCACGGGCGGAGGCCTCC
TCTATGGCAGGGAGTCCGCTCCCCCTCCGACGGCTCCCTTCAACCTGGCGGACATCAACGACGTGA
GCCCCTCGGGCGGCTTCGTGGCCGAGCTCCTGCGGCCAGAATTGGACCCGGTGTACATTCCGCCGC
AGCAGCCGCAGCCGCCAGGTGGCGGGCTGATGGGCAAGTTCGTGCTGAAGGCGTCGCTGAGCGCC
CCTGGCAGCGAGTACGGCAGCCCGTCGGTCATCAGCGTCAGCAAAGGCAGCCCTGACGGCAGCCA
CCCGGTGGTGGTGGCGCCCTACAACGGCGGGCCGCCGCGCACGTGCCCCAAGATCAAGCAGGAGG
CGGTCTCTTCGTGCACCCACTTGGGCGCTGGACCCCCTCTCAGCAATGGCCACCGGCCGGCTGCAC
ACGACTTCCCCCTGGGGCGGCAGCTCCCCAGCAGGACTACCCCGACCCTGGGTCTTGAGGAAGTG
CTGAGCAGCAGGGACTGTCACCCTGCCCTGCCGCTTCCTCCCGGCTTCCATCCCCACCCGGGGCCC
AATTACCCATCCTTCCTGCCCGATCAGATGCAGCCGCAAGTCCCGCCGCTCCATTACCAAGAGCTC
ATGCCACCCGGTTCCTGCATGCCAGAGGAGCCCAAGCCAAAGAGGGGAAGACGATCGTGGCCCCG
GAAAAGGACCGCCACCCACACTTGTGATTACGCGGGCTGCGGCAAAACCTACACAAAGAGTTCCC
ATCTCAAGGCACACCTGCGAACCCACACAGGTGAGAAACCTTACCACTGTGACTGGGACGGCTGT
GGATGGAAATTCGCCCGCTCAGATGAACTGACCAGGCACTACCGTAAACACACGGGGCACCGCCC
GTTCCAGTGCCAAAAATGCGACCGAGCATTTTCCAGGTCGGACCACCTCGCCTTACACATGAAGAG
GCATTTTTAAATGACTAGTGCGCGCAGCGGCCGACCATGGCCCAACTTGTTTATTGCAGCTTATAA
TGGTTACAAATAAAGCAATAGCATCACAAATTTCACAAATAAAGCATTTTTTTCACTGCATTCTAG
TTGTGGTTTGTCCAAACTCATCAATGTATCTTATCATGTCTGGATCTCGGTACCGGATCCAAATTCC
CGATAAGGATCTTCCTAGAGCATGGCTACGTAGATAAGTAGCATGGCGGGTTAATCATTAACTACA
AGGAACCCCTAGTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCGAGCGCGCAGC
CTTAATTAACCTAATTCACTGGCCGTCGTTTTACAACGTCGTGACTGGGAAAACCCTGGCGTTACC
CAACTTAATCGCCTTGCAGCACATCCCCCTTTCGCCAGCTGGCGTAATAGCGAAGAGGCCCGCACC
GATCGCCCTTCCCAACAGTTGCGCAGCCTGAATGGCGAATGGGACGCGCCCTGTAGCGGCGCATT
AAGCGCGGCGGGTGTGGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGTTGGAGTCCACG
TTCTTTAATAGTGGACTCTTGTTCCAAACTGGAACAACACTCAACCCTATCTCGGTCTATTCTTTTG
ATTTATAAGGGATTTTGCCGATTTCGGCCTATTGGTTAAAAAATGAGCTGATTTAACAAAAATTTA
ACGCGAATTTTAACAAAATATTAACGTTTATAATTTCAGGTGGCATCTTTCGGGGAAATGTGCGCG
GAACCCCTATTTGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAATAGTGGTAAGATCCTT
GAGAGTTTTCGCCCCGAAGAACGTTTTCCAATGATGAGCACTTTTAAAGTTCTGCTATGTGGCGCG
GTATTATCCCGTATTGACGCCGGGCAAGAGCAACTCGGTCGCCGCATACACTATTCTCAGAATGAC
TTGGTTGAGTACTCACCAGTCACAGAAAAGCATCTTACGGATGGCATGACAGTAAGAGAA

TRE-Fluc-SV40 (SEQ ID NO: 107):

TTATGCAGTGCTGCCATAACCATGAGTGATAACACTGCGGCCAACTTACTTCTGACAA
CGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTA
GTAATGGTAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAGATTTAATTAAGGCCTTAATTAGG
CTGCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCTT
GTAGTTAATGATTAACCCGCCATGCTACTTATCTACGTAGCCATGCTCTAGGAAGATCGGAATTCT
TTACTCCCTATCAGTGATAGAGAACGTATGAAGAGTTTACTCCCTATCAGTGATAGAGAACGTATG
CAGACTTTACTCCCTATCAGTGATAGAGAACGTATAAGGAGTTTACTCCCTATCAGTGATAGAGAA
CGTATGACCAGTTTACTCCCTATCAGTGATAGAGAACGTATCTACAGTTTACTCCCTATCAGTGAT
AGAGAACGTATATCCAGTTTACTCCCTATCAGTGATAGAGAACGTATAAGCTTTAGGCGTGTACGG
TGGGCGCCTATAAAAGCAGAGCTCGTTTAGTGAACCGTCAGATCGCCTGGAGCAATTCCACAACA
CTTTTGTCTTATACCAACTTTCCGTACCACTTCCTACCCTCGTAAAGCGGCCGCATGGAAGACGCCA
AAAACATAAAGAAAGGCCCGGCGCCATTCTATCCGCTGGAAGATGGAACCGCTGGAGAGCAACTG
CATAAGGCTATGAAGAGATACGCCCTGGTTCCTGGAACAATTGCTTTTACAGATGCACATATCGAG
GTGGACATCACTTACGCTGAGTACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTG
TTGGGCGCGTTATTTATCGGAGTTGCAGTTGCGCCCGCGAACGACATTTATAATGAACGTGAATTG
CTCAACAGTATGGGCATTTCGCAGCCTACCGTGGTGTTCGTTTCCAAAAAGGGGTTGCAAAAAATT
TTGAACGTGCAAAAAAAGCTCCCAATCATCCAAAAAATTATTATCATGGATTCTAAAACGGATTAC
CAGGGATTTCAGTCGATGTACACGTTCGTCACATCTCATCTACCTCCCGGTTTTAATGAATACGATT
TTGTGCCAGAGTCCTTCGATAGGGACAAGACAATTGCACTGATCATGAACTCCTCTGGATCTACTG
GTCTGCCTAAAGGTGTCGCTCTGCCTCATAGAACTGCCTGCGTGAGATTCTCGCATGCCAGAGATC
CTATTTTTGGCAATCAAATCATTCCGGATACTGCGATTTTAAGTGTTGTTCCATTCCATCACGGTTT
TGGAATGTTTACTACACTCGGATATTTGATATGTGGATTTCGAGTCGTCTTAATGTATAGATTTGAA
GAAGAGCTGTTTCTGAGGAGCCTTCAGGATTACAAGATTCAAAGTGCGCTGCTGGTGCCAACCCTA
TTCTCCTTCTTCGCCAAAAGCACTCTGATTGACAAATACGATTTATCTAATTTACACGAAATTGCTT
CTGGTGGCGCTCCCCTCTCTAAGGAAGTCGGGGAAGCGGTTGCCAAGAGGTTCCATCTGCCAGGTA
TCAGGCAAGGATATGGGCTCACTGAGACTACATCAGCTATTCTGATTACACCCGAGGGGGATGAT
AAACCGGGCGCGGTCGGTAAAGTTGTTCCATTTTTTGAAGCGAAGGTTGTGGATCTGGATACCGGG
AAAACGCTGGGCGTTAATCAAAGAGGCGAACTGTGTGTGAGAGGTCCTATGATTATGTCCGGTTAT
GTAAACAATCCGGAAGCGACCAACGCCTTGATTGACAAGGATGGATGGCTACATTCTGGAGACAT
AGCTTACTGGGACGAAGACGAACACTTCTTCATCGTTGACCGCCTGAAGTCTCTGATTAAGTACAA
AGGCTATCAGGTGGCTCCCGCTGAATTGGAATCCATCTTGCTCCAACACCCCAACATCTTCGACGC
AGGTGTCGCAGGTCTTCCCGACGATGACGCCGGTGAACTTCCCGCCGCCGTTGTTGTTTTGGAGCA
CGGAAAGACGATGACGGAAAAAGAGATCGTGGATTACGTCGCCAGTCAAGTAACAACCGCGAAA
AAGTTGCGCGGAGGAGTTGTGTTTGTGGACGAAGTACCGAAAGGTCTTACCGGAAAACTCGACGC
AAGAAAAATCAGAGAGATCCTCATAAAGGCCAAGAAGGGCGGAAAGATCGCCGTGTAAACTAGT
GCGCGCAGCGGCCGACCATGGCCCAACTTGTTTATTGCAGCTTATAATGGTTACAAATAAAGCAAT
AGCATCACAAATTTCACAAATAAAGCATTTTTTTCACTGCATTCTAGTTGTGGTTTGTCCAAACTCA
TCAATGTATCTTATCATGTCTGGATCTCGGTACCGGATCCAAATTCCCGATAAGGATCTTCCTAGA
GCATGGCTACGTAGATAAGTAGCATGGCGGGTTAATCATTAACTACAAGGAACCCCTAGTGATGG
AGTTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCCTTAATTAACCTAATTCAC
TGGCCGTCGTTTTACAACGTCGTGACTGGGAAAACCCTGGCGTTACCCAACTTAATCGCCTTGCAG
CACATCCCCCTTTCGCCAGCTGGCGTAATAGCGAAGAGGCCCGCACCGATCGCCCTTCCCAACAGT
TGCGCAGCCTGAATGGCGAATGGGACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTTTAGGGTTCCG
ATTTAGTGCTTTACGGCACCTCGACCCCAAAAAACTTGATTAGGGTGATGGTTCACGTAGTGGGCC
ATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTTTAACAAAATAT
TAACGTTTATAATTTCAGGTGGCATCTTTCGGGGAAATGTGCGCGGAACCCCTATTTGTTTATTTTT
CTAAATACATTCAAATATGTATCCGCTCATGAGACAATAACCCTGATAAATGCTTCAATAATATTG
AAAAAGGAAGAGTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATCAGTTGGGTGCA
CGAGTGGGTTACATCGAACTGGATCTCAATAGTGGTAAGATCCTTGAGAGTTTTCGCCCCGAAGAA
CGTTTTCCAATGATGAGCACTTTTAAAGTTCTGCTATGTGGCGCGGTATTATCCCGTATTGACGCCG
GGCAAGAGCAACTCGGTCGCCGCATACACTATTCTCAGAATGACTTGGTTGAGTACTCACCAGTCA
CAGAAAAGCATCTTACGGATGGCATGACAGTAAGAGAA

shRNA against mouse KDM1a (SEQ ID NO: 108):

CACAAGTCAAACCTTTAT

shRNA against human Tet1-1 (SEQ ID NO: 109):

GGACGTAATCCAGAAAGAAGA

shRNA against human Tet1-2 (SEQ ID NO: 110):

TTGTGCCTCTGGAGGTTATAA

shRNA against human Tet3-1 (SEQ ID NO: 111):

GGAAATAAAGGCTGGTGAAGG

shRNA against human Tet3-2 (SEQ ID NO: 112):

GAAAGATGAAGGTCCATATTA

shRNA against mouse Tet1-2 (SEQ ID NO: 113):

GCAGATGGCCGTGACACAAAT

shRNA against mouse Tet1-1 (SEQ ID NO: 114):

GCTCATGGAGACTAGGTTTGG

shRNA against both mouse and human Tet2 (SEQ ID NO: 115):

GGATGTAAGTTTGCCAGAAGC

shRNA against mouse Tet3 (SEQ ID NO: 116):

GCTCCAACGAGAAGCTATTTG

shRNA against scramble sequence (no target in genome) (SEQ ID NO: 117):

GTTCAGATGTGCGGCGAGT

Amino acid sequence encoding P2A (SEQ ID NO: 118):

GSGATNFSLLKQAGDVEENPGP

Nucleic acid sequence encoding P2A (SEQ ID NO: 119):

GGCAGCGGCGCCACGAACTTCTCTCTGTTAAAGCAAGCAGGAGATGTT
GAAGAAAACCCCGGGCCT

Nucleic acid sequence encoding T2A (SEQ ID NO: 120)

(SEQ ID NO: 120)
GGCTCCGGCGAGGGCAGGGGAAGTCTTCTAACATGCGGGGACGTGGAG
GAAAATCCCGGCCCA

SEQ ID NO: 121:

TTATGCAGTGCTGCCATAACCATGAGTGATAACACTGCGGCCAACTTACTTCTGACAA
CGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTA
GTAATGGTAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAGATTTAATTAAGGCCTTAATTAGG
CTGCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCTT
GTAGTTAATGATTAACCCGCCATGCTACTTATCTACGTAGCCATGCTCTAGGAAGATCGGAATTCT
TTACTCCCTATCAGTGATAGAGAACGTATGAAGAGTTTACTCCCTATCAGTGATAGAGAACGTATG
CAGACTTTACTCCCTATCAGTGATAGAGAACGTATAAGGAGTTTACTCCCTATCAGTGATAGAGAA
CGTATGACCAGTTTACTCCCTATCAGTGATAGAGAACGTATCTACAGTTTACTCCCTATCAGTGAT
AGAGAACGTATATCCAGTTTACTCCCTATCAGTGATAGAGAACGTATAAGCTTTAGGCGTGTACGG
TGGGCGCCTATAAAAGCAGAGCTCGTTTAGTGAACCGTCAGATCGCCTGGAGCAATTCCACAACA
CTTTTGTCTTATACCAACTTTCCGTACCACTTCCTACCCTCGTAAAGCGGCCGCGCCACCATGGCGG
GACACCTGGCTTCGGATTTCGCCTTCTCGCCCCCTCCAGGTGGTGGAGGTGATGGGCCAGGGGGGC
CGGAGCCGGGCTGGGTTGATCCTCGGACCTGGCTAAGCTTCCAAGGCCCTCCTGGAGGGCCAGGA
ATCGGGCCGGGGGTTGGGCCAGGCTCTGAGGTGTGGGGGATTCCCCCATGCCCCCCGCCGTATGA
GTTCTGTGGGGGGATGGCGTACTGTGGGCCCCAGGTTGGAGTGGGGCTAGTGCCCCAAGGCGGCT
TGGAGACCTCTCAGCCTGAGGGCGAAGCAGGAGTCGGGGTGGAGAGCAACTCCGATGGGGCCTCC
CCGGAGCCCTGCACCGTCACCCCTGGTGCCGTGAAGCTGGAGAAGGAGAAGCTGGAGCAAAACCC
GGAGGAGTCCCAGGACATCAAAGCTCTGCAGAAAGAACTCGAGCAATTTGCCAAGCTCCTGAAGC
AGAAGAGGATCACCCTGGGATATACACAGGCCGATGTGGGGCTCACCCTGGGGGTTCTATTTGGG
AAGGTATTCAGCCAAACGACCATCTGCCGCTTTGAGGCTCTGCAGCTTAGCTTCAAGAACATGTGT
AAGCTGCGGCCCTTGCTGCAGAAGTGGGTGGAGGAAGCTGACAACAATGAAAATCTTCAGGAGAT
ATGCAAAGCAGAAACCCTCGTGCAGGCCCGAAAGAGAAAGCGAACCAGTATCGAGAACCGAGTG
AGAGGCAACCTGGAGAATTTGTTCCTGCAGTGCCCGAAACCCACACTGCAGCAGATCAGCCACAT
CGCCCAGCAGCTTGGGCTCGAGAAGGATGTGGTCCGAGTGTGGTTCTGTAACCGGCGCCAGAAGG
GCAAGCGATCAAGCAGCGACTATGCACAACGAGAGGATTTTGAGGCTGCTGGGTCTCCTTTCTCAG
GGGGACCAGTGTCCTTTCCTCTGGCCCCAGGGCCCCATTTTGGTACCCCAGGCTATGGGAGCCCTC
ACTTCACTGCACTGTACTCCTCGGTCCCTTTCCCTGAGGGGGAAGCCTTTCCCCCTGTCTCTGTCAC
CACTCTGGGCTCTCCCATGCATTCAAACGCTAGCGGCAGCGGCGCCACGAACTTCTCTCTGTTAAA
GCAAGCAGGAGATGTTGAAGAAAACCCCGGGCCTGCATGCATGTACAACATGATGGAGACGGAG
CTGAAGCCGCCGGGCCCGCAGCAAACTTCGGGGGGCGGCGGCGGCAACTCCACCGCGGCGGCGGC
CGGCGGCAACCAGAAAAACAGCCCGGACCGCGTCAAGCGGCCCATGAATGCCTTCATGGTGTGGT
CCCGCGGGCAGCGGCGCAAGATGGCCCAGGAGAACCCCAAGATGCACAACTCGGAGATCAGCAA
GCGCCTGGGCGCCGAGTGGAAACTTTTGTCGGAGACGGAGAAGCGGCCGTTCATCGACGAGGCTA
AGCGGCTGCGAGCGCTGCACATGAAGGAGCACCCGGATTATAAATACCGGCCCCGGCGGAAAACC
AAGACGCTCATGAAGAAGGATAAGTACACGCTGCCCGGCGGGCTGCTGGCCCCCGGCGGCAATAG
CATGGCGAGCGGGGTCGGGGTGGGCGCCGGCCTGGGCGCGGGCGTGAACCAGCGCATGGACAGTT
ACGCGCACATGAACGGCTGGAGCAACGGCAGCTACAGCATGATGCAGGACCAGCTGGGCTACCCG
CAGCACCCGGGCCTCAATGCGCACGGCGCAGCGCAGATGCAGCCCATGCACCGCTACGACGTGAG
CGCCCTGCAGTACAACTCCATGACCAGCTCGCAGACCTACATGAACGGCTCGCCCACCTACAGCAT
GTCCTACTCGCAGCAGGGCACCCCTGGCATGGCTCTTGGCTCCATGGGTTCGGTGGTCAAGTCCGA
GGCCAGCTCCAGCCCCCCTGTGGTTACCTCTTCCTCCCACTCCAGGGCGCCCTGCCAGGCCGGGGA
CCTCCGGGACATGATCAGCATGTATCTCCCCGGCGCCGAGGTGCCGGAACCCGCCGCCCCCAGCA
GACTTCACATGTCCCAGCACTACCAGAGCGGCCCGGTGCCCGGCACGGCCATTAACGGCACACTG
CCCCTCTCACACATGGCATGCGGCTCCGGCGAGGGCAGGGGAAGTCTTCTAACATGCGGGGACGT
GGAGGAAAATCCCGGCCCACTCGAGATGGCTGTCAGCGACGCGCTGCTCCCATCTTTCTCCACGTT
CGCGTCTGGCCCGGCGGGAAGGGAGAAGACACTGCGTCAAGCAGGTGCCCCGAATAACCGCTGGC
GGGAGGAGCTCTCCCACATGAAGCGACTTCCCCCAGTGCTTCCCGGCCGCCCCTATGACCTGGCGG
CGGCGACCGTGGCCACAGACCTGGAGAGCGGCGGAGCCGGTGCGGCTTGCGGCGGTAGCAACCTG
GCGCCCCTACCTCGGAGAGAGACCGAGGAGTTCAACGATCTCCTGGACCTGGACTTTATTCTCTCC
AATTCGCTGACCCATCCTCCGGAGTCAGTGGCCGCCACCGTGTCCTCGTCAGCGTCAGCCTCCTCTT
CGTCGTCGCCGTCGAGCAGCGGCCCTGCCAGCGCGCCCTCCACCTGCAGCTTCACCTATCCGATCC
GGGCCGGGAACGACCCGGGCGTGGCGCCGGGCGGCACGGGCGGAGGCCTCCTCTATGGCAGGGA
GTCCGCTCCCCCTCCGACGGCTCCCTTCAACCTGGCGGACATCAACGACGTGAGCCCCTCGGGCGG
CTTCGTGGCCGAGCTCCTGCGGCCAGAATTGGACCCGGTGTACATTCCGCCGCAGCAGCCGCAGCC
GCCAGGTGGCGGGCTGATGGGCAAGTTCGTGCTGAAGGCGTCGCTGAGCGCCCCTGGCAGCGAGT
ACGGCAGCCCGTCGGTCATCAGCGTCAGCAAAGGCAGCCCTGACGGCAGCCACCCGGTGGTGGTG
GCGCCCTACAACGGCGGGCCGCCGCGCACGTGCCCCAAGATCAAGCAGGAGGCGGTCTCTTCGTG
CACCCACTTGGGCGCTGGACCCCCTCTCAGCAATGGCCACCGGCCGGCTGCACACGACTTCCCCCT
GGGGCGGCAGCTCCCCAGCAGGACTACCCCGACCCTGGGTCTTGAGGAAGTGCTGAGCAGCAGGG
ACTGTCACCCTGCCCTGCCGCTTCCTCCCGGCTTCCATCCCCACCCGGGGCCCAATTACCCATCCTT
CCTGCCCGATCAGATGCAGCCGCAAGTCCCGCCGCTCCATTACCAAGAGCTCATGCCACCCGGTTC
CTGCATGCCAGAGGAGCCCAAGCCAAAGAGGGGAAGACGATCGTGGCCCCGGAAAAGGACCGCC
ACCCACACTTGTGATTACGCGGGCTGCGGCAAAACCTACACAAAGAGTTCCCATCTCAAGGCACA
CCTGCGAACCCACACAGGTGAGAAACCTTACCACTGTGACTGGGACGGCTGTGGATGGAAATTCG
CCCGCTCAGATGAACTGACCAGGCACTACCGTAAACACACGGGGCACCGCCCGTTCCAGTGCCAA
AAATGCGACCGAGCATTTTCCAGGTCGGACCACCTCGCCTTACACATGAAGAGGCATTTTTAAATG
ACTAGTGCGCGCAGCGGCCGACCATGGCCCAACTTGTTTATTGCAGCTTATAATGGTTACAAATAA
AGCAATAGCATCACAAATTTCACAAATAAAGCATTTTTTTCACTGCATTCTAGTTGTGGTTTGTCCA
AACTCATCAATGTATCTTATCATGTCTGGATCTCGGTACCGGATCCAAATTCCCGATAAGGATCTTC
CTAGAGCATGGCTACGTAGATAAGTAGCATGGCGGGTTAATCATTAACTACAAGGAACCCCTAGT
GATGGAGTTGGCCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGCCTTAATTAACCTA
ATTCACTGGCCGTCGTTTTACAACGTCGTGACTGGGAAAACCCTGGCGTTACCCAACTTAATCGCC
TTGCAGCACATCCCCCTTTCGCCAGCTGGCGTAATAGCGAAGAGGCCCGCACCGATCGCCCTTCCC
AACAGTTGCGCAGCCTGAATGGCGAATGGGACGCGCCCTGTAGCGGCGCATTAAGCGCGGCGGGT
GTGGTGGTTACGCGCAGCGTGACCGCTACACTTGCCAGCGCCCTAGCGCCCGCTCCTTTCGCTTTCT
TCCCTTCCTTTCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGGTTCACGTAG
TGGGCCATCGCCCTGATAGACGGTTTTTCGCCCTTTGACGTTGGAGTCCACGTTCTTTAATAGTGGA
CTCTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAACA
AAATATTAACGTTTATAATTTCAGGTGGCATCTTTCGGGGAAATGTGCGCGGAACCCCTATTTGTTT
ATTTTTCTAAATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTG
GGTGCACGAGTGGGTTACATCGAACTGGATCTCAATAGTGGTAAGATCCTTGAGAGTTTTCGCCCC
GAAGAACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAA

Thy1.2 promoter (RGC-specific) (SEQ ID NO: 122):

AATTCAGAGACCGGGAACCAAACTAGCCTTTAAAAAACATAAGTACAGGAGCCAGCAAGATGGCT
CAGTGGGTAAAGGTGCCTACCAGCAAGCCTGACAGCCTGAGTTCAGTCCCCACGAACTACGTGGT
AGGAGAGGACCAACCAACTCTGGAAATCTGTTCTGCAAACACATGCTCACACACACACACACAAA
TAGTATAAACAATTTTAAATTTCATTTAAAAATAATTTGTAAACAAAATCATTAGCACAGGTTTTA
GAAAGAGCCTCTTGGTGACATCAAGTTGATGCTGTAGATGGGGTATCATTCCTGAGGACCCAAAA
CCGGGTCTCAGCCTTTCCCCATTCTGAGAGTTCTCTCTTTTCTCAGCCACTAGCTGAAGAGTAGAGT
GGCTCAGCACTGGGCTCTTGAGTTCCCAAGTCCTACAACTGGTCAGCCTGACTACTAACCAGCCAT
GAAGAAACAAGGAGTGGATGGGCTGAGTCTGCTGGGATGGGAGTGGAGTTAGTAAGTGGCCATG
GATGTAATGACCCCAGCAATGCTGGCTAGAAGGCATGCCTCCTTTCCTTGTCTGGAGACGGAACGG
GAGGGATCATCTTGTACTCACAGAAGGGAGAACATTCTAGCTGGTTGGGCCAAAATGTGCAAGTT
CACCTGGAGGTGGTGGTGCATGCTTTTAACTCCAGTACTCAGGAGGCAGGGCCAGGTGGATCTCTG
TGAGTTCAAGACCAGCCTGCACTATGGAGAGAGTTTTGGGACAGCCAGAGTTACACAGAAAAATC
CTGGTGGAAAATCTGAAAGAAAGAGAGAAAGAAAGAAAGAAAGAAAGGAAGAAAGAAAGAAAG
AGTGGCAGGCAGGCAGGCAGGAGGAAGGAAGGAAGGAAGGAAGGAAGGAAGGAAGGAAGGAA
GGAAAATAGGTGCGACTTCAAGATCCGGAGTTACAAGCAGAATGCACTGTTTCCCTAACAGGGCC
AAGTGTTTTGAGTAACTGAAGGTGGGCATGATGCCTGGGAAGCAGAAACAAGCCAGGCAGATGCA
CCCCTTGCCTTGCTTCCGAAGGGCTGCAGTAGCATGGAAAACATGGAAAACAACCAATCCATTCCC
TTTGCTGATATAACAGGCTCCAAAGCCAAAACCTGTCACTGGAGGCTCAAGAGCAGATCTCCAGC
CAAGAGGCAAAGGAATGGGGGAAGCTGGAGGGCCTCCCTCTGGTTATCCAGGCTTCTGAAGGTTC
AAGCAAAGAAAGGGTTACAACCTTAAAAGGAGAGCGTCCCGGGGTATGGGTAGAAGACTGCTCC
ACCCCGACCCCCAGGGTCCCTAACCGTCTTTTCCCTGGGCGAGTCAGCCCAATCACAGGACTGAGA
GTGCCTCTTTAGTAGCAGCAAGCCACTTCGGACACCCAAATGGAACACCTCCAGTCAGCCCTCGCC
GACCACCCCACCCCCTCCATCCTTTTCCCTCAGCCTCCGATTGGCTGAATCTAGAGTCCCTCCCTGC
TCCCCCCTCTCTCCCCACCCCTGGTGAAAACTGCGGGCTTCAGCGCTGGGTGCAGCAACTGGAGGC
GTTGGCGCACCAGGAGGAGGCTGCAGCTAGGGGAGTCCAGGTGAGAGCAGGCCGACGGGAGGGA
CCCGCACATGCAAGGACCGCCGCAGGGCGAGGATGCAAGCCTTCCCCAGCTACAGTTTTGGGAAA
GGATACCAGGGCGCTCCTATATGGGGGCGCGGGAACTGGGGAAAGAAGGTGCTCCCAGGTCGAG
GTGGGAGAGGAAGGCAGTGCGGGGTCACGGGCTTTCTCCCTGCTAACGGACGCTTTCGAAGAGTG
GGTGCCGGAGGAGAACCATGAGGAAGGACATCAAGGACAGCCTTTGGTCCCCAAGCTCAAATCGC
TTTAGTGGTGCGAATAGAGGGAGGAGGTGGGTGGCAAACTGGAGGGAGTCCCCAGCGGGTGACCT
CGTGGCTGGCTGGGTGCGGGGCACCGCAGGTAAGAAAACCGCAATGTTGCGGGAGGGGACTGGGT
GGCAGGCGCGGGGGAGGGGAAAGCTAGAAAGGATGCGAGGGAGCGGAGGGGGGAGGGAGCGGG
AGAATCTCAACTGGTAGAGGAAGATTAAAATGAGGAAATAGCATCAGGGTGGGGTTAGCCAAGCC
GGGCCTCAGGGAAAGGGCGCAAAGTTTGTCTGGGTGTGGGCTTAGGTGGGCTGGGTATGAGATTC
GGGGCGCCGAAAACACTGCTGCGCCTCTGCCAAATCACGCTACCCCTGTATCTAGTTCTGCCAGGC
TTCTCCAGCCCCAGCCCCAATTCTTTTCTCTAGTGTTCCCCCTTCCCTCCCCTGAATCTCAAGCCCAC
ACTCCCTCCTCCATAACCCACTGTTATCAAATCTAAGTCATTTGCCACCCAACAACCATCAGGAGG
CGGAAGCAGACGGGAGGAGTTTGAGATCAACTTGGGCTACATCACGAGTTCCAGGCTCACCAAGG
CTTCTTAAGGAGACCTTGTCTCTAAAATTAATTAATTAATTAATTAATAGTCCCCTTTCTCTGCCAC
AGAACCTTGGGATCTGGCTCCTGGTCGCAGCTCCCCCCACCCCAGGCTGACATTCACTGCCATAGC
CCATCCGGAAATCCTAGTCTATTTCCCCATGGATCTTGAACTGCAGAGAGAATGGCAGAGTGGCCC
GCCCTGTGCAAAGGATGTTCCTAGCCTAGGTGGAGCTCGCGAACTCGCAGACTGTGCCTCTCTTGG
GCAAGGACAGGCTAGACAGCCTGCCGGTGTGTTGAGCTAGGGCACTGTGGGGAAGGCAGAGAAC
CTGTGCAGGGCAGCAATGAACACAGGACCAGAAAACTGCAGCCCTAGGAACACTCAAGAGCTGG
CCATTTGCAAGCATCTCTGGCCTCCGTGCTTCTCACTCATGTCCCATGTCTTATACAGGCCTCTGTG
GCACCTCGCTTGCCTGATCTCATCCCTAGCCGTTAAGCTTTCTGCATGACTTATCACTTGGGGCATA
ATGCTGGATACCTACCATTTTCTTAGACCCCATCAAAATCCTATTTGAGTGTACGGTTCGGAGAAC
CTCATTTATCCGGTAAATGTCTTTTACTCTGCTCTCAGGGAGCTGAGGCAGGACATCCTGAGATAC
ATTGGGAGAGGAGATACAGTTTCAATAAAATAATAGGTTGGGTGGAGGTACATGCCTATAATGCC
ACCACTCAGGAAATGGTGGCAGCTTCGTGAGTTTGAGGCCAACCCAAGAAACATAGTGAAACCCT
GTCAGTAAATAAGTAAGCAAGTATTTGAGTATCTACTATATGCTAGGGCTGACCTGGACATTAGGG
GTCATCTTCTGAACAAACTAGTGCTTGAGGGAGGTATTTGGGGTTTTTGTTTGTTTAATGGATCTGA
ATGAGTTCCAGAGACTGGCTACACAGCGATATGACTGAGCTTAACACCCCTAAAGCATACAGTCA
GACCAATTAGACAATAAAAGGTATGTATAGCTTACCAAATAAAAAAATTGTATTTTCAAGAGAGT
GTCTGTCTGTGTAGCCCTGGCTGTTCTTGAACTCACTCTGTAGACCAGGCTGGCCTGGAAATCCATC
TGCCTGCCTCTGCCTCTCTGCCTCTCTGCCTCTCTGCCTCTCTCTCTGCCTCTCTCTGCCTCTCTCTGC
CCCTCTCTGCCCCTCTCTGCCCCTCTCTGCCGCCCTCTGCCTTTTGCCCTCTGCCCTCTGTTCTCTGG
CCTCTGCCCTCTGCCCTCTGGCCTCTGGCCTCTGCCTCTGCCTCTTGAGTGCTGGAATCAAAGGTGT
GAGCTCTGTAGGTCTTAAGTTCCAGAAGAAAGTAATGAAGTCACCCAGCAGGGAGGTGCTCAGGG
ACAGCACAGACACACACCCAGGACATAGGCTCCCACTTCCTTGGCTTTCTCTGAGTGGCAAAGGAC
CTTAGGCAGTGTCACTCCCTAAGAGAAGGGGATAAAGAGAGGGGCTGAGGTATTCATCATGTGCT
CCGTGGATCTCAAGCCCTCAAGGTAAATGGGGACCCACCTGTCCTACCAGCTGGCTGACCTGTAGC
TTTCCCCACCACAGAATCCAAGTCGGAACTCTTGGCACCTAGAGGATCTCGAGGTCCTTCCTCTGC
AGAGGTCTTGCTTCTCCCGGTCAGCTGACTCCCTCCCCAAGTCCTTCAAATATCTCAGAACATGGG
GAGAAACGGGGACCTTGTCCCTCCTAAGGAACCCCAGTGCTGCATGCCATCATCCCCCCCACCCTC
GCCCCCACCCCCGCCACTTCTCCCTCCATGCATACCACTAGCTGTCATTTTGTACTCTGTATTTATTC
CAGGGCTGCTTCTGATTATTTAGTTTGTTCTTTCCCTGGAGACCTGTTAGAACATAAGGGCGTATGG
TGGGTAGGGGAGGCAGGATATCAGTCCCTGGGGCGAGTTCCTCCCTGCCAACCAAGCCAGATGCC
TGAAAGAGATATGGATGAGGGAAGTTGGACTGTGCCTGTACCTGGTACAGTCATACTCTGTTGAA
AGAATCATCGGGGAGGGGGGGGGGCTCAAGAGGGGAGAGCTCTGCTGAGCCTTTGTGGACCATCC
AATGAGGATGAGGGCTTAGATTCTACCAGGTCATTCTCAGCCACCACACACAAGCGCTCTGCCATC
ACTGAAGAAGCCCCCTAGGGCTCTTGGGCCAGGGCACACTCAGTAAAGATGCAGGTTCAGTCAGG
GAATGATGGGGAAAGGGGTAGGAGGTGGGGGAGGGATCACCCCCTCCTCTAAAACACGAGCCTG
CTGTCTCCAAAGGCCTCTGCCTGTAGTGAGGGTGGCAGAAGAAGACAAGGAGCCAGAACTCTGAC
TCCAGGATCTAAGTCCGTGCAGGAAGGGGATCCTAGAACCATCTGGTTGGACCCAGCTTACCAAG
GGAGAGCCTTTATTCTTCTTTCCCTTGCCCCTCTGTGCCAGCCCCTCTTGCTGTCCCTGATCCCCCAG
ACAGCGAGAGTCTTGCAACCTGCCTCTTCCAAGACCTCCTAATCTCAGGGGCAGGCGGTGGAGTG
AGATCCGGCGTGCACACTTTTTGGAAGATAGCTTTCCCAAGGATCCTCTCCCCCACTGGCAGCTCT
GCCTGTCCCATCACCATGTATAATACCACCACTGCTACAGCATCTCACCGAGGAAAGAAAACTGCA
CAATAAAACCAAGCCTCTGGAGTGTGTCCTGGTGTCTGTCTCTTCTGTGTCCTGGCGTCTGTCTCTT
CTGTGTTCTTCCAAGGTCAGAAACAAAAACCACACACTTCAACCTGGATGGCTCGGCTGAGCACTT
CTGTGTGCAGAAGGTCCAACCAGACTCTGGGGTACCCCGGCCCTCCCTATTCCCTTGCCTCCTGTCT
CCCGCTTTTTATAGCTCCCTATGCTGGGCTTCTCTGGAGAGTGAAATCTTTGCCCAAATCAATGCGC
ATTCTCTCTGCTGAGTCATCTGGCGACAGCAGTTGAGTTCACCCGCCAACACATGGGCCCAGCTAT
GTAGCCGAACCCTGGCTCTGGAAGTGCCAGGGACTTTGTGCATAAGTATGTACCATGCCCTTTTTT
CACAGTCCTAGCTCTGCAGAAGTGCAGCCTGAAGGCCTGTCTGCTGAGAGGACATGCCCTGGAGC
CCTGAAACAGGCACAGTGGGAGGAGGAACGGAGGATGACAGGCATCAGGCCCTCAGTCCAAAAG
CAACCACTTGAGAATGGGCTGGAGTACGAAACATGGGGTCCCGTCCCTGGATCCCTCCTCAAAGA
GTAATAAGTAAAATATAAACAGGTACCCCAGGCCGTTCTGGGTTTGGGTTGTAATGGGATCCATTT
GCAGAGAACTATTGAGACAGCCCAGCCGTACTGTGACAGGCAATGTGGGGGAGGAGGTTGAATCA
CTTGGTATTTAGCATGAATAGAATAATTCCCTGAACATTTTTCTTAAACATCCATATCTAAATTACC
ACCACTCGCTCCCAGTCTTCCTGCCTTTGCGCCAGCCTCCTGTCTGGCCATGCCTGAAGAAGGCTG
GAGAAGCCACCCACCTCAGGCCATGACACTGCCAGCCACTTGGCAGGTGCAGCCAAACCTGAGCT
GTCCCAGAAAGGGACATTCTCAAGACCCAGGCACCCTGATCAGCACTGACTTGGAGCTACAAGTG
TCATGCCAGAAAAGTCTCTAAGAAAACCTTTTCAGGGAAAAGGGGGTGACTCAACACCGGGCAAG
TTTGGGAAGCCCCACCCTTCGAGTGATGGAAGAGCAGATAGGAAGCCTCAGAAGAGAGACACCGG
CACCCAGGTAACGTTCCTCATGTGGTCTCTGTCACACTAGGTGCTCTTCCCTGGACATCTCCGTGAC
CACACTCTCAGTTCTTAGGGAGATGCGGGTGCTCTCTGAGGCTATCTCAGAGTTGCAGATTCTGAG
GCCTAGAGTGACTACAGTCAGCCTAGGAAGCCACAGAGGACTGTGGACCAGGAGGGCAGAAGAG
GAGAAGGGAAGAAAAACCATCAGATAGGACTTGCAATGAAACTAACCCAAGACAATCATAATGC
AGACAGGAATGTTAAAGGCGTTCAGCAGC

pLVX-rtTA-hOSK-all-in-one (human) (SEQ ID NO: 123) as depicted in FIG. 1A:

TGGAAGGGCTAATTCACTCCCAAAGAAGACAAGATATCCTTGATCTGTGGATCTACC
ACACACAAGGCTACTTCCCTGATTAGCAGAACTACACACCAGGGCCAGGGGTCAGATATCCACTG
ACCTTTGGATGGTGCTACAAGCTAGTACCAGTTGAGCCAGATAAGGTAGAAGAGGCCAATAAAGG
AGAGAACACCAGCTTGTTACACCCTGTGAGCCTGCATGGGATGGATGACCCGGAGAGAGAAGTGT
TAGAGTGGAGGTTTGACAGCCGCCTAGCATTTCATCACGTGGCCCGAGAGCTGCATCCGGAGTACT
TCAAGAACTGCTGATATCGAGCTTGCTACAAGGGACTTTCCGCTGGGGACTTTCCAGGGAGGCGTG
GCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTG
CTTAAGCCTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCCGAACAG
GGACTTGAAAGCGAAAGGGAAACCAGAGGAGCTCTCTCGACGCAGGACTCGGCTTGCTGAAGCGC
GCACGGCAAGAGGCGAGGGGCGGCGACTGGTGAGTACGCCAAAAATTTTGACTAGCGGAGGCTA
GAAGGAGAGAGATGGGTGCGAGAGCGTCAGTATTAAGCGGGGGAGAATTAGATCGCGATGGGAA
AAAATTCGGTTAAGGCCAGGGGGAAAGAAAAAATATAAATTAAAACATATAGTATGGGCAAGCA
GGGAGCTAGAACGATTCGCAGTTAATCCTGGCCTGTTAGAAACATCAGAAGGCTGTAGACAAATA
CTGGGACAGCTACAACCATCCCTTCAGACAGGATCAGAAGAACTTAGATCATTATATAATACAGT
AGCAACCCTCTATTGTGTGCATCAAAGGATAGAGATAAAAGACACCAAGGAAGCTTTAGACAAGA
TAGAGGAAGAGCAAAACAAAAGTAAGACCACCGCACAGCAAGCGGCCGGCCGCTGATCTTCAGA
CCTGGAGGAGGAGATATGAGGGACAATTGGAGAAGTGAATTATATAAATATAAAGTAGTAAAAAT
TGAACCATTAGGAGTAGCACCCACCAAGGCAAAGAGAAGAGTGGTGCAGAGAGAAAAAAGAGCA
GTGGGAATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACAATTTGC
TGAGGGCTATTGAGGCGCAACAGCATCTGTTGCAACTCACAGTCTGGGGCATCAAGCAGCTCCAG
GCAAGAATCCTGGCTGTGGAAAGATACCTAAAGGATCAACAGCTCCTGGGGATTTGGGGTTGCTC
TGGAAAACTCATTTGCACCACTGCTGTGCCTTGGAATGCTAGTTGGAGTAATAAATCTCTGGAACA
GATTTGGAATCACACGACCTGGATGGAGTGGGACAGAGAAATTAACAATTACACAAGCTTAATAC
ACTCCTTAATTGAAGAATCGCAAAACCAGCAAGAAAAGAATGAACAAGAATTATTGGAATTAGAT
AAATGGGCAAGTTTGTGGAATTGGTTTAACATAACAAATTGGCTGTGGTATATAAAATTATTCATA
ATGATAGTAGGAGGCTTGGTAGGTTTAAGAATAGTTTTTGCTGTACTTTCTATAGTGAATAGAGTT
AGGCAGGGATATTCACCATTATCGTTTCAGACCCACCTCCCAACCCCGAGGGGACCCGACAGGCC
CGAAGGAATAGAAGAAGAAGGTGGAGAGAGAGACAGAGACAGATCCATTCGATTAGTGAACGGA
TCTCGACGGTATCGCCTTTAAAAGAAAAGGGGGGATTGGGGGGTACAGTGCAGGGGAAAGAATA
GTAGACATAATAGCAACAGACATACAAACTAAAGAATTACAAAAACAAATTACAAAAATTCAAA
ATTTTCGGGTTTATTACAGGGACAGCAGAGATCCAGTTTATCGATTAGTTACCCGGGAGCATGTCA
AGGTCAAAATCGTCAAGAGCGTCAGCAGGCAGCATATCAAGGTCAAAGTCGTCAAGGGCATCGGC
TGGGAGCATGTCTAAGTCAAAATCGTCAAGGGCGTCGGCCGGCCCGCCGCTTTCGCACTTTAGCTG
TTTCTCCAGGCCACATATGATTAGTTCCAGGCCGAAAAGGAAGGCAGGTTCGGCTCCCTGCCGGTC
GAACAGCTCAATTGCTTGTCTCAGAAGTGGGGGCATAGAATCGGTGGTAGGTGTCTCTCTTTCCTC
TTTTGCTACTTGATGCTCCTGTTCCTCCAATACGCAGCCCAGTGTAAAGTGGCCCACGGCGGACAG
AGCGTACAGTGCGTTCTCCAGGGAGAAGCCTTGCTGACACAGGAACGCGAGCTGATTTTCCAGGG
TTTCGTACTGTTTCTCTGTTGGGCGGGTGCCGAGATGCACTTTAGCCCCGTCGCGATGTGAGAGGA
GAGCACAGCGGAATGACTTGGCGTTGTTCCGCAGAAAGTCTTGCCATGACTCGCCTTCCAGGGGGC
AGAAGTGGGTATGATGCCTGTCCAGCATCTCGATTGGCAGGGCATCGAGCAGGGCCCGCTTGTTCT
TCACGTGCCAGTACAGGGTAGGCTGCTCAACTCCCAGCTTTTGAGCGAGTTTCCTTGTCGTCAGGC
CTTCGATACCGACTCCATTGAGTAATTCCAGAGCGCCGTTTATGACTTTGCTCTTGTCCAGTCTAGA
CATGGTGGATCCGTCTAACAAAAAAGCCAAAAACGGCCAGAATTTAGCGGACAATTTACTAGTCT
AACACTGAAAATTACATATTGACCCAAATGATTACATTTCAAAAGGTGCCTAAAAAACTTCACAA
AACACACTCGCCAACCCCGAGCGCATAGTTCAAAACCGGAGCTTCAGCTACTTAAGAAGATAGGT
ACATAAAACCGACCAAAGAAACTGACGCCTCACTTATCCCTCCCCTCACCAGAGGTCCGGCGCCTG
TCGATTCAGGAGAGCCTACCCTAGGCCCGAACCCTGCGTCCTGCGACGGAGAAAAGCCTACCGCA
CACCTACCGGCAGGTGGCCCCACCCTGCATTATAAGCCAACAGAACGGGTGACGTCACGACACGA
CGAGGGCGCGCGCTCCCAAAGGTACGGGTGCACTGCCCAACGGCACCGCCATAACTGCCGCCCCC
GCAACAGACGACAAACCGAGTTCTCCAGTCAGTGACAAACTTCACGTCAGGGTCCCCAGATGGTG
CCCCAGCCCATCTCACCCGAATAAGAGCTTTCCCGCATTAGCGAAGGCCTCAAGACCTTGGGTTCT
TGCCGCCCACCATGCCCCCCACCTTGTTTCAACGACCTCACAGCCCGCCTCACAAGCGTCTTCCATT
CAAGACTCGGGAACAGCCGCCATTTTGCTGCGCTCCCCCCAACCCCCAGTTCAGGGCAACCTTGCT
CGCGGACCCAGACTACAGCCCTTGGCGGTCTCTCCACACGCTTCCGTCCCACCGAGCGGCCCGGCG
GCCACGAAAGCCCCGGCCAGCCCAGCAGCCCGCTACTCACCAAGTGACGATCACAGCGATCCACA
AACAAGAACCGCGACCCAAATCCCGGCTGCGACGGAACTAGCTGTGCCACACCCGGCGCGTCCTT
ATATAATCATCGGCGTTCACCGCCCCACGGAGATCCCTCCGCAGAATCGCCGAGAAGGGACTACTT
TTCCTCGCCTGTTCCGCTCTCTGGAAAGAAAACCAGTGCCCTAGAGTCACCCAAGTCCCGTCCTAA
AATGTCCTTCTGCTGATACTGGGGTTCTAAGGCCGAGTCTTATGAGCAGCGGGCCGCTGTCCTGAG
CGTCCGGGCGGAAGGATCAGGACGCTCGCTGCGCCCTTCGTCTGACGTGGCAGCGCTCGCCGTGA
GGAGGGGGGCGCCCGCGGGAGGCGCCAAAACCCGGCGCGGAGGCCAGATCGGCGCATCGATGAG
GCCCTTTCGTCTTCACTCGAGTTTACTCCCTATCAGTGATAGAGAACGTATGTCGAGTTTACTCCCT
ATCAGTGATAGAGAACGATGTCGAGTTTACTCCCTATCAGTGATAGAGAACGTATGTCGAGTTTAC
TCCCTATCAGTGATAGAGAACGTATGTCGAGTTTACTCCCTATCAGTGATAGAGAACGTATGTCGA
GTTTATCCCTATCAGTGATAGAGAACGTATGTCGAGTTTACTCCCTATCAGTGATAGAGAACGTAT
GTCGAGGTAGGCGTGTACGGTGGGAGGCCTATATAAGCAGAGCTCGTTTAGTGAACCGTCAGATC
GCCTGGAGAAGGATCCGCGGCCGCGCCGCCACCATGGCGGGACACCTGGCTTCGGATTTCGCCTTC
TCGCCCCCTCCAGGTGGTGGAGGTGATGGGCCAGGGGGGCCGGAGCCGGGCTGGGTTGATCCTCG
GACCTGGCTAAGCTTCCAAGGCCCTCCTGGAGGGCCAGGAATCGGGCCGGGGGTTGGGCCAGGCT
CTGAGGTGTGGGGGATTCCCCCATGCCCCCCGCCGTATGAGTTCTGTGGGGGGATGGCGTACTGTG
GGCCCCAGGTTGGAGTGGGGCTAGTGCCCCAAGGCGGCTTGGAGACCTCTCAGCCTGAGGGCGAA
GCAGGAGTCGGGGTGGAGAGCAACTCCGATGGGGCCTCCCCGGAGCCCTGCACCGTCACCCCTGG
TGCCGTGAAGCTGGAGAAGGAGAAGCTGGAGCAAAACCCGGAGGAGTCCCAGGACATCAAAGCT
CTGCAGAAAGAACTCGAGCAATTTGCCAAGCTCCTGAAGCAGAAGAGGATCACCCTGGGATATAC
ACAGGCCGATGTGGGGCTCACCCTGGGGGTTCTATTTGGGAAGGTATTCAGCCAAACGACCATCTG
CCGCTTTGAGGCTCTGCAGCTTAGCTTCAAGAACATGTGTAAGCTGCGGCCCTTGCTGCAGAAGTG
GGTGGAGGAAGCTGACAACAATGAAAATCTTCAGGAGATATGCAAAGCAGAAACCCTCGTGCAG
GCCCGAAAGAGAAAGCGAACCAGTATCGAGAACCGAGTGAGAGGCAACCTGGAGAATTTGTTCCT
GCAGTGCCCGAAACCCACACTGCAGCAGATCAGCCACATCGCCCAGCAGCTTGGGCTCGAGAAGG
ATGTGGTCCGAGTGTGGTTCTGTAACCGGCGCCAGAAGGGCAAGCGATCAAGCAGCGACTATGCA
CAACGAGAGGATTTTGAGGCTGCTGGGTCTCCTTTCTCAGGGGGACCAGTGTCCTTTCCTCTGGCC
CCAGGGCCCCATTTTGGTACCCCAGGCTATGGGAGCCCTCACTTCACTGCACTGTACTCCTCGGTC
CCTTTCCCTGAGGGGGAAGCCTTTCCCCCTGTCTCTGTCACCACTCTGGGCTCTCCCATGCATTCAA
ACGCTAGCGGCAGCGGCGCCACGAACTTCTCTCTGTTAAAGCAAGCAGGAGATGTTGAAGAAAAC
CCCGGGCCTGCATGCATGTACAACATGATGGAGACGGAGCTGAAGCCGCCGGGCCCGCAGCAAAC
TTCGGGGGGCGGCGGCGGCAACTCCACCGCGGCGGCGGCCGGCGGCAACCAGAAAAACAGCCCG
GACCGCGTCAAGCGGCCCATGAATGCCTTCATGGTGTGGTCCCGCGGGCAGCGGCGCAAGATGGC
CCAGGAGAACCCCAAGATGCACAACTCGGAGATCAGCAAGCGCCTGGGCGCCGAGTGGAAACTTT
TGTCGGAGACGGAGAAGCGGCCGTTCATCGACGAGGCTAAGCGGCTGCGAGCGCTGCACATGAAG
GAGCACCCGGATTATAAATACCGGCCCCGGCGGAAAACCAAGACGCTCATGAAGAAGGATAAGT
ACACGCTGCCCGGCGGGCTGCTGGCCCCCGGCGGCAATAGCATGGCGAGCGGGGTCGGGGTGGGC
GCCGGCCTGGGCGCGGGCGTGAACCAGCGCATGGACAGTTACGCGCACATGAACGGCTGGAGCAA
CGGCAGCTACAGCATGATGCAGGACCAGCTGGGCTACCCGCAGCACCCGGGCCTCAATGCGCACG
GCGCAGCGCAGATGCAGCCCATGCACCGCTACGACGTGAGCGCCCTGCAGTACAACTCCATGACC
AGCTCGCAGACCTACATGAACGGCTCGCCCACCTACAGCATGTCCTACTCGCAGCAGGGCACCCCT
GGCATGGCTCTTGGCTCCATGGGTTCGGTGGTCAAGTCCGAGGCCAGCTCCAGCCCCCCTGTGGTT
ACCTCTTCCTCCCACTCCAGGGCGCCCTGCCAGGCCGGGGACCTCCGGGACATGATCAGCATGTAT
CTCCCCGGCGCCGAGGTGCCGGAACCCGCCGCCCCCAGCAGACTTCACATGTCCCAGCACTACCA
GAGCGGCCCGGTGCCCGGCACGGCCATTAACGGCACACTGCCCCTCTCACACATGGCATGCGGCT
CCGGCGAGGGCAGGGGAAGTCTTCTAACATGCGGGGACGTGGAGGAAAATCCCGGCCCACTCGAG
ATGGCTGTCAGCGACGCGCTGCTCCCATCTTTCTCCACGTTCGCGTCTGGCCCGGCGGGAAGGGAG
AAGACACTGCGTCAAGCAGGTGCCCCGAATAACCGCTGGCGGGAGGAGCTCTCCCACATGAAGCG
ACTTCCCCCAGTGCTTCCCGGCCGCCCCTATGACCTGGCGGCGGCGACCGTGGCCACAGACCTGGA
GAGCGGCGGAGCCGGTGCGGCTTGCGGCGGTAGCAACCTGGCGCCCCTACCTCGGAGAGAGACCG
AGGAGTTCAACGATCTCCTGGACCTGGACTTTATTCTCTCCAATTCGCTGACCCATCCTCCGGAGTC
AGTGGCCGCCACCGTGTCCTCGTCAGCGTCAGCCTCCTCTTCGTCGTCGCCGTCGAGCAGCGGCCC
TGCCAGCGCGCCCTCCACCTGCAGCTTCACCTATCCGATCCGGGCCGGGAACGACCCGGGCGTGGC
GCCGGGCGGCACGGGCGGAGGCCTCCTCTATGGCAGGGAGTCCGCTCCCCCTCCGACGGCTCCCTT
CAACCTGGCGGACATCAACGACGTGAGCCCCTCGGGCGGCTTCGTGGCCGAGCTCCTGCGGCCAG
AATTGGACCCGGTGTACATTCCGCCGCAGCAGCCGCAGCCGCCAGGTGGCGGGCTGATGGGCAAG
TTCGTGCTGAAGGCGTCGCTGAGCGCCCCTGGCAGCGAGTACGGCAGCCCGTCGGTCATCAGCGTC
AGCAAAGGCAGCCCTGACGGCAGCCACCCGGTGGTGGTGGCGCCCTACAACGGCGGGCCGCCGCG
CACGTGCCCCAAGATCAAGCAGGAGGCGGTCTCTTCGTGCACCCACTTGGGCGCTGGACCCCCTCT
CAGCAATGGCCACCGGCCGGCTGCACACGACTTCCCCCTGGGGCGGCAGCTCCCCAGCAGGACTA
CCCCGACCCTGGGTCTTGAGGAAGTGCTGAGCAGCAGGGACTGTCACCCTGCCCTGCCGCTTCCTC
CCGGCTTCCATCCCCACCCGGGGCCCAATTACCCATCCTTCCTGCCCGATCAGATGCAGCCGCAAG
TCCCGCCGCTCCATTACCAAGAGCTCATGCCACCCGGTTCCTGCATGCCAGAGGAGCCCAAGCCAA
AGAGGGGAAGACGATCGTGGCCCCGGAAAAGGACCGCCACCCACACTTGTGATTACGCGGGCTGC
GGCAAAACCTACACAAAGAGTTCCCATCTCAAGGCACACCTGCGAACCCACACAGGTGAGAAACC
TTACCACTGTGACTGGGACGGCTGTGGATGGAAATTCGCCCGCTCAGATGAACTGACCAGGCACT
ACCGTAAACACACGGGGCACCGCCCGTTCCAGTGCCAAAAATGCGACCGAGCATTTTCCAGGTCG
GACCACCTCGCCTTACACATGAAGAGGCATTTTTAATGAACGCGTGAATTCTACCGGGTAGGGGA
GGCGCTTTTCCCAAGGCAGTCTGGAGCATGCGCTTTAGCAGCCCCGCTGGGCACTTGGCGCTACAC
AAGTGGCCTCTGGCCTCGCACACATTCCACATCCACCGGTAGGCGCCAACCGGCTCCGTTCTTTGG
TGGCCCCTTCGCGCCACCTTCTACTCCTCCCCTAGTCAGGAAGTTCCCCCCCGCCCCGCAGCTCGCG
TCGTGCAGGACGTGACAAATGGAAGTAGCACGTCTCACTAGTCTCGTGCAGATGGACAGCACCGC
TGAGCAATGGAAGCGGGTAGGCCTTTGGGGCAGCGGCCAATAGCAGCTTTGCTCCTTCGCTTTCTG
GGCTCAGAGGCTGGGAAGGGGTGGGTCCGGGGGCGGGCTCAGGGGGGGGCTCAGGGGCGGGGCG
GGCGCCCGAAGGTCCTCCGGAGGCCCGGCATTCTGCACGCTTCAAAAGCGCACGTCTGCCGCGCT
GTTCTCCTCTTCCTCATCTCCGGGCCTTTCGACCTGCAGCCCAAGCTTACCCGTACGTGTACAGCCA
CCATGATTGAACAAGATGGATTGCACGCAGGTTCTCCGGCCGCTTGGGTGGAGAGGCTATTCGGCT
ATGACTGGGCACAACAGACAATCGGCTGCTCTGATGCCGCCGTGTTCCGGCTGTCAGCGCAGGGG
CGCCCGGTTCTTTTTGTCAAGACCGACCTGTCCGGTGCCCTGAATGAACTGCAAGACGAGGCAGCG
CGGCTATCGTGGCTGGCCACGACGGGCGTTCCTTGCGCAGCTGTGCTCGACGTTGTCACTGAAGCG
GGAAGGGACTGGCTGCTATTGGGCGAAGTGCCGGGGCAGGATCTCCTGTCATCTCACCTTGCTCCT
GCCGAGAAAGTATCCATCATGGCTGATGCAATGCGGCGGCTGCATACGCTTGATCCGGCTACCTGC
CCATTCGACCACCAAGCGAAACATCGCATCGAGCGAGCACGTACTCGGATGGAAGCCGGTCTTGT
CGATCAGGATGATCTGGACGAAGAGCATCAGGGGCTCGCGCCAGCCGAACTGTTCGCCAGGCTCA
AGGCGAGCATGCCCGACGGCGAGGATCTCGTCGTGACCCATGGCGATGCCTGCTTGCCGAATATC
ATGGTGGAAAATGGCCGCTTTTCTGGATTCATCGACTGTGGCCGGCTGGGTGTGGCGGACCGCTAT
CAGGACATAGCGTTGGCTACCCGTGATATTGCTGAAGAGCTTGGCGGCGAATGGGCTGACCGCTTC
CTCGTGCTTTACGGTATCGCCGCTCCCGATTCGCAGCGCATCGCCTTCTATCGCCTTCTTGACGAGT
TCTTCTGATAACCTGCAGGACCTGGTGCATGACCCGCAAGCCCGGTGCCTGACGGGCGCGTCTGGA
ACAATCAACCTCTGGATTACAAAATTTGTGAAAGATTGACTGGTATTCTTAACTATGTTGCTCCTTT
TACGCTATGTGGATACGCTGCTTTAATGCCTTTGTATCATGCTATTGCTTCCCGTATGGCTTTCATTT
TCTCCTCCTTGTATAAATCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCTGCCTT
GCCCGCTGCTGGACAGGGGCTCGGCTGTTGGGCACTGACAATTCCGTGGTGTTGTCGGGGAAGCTG
ACGTCCTTTCCATGGCTGCTCGCCTGTGTTGCCACCTGGATTCTGCGCGGGACGTCCTTCTGCTACG
TCCCTTCGGCCCTCAATCCAGCGGACCTTCCTTCCCGCGGCCTGCTGCCGGCTCTGCGGCCTCTTCC
GCGTCTTCGCCTTCGCCCTCAGACGAGTCGGATCTCCCTTTGGGCCGCCTCCCCGCCTGGAATTAAT
TCTGCAGTCGAGACCTAGAAAAACATGGAGCAATCACAAGTAGCAATACAGCAGCTACCAATGCT
GATTGTGCCTGGCTAGAAGCACAAGAGGAGGAGGAGGTGGGTTTTCCAGTCACACCTCAGGTACC
TTTAAGACCAATGACTTACAAGGCAGCTGTAGATCTTAGCCACTTTTTAAAAGAAAAGAGGGGAC
TGGAAGGGCTAATTCACTCCCAACGAAGACAAGATATCCTTGATCTGTGGATCTACCACACACAA
GGCTACTTCCCTGATTAGCAGAACTACACACCAGGGCCAGGGGTCAGATATCCACTGACCTTTGGA
TGGTGCTACAAGCTAGTACCAGTTGAGCCAGATAAGGTAGAAGAGGCCAATAAAGGAGAGAACA
CCAGCTTGTTACACCCTGTGAGCCTGCATGGGATGGATGACCCGGAGAGAGAAGTGTTAGAGTGG
AGGTTTGACAGCCGCCTAGCATTTCATCACGTGGCCCGAGAGCTGCATCCGGAGTACTTCAAGAAC
TGCTGATATCGAGCTTGCTACAAGGGACTTTCCGCTGGGGACTTTCCAGGGAGGCGTGGCCTGGGC
GGGACTGGGGAGTGGCGAGCCCTCAGATCCTGCATATAAGCAGCTGCTTTTTGCCTGTACTGGGTC
TCTCTGGTTAGACCAGATCTGAGCCTGGGAGCTCTCTGGCTAACTAGGGAACCCACTGCTTAAGCC
TCAATAAAGCTTGCCTTGAGTGCTTCAAGTAGTGTGTGCCCGTCTGTTGTGTGACTCTGGTAACTAG
AGATCCCTCAGACCCTTTTAGTCAGTGTGGAAAATCTCTAGCAGTAGTAGTTCATGTCATCTTATTA
TTCAGTATTTATAACTTGCAAAGAAATGAATATCAGAGAGTGAGAGGCCTTGACATTGCTAGCGTT
TACCGTCGACCTCTAGCTAGAGCTTGGCGTAATCATGGTCATAGCTGTTTCCTGTGTGAAATTGTTA
TCCGCTCACAATTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCTCTTCC
GCTTCCTCGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACCGCT
GCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGG
TGGCCTAACTACGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTATCAAAAAG
GATCTTCACCTAGATCCTTTTAAATTAAAAATGAAGTTTTAAATCAATCTAAAGTATATATGAGTA
AACTTGGTCTGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCAGCAATAAACCA
GCCAGCCGGAAGGGCCGAGCGCAGAAGTGGTCCTGCAACTTTATCCGCCTCCATCCAGTCTATTAA
TTGTTGCCGGGAAGCTAGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACATAGCAG
AACTTTAAAAGTGCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTTCC
CCGAAAAGTGCCACCTGACGTCGACGGATCGGGAGATCAACTTGTTTATTGCAGCTTATAATGGTT
ACAAATAAAGCAATAGCATCACAAATTTCACAAATAAAGCATTTTTTTCACTGCATTCTAGTTGTG
GTTTGTCCAAACTCATCAATGTATCTTATCATGTCTGGATCAACTGGATAACTCAAGCTAACCAAA
ATCATCCCAAACTTCCCACCCCATACCCTATTACCACTGCCAATTACCTGTGGTTTCATTTACTCTA
AACCTGTGATTCCTCTGAATTATTTTCATTTTAAAGAAATTGTATTTGTTAAATATGTACTACAAAC
TTAGTAGT

FIG. 1A: rtTA Advanced in reverse complement (from pLVX-rtTA-hOSK-all-in-one (human)) (SEQ ID NO: 128):

TTAGTTACCCGGGAGCATGTCAAGGTCAAAATCGTCAAGAGCGTCAGCAGGCAGCAT
ATCAAGGTCAAAGTCGTCAAGGGCATCGGCTGGGAGCATGTCTAAGTCAAAATCGTCAAGGGCGT
CGGCCGGCCCGCCGCTTTCGCACTTTAGCTGTTTCTCCAGGCCACATATGATTAGTTCCAGGCCGA
AAAGGAAGGCAGGTTCGGCTCCCTGCCGGTCGAACAGCTCAATTGCTTGTCTCAGAAGTGGGGGC
ATAGAATCGGTGGTAGGTGTCTCTCTTTCCTCTTTTGCTACTTGATGCTCCTGTTCCTCCAATACGC
AGCCCAGTGTAAAGTGGCCCACGGCGGACAGAGCGTACAGTGCGTTCTCCAGGGAGAAGCCTTGC
TGACACAGGAACGCGAGCTGATTTTCCAGGGTTTCGTACTGTTTCTCTGTTGGGCGGGTGCCGAGA
TGCACTTTAGCCCCGTCGCGATGTGAGAGGAGAGCACAGCGGAATGACTTGGCGTTGTTCCGCAG
AAAGTCTTGCCATGACTCGCCTTCCAGGGGGCAGAAGTGGGTATGATGCCTGTCCAGCATCTCGAT
TGGCAGGGCATCGAGCAGGGCCCGCTTGTTCTTCACGTGCCAGTACAGGGTAGGCTGCTCAACTCC
CAGCTTTTGAGCGAGTTTCCTTGTCGTCAGGCCTTCGATACCGACTCCATTGAGTAATTCCAGAGC
GCCGTTTATGACTTTGCTCTTGTCCAGTCTAGACAT

FIG. 1A: Amino acid sequence of rtTA Advanced (SEQ ID NO: 129):

MSRLDKSKVINGALELLNGVGIEGLTTRKLAQKLGVEQPTLYWHVKNKRALLDALPIEM
LDRHHTHFCPLEGESWQDFLRNNAKSFRCALLSHRDGAKVHLGTRPTEKQYETLENQLAFLCQQGFSL
ENALYALSAVGHFTLGCVLEEQEHQVAKEERETPTTDSMPPLLRQAIELFDROGAEPAFLFGLELIICGL
EKQLKCESGGPADALDDFDLDMLPADALDDFDLDMLPADALDDFDLDMLPGN

FIG. 1A: UbC promoter in reverse complement (from pLVX-rtTA-hOSK-all-in-one (human)) (SEQ ID NO: 130):

GTCTAACAAAAAAGCCAAAAACGGCCAGAATTTAGCGGACAATTTACTAGTCTAACA
CTGAAAATTACATATTGACCCAAATGATTACATTTCAAAAGGTGCCTAAAAAACTTCACAAAACAC
ACTCGCCAACCCCGAGCGCATAGTTCAAAACCGGAGCTTCAGCTACTTAAGAAGATAGGTACATA
AAACCGACCAAAGAAACTGACGCCTCACTTATCCCTCCCCTCACCAGAGGTCCGGCGCCTGTCGAT
TCAGGAGAGCCTACCCTAGGCCCGAACCCTGCGTCCTGCGACGGAGAAAAGCCTACCGCACACCT
ACCGGCAGGTGGCCCCACCCTGCATTATAAGCCAACAGAACGGGTGACGTCACGACACGACGAGG
GCGCGCGCTCCCAAAGGTACGGGTGCACTGCCCAACGGCACCGCCATAACTGCCGCCCCCGCAAC
AGACGACAAACCGAGTTCTCCAGTCAGTGACAAACTTCACGTCAGGGTCCCCAGATGGTGCCCCA
GCCCATCTCACCCGAATAAGAGCTTTCCCGCATTAGCGAAGGCCTCAAGACCTTGGGTTCTTGCCG
CCCACCATGCCCCCCACCTTGTTTCAACGACCTCACAGCCCGCCTCACAAGCGTCTTCCATTCAAG
ACTCGGGAACAGCCGCCATTTTGCTGCGCTCCCCCCAACCCCCAGTTCAGGGCAACCTTGCTCGCG
GACCCAGACTACAGCCCTTGGCGGTCTCTCCACACGCTTCCGTCCCACCGAGCGGCCCGGCGGCCA
CGAAAGCCCCGGCCAGCCCAGCAGCCCGCTACTCACCAAGTGACGATCACAGCGATCCACAAACA
AGAACCGCGACCCAAATCCCGGCTGCGACGGAACTAGCTGTGCCACACCCGGCGCGTCCTTATAT
AATCATCGGCGTTCACCGCCCCACGGAGATCCCTCCGCAGAATCGCCGAGAAGGGACTACTTTTCC
TCGCCTGTTCCGCTCTCTGGAAAGAAAACCAGTGCCCTAGAGTCACCCAAGTCCCGTCCTAAAATG
TCCTTCTGCTGATACTGGGGTTCTAAGGCCGAGTCTTATGAGCAGCGGGCCGCTGTCCTGAGCGTC
CGGGCGGAAGGATCAGGACGCTCGCTGCGCCCTTCGTCTGACGTGGCAGCGCTCGCCGTGAGGAG
GGGGGCGCCCGCGGGAGGCGCCAAAACCCGGCGCGGAGGCC

FIG. 1A: human KLF4 (from pLVX-rtTA-hOSK-all-in-one (human) (SEQ ID NO: 131):

ATGGCTGTCAGCGACGCGCTGCTCCCATCTTTCTCCACGTTCGCGTCTGGCCCGGCGG
GAAGGGAGAAGACACTGCGTCAAGCAGGTGCCCCGAATAACCGCTGGCGGGAGGAGCTCTCCCAC
ATGAAGCGACTTCCCCCAGTGCTTCCCGGCCGCCCCTATGACCTGGCGGCGGCGACCGTGGCCACA
GACCTGGAGAGCGGCGGAGCCGGTGCGGCTTGCGGCGGTAGCAACCTGGCGCCCCTACCTCGGAG
AGAGACCGAGGAGTTCAACGATCTCCTGGACCTGGACTTTATTCTCTCCAATTCGCTGACCCATCC
TCCGGAGTCAGTGGCCGCCACCGTGTCCTCGTCAGCGTCAGCCTCCTCTTCGTCGTCGCCGTCGAG
CAGCGGCCCTGCCAGCGCGCCCTCCACCTGCAGCTTCACCTATCCGATCCGGGCCGGGAACGACCC
GGGCGTGGCGCCGGGCGGCACGGGCGGAGGCCTCCTCTATGGCAGGGAGTCCGCTCCCCCTCCGA
CGGCTCCCTTCAACCTGGCGGACATCAACGACGTGAGCCCCTCGGGCGGCTTCGTGGCCGAGCTCC
TGCGGCCAGAATTGGACCCGGTGTACATTCCGCCGCAGCAGCCGCAGCCGCCAGGTGGCGGGCTG
ATGGGCAAGTTCGTGCTGAAGGCGTCGCTGAGCGCCCCTGGCAGCGAGTACGGCAGCCCGTCGGT
CATCAGCGTCAGCAAAGGCAGCCCTGACGGCAGCCACCCGGTGGTGGTGGCGCCCTACAACGGCG
GGCCGCCGCGCACGTGCCCCAAGATCAAGCAGGAGGCGGTCTCTTCGTGCACCCACTTGGGCGCT
GGACCCCCTCTCAGCAATGGCCACCGGCCGGCTGCACACGACTTCCCCCTGGGGCGGCAGCTCCCC
AGCAGGACTACCCCGACCCTGGGTCTTGAGGAAGTGCTGAGCAGCAGGGACTGTCACCCTGCCCT
GCCGCTTCCTCCCGGCTTCCATCCCCACCCGGGGCCCAATTACCCATCCTTCCTGCCCGATCAGATG
CAGCCGCAAGTCCCGCCGCTCCATTACCAAGAGCTCATGCCACCCGGTTCCTGCATGCCAGAGGAG
CCCAAGCCAAAGAGGGGAAGACGATCGTGGCCCCGGAAAAGGACCGCCACCCACACTTGTGATTA
CGCGGGCTGCGGCAAAACCTACACAAAGAGTTCCCATCTCAAGGCACACCTGCGAACCCACACAG
GTGAGAAACCTTACCACTGTGACTGGGACGGCTGTGGATGGAAATTCGCCCGCTCAGATGAACTG
ACCAGGCACTACCGTAAACACACGGGGCACCGCCCGTTCCAGTGCCAAAAATGCGACCGAGCATT
TTCCAGGTCGGACCACCTCGCCTTACACATGAAGAGGCATTTTTAA

FIG. 1A: PGK promoter (from pLVX-rtTA-hOSK-all-in-one (human)) (SEQ ID NO: 132):

GGGTAGGGGAGGCGCTTTTCCCAAGGCAGTCTGGAGCATGCGCTTTAGCAGCCCCGC
TGGGCACTTGGCGCTACACAAGTGGCCTCTGGCCTCGCACACATTCCACATCCACCGGTAGGCGCC
AACCGGCTCCGTTCTTTGGTGGCCCCTTCGCGCCACCTTCTACTCCTCCCCTAGTCAGGAAGTTCCC
CCCCGCCCCGCAGCTCGCGTCGTGCAGGACGTGACAAATGGAAGTAGCACGTCTCACTAGTCTCGT
GCAGATGGACAGCACCGCTGAGCAATGGAAGCGGGTAGGCCTTTGGGGCAGCGGCCAATAGCAG
CTTTGCTCCTTCGCTTTCTGGGCTCAGAGGCTGGGAAGGGGTGGGTCCGGGGGCGGGCTCAGGGGC
GGGCTCAGGGGCGGGGCGGGCGCCCGAAGGTCCTCCGGAGGCCCGGCATTCTGCACGCTTCAAAA
GCGCACGTCTGCCGCGCTGTTCTCCTCTTCCTCATCTCCGGGCCTTTCG

FIG. 1A: Neomycin resistance gene (from pLVX-rtTA-hOSK-all-in-one (human) (SEQ ID NO: 133):

ATGATTGAACAAGATGGATTGCACGCAGGTTCTCCGGCCGCTTGGGTGGAGAGGCTA
TTCGGCTATGACTGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAACTGCAAGACGAG
GCAGCGCGGCTATCGTGGCTGGCCACGACGGGCGTTCCTTGCGCAGCTGTGCTCGACGTTGTCACT
GAAGCGGGAAGGGACTGGCTGCTATTGGGCGAAGTGCCGGGGCAGGATCTCCTGTCATCTCACCT
TGCTCCTGCCGAGAAAGTATCCATCATGGCTGATGCAATGCGGCGGCTGCATACGCTTGATCCGGC
TACCTGCCCATTCGACCACCAAGCGAAACATCGCATCGAGCGAGCACGTACTCGGATGGAAGCCG
GTCTTGTCGATCAGGATGATCTGGACGAAGAGCATCAGGGGCTCGCGCCAGCCGAACTGTTCGCC
AGGCTCAAGGCGAGCATGCCCGACGGCGAGGATCTCGTCGTGACCCATGGCGATGCCTGCTTGCC
GAATATCATGGTGGAAAATGGCCGCTTTTCTGGATTCATCGACTGTGGCCGGCTGGGTGTGGCGGA
CCGCTATCAGGACATAGCGTTGGCTACCCGTGATATTGCTGAAGAGCTTGGCGGCGAATGGGCTG
ACCGCTTCCTCGTGCTTTACGGTATCGCCGCTCCCGATTCGCAGCGCATCGCCTTCTATCGCCTTCT
TGACGAGTTCTTCTGA

FIG. 1A: Amino acid sequence of Neomycin resistance gene (SEQ ID NO: 134):

MIEQDGLHAGSPAAWVERLFGYDWAQQTIGCSDAAVFRLSAQGRPVLFVKTDLSGALNE
LQDEAARLSWLATTGVPCAAVLDVVTEAGRDWLLLGEVPGQDLLSSHLAPAEKVSIMADAMRRLHTL
DPATCPFDHQAKHRIERARTRMEAGLVDQDDLDEEHQGLAPAELFARLKASMPDGEDLVVTHGDACL
PNIMVENGRFSGFIDCGRLGVADRYQDIALATRDIAEELGGEWADRFLVLYGIAAPDSQRIAFYRLLDE
FF

FIG. 1A: WPRE (from pLVX-rtTA-hOSK-all-in-one (human)) (SEQ ID NO: 135):

AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCACCAC
CTGTCAGCTCCTTTCCGGGACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCCGTGGTGTTGTCGGGG
AAGCTGACGTCCTTTCCATGGCTGCTCGCCTGTGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGCGGCC
TCTTCCGCGTCTTCGCCTTCGCCCTCAGACGAGTCGGATCTCCCTTTGGGCCGCCTCCCCGC

FIG. 2A CMV promoter (from pAAV-CMV-tTA) (SEQ ID NO: 136):

ATAGTAATCAATTACGGGGTCATTAGTTCATAGCCCATATATGGAGTTCCGCGTTACA
TAACTTACGGTAAATGGCCCGCCTGGCTGACCGCCCAACGACCCCCGCCCATTGACGTCAATAATG
ACGTATGTTCCCATAGTAACGTCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAAT
GACGGTAAATGGCCCGCCTGGCATTATGCCCAGTACATGACCTTATGGGACTTTCCTACTTGGCAG
TACATCTACGTATTAGTCATCGCTATTACCATGGTGATGCGGTTTTGGCAGTACATCAATGGGCGT
GGATAGCGGTTTGACTCACGGGGATTTCCAAGTCTCCACCCCATTGACGTCAATGGGAGTTTGTTT
TGCACCAAAATCAACGGGACTTTCCAAAATGTCGTAACAACTCCGCCCCATTGACGCAAATGGGC
GGTAGGCGTGTACGGTGGGAGGTCTATATAAGCAGAGCTCGTTTAGTGAACCGTCAGATCGCCTG
GAGACGCCATCCACGCTGTTTTGACCTCCATAGAAGACACCGGGACCGATCCAGCCT

FIG. 2A: tTA Advanced (from pAAV-CMV-tTA) (SEQ ID NO: 137):

ATGTCTAGACTGGACAAGAGCAAAGTCATAAACTCTGCTCTGGAATTACTCAATGAA
GTCGGTATCGAAGGCCTGACGACAAGGAAACTCGCTCAAAAGCTGGGAGTTGAGCAGCCTACCCT
GTACTGGCACGTGAAGAACAAGCGGGCCCTGCTCGATGCCCTGGCAATCGAGATGCTGGACAGGC
ATCATACCCACTTCTGCCCCCTGGAAGGCGAGTCATGGCAAGACTTTCTGCGGAACAACGCCAAGT
CATTCCGCTGTGCTCTCCTCTCACATCGCGACGGGGCTAAAGTGCATCTCGGCACCCGCCCAACAG
AGAAACAGTACGAAACCCTGGAAAATCAGCTCGCGTTCCTGTGTCAGCAAGGCTTCTCCCTGGAG
AACGCACTGTACGCTCTGTCCGCCGTGGGCCACTTTACACTGGGCTGCGTATTGGAGGATCAGGAG
CATCAAGTAGCAAAAGAGGAAAGAGAGACACCTACCACCGATTCTATGCCCCCACTTCTGAGACA
AGCAATTGAGCTGTTCGACCATCAGGGAGCCGAACCTGCCTTCCTTTTCGGCCTGGAACTAATCAT
ATGTGGCCTGGAGAAACAGCTAAAGTGCGAAAGCGGCGGGCCGGCCGACGCCCTTGACGATTTTG
ACTTAGACATGCTCCCAGCCGATGCCCTTGACGACTTTGACCTTGATATGCTGCCTGCTGACGCTCT
TGACGATTTTGACCTTGACATGCTCCCCGGATGA

FIG. 2A Amino acid sequence of tTA Advanced (SEQ ID NO: 138):

MSRLDKSKVINSALELLNEVGIEGLTTRKLAQKLGVEQPTLYWHVKNKRALLDALAIEM
LDRHHTHFCPLEGESWQDFLRNNAKSFRCALLSHRDGAKVHLGTRPTEKQYETLENQLAFLCQQGFSL
ENALYALSAVGHFTLGCVLEDQEHQVAKEERETPTTDSMPPLLRQAIELFDHQGAEPAFLFGLELIICGL
EKQLKCESGGPADALDDFDLDMLPADALDDFDLDMLPADALDDFDLDMLPG

FIG. 2A: hGH pA (from pAAV-CMV-tTA) (SEQ ID NO: 139):

ACGGGTGGCATCCCTGTGACCCCTCCCCAGTGCCTCTCCTGGCCCTGGAAGTTGCCAC
TCCAGTGCCCACCAGCCTTGTCCTAATAAAATTAAGTTGCATCATTTTGTCTGACTAGGTGTCCTTC
TATAATATTATGGGGTGGAGGGGGGTGGTATGGAGCAAGGGGCAAGTTGGGAAGACAACCTGTAG
GGCCTGCGGGGTCTATTGGGAACCAAGCTGGAGTGCAGTGGCACAATCTTGGCTCACTGCAATCTC
CGCCTCCTGGGTTCAAGCGATTCTCCTGCCTCAGCCTCCCGAGTTGTTGGGATTCCAGGCATGCAT
GACCAGGCTCAGCTAATTTTTGTTTTTTTGGTAGAGACGGGGTTTCACCATATTGGCCAGGCTGGTC
TCCAACTCCTAATCTCAGGTGATCTACCCACCTTGGCCTCCCAAATTGCTGGGATTACAGGCGTGA
ACCACTGCTCCCTTCCCTGTCCTT

FIG. 2A: pAAV-TRE3G-EGFP (SEQ ID NO: 140):

TTATGCAGTGCTGCCATAACCATGAGTGATAACACTGCGGCCAACTTACTTCTGACAA
CGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTA
GTAATGGTAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAGATTTAATTAAGGCCTTAATTAGG
CTGCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCTT
GTAGTTAATGATTAACCCGCCATGCTACTTATCTACGTAGCCATGCTCTAGGAAGATCGGAATTCT
TTACTCCCTATCAGTGATAGAGAACGTATGAAGAGTTTACTCCCTATCAGTGATAGAGAACGTATG
CAGACTTTACTCCCTATCAGTGATAGAGAACGTATAAGGAGTTTACTCCCTATCAGTGATAGAGAA
CGTATGACCAGTTTACTCCCTATCAGTGATAGAGAACGTATCTACAGTTTACTCCCTATCAGTGAT
AGAGAACGTATATCCAGTTTACTCCCTATCAGTGATAGAGAACGTATAAGCTTTAGGCGTGTACGG
TGGGCGCCTATAAAAGCAGAGCTCGTTTAGTGAACCGTCAGATCGCCTGGAGCAATTCCACAACA
CTTTTGTCTTATACCAACTTTCCGTACCACTTCCTACCCTCGTAAAGCGGCCGCTCGCCACCATGGT
GAGCAAGGGCGAGGAGCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCCTGACCTAC
GGCGTGCAGTGCTTCAGCCGCTACCCCGACCACATGAAGCAGCACGACTTCTTCAAGTCCGCCATG
CCCGAAGGCTACGTCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAGG
AGGACGGCAACATCCTGGGGCACAAGCTGGAGTACAACTACAACAGCCACAACGTCTATATCATG
GCCGACAAGCAGAAGAACGGCATCAAGGTGAACTTCAAGATCCGCCACAACATCGAGGACGGCA
GCGTGCAGCTCGCCGACCACTACCAGCAGAACACCCCCATCGGCGACGGCCCCGTGCTGCTGCCC
GACAACCACTACCTGAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGTAAA
GCGGACTAGTGCGCGCAGCGGCCGACCATGGCCCAACTTGTTTATTGCAGCTTATAATGGTTACAA
ATAAAGCAATAGCATCACAAATTTCACAAATAAAGCATTTTTTTCACTGCATTCTAGTTGTGGTTTG
TCCAAACTCATCAATGTATCTTATCATGTCTGGATCTCGGTACCGGATCCAAATTCCCGATAAGGA
TCTTCCTAGAGCATGGCTACGTAGATAAGTAGCATGGCGGGTTAATCATTAACTACAAGGAACCCC
TAGTGATGGAGTTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCAGCCTTAATTAA
CCTAATTCACTGGCCGTCGTTTTACAACGTCGTGACTGGGAAAACCCTGGCGTTACCCAACTTAAT
CGCCTTGCAGCACATCCCCCTTTCGCCAGCTGGCGTAATAGCGAAGAGGCCCGCACCGATCGCCCT
TCCCAACAGTTGCGCAGCCTGAATGGCGAATGGGACGCGCCCTGTAGCGGCGCATTAAGCGCGGC
GGGTGTGGTGGTTACGCGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAG
TGGACTCTTGTTCCAAACTGGAACAACACTCAACCCTATCTCGGTCTATTCTTTTGATTTATAAGGG
ATTTTGCCGATTTCGGCCTATTGGTTAAAAAATGAGCTGATTTAACAAAAATTTAACGCGAATTTT
AACAAAATATTAACGTTTATAATTTCAGGTGGCATCTTTCGGGGAAATGTGCGCGGAACCCCTATT
TGTTTATTTTTCTAAATACATTCAAATATGTATCCGCTCATGAGACAATAACCCTGATAAATGCTTC
AATAATATTGAAAAAGGAAGAGTATGAGTATTCAACATTTCCGTGTCGCCCTTATTCCCTTTTTTGC
GGCATTTTGCCTTCCTGTTTTTGCTCACCCAGAAACGCTGGTGAAAGTAAAAGATGCTGAAGATCA
GTTGGGTGCACGAGTGGGTTACATCGAACTGGATCTCAATAGTGGTAAGATCCTTGAGAGTTTTCG
CCCCGAAGAACGTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATGACTTGGTTGAGTA
CTCACCAGTCACAGAAAAGCATCTTACGGATGGCATGACAGTAAGAGAA

FIG. 2A: EGPP (from pAAV-TRE3G-EGFP) (SEQ ID NO: 141):

ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCACGACTTCTT
CAAGTCCGCCATGCCCGAAGGCTACGTCCAGGAGCGCACCATCTTCTTCAAGGACGACGGCAACT
ACAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACAA
CGTCTATATCATGGCCGACAAGCAGAAGAACGGCATCAAGGTGAACTTCAAGATCCGCCACAACA
TCGAGGACGGCAGCGTGCAGCTCGCCGACCACTACCAGCAGAACACCCCCATCGGCGACGGCCCC
GTGCTGCTGCCCGACAACCACTACCTGAGCACCCAGTCCGCCCTGAGCAAAGACCCCAACGAGAA
GCGCGATCACATGGTCCTGCTGGAGTTCGTGACCGCCGCCGGGATCACTCTCGGCATGGACGAGCT
GTACAAGTAA

FIG. 2A Amino acid sequence of EGPP (SEQ ID NO: 142):

MVSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDTLVNRIELKG
IDFKEDGNILGHKLEYNYNSHNVYIMADKQKNGIKVNFKIRHNIEDGSVQLADHYQQNTPIGDGPVLLP
DNHYLSTQSALSKDPNEKRDHMVLLEFVTAAGITLGMDELYK

FIG. 2A: SV40 pA (from pAAV-TRE3G-BGFP) (SEQ ID NO: 143):

CTAGTGCGCGCAGCGGCCGACCATGGCCCAACTTGTTTATTGCAGCTTATAATGGTTA
CAAATAAAGCAATAGCATCACAAATTTCACAAATAAAGCATTTTTTTCACTGCATTCTAGTTGTGG
TTTGTCCAAACTCATCAATGTATCTTATCATGTCTGGATCTCGGTACCGGATC

P2A (from pAAV-TRE3G-OSK (mouse) (SEQ ID NO: 144):

GGCAGCGGCGCCACGAACTTCTCTCTGTTAAAGCAAGCAGGAGATGTT
GAAGAAAACCCCGGGCCTG

Mouse Klf4 (from pAAV-TRE3G-OSK (mouse)) (SEQ ID NO: 145):

ATGAGGCAGCCACCTGGCGAGTCTGACATGGCTGTCAGCGACGCTCTGCTCCCGTCCT
TCTCCACGTTCGCGTCCGGCCCGGCGGGAAGGGAGAAGACACTGCGTCCAGCAGGTGCCCCGACT
AACCGTTGGCGTGAGGAACTCTCTCACATGAAGCGACTTCCCCCACTTCCCGGCCGCCCCTACGAC
CTGGCGGCGACGGTGGCCACAGACCTGGAGAGTGGCGGAGCTGGTGCAGCTTGCAGCAGTAACAA
CCCGGCCCTCCTAGCCCGGAGGGAGACCGAGGAGTTCAACGACCTCCTGGACCTAGACTTTATCCT
TTCCAACTCGCTAACCCACCAGGAATCGGTGGCCGCCACCGTGACCACCTCGGCGTCAGCTTCATC
CTCGTCTTCCCCAGCGAGCAGCGGCCCTGCCAGCGCGCCCTCCACCTGCAGCTTCAGCTATCCGAT
CCGGGCCGGGGGTGACCCGGGCGTGGCTGCCAGCAACACAGGTGGAGGGCTCCTCTACAGCCGAG
AATCTGCGCCACCTCCCACGGCCCCCTTCAACCTGGCGGACATCAATGACGTGAGCCCCTCGGGCG
GCTTCGTGGCTGAGCTCCTGCGGCCGGAGTTGGACCCAGTATACATTCCGCCACAGCAGCCTCAGC
CGCCAGGTGGCGGGCTGATGGGCAAGTTTGTGCTGAAGGCGTCTCTGACCACCCCTGGCAGCGAG
TACAGCAGCCCTTCGGTCATCAGTGTTAGCAAAGGAAGCCCAGACGGCAGCCACCCCGTGGTAGT
GGCGCCCTACAGCGGTGGCCCGCCGCGCATGTGCCCCAAGATTAAGCAAGAGGCGGTCCCGTCCT
GCACGGTCAGCCGGTCCCTAGAGGCCCATTTGAGCGCTGGACCCCAGCTCAGCAACGGCCACCGG
CCCAACACACACGACTTCCCCCTGGGGCGGCAGCTCCCCACCAGGACTACCCCTACACTGAGTCCC
GAGGAACTGCTGAACAGCAGGGACTGTCACCCTGGCCTGCCTCTTCCCCCAGGATTCCATCCCCAT
CCGGGGCCCAACTACCCTCCTTTCCTGCCAGACCAGATGCAGTCACAAGTCCCCTCTCTCCATTATC
AAGAGCTCATGCCACCGGGTTCCTGCCTGCCAGAGGAGCCCAAGCCAAAGAGGGGAAGAAGGTC
GTGGCCCCGGAAAAGAACAGCCACCCACACTTGTGACTATGCAGGCTGTGGCAAAACCTATACCA
AGAGTTCTCATCTCAAGGCACACCTGCGAACTCACACAGGCGAGAAACCTTACCACTGTGACTGG
GACGGCTGTGGGTGGAAATTCGCCCGCTCCGATGAACTGACCAGGCACTACCGCAAACACACAGG
GCACCGGCCCTTTCAGTGCCAGAAGTGCGACAGGGCCTTTTCCAGGTCGGACCACCTTGCCTTACA
CATGAAGAGGCACTAA

pAAV-CaMKIIα-tTA2 (SEQ ID NO: 124) as depicted in FIG. 3A:

CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTCCATCACTAG
GGGTTCCTGCGGCCGCACGCGTTTAACATTATGGCCTTAGGTCACTTCATCTCCATGGGGTTCTTCT
TCTGATTTTCTAGAAAATGAGATGGGGGTGCAGAGAGCTTCCTCAGTGACCTGCCCAGGGTCACAT
CAGAAATGTCAGAGCTAGAACTTGAACTCAGATTACTAATCTTAAATTCCATGCCTTGGGGGCATG
CAAGTACGATATACAGAAGGAGTGAACTCATTAGGGCAGATGACCAATGAGTTTAGGAAAGAAG
AGTCCAGGGCAGGGTACATCTACACCACCCGCCCAGCCCTGGGTGAGTCCAGCCACGTTCACCTCA
TTATAGTTGCCTCTCTCCAGTCCTACCTTGACGGGAAGCACAAGCAGAAACTGGGACAGGAGCCCC
AGGAGACCAAATCTTCATGGTCCCTCTGGGAGGATGGGTGGGGAGAGCTGTGGCAGAGGCCTCAG
GAGGGGCCCTGCTGCTCAGTGGTGACAGATAGGGGTGAGAAAGCAGACAGAGTCATTCCGTCAGC
ATTCTGGGTCTGTTTGGTACTTCTTCTCACGCTAAGGTGGCGGTGTGATATGCACAATGGCTAAAA
AGCAGGGAGAGCTGGAAAGAAACAAGGACAGAGACAGAGGCCAAGTCAACCAGACCAATTCCCA
GAGGAAGCAAAGAAACCATTACAGAGACTACAAGGGGGAAGGGAAGGAGAGATGAATTAGCTTC
CCCTGTAAACCTTAGAACCCAGCTGTTGCCAGGGCAACGGGGCAATACCTGTCTCTTCAGAGGAG
ATGAAGTTGCCAGGGTAACTACATCCTGTCTTTCTCAAGGACCATCCCAGAATGTGGCACCCACTA
GCCGTTACCATAGCAACTGCCTCTTTGCCCCACTTAATCCCATCCCGTCTGTTAAAAGGGCCCTATA
GTTGGAGGTGGGGGAGGTAGGAAGAGCGATGATCACTTGTGGACTAAGTTTGTTCGCATCCCCTTC
TCCAACCCCCTCAGTACATCACCCTGGGGGAACAGGGTCCACTTGCTCCTGGGCCCACACAGTCCT
GCAGTATTGTGTATATAAGGCCAGGGCAAAGAGGAGCAGGTTTTAAAGTGAAAGGCAGGCAGGTG
TTGGGGAGGCAGTTACCGGGGCAACGGGAACAGGGCGTTTCGGAGGTGGTTGCCATGGGGACCTG
GATGCTGACGAAGGCTCGCGAGGCTGTGAGCAGCCACAGTGCCCTGCTCAGAAGCCCCAAGCTCG
TCAGTCAAGCCGGTTCTCCGTTTGCACTCAGGAGCACGGGCAGGCGAGTGGCCCCTAGTTCTGGGG
GCAGCTCTAGAGCGGTACCGGATCCGCCACCATGTCTAGACTGGACAAGAGCAAAGTCATAAACT
CTGCTCTGGAATTACTCAATGAAGTCGGTATCGAAGGCCTGACGACAAGGAAACTCGCTCAAAAG
CTGGGAGTTGAGCAGCCTACCCTGTACTGGCACGTGAAGAACAAGCGGGCCCTGCTCGATGCCCT
GGCAATCGAGATGCTGGACAGGCATCATACCCACTTCTGCCCCCTGGAAGGCGAGTCATGGCAAG
ACTTTCTGCGGAACAACGCCAAGTCATTCCGCTGTGCTCTCCTCTCACATCGCGACGGGGCTAAAG
TGCATCTCGGCACCCGCCCAACAGAGAAACAGTACGAAACCCTGGAAAATCAGCTCGCGTTCCTG
TGTCAGCAAGGCTTCTCCCTGGAGAACGCACTGTACGCTCTGTCCGCCGTGGGCCACTTTACACTG
GGCTGCGTATTGGAGGATCAGGAGCATCAAGTAGCAAAAGAGGAAAGAGAGACACCTACCACCG
ATTCTATGCCCCCACTTCTGAGACAAGCAATTGAGCTGTTCGACCATCAGGGAGCCGAACCTGCCT
TCCTTTTCGGCCTGGAACTAATCATATGTGGCCTGGAGAAACAGCTAAAGTGCGAAAGCGGCGGG
CCGGCCGACGCCCTTGACGATTTTGACTTAGACATGCTCCCAGCCGATGCCCTTGACGACTTTGAC
CTTGATATGCTGCCTGCTGACGCTCTTGACGATTTTGACCTTGACATGCTCCCCGGATGAGAATTCG
ATATCAAGCTTATCGATAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCC
CGTATGGCTTTCATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACTGGTTGGGGCAT
TGCCACCACCTGTCAGCTCCTTTCCGGAACTTTCGCTTTCCCCCTCCCTATTGCCACGGCGGAACTC
ATCGCCGCCTGCCTTGCCCGCTGCTGGACAGGGGCTCGGCTGTTGGGCACTGACAATTCCGTGGTG
TTGTCGGGGAAATCATCGTCCTTTCCTTGGCTGCTCGCCTATGTTGCCACCTGGATTCTGCGCGGGA
CGTCCTTCTGCTACGTCCCTTCGGCCCTCAATCCAGCGGACCTTCCTTCCCGCGGCCTGCTGCCGGC
TCTGCGGCCTCTTCCGCGTCTTCGCCTTCGCCCTCAGACGAGTCGGATCTCCCTTTGGGCCGCCTCC
CCGCATCGATACCGAGCGCTGCTCGAGAGATCTACGGGTGGCATCCCTGTGACCCCTCCCCAGTGC
CTCTCCTGGCCCTGGAAGTTGCCACTCCAGTGCCCACCAGCCTTGTCCTAATAAAATTAAGTTGCA
TCATTTTGTCTGACTAGGTGTCCTTCTATAATATTATGGGGTGGAGGGGGGTGGTATGGAGCAAGG
GGCAAGTTGGGAAGACAACCTGTAGGGCCTGCGGGGTCTATTGGGAACCAAGCTGGAGTGCAGTG
GCACAATCTTGGCTCACTGCAATCTCCGCCTCCTGGGTTCAAGCGATTCTCCTGCCTCAGCCTCCCG
AGTTGTTGGGATTCCAGGCATGCATGACCAGGCTCAGCTAATTTTTGTTTTTTTGGTAGAGACGGG
GTTTCACCATATTGGCCAGGCTGGTCTCCAACTCCTAATCTCAGGTGATCTACCCACCTTGGCCTCC
CAAATTGCTGGGATTACAGGCGTGAACCACTGCTCCCTTCCCTGTCCTTCTGATTTTGTAGGTAACC
ACGTGCGGACCGAGCGGCCGCAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGCGGCCTCA
GTGAGCGAGCGAGCGCGCAGCTGCCTGCAGGGGCGCCTGATGCGGTATTTTCTCCTTACGCATCTG
TGCGGTATTTCACACCGCATACGTCAAAGCAACCATAGTACGCGCCCTGTAGCGGCGCATTAAGCG
CGGCGGGTGTGGTGGTTACGCGCAGCGTGACCGCTACACTTGCCAGCGCCTTAGCGCCCGCTCCTT
TCGCTTTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTTGGGTGATGG
TTCACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACTCTATCTCGGGCTATTCTTTTGATTTAT
AAGGGATTTTGCCGATTTCGGTCTATTGGTTAAAAAATGAGCTGATTTAACAAAAATTTAACGCGA
ATTTTAACAAAATATTAACGTTTACAATTTTATGGTGCACTCTCAGTACAATCTGCTCTGATGCCGC
ATAGTTAAGCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAATGTCATGAT
AATAATGGTTTCTTAGACGTCAGGTGGCACTTTTCGGGGAAATGTGCGCGGAACCCCTATTTGTTT
ATTTTTCTAAATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGCCATAAC
CATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCC
GGTGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGA
AAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGT

FIG. 3A: CaMKIIα promoter (from pAAV-CaMKIIα-tTA2) (SEQ ID NO: 146):

TGCGGCCGCACGCGTTTAACATTATGGCCTTAGGTCACTTCATCTCCATGGGGTTCTT
CTTCTGATTTTCTAGAAAATGAGATGGGGGTGCAGAGAGCTTCCTCAGTGACCTGCCCAGGGTCAC
ATCAGAAATGTCAGAGCTAGAACTTGAACTCAGATTACTAATCTTAAATTCCATGCCTTGGGGGCA
TGCAAGTACGATATACAGAAGGAGTGAACTCATTAGGGCAGATGACCAATGAGTTTAGGAAAGAA
GAGTCCAGGGCAGGGTACATCTACACCACCCGCCCAGCCCTGGGTGAGTCCAGCCACGTTCACCTC
ATTATAGTTGCCTCTCTCCAGTCCTACCTTGACGGGAAGCACAAGCAGAAACTGGGACAGGAGCC
CCAGGAGACCAAATCTTCATGGTCCCTCTGGGAGGATGGGTGGGGAGAGCTGTGGCAGAGGCCTC
AGGAGGGGCCCTGCTGCTCAGTGGTGACAGATAGGGGTGAGAAAGCAGACAGAGTCATTCCGTCA
GCATTCTGGGTCTGTTTGGTACTTCTTCTCACGCTAAGGTGGCGGTGTGATATGCACAATGGCTAA
AAAGCAGGGAGAGCTGGAAAGAAACAAGGACAGAGACAGAGGCCAAGTCAACCAGACCAATTCC
CAGAGGAAGCAAAGAAACCATTACAGAGACTACAAGGGGGAAGGGAAGGAGAGATGAATTAGCT
TCCCCTGTAAACCTTAGAACCCAGCTGTTGCCAGGGCAACGGGGCAATACCTGTCTCTTCAGAGGA
GATGAAGTTGCCAGGGTAACTACATCCTGTCTTTCTCAAGGACCATCCCAGAATGTGGCACCCACT
AGCCGTTACCATAGCAACTGCCTCTTTGCCCCACTTAATCCCATCCCGTCTGTTAAAAGGGCCCTAT
AGTTGGAGGTGGGGGAGGTAGGAAGAGCGATGATCACTTGTGGACTAAGTTTGTTCGCATCCCCTT
CTCCAACCCCCTCAGTACATCACCCTGGGGGAACAGGGTCCACTTGCTCCTGGGCCCACACAGTCC
TGCAGTATTGTGTATATAAGGCCAGGGCAAAGAGGAGCAGGTTTTAAAGTGAAAGGCAGGCAGGT
GTTGGGGAGGCAGTTACCGGGGCAACGGGAACAGGGCGTTTCGGAGGTGGTTGCCATGGGGACCT
GGATGCTGACGAAGGCTCGCGAGGCTGTGAGCAGCCACAGTGCCCTGCTCAGAAGCCCCAAGCTC
GTCAGTCAAGCCGGTTCTCCGTTTGCACTCAGGAGCACGGGCAGGCGAGTGGCCCCTAGTTCTGGG
GGCAGCTCTAGAGCGGTACC

FIG. 3A: WPRE (from pAAV-CaMKIIα-tTA2) (SEQ ID NO: 147):

AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCACCAC
CTGTCAGCTCCTTTCCGGAACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCCGTGGTGTTGTCGGGG
AAATCATCGTCCTTTCCTTGGCTGCTCGCCTATGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGCGGCC
TCTTCCGCGTCTTCGCCTTCGCCCTCAGACGAGTCGGATCTCCCTTTGGGCCGCCTCCCCGC

FIG. 3A: hGH pA (from pAAV-CaMKIIα-tTA2) (SEQ ID NO: 148):

GGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTA
TAATATTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGGATTCCAGGCATGCATG
ACCAGGCTCAGCTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATTACAGGCGTGAA
CCACTGCTCCCTTCCCTGTCCTT

pAAV-CaMKIIα-rtTA2S-M2 (SEQ ID NO: 125) as a non-limiting example of the vector depicted in FIG. 3B and FIG. 3C:

CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTCCATCACTAG
GGGTTCCTGCGGCCGCACGCGTTTAACATTATGGCCTTAGGTCACTTCATCTCCATGGGGTTCTTCT
TCTGATTTTCTAGAAAATGAGATGGGGGTGCAGAGAGCTTCCTCAGTGACCTGCCCAGGGTCACAT
CAGAAATGTCAGAGCTAGAACTTGAACTCAGATTACTAATCTTAAATTCCATGCCTTGGGGGCATG
CAAGTACGATATACAGAAGGAGTGAACTCATTAGGGCAGATGACCAATGAGTTTAGGAAAGAAG
AGTCCAGGGCAGGGTACATCTACACCACCCGCCCAGCCCTGGGTGAGTCCAGCCACGTTCACCTCA
TTATAGTTGCCTCTCTCCAGTCCTACCTTGACGGGAAGCACAAGCAGAAACTGGGACAGGAGCCCC
AGGAGACCAAATCTTCATGGTCCCTCTGGGAGGATGGGTGGGGAGAGCTGTGGCAGAGGCCTCAG
GAGGGGCCCTGCTGCTCAGTGGTGACAGATAGGGGTGAGAAAGCAGACAGAGTCATTCCGTCAGC
ATTCTGGGTCTGTTTGGTACTTCTTCTCACGCTAAGGTGGCGGTGTGATATGCACAATGGCTAAAA
AGCAGGGAGAGCTGGAAAGAAACAAGGACAGAGACAGAGGCCAAGTCAACCAGACCAATTCCCA
GAGGAAGCAAAGAAACCATTACAGAGACTACAAGGGGGAAGGGAAGGAGAGATGAATTAGCTTC
CCCTGTAAACCTTAGAACCCAGCTGTTGCCAGGGCAACGGGGCAATACCTGTCTCTTCAGAGGAG
ATGAAGTTGCCAGGGTAACTACATCCTGTCTTTCTCAAGGACCATCCCAGAATGTGGCACCCACTA
GCCGTTACCATAGCAACTGCCTCTTTGCCCCACTTAATCCCATCCCGTCTGTTAAAAGGGCCCTATA
GTTGGAGGTGGGGGAGGTAGGAAGAGCGATGATCACTTGTGGACTAAGTTTGTTCGCATCCCCTTC
TCCAACCCCCTCAGTACATCACCCTGGGGGAACAGGGTCCACTTGCTCCTGGGCCCACACAGTCCT
GCAGTATTGTGTATATAAGGCCAGGGCAAAGAGGAGCAGGTTTTAAAGTGAAAGGCAGGCAGGTG
TTGGGGAGGCAGTTACCGGGGCAACGGGAACAGGGCGTTTCGGAGGTGGTTGCCATGGGGACCTG
GATGCTGACGAAGGCTCGCGAGGCTGTGAGCAGCCACAGTGCCCTGCTCAGAAGCCCCAAGCTCG
TCAGTCAAGCCGGTTCTCCGTTTGCACTCAGGAGCACGGGCAGGCGAGTGGCCCCTAGTTCTGGGG
GCAGCTCTAGAGCGGTACCGGATCCGCCACCATGTCTAGACTGGACAAGAGCAAAGTCATAAACG
GCGCTCTGGAATTACTCAATGGAGTCGGTATCGAAGGCCTGACGACAAGGAAACTCGCTCAAAAG
CTGGGAGTTGAGCAGCCTACCCTGTACTGGCACGTGAAGAACAAGCGGGCCCTGCTCGATGCCCT
GCCAATCGAGATGCTGGACAGGCATCATACCCACTTCTGCCCCCTGGAAGGCGAGTCATGGCAAG
ACTTTCTGCGGAACAACGCCAAGTCATTCCGCTGTGCTCTCCTCTCACATCGCGACGGGGCTAAAG
TGCATCTCGGCACCCGCCCAACAGAGAAACAGTACGAAACCCTGGAAAATCAGCTCGCGTTCCTG
TGTCAGCAAGGCTTCTCCCTGGAGAACGCACTGTACGCTCTGTCCGCCGTGGGCCACTTTACACTG
GGCTGCGTATTGGAGGAACAGGAGCATCAAGTAGCAAAAGAGGAAAGAGAGACACCTACCACCG
ATTCTATGCCCCCACTTCTGAGACAAGCAATTGAGCTGTTCGACCGGCAGGGAGCCGAACCTGCCT
TCCTTTTCGGCCTGGAACTAATCATATGTGGCCTGGAGAAACAGCTAAAGTGCGAAAGCGGCGGG
CCGGCCGACGCCCTTGACGATTTTGACTTAGACATGCTCCCAGCCGATGCCCTTGACGACTTTGAC
CTTGATATGCTGCCTGCTGACGCTCTTGACGATTTTGACCTTGACATGCTCCCCGGGTAAGAATTCG
ATATCAAGCTTATCGATAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCC
CGTATGGCTTTCATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACTGGTTGGGGCAT
TGCCACCACCTGTCAGCTCCTTTCCGGAACTTTCGCTTTCCCCCTCCCTATTGCCACGGCGGAACTC
ATCGCCGCCTGCCTTGCCCGCTGCTGGACAGGGGCTCGGCTGTTGGGCACTGACAATTCCGTGGTG
TTGTCGGGGAAATCATCGTCCTTTCCTTGGCTGCTCGCCTATGTTGCCACCTGGATTCTGCGCGGGA
CGTCCTTCTGCTACGTCCCTTCGGCCCTCAATCCAGCGGACCTTCCTTCCCGCGGCCTGCTGCCGGC
TCTGCGGCCTCTTCCGCGTCTTCGCCTTCGCCCTCAGACGAGTCGGATCTCCCTTTGGGCCGCCTCC
CCGCATCGATACCGAGCGCTGCTCGAGAGATCTACGGGTGGCATCCCTGTGACCCCTCCCCAGTGC
CTCTCCTGGCCCTGGAAGTTGCCACTCCAGTGCCCACCAGCCTTGTCCTAATAAAATTAAGTTGCA
TCATTTTGTCTGACTAGGTGTCCTTCTATAATATTATGGGGTGGAGGGGGGTGGTATGGAGCAAGG
GGCAAGTTGGGAAGACAACCTGTAGGGCCTGCGGGGTCTATTGGGAACCAAGCTGGAGTGCAGTG
GCACAATCTTGGCTCACTGCAATCTCCGCCTCCTGGGTTCAAGCGATTCTCCTGCCTCAGCCTCCCG
AGTTGTTGGGATTCCAGGCATGCATGACCAGGCTCAGCTAATTTTTGTTTTTTTGGTAGAGACGGG
GTTTCACCATATTGGCCAGGCTGGTCTCCAACTCCTAATCTCAGGTGATCTACCCACCTTGGCCTCC
CAAATTGCTGGGATTACAGGCGTGAACCACTGCTCCCTTCCCTGTCCTTCTGATTTTGTAGGTAACC
ACGTGCGGACCGAGCGGCCGCAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGCGGCCTCA
GTGAGCGAGCGAGCGCGCAGCTGCCTGCAGGGGCGCCTGATGCGGTATTTTCTCCTTACGCATCTG
TGCGGTATTTCACACCGCATACGTCAAAGCAACCATAGTACGCGCCCTGTAGCGGCGCATTAAGCG
CGGCGGGTGTGGTGGTTACGCGCAGCGTGACCGCTACACTTGCCAGCGCCTTAGCGCCCGCTCCTT
TCGCTTTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTTGGGTGATGG
TTCACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACTCTATCTCGGGCTATTCTTTTGATTTAT
AAGGGATTTTGCCGATTTCGGTCTATTGGTTAAAAAATGAGCTGATTTAACAAAAATTTAACGCGA
ATTTTAACAAAATATTAACGTTTACAATTTTATGGTGCACTCTCAGTACAATCTGCTCTGATGCCGC
ATAGTTAAGCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAATGTCATGAT
AATAATGGTTTCTTAGACGTCAGGTGGCACTTTTCGGGGAAATGTGCGCGGAACCCCTATTTGTTT
ATTTTTCTAAATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGCCATAAC
CATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCC
GGTGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGA
AAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGT

pAAV-CaMKIIα-rtTA3 (SEQ ID NO: 126) as a non-limiting example of the vector depicted in FIG. 3B:

CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTCCATCACTAG
GGGTTCCTGCGGCCGCACGCGTTTAACATTATGGCCTTAGGTCACTTCATCTCCATGGGGTTCTTCT
TCTGATTTTCTAGAAAATGAGATGGGGGTGCAGAGAGCTTCCTCAGTGACCTGCCCAGGGTCACAT
CAGAAATGTCAGAGCTAGAACTTGAACTCAGATTACTAATCTTAAATTCCATGCCTTGGGGGCATG
CAAGTACGATATACAGAAGGAGTGAACTCATTAGGGCAGATGACCAATGAGTTTAGGAAAGAAG
AGTCCAGGGCAGGGTACATCTACACCACCCGCCCAGCCCTGGGTGAGTCCAGCCACGTTCACCTCA
TTATAGTTGCCTCTCTCCAGTCCTACCTTGACGGGAAGCACAAGCAGAAACTGGGACAGGAGCCCC
AGGAGACCAAATCTTCATGGTCCCTCTGGGAGGATGGGTGGGGAGAGCTGTGGCAGAGGCCTCAG
GAGGGGCCCTGCTGCTCAGTGGTGACAGATAGGGGTGAGAAAGCAGACAGAGTCATTCCGTCAGC
ATTCTGGGTCTGTTTGGTACTTCTTCTCACGCTAAGGTGGCGGTGTGATATGCACAATGGCTAAAA
AGCAGGGAGAGCTGGAAAGAAACAAGGACAGAGACAGAGGCCAAGTCAACCAGACCAATTCCCA
GAGGAAGCAAAGAAACCATTACAGAGACTACAAGGGGGAAGGGAAGGAGAGATGAATTAGCTTC
CCCTGTAAACCTTAGAACCCAGCTGTTGCCAGGGCAACGGGGCAATACCTGTCTCTTCAGAGGAG
ATGAAGTTGCCAGGGTAACTACATCCTGTCTTTCTCAAGGACCATCCCAGAATGTGGCACCCACTA
GCCGTTACCATAGCAACTGCCTCTTTGCCCCACTTAATCCCATCCCGTCTGTTAAAAGGGCCCTATA
GTTGGAGGTGGGGGAGGTAGGAAGAGCGATGATCACTTGTGGACTAAGTTTGTTCGCATCCCCTTC
TCCAACCCCCTCAGTACATCACCCTGGGGGAACAGGGTCCACTTGCTCCTGGGCCCACACAGTCCT
GCAGTATTGTGTATATAAGGCCAGGGCAAAGAGGAGCAGGTTTTAAAGTGAAAGGCAGGCAGGTG
TTGGGGAGGCAGTTACCGGGGCAACGGGAACAGGGCGTTTCGGAGGTGGTTGCCATGGGGACCTG
GATGCTGACGAAGGCTCGCGAGGCTGTGAGCAGCCACAGTGCCCTGCTCAGAAGCCCCAAGCTCG
TCAGTCAAGCCGGTTCTCCGTTTGCACTCAGGAGCACGGGCAGGCGAGTGGCCCCTAGTTCTGGGG
GCAGCTCTAGAGCGGTACCGGATCCGCCACCATGTCTAGGCTGGACAAGAGCAAAGTCATAAACG
GAGCTCTGGAATTACTCAATGGTGTCGGTATCGAAGGCCTGACGACAAGGAAACTCGCTCAAAAG
CTGGGAGTTGAGCAGCCTACCCTGTACTGGCACGTGAAGAACAAGCGGGCCCTGCTCGATGCCCT
GCCAATCGAGATGCTGGACAGGCATCATACCCACTTCTGCCCCCTGGAAGGCGAGTCATGGCAAG
ACTTTCTGCGGAACAACGCCAAGTCATACCGCTGTGCTCTCCTCTCACATCGCGACGGGGCTAAAG
TGCATCTCGGCACCCGCCCAACAGAGAAACAGTACGAAACCCTGGAAAATCAGCTCGCGTTCCTG
TGTCAGCAAGGCTTCTCCCTGGAGAACGCACTGTACGCTCTGTCCGCCGTGGGCCACTTTACACTG
GGCTGCGTATTGGAGGAACAGGAGCATCAAGTAGCAAAAGAGGAAAGAGAGACACCTACCACCG
ATTCTATGCCCCCACTTCTGAGACAAGCAATTGAGCTGTTCGACCGGCAGGGAGCCGAACCTGCCT
TCCTTTTCGGCCTGGAACTAATCATATGTGGCCTGGAGAAACAGCTAAAGTGCGAAAGCGGCGGG
CCGACCGACGCCCTTGACGATTTTGACTTAGACATGCTCCCAGCCGATGCCCTTGACGATTTTGAC
CTTGACATGCTCCCCGGGTAAGAATTCGATATCAAGCTTATCGATAATCAACCTCTGGATTACAAA
ATTTGTGAAAGATTGACTGGTATTCTTAACTATGTTGCTCCTTTTACGCTATGTGGATACGCTGCTT
TAATGCCTTTGTATCATGCTATTGCTTCCCGTATGGCTTTCATTTTCTCCTCCTTGTATAAATCCTGG
TTGCTGTCTCTTTATGAGGAGTTGTGGCCCGTTGTCAGGCAACGTGGCGTGGTGTGCACTGTGTTTG
CTGACGCAACCCCCACTGGTTGGGGCATTGCCACCACCTGTCAGCTCCTTTCCGGAACTTTCGCTTT
CCCCCTCCCTATTGCCACGGCGGAACTCATCGCCGCCTGCCTTGCCCGCTGCTGGACAGGGGCTCG
GCTGTTGGGCACTGACAATTCCGTGGTGTTGTCGGGGAAATCATCGTCCTTTCCTTGGCTGCTCGCC
TATGTTGCCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCCTCAGAC
GAGTCGGATCTCCCTTTGGGCCGCCTCCCCGCATCGATACCGAGCGCTGCTCGAGAGATCTACGGG
TGGCATCCCTGTGACCCCTCCCCAGTGCCTCTCCTGGCCCTGGAAGTTGCCACTCCAGTGCCCACC
AGCCTTGTCCTAATAAAATTAAGTTGCATCATTTTGTCTGACTAGGTGTCCTTCTATAATATTATGG
GGTGGAGGGGGGTGGTATGGAGCAAGGGGCAAGTTGGGAAGACAACCTGTAGGGCCTGCGGGGT
CTATTGGGAACCAAGCTGGAGTGCAGTGGCACAATCTTGGCTCACTGCAATCTCCGCCTCCTGGGT
TCAAGCGATTCTCCTGCCTCAGCCTCCCGAGTTGTTGGGATTCCAGGCATGCATGACCAGGCTCAG
CTAATTTTTGTTTTTTTGGTAGAGACGGGGTTTCACCATATTGGCCAGGCTGGTCTCCAACTCCTAA
TCTCAGGTGATCTACCCACCTTGGCCTCCCAAATTGCTGGGATTACAGGCGTGAACCACTGCTCCC
TTCCCTGTCCTTCTGATTTTGTAGGTAACCACGTGCGGACCGAGCGGCCGCAGGAACCCCTAGTGA
TGGAGTTGGCCACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGCTGCCTGCAGGGGCGC
CTGATGCGGTATTTTCTCCTTACGCATCTGTGCGGTATTTCACACCGCATACGTCAAAGCAACCATA
GTACGCGCCCTGTAGCGGCGCATTAAGCGCGGCGGGTGTGGTGGTTACGCGCAGCGTGACCGCTA
CACTTGCCAGCGCCTTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTTACGGCACCTCG
ACCCCAAAAAACTTGATTTGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAACAACACTCA
ACTCTATCTCGGGCTATTCTTTTGATTTATAAGGGATTTTGCCGATTTCGGTCTATTGGTTAAAAAA
TGAGCTGATTTAACAAAAATTTAACGCGAATTTTAACAAAATATTAACGTTTACAATTTTATGGTG
CACTCTCAGTACAATCTGCTCTGATGCCGCATAGTTAAGCCAGCCCCGACACCCGCCAACACCCGC
TGACGCGCCCTGACGGGCTTGTCTGCTCCCGGCATCCGCTTACAGACAAGCTGTGACCGTCTCCGG
GAGCTGCATGTGTCAGAGGTTTTCACCGTCATCACCGAAACGCGCGAGACGAAAGGGCCTCGTGA
TACGCCTATTTTTATAGGTTAATGTCATGATAATAATGGTTTCTTAGACGTCAGGTGGCACTTTTCG
GGGAAATGTGCGCGGAACCCCTATTTGTTTATTTTTCTAAATACATTCAAATATGTATCCGCTCATG
AGACAATAACCCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATGAGCACTTTTAAAGTT
CTGCTATGTGGCGCGGTATTATCCCGTATTGACGCCGGGCAAGAGCAACTCGGTCGCCGCATACAC
TATTCTCAGAATGACTTGGTTGAGTACTCACCAGTCACAGAAAAGCATCTTACGGATGGCATGACA
GTAAGAGAATTATGCAGTGCTGCCATAACCATGAGTGATAACACTGCGGCCAACTTACTTCTGACA
ACGATCGGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATGCCTGT
AGCAATGGCAACAACGTTGCGCAAACTATTAACTGGCGAACTACTTACTCTAGCTTCCCGGCAACA
ATTAATAGACTGGATGGAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGCAACTATGGATG
AACGAAATAGACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTCTTTTTCCGAA
GGTAACTGGCTTCAGCAGAGCGCAGATACCAAATACTGTTCTTCTAGTGTAGCCGTAGTTAGGCCA
CCACTTCAAGAACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTGGCCTTTTGCT
GGCCTTTTGCTCACATGT

FIG. 3B: CaMKIIα promoter (from pAAV-CaMKIIα-rtTA2S-M2) (SEQ ID NO: 149):

TGCGGCCGCACGCGTTTAACATTATGGCCTTAGGTCACTTCATCTCCATGGGGTTCTT
CTTCTGATTTTCTAGAAAATGAGATGGGGGTGCAGAGAGCTTCCTCAGTGACCTGCCCAGGGTCAC
ATCAGAAATGTCAGAGCTAGAACTTGAACTCAGATTACTAATCTTAAATTCCATGCCTTGGGGGCA
TGCAAGTACGATATACAGAAGGAGTGAACTCATTAGGGCAGATGACCAATGAGTTTAGGAAAGAA
GAGTCCAGGGCAGGGTACATCTACACCACCCGCCCAGCCCTGGGTGAGTCCAGCCACGTTCACCTC
ATTATAGTTGCCTCTCTCCAGTCCTACCTTGACGGGAAGCACAAGCAGAAACTGGGACAGGAGCC
CCAGGAGACCAAATCTTCATGGTCCCTCTGGGAGGATGGGTGGGGAGAGCTGTGGCAGAGGCCTC
AGGAGGGGCCCTGCTGCTCAGTGGTGACAGATAGGGGTGAGAAAGCAGACAGAGTCATTCCGTCA
GCATTCTGGGTCTGTTTGGTACTTCTTCTCACGCTAAGGTGGCGGTGTGATATGCACAATGGCTAA
AAAGCAGGGAGAGCTGGAAAGAAACAAGGACAGAGACAGAGGCCAAGTCAACCAGACCAATTCC
CAGAGGAAGCAAAGAAACCATTACAGAGACTACAAGGGGGAAGGGAAGGAGAGATGAATTAGCT
TCCCCTGTAAACCTTAGAACCCAGCTGTTGCCAGGGCAACGGGGCAATACCTGTCTCTTCAGAGGA
GATGAAGTTGCCAGGGTAACTACATCCTGTCTTTCTCAAGGACCATCCCAGAATGTGGCACCCACT
AGCCGTTACCATAGCAACTGCCTCTTTGCCCCACTTAATCCCATCCCGTCTGTTAAAAGGGCCCTAT
AGTTGGAGGTGGGGGAGGTAGGAAGAGCGATGATCACTTGTGGACTAAGTTTGTTCGCATCCCCTT
CTCCAACCCCCTCAGTACATCACCCTGGGGGAACAGGGTCCACTTGCTCCTGGGCCCACACAGTCC
TGCAGTATTGTGTATATAAGGCCAGGGCAAAGAGGAGCAGGTTTTAAAGTGAAAGGCAGGCAGGT
GTTGGGGAGGCAGTTACCGGGGCAACGGGAACAGGGCGTTTCGGAGGTGGTTGCCATGGGGACCT
GGATGCTGACGAAGGCTCGCGAGGCTGTGAGCAGCCACAGTGCCCTGCTCAGAAGCCCCAAGCTC
GTCAGTCAAGCCGGTTCTCCGTTTGCACTCAGGAGCACGGGCAGGCGAGTGGCCCCTAGTTCTGGG
GGCAGCTCTAGAGCGGTACC

FIG. 3B: rtTA2S-M2 (from pAAV-CaMKIIα-rtTA2S-M2) (SEQ ID NO: 14):

ATGTCTAGACTGGACAAGAGCAAAGTCATAAACGGCGCTCTGGAATTACTCAATGGA
GTCGGTATCGAAGGCCTGACGACAAGGAAACTCGCTCAAAAGCTGGGAGTTGAGCAGCCTACCCT
GTACTGGCACGTGAAGAACAAGCGGGCCCTGCTCGATGCCCTGCCAATCGAGATGCTGGACAGGC
ATCATACCCACTTCTGCCCCCTGGAAGGCGAGTCATGGCAAGACTTTCTGCGGAACAACGCCAAGT
CATTCCGCTGTGCTCTCCTCTCACATCGCGACGGGGCTAAAGTGCATCTCGGCACCCGCCCAACAG
AGAAACAGTACGAAACCCTGGAAAATCAGCTCGCGTTCCTGTGTCAGCAAGGCTTCTCCCTGGAG
AACGCACTGTACGCTCTGTCCGCCGTGGGCCACTTTACACTGGGCTGCGTATTGGAGGAACAGGAG
CATCAAGTAGCAAAAGAGGAAAGAGAGACACCTACCACCGATTCTATGCCCCCACTTCTGAGACA
AGCAATTGAGCTGTTCGACCGGCAGGGAGCCGAACCTGCCTTCCTTTTCGGCCTGGAACTAATCAT
ATGTGGCCTGGAGAAACAGCTAAAGTGCGAAAGCGGCGGGCCGGCCGACGCCCTTGACGATTTTG
ACTTAGACATGCTCCCAGCCGATGCCCTTGACGACTTTGACCTTGATATGCTGCCTGCTGACGCTCT
TGACGATTTTGACCTTGACATGCTCCCCGGGTAA

Amino acid sequence of rtTA2S-M2 (or M2-rtTA) (SEQ ID NO: 15):

MSRLDKSKVINGALELLNGVGIEGLTTRKLAQKLGVEQPTLYWHVKNKRALLDALPIEM
LDRHHTHFCPLEGESWQDFLRNNAKSFRCALLSHRDGAKVHLGTRPTEKQYETLENQLAFLCQQGFSL
ENALYALSAVGHFTLGCVLEEQEHQVAKEERETPTTDSMPPLLRQAIELFDROGAEPAFLFGLELIICGL
EKQLKCESGGPADALDDFDLDMLPADALDDFDLDMLPADALDDFDLDMLPG

FIG. 3B: WPRE (from pAAV-CaMKIIα-rtTA2S-M2) (SEQ ID NO: 152):

AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCACCAC
CTGTCAGCTCCTTTCCGGAACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCCGTGGTGTTGTCGGGG
AAATCATCGTCCTTTCCTTGGCTGCTCGCCTATGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGCGGCC
TCTTCCGCGTCTTCGCCTTCGCCCTCAGACGAGTCGGATCTCCCTTTGGGCCGCCTCCCCGC

FIG. 3B: hGH pA (from pAAV-CaMKIIα-rtTA2S-M2) (SEQ ID NO: 153):

GGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTA
TAATATTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGGATTCCAGGCATGCATG
ACCAGGCTCAGCTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATTACAGGCGTGAA
CCACTGCTCCCTTCCCTGTCCTT

FIG. 3B: CaMKIIα promoter (from pAAV-CaMKIIα-rtTA3) (SEQ ID NO: 154):

TGCGGCCGCACGCGTTTAACATTATGGCCTTAGGTCACTTCATCTCCATGGGGTTCTT
CTTCTGATTTTCTAGAAAATGAGATGGGGGTGCAGAGAGCTTCCTCAGTGACCTGCCCAGGGTCAC
ATCAGAAATGTCAGAGCTAGAACTTGAACTCAGATTACTAATCTTAAATTCCATGCCTTGGGGGCA
TGCAAGTACGATATACAGAAGGAGTGAACTCATTAGGGCAGATGACCAATGAGTTTAGGAAAGAA
GAGTCCAGGGCAGGGTACATCTACACCACCCGCCCAGCCCTGGGTGAGTCCAGCCACGTTCACCTC
ATTATAGTTGCCTCTCTCCAGTCCTACCTTGACGGGAAGCACAAGCAGAAACTGGGACAGGAGCC
CCAGGAGACCAAATCTTCATGGTCCCTCTGGGAGGATGGGTGGGGAGAGCTGTGGCAGAGGCCTC
AGGAGGGGCCCTGCTGCTCAGTGGTGACAGATAGGGGTGAGAAAGCAGACAGAGTCATTCCGTCA
GCATTCTGGGTCTGTTTGGTACTTCTTCTCACGCTAAGGTGGCGGTGTGATATGCACAATGGCTAA
AAAGCAGGGAGAGCTGGAAAGAAACAAGGACAGAGACAGAGGCCAAGTCAACCAGACCAATTCC
CAGAGGAAGCAAAGAAACCATTACAGAGACTACAAGGGGGAAGGGAAGGAGAGATGAATTAGCT
TCCCCTGTAAACCTTAGAACCCAGCTGTTGCCAGGGCAACGGGGCAATACCTGTCTCTTCAGAGGA
GATGAAGTTGCCAGGGTAACTACATCCTGTCTTTCTCAAGGACCATCCCAGAATGTGGCACCCACT
AGCCGTTACCATAGCAACTGCCTCTTTGCCCCACTTAATCCCATCCCGTCTGTTAAAAGGGCCCTAT
AGTTGGAGGTGGGGGAGGTAGGAAGAGCGATGATCACTTGTGGACTAAGTTTGTTCGCATCCCCTT
CTCCAACCCCCTCAGTACATCACCCTGGGGGAACAGGGTCCACTTGCTCCTGGGCCCACACAGTCC
TGCAGTATTGTGTATATAAGGCCAGGGCAAAGAGGAGCAGGTTTTAAAGTGAAAGGCAGGCAGGT
GTTGGGGAGGCAGTTACCGGGGCAACGGGAACAGGGCGTTTCGGAGGTGGTTGCCATGGGGACCT
GGATGCTGACGAAGGCTCGCGAGGCTGTGAGCAGCCACAGTGCCCTGCTCAGAAGCCCCAAGCTC
GTCAGTCAAGCCGGTTCTCCGTTTGCACTCAGGAGCACGGGCAGGCGAGTGGCCCCTAGTTCTGGG
GGCAGCTCTAGAGCGGTACC

FIG. 3B: WPRE (from pAAV-CaMKIIα-rtTA3) (SEQ ID NO: 155):

AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCACCAC
CTGTCAGCTCCTTTCCGGAACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCCGTGGTGTTGTCGGGG
AAATCATCGTCCTTTCCTTGGCTGCTCGCCTATGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGCGGCC
TCTTCCGCGTCTTCGCCTTCGCCCTCAGACGAGTCGGATCTCCCTTTGGGCCGCCTCCCCGC

FIG. 3B: hGH pA (from pAAV-CaMKIIα-rtTA3) (SEQ ID NO: 156):

GGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTA
TAATATTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGGATTCCAGGCATGCATG
ACCAGGCTCAGCTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATTACAGGCGTGAA
CCACTGCTCCCTTCCCTGTCCTT

pAAV-TRE3G-OSK-SV40 (mouse)_(SEQ ID NO: 16) as used in FIG. 3C:

TTATGCAGTGCTGCCATAACCATGAGTGATAACACTGCGGCCAACTTACTTCTGACAA
CGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTA
GTAATGGTAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAAGC
GGAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCACT
CATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAGATTTAATTAAGGCCTTAATTAGG
CTGCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCTT
GTAGTTAATGATTAACCCGCCATGCTACTTATCTACGTAGCCATGCTCTAGGAAGATCGGAATTCT
TTACTCCCTATCAGTGATAGAGAACGTATGAAGAGTTTACTCCCTATCAGTGATAGAGAACGTATG
CAGACTTTACTCCCTATCAGTGATAGAGAACGTATAAGGAGTTTACTCCCTATCAGTGATAGAGAA
CGTATGACCAGTTTACTCCCTATCAGTGATAGAGAACGTATCTACAGTTTACTCCCTATCAGTGAT
AGAGAACGTATATCCAGTTTACTCCCTATCAGTGATAGAGAACGTATAAGCTTTAGGCGTGTACGG
TGGGCGCCTATAAAAGCAGAGCTCGTTTAGTGAACCGTCAGATCGCCTGGAGCAATTCCACAACA
CTTTTGTCTTATACCAACTTTCCGTACCACTTCCTACCCTCGTAAAGCGGCCGCGCCACCATGGCTG
GACACCTGGCTTCAGACTTCGCCTTCTCACCCCCACCAGGTGGGGGTGATGGGTCAGCAGGGCTGG
AGCCGGGCTGGGTGGATCCTCGAACCTGGCTAAGCTTCCAAGGGCCTCCAGGTGGGCCTGGAATC
GGACCAGGCTCAGAGGTATTGGGGATCTCCCCATGTCCGCCCGCATACGAGTTCTGCGGAGGGAT
GGCATACTGTGGACCTCAGGTTGGACTGGGCCTAGTCCCCCAAGTTGGCGTGGAGACTTTGCAGCC
TGAGGGCCAGGCAGGAGCACGAGTGGAAAGCAACTCAGAGGGAACCTCCTCTGAGCCCTGTGCCG
ACCGCCCCAATGCCGTGAAGTTGGAGAAGGTGGAACCAACTCCCGAGGAGTCCCAGGACATGAAA
GCCCTGCAGAAGGAGCTAGAACAGTTTGCCAAGCTGCTGAAGCAGAAGAGGATCACCTTGGGGTA
CACCCAGGCCGACGTGGGGCTCACCCTGGGCGTTCTCTTTGGAAAGGTGTTCAGCCAGACCACCAT
CTGTCGCTTCGAGGCCTTGCAGCTCAGCCTTAAGAACATGTGTAAGCTGCGGCCCCTGCTGGAGAA
GTGGGTGGAGGAAGCCGACAACAATGAGAACCTTCAGGAGATATGCAAATCGGAGACCCTGGTGC
AGGCCCGGAAGAGAAAGCGAACTAGCATTGAGAACCGTGTGAGGTGGAGTCTGGAGACCATGTTT
CTGAAGTGCCCGAAGCCCTCCCTACAGCAGATCACTCACATCGCCAATCAGCTTGGGCTAGAGAA
GGATGTGGTTCGAGTATGGTTCTGTAACCGGCGCCAGAAGGGCAAAAGATCAAGTATTGAGTATT
CCCAACGAGAAGAGTATGAGGCTACAGGGACACCTTTCCCAGGGGGGGCTGTATCCTTTCCTCTGC
CCCCAGGTCCCCACTTTGGCACCCCAGGCTATGGAAGCCCCCACTTCACCACACTCTACTCAGTCC
CTTTTCCTGAGGGCGAGGCCTTTCCCTCTGTTCCCGTCACTGCTCTGGGCTCTCCCATGCATTCAAA
CGCTAGCGGCAGCGGCGCCACGAACTTCTCTCTGTTAAAGCAAGCAGGAGATGTTGAAGAAAACC
CCGGGCCTGCATGCATGTATAACATGATGGAGACGGAGCTGAAGCCGCCGGGCCCGCAGCAAGCT
TCGGGGGGCGGCGGCGGAGGAGGCAACGCCACGGCGGCGGCGACCGGCGGCAACCAGAAGAACA
GCCCGGACCGCGTCAAGAGGCCCATGAACGCCTTCATGGTATGGTCCCGGGGGCAGCGGCGTAAG
ATGGCCCAGGAGAACCCCAAGATGCACAACTCGGAGATCAGCAAGCGCCTGGGCGCGGAGTGGA
AACTTTTGTCCGAGACCGAGAAGCGGCCGTTCATCGACGAGGCCAAGCGGCTGCGCGCTCTGCAC
ATGAAGGAGCACCCGGATTATAAATACCGGCCGCGGCGGAAAACCAAGACGCTCATGAAGAAGG
ATAAGTACACGCTTCCCGGAGGCTTGCTGGCCCCCGGCGGGAACAGCATGGCGAGCGGGGTTGGG
GTGGGCGCCGGCCTGGGTGCGGGCGTGAACCAGCGCATGGACAGCTACGCGCACATGAACGGCTG
GAGCAACGGCAGCTACAGCATGATGCAGGAGCAGCTGGGCTACCCGCAGCACCCGGGCCTCAACG
CTCACGGCGCGGCACAGATGCAACCGATGCACCGCTACGACGTCAGCGCCCTGCAGTACAACTCC
ATGACCAGCTCGCAGACCTACATGAACGGCTCGCCCACCTACAGCATGTCCTACTCGCAGCAGGG
CACCCCCGGTATGGCGCTGGGCTCCATGGGCTCTGTGGTCAAGTCCGAGGCCAGCTCCAGCCCCCC
CGTGGTTACCTCTTCCTCCCACTCCAGGGCGCCCTGCCAGGCCGGGGACCTCCGGGACATGATCAG
CATGTACCTCCCCGGCGCCGAGGTGCCGGAGCCCGCTGCGCCCAGTAGACTGCACATGGCCCAGC
ACTACCAGAGCGGCCCGGTGCCCGGCACGGCCATTAACGGCACACTGCCCCTGTCGCACATGGCA
TGCGGCTCCGGCGAGGGCAGGGGAAGTCTTCTAACATGCGGGGACGTGGAGGAAAATCCCGGCCC
ACTCGAGATGAGGCAGCCACCTGGCGAGTCTGACATGGCTGTCAGCGACGCTCTGCTCCCGTCCTT
CTCCACGTTCGCGTCCGGCCCGGCGGGAAGGGAGAAGACACTGCGTCCAGCAGGTGCCCCGACTA
ACCGTTGGCGTGAGGAACTCTCTCACATGAAGCGACTTCCCCCACTTCCCGGCCGCCCCTACGACC
TGGCGGCGACGGTGGCCACAGACCTGGAGAGTGGCGGAGCTGGTGCAGCTTGCAGCAGTAACAAC
CCGGCCCTCCTAGCCCGGAGGGAGACCGAGGAGTTCAACGACCTCCTGGACCTAGACTTTATCCTT
TCCAACTCGCTAACCCACCAGGAATCGGTGGCCGCCACCGTGACCACCTCGGCGTCAGCTTCATCC
TCGTCTTCCCCAGCGAGCAGCGGCCCTGCCAGCGCGCCCTCCACCTGCAGCTTCAGCTATCCGATC
CGGGCCGGGGGTGACCCGGGCGTGGCTGCCAGCAACACAGGTGGAGGGCTCCTCTACAGCCGAGA
ATCTGCGCCACCTCCCACGGCCCCCTTCAACCTGGCGGACATCAATGACGTGAGCCCCTCGGGCGG
CTTCGTGGCTGAGCTCCTGCGGCCGGAGTTGGACCCAGTATACATTCCGCCACAGCAGCCTCAGCC
GCCAGGTGGCGGGCTGATGGGCAAGTTTGTGCTGAAGGCGTCTCTGACCACCCCTGGCAGCGAGT
ACAGCAGCCCTTCGGTCATCAGTGTTAGCAAAGGAAGCCCAGACGGCAGCCACCCCGTGGTAGTG
GCGCCCTACAGCGGTGGCCCGCCGCGCATGTGCCCCAAGATTAAGCAAGAGGCGGTCCCGTCCTG
CACGGTCAGCCGGTCCCTAGAGGCCCATTTGAGCGCTGGACCCCAGCTCAGCAACGGCCACCGGC
CCAACACACACGACTTCCCCCTGGGGCGGCAGCTCCCCACCAGGACTACCCCTACACTGAGTCCCG
AGGAACTGCTGAACAGCAGGGACTGTCACCCTGGCCTGCCTCTTCCCCCAGGATTCCATCCCCATC
CGGGGCCCAACTACCCTCCTTTCCTGCCAGACCAGATGCAGTCACAAGTCCCCTCTCTCCATTATC
AAGAGCTCATGCCACCGGGTTCCTGCCTGCCAGAGGAGCCCAAGCCAAAGAGGGGAAGAAGGTC
GTGGCCCCGGAAAAGAACAGCCACCCACACTTGTGACTATGCAGGCTGTGGCAAAACCTATACCA
AGAGTTCTCATCTCAAGGCACACCTGCGAACTCACACAGGCGAGAAACCTTACCACTGTGACTGG
GACGGCTGTGGGTGGAAATTCGCCCGCTCCGATGAACTGACCAGGCACTACCGCAAACACACAGG
GCACCGGCCCTTTCAGTGCCAGAAGTGCGACAGGGCCTTTTCCAGGTCGGACCACCTTGCCTTACA
CATGAAGAGGCACTAAATGACTAGTGCGCGCAGCGGCCGACCATGGCCCAACTTGTTTATTGCAG
CTTATAATGGTTACAAATAAAGCAATAGCATCACAAATTTCACAAATAAAGCATTTTTTTCACTGC
ATTCTAGTTGTGGTTTGTCCAAACTCATCAATGTATCTTATCATGTCTGGATCTCGGTACCGGATCC
AAATTCCCGATAAGGATCTTCCTAGAGCATGGCTACGTAGATAAGTAGCATGGCGGGTTAATCATT
AACTACAAGGAACCCCTAGTGATGGAGTTGGCCACTCCCTCTCTGCGCGCTCGCTCGCTCACTGAG
GCCGGGCGACCAAAGGTCGCCCGACGCCCGGGCTTTGCCCGGGCGGCCTCAGTGAGCGAGCGAGC
GCGCAGCCTTAATTAACCTAATTCACTGGCCGTCGTTTTACAACGTCGTGACTGGGAAAACCCTGG
CGTTACCCAACTTAATCGCCTTGCAGCACATCCCCCTTTCGCCAGCTGGCGTAATAGCGAAGAGGC
CCGCACCGATCGCCCTTCCCAACAGTTGCGCAGCCTGAATGGCGAATGGGACGCGCCCTGTAGCG
GCGCATTAAGCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCGTCAAGCTCT
AAATCGGGGGCTCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGTTGGA
GTCCACGTTCTTTAATAGTGGACTCTTGTTCCAAACTGGAACAACACTCAACCCTATCTCGGTCTAT
TCTTTTGATTTATAAGGGATTTTGCCGATTTCGGCCTATTGGTTAAAAAATGAGCTGATTTAACAAA
AATTTAACGCGAATTTTAACAAAATATTAACGTTTATAATTTCAGGTGGCATCTTTCGGGGAAATG
TGCGCGGAACCCCTATTTGTTTATTTTTCTAAATACATTCAAATATGTATCCGCTCATGAGACAATA
ACCCTGATAAATGCTTCAATAATATTGAAAAAGGAAGAGTATGAGTATTCAACATTTCCGTGTCGC
CCTTATTCCCTTTTTTGCGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGATCTCAATAGTGGTAA
GATCCTTGAGAGTTTTCGCCCCGAAGAACGTTTTCCAATGATGAGCACTTTTAAAGTTCTGCTATGT
GGCGCGGTATTATCCCGTATTGACGCCGGGCAAGAGCAACTCGGTCGCCGCATACACTATTCTCAG
AATGACTTGGTTGAGTACTCACCAGTCACAGAAAAGCATCTTACGGATGGCATGACAGTAAGAGA
A

pAAV-ihSyn1_(SEQ ID NO: 127) as used in FIG. 3C:

CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTCCATCACTAG
GGGTTCCTGCGGCCGCACGCGTGTGTCTAGACTGCAGAGGGCCCTGCGTATGAGTGCAAGTGGGTT
TTAGGACCAGGATGAGGCGGGGTGGGGGTGCCTACCTGACGACCGACCCCGACCCACTGGACAAG
CACCCAACCCCCATTCCCCAAATTGCGCATCCCCTATCAGAGAGGGGGAGGGGAAACAGGATGCG
GCGAGGCGCGTGCGCACTGCCAGCTTCAGCACCGCGGACAGTGCCTTCGCCCCCGCCTGGCGGCG
CGCGCCACCGCCGCCTCAGCACTGAAGGCGCGCTGACGTCACTCGCCGGTCCCCCGCAAACTCCCC
TTCCCGGCCACCTTGGTCGCGTCCGCGCCGCCGCCGGCCCAGCCGGACCGCACCACGCGAGGCGC
GAGATAGGGGGGCACGGGCGCGACCATCTGCGCTGCGGCGCCGGCGACTCAGCGCTGCCTCAGTC
TGCGGTGGGCAGCGGAGGAGTCGTGTCGTGCCTGAGAGCGCAGTCGAGAACAGATCTCTAAAACA
GGTAAGTCCCATTAATCTCCCTATCAGTGATAGAGAAGGTCTGAAGAGTTTACTCCCTATCAGTGA
TAGAGATTAATTCCTCTACTAACCTTGTTCATCTTTTCTTTTTTTTTCTACAGGTCCTGGGTGATTAA
CAGCTTAAGGGCCCGGATCCGGTACCGCCACCATGAGTCGGCTGGATAAATCTAAAGTCATAAAC
GGCGCTCTGGAATTACTCAATGAAGTCGGTATCGAAGGCCTGACGACAAGGAAACTCGCTCAAAA
GCTGGGAGTTGAGCAGCCTACCCTGTACTGGCACGTGAAGAACAAGCGGGCCCTGCTCGATGCCC
TGGCCATCGAGATGCTGGACAGGCATCATACCCACTTCTGCCCCCTGGAAGGCGAGTCATGGCAA
GACTTTCTGCGGAACAACGCCAAGTCATTCCGCTGTGCTCTCCTCTCACATCGCGACGGGGCTAAA
GTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACT
GGGCTGCGTATTGGAGGAACAGGAGCATCAAGTAGCAAAAGAGGAAAGAGAGACACCTACCACC
GATTCTATGCCCCCACTTCTGAGACAAGCAATTGAGCTGTTCGACCGGCAGGGAGCCGAACCTGCC
TTCCTTTTCGGCCTGGAACTAATCATATGTGGCCTGGAGAAACAGCTAAAGTGCGAAAGCGGCGG
GCCGGCCGACGCCCTTGACGATTTTGACTTAGACATGCTCCCAGCCGATGCCCTTGACGACTTTGA
CCTTGATATGCTGCCTGCTGACGCTCTTGACGATTTTGACCTTGACATGCTCCCCGGGTAAGAATTC
GATATCAAGCTTATCGATAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTATTGCTTC
CCGTATGGCTTTCATTTTCTCCTCCTTGTATAAATCCTGGTTGCTGTCTCTTTATGAGGAGTTGTGGC
CCGTTGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCGTGGT
GTTGTCGGGGAAATCATCGTCCTTTCCTTGGCTGCTCGCCTATGTTGCCACCTGGATTCTGCGCGGG
ACGTCCTTCTGCTACGTCCCTTCGGCCCTCAATCCAGCGGACCTTCCTTCCCGCGGCCTGCTGCCGG
CTCTGCGGCCTCTTCCGCGTCTTCGCCTTCGCCCTCAGACGAGTCGGATCTCCCTTTGGGCCGCCTC
CCCGCATCGATACCGAGCGCTGCTCGAGAGATCTACGGGTGGCATCCCTGTGACCCCTCCCCAGTG
CCTCTCCTGGCCCTGGAAGTTGCCACTCCAGTGCCCACCAGCCTTGTCCTAATAAAATTAAGTTGC
ATCATTTTGTCTGACTAGGTGTCCTTCTATAATATTATGGGGTGGAGGGGGGTGGTATGGAGCAAG
GGGCAAGTTGGGAAGACAACCTGTAGGGCCTGCGGGGTCTATTGGGAACCAAGCTGGAGTGCAGT
GGCACAATCTTGGCTCACTGCAATCTCCGCCTCCTGGGTTCAAGCGATTCTCCTGCCTCAGCCTCCC
GAGTTGTTGGGATTCCAGGCATGCATGACCAGGCTCAGCTAATTTTTGTTTTTTTGGTAGAGACGG
GGTTTCACCATATTGGCCAGGCTGGTCTCCAACTCCTAATCTCAGGTGATCTACCCACCTTGGCCTC
CCAAATTGCTGGGATTACAGGCGTGAACCACTGCTCCCTTCCCTGTCCTTCTGATTTTGTAGGTAAC
CACGTGCGGACCGAGCGGCCGCAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGCCTC
AGTGAGCGAGCGAGCGCGCAGCTGCCTGCAGGGGCGCCTGATGCGGTATTTTCTCCTTACGCATCT
GTGCGGTATTTCACACCGCATACGTCAAAGCAACCATAGTACGCGCCCTGTAGCGGCGCATTAAGC
GCGGCGGGTGTGGTGGTTACGCGCAGCGTGACCGCTACACTTGCCAGCGCCTTAGCGCCCGCTCCT
TTCGCTTTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTTGGGTGATGG
TTCACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACTCTATCTCGGGCTATTCTTTTGATTTAT
AAGGGATTTTGCCGATTTCGGTCTATTGGTTAAAAAATGAGCTGATTTAACAAAAATTTAACGCGA
ATTTTAACAAAATATTAACGTTTACAATTTTATGGTGCACTCTCAGTACAATCTGCTCTGATGCCGC
ATAGTTAAGCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAATGTCATGAT
AATAATGGTTTCTTAGACGTCAGGTGGCACTTTTCGGGGAAATGTGCGCGGAACCCCTATTTGTTT
ATTTTTCTAAATACATTCAAATATGTATCCGCTCATGAGACAATAACCCTGATAAATGCTTCAATA
ATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTG
GGTGCACGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGCCATAAC
CATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCC
GGTGAGCGTGGGTCTCGCGGTATCATTGCAGCACTGGGGCCAGATGGTAAGCCCTCCCGTATCGTA
GTTATCTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTCAGCAGAGCGCAGATAC
CAAATACTGTTCTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGA
AAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGT

FIG. 3C: Synapsin-I promoter (from pAAV-ihSyn1-tTA) (SEQ ID NO: 157):

AGTGCAAGTGGGTTTTAGGACCAGGATGAGGCGGGGTGGGGGTGCCTACCTGACGAC
CGACCCCGACCCACTGGACAAGCACCCAACCCCCATTCCCCAAATTGCGCATCCCCTATCAGAGAG
GGGGAGGGGAAACAGGATGCGGCGAGGCGCGTGCGCACTGCCAGCTTCAGCACCGCGGACAGTG
CCTTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGCC
GGACCGCACCACGCGAGGCGCGAGATAGGGGGGCACGGGCGCGACCATCTGCGCTGCGGCGCCG
GCGACTCAGCGCTGCCTCAGTCTGCGGTGGGCAGCGGAGGAGTCGTGTCGTGCCTGAGAGCGCAG

FIG. 3C: tTA (from pAAV-ihSyn1-tTA) (SEQ ID NO: 158):

ATGAGTCGGCTGGATAAATCTAAAGTCATAAACGGCGCTCTGGAATTACTCAATGAA
GTCGGTATCGAAGGCCTGACGACAAGGAAACTCGCTCAAAAGCTGGGAGTTGAGCAGCCTACCCT
GTACTGGCACGTGAAGAACAAGCGGGCCCTGCTCGATGCCCTGGCCATCGAGATGCTGGACAGGC
ATCATACCCACTTCTGCCCCCTGGAAGGCGAGTCATGGCAAGACTTTCTGCGGAACAACGCCAAGT
CATTCCGCTGTGCTCTCCTCTCACATCGCGACGGGGCTAAAGTGCATCTCGGCACCCGCCCAACAG
AGAAACAGTACGAAACCCTGGAAAATCAGCTCGCGTTCCTGTGTCAGCAAGGCTTCTCCCTGGAG
AACGCACTGTACGCTCTGTCCGCCGTGGGCCACTTTACACTGGGCTGCGTATTGGAGGAACAGGAG
CATCAAGTAGCAAAAGAGGAAAGAGAGACACCTACCACCGATTCTATGCCCCCACTTCTGAGACA
AGCAATTGAGCTGTTCGACCGGCAGGGAGCCGAACCTGCCTTCCTTTTCGGCCTGGAACTAATCAT
ATGTGGCCTGGAGAAACAGCTAAAGTGCGAAAGCGGCGGGCCGGCCGACGCCCTTGACGATTTTG
ACTTAGACATGCTCCCAGCCGATGCCCTTGACGACTTTGACCTTGATATGCTGCCTGCTGACGCTCT
TGACGATTTTGACCTTGACATGCTCCCCGGGTAA

Amino acid sequence of tTA (SEQ ID NO: 159):

MSRLDKSKVINGALELLNEVGIEGLTTRKLAQKLGVEQPTLYWHVKNKRALLDALAIEM
LDRHHTHFCPLEGESWQDFLRNNAKSFRCALLSHRDGAKVHLGTRPTEKQYETLENQLAFLCQQGFSL
ENALYALSAVGHFTLGCVLEEQEHQVAKEERETPTTDSMPPLLRQAIELFDROGAEPAFLFGLELIICGL
EKQLKCESGGPADALDDFDLDMLPADALDDFDLDMLPADALDDFDLDMLPG

FIG. 3C: WPRE (from pAAV-ihSyn1-tTA) (SEQ ID NO: 160):

AATCAACCTCTGGATTACAAAATTTGTGAAAGATTGACTGGTATTCTTAACTATGTTG
CTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCACCAC
CTGTCAGCTCCTTTCCGGGACTTTCGCTTTCCCCCTCCCTATTGCCACGGCGGAACTCATCGCCGCC
TGCCTTGCCCGCTGCTGGACAGGGGCTCGGCTGTTGGGCACTGACAATTCCGTGGTGTTGTCGGGG
AAATCATCGTCCTTTCCTTGGCTGCTCGCCTATGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGCGGCC
TCTTCCGCGTCTTCGCCTTCGCCCTCAGACGAGTCGGATCTCCCTTTGGGCCGCCTCCCCGC

FIG. 3C: hGH pA (from pAAV-ihSyn1-tTA) (SEQ ID NO: 161):

GGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTA
TAATATTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGCAATCTCC
GCCTCCTGGGTTCAAGCGATTCTCCTGCCTCAGCCTCCCGAGTTGTTGGGATTCCAGGCATGCATG
ACCAGGCTCAGCTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATTACAGGCGTGAA
CCACTGCTCCCTTCCCTGTCCTT

CAG promoter (SEQ ID NO: 162):

GACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAACGA
CCCCCGCCCATTGACGTCAATAATGACGTATGTTCCCATAGTAACGCCAATAGGGACTTTCCATTG
ACGTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGGTCGAGGT
GAGCCCCACGTTCTGCTTCACTCTCCCCATCTCCCCCCCCTCCCCACCCCCAATTTTGTATTTATTTA
TTTTTTAATTATTTTGTGCAGCGATGGGGGCGGGGGGGGGGGGGGCGCGCGCCAGGCGGGGCGGG
GCGGGGCGAGGGGCGGGGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGCGAAGCGCGC
GGCGGGCGGGAGTCGCTGCGTTGCCTTCGCCCCGTGCCCCGCTCCGCGCCGCCTCGCGCCGCCCGC
CCCGGCTCTGACTGACCGCGTTACTCCCACAGGTGAGCGGGCGGGACGGCCCTTCTCCTCCGGGCT
GTAATTAGCGCTTGGTTTAATGACGGCTCGTTTCTTTTCTGTGGCTGCGTGAAAGCCTTAAAGGGCT
CCGGGAGGGCCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCGGGGCTTTGT
GCGCTCCGCGTGTGCGCGAGGGGAGCGCGGCCGGGGGCGGTGCCCCGCGGTGCGGGGGGGCTGC
GAGGGGAACAAAGGCTGCGTGCGGGGTGTGTGCGTGGGGGGGTGAGCAGGGGGTGTGGGCGCGG
CGGTCGGGCTGTAACCCCCCCCTGCACCCCCCTCCCCGAGTTGCTGAGCACGGCCCGGCTTCGGGT
GCGGGGCTCCGTGCGGGGCGTGGCGCGGGGCTCGCCGTGCCGGGCGGGGGGTGGCGGCAGGTGG
GGGTGCCGGGCGGGGCGGGGCCGCCTCGGGCCGGGGAGGGCTCGGGGGAGGGGCGCGGCGGCCC
CGGAGCGCCGGCGGCTGTCGAGGCGCGGCGAGCCGCAGCCATTGCCTTTTATGGTAATCGTGCGA
GAGGGCGCAGGGACTTCCTTTGTCCCAAATCTGGCGGAGCCGAAATCTGGGAGGCGCCGCCGCAC
CCCCTCTAGCGGGCGCGGGCGAAGCGGTGCGGCGCCGGCAGGAAGGAAATGGGCGGGGAGGGCC
TTCGTGCGTCGCCGCGCCGCCGTCCCCTTCTCCATCTCCAGCCTCGGGGCTGCCGCAGGGGGACGG
CTGCCTTCGGGGGGGACGGGGCAGGGCGGGGTTCGGCTTCTGGCGTGTGACCGGCGGCTCTAGAG
CCTCTGCTAACCATGTTCATGCCTTCTTCTTTTTCCTACAGCTCCTGGGCAACGTGCTGGTTATTGTG
CTGTCTCATCATTTTGGCAAA

FIG. 4A: The OKS-mCherry sequence in the Col1a1-TetO-OKS transgenic mice (SEQ ID NO: 163) (The mouse OCT4 sequence is the same as SEQ ID NO: 1. The mouse Sox2 sequence is the same as SEQ ID NO: 3. The mouse KLF4 sequence is the same as SEQ ID NO: 5):

ATGGCTGGACACCTGGCTTCAGACTTCGCCTTCTCACCCCCACCAGGTGGGGGTGATG
GGTCAGCAGGGCTGGAGCCGGGCTGGGTGGATCCTCGAACCTGGCTAAGCTTCCAAGGGCCTCCA
GGTGGGCCTGGAATCGGACCAGGCTCAGAGGTATTGGGGATCTCCCCATGTCCGCCCGCATACGA
GTTCTGCGGAGGGATGGCATACTGTGGACCTCAGGTTGGACTGGGCCTAGTCCCCCAAGTTGGCGT
GGAGACTTTGCAGCCTGAGGGCCAGGCAGGAGCACGAGTGGAAAGCAACTCAGAGGGAACCTCC
TCTGAGCCCTGTGCCGACCGCCCCAATGCCGTGAAGTTGGAGAAGGTGGAACCAACTCCCGAGGA
GTCCCAGGACATGAAAGCCCTGCAGAAGGAGCTAGAACAGTTTGCCAAGCTGCTGAAGCAGAAGA
GGATCACCTTGGGGTACACCCAGGCCGACGTGGGGCTCACCCTGGGCGTTCTCTTTGGAAAGGTGT
TCAGCCAGACCACCATCTGTCGCTTCGAGGCCTTGCAGCTCAGCCTTAAGAACATGTGTAAGCTGC
GGCCCCTGCTGGAGAAGTGGGTGGAGGAAGCCGACAACAATGAGAACCTTCAGGAGATATGCAA
ATCGGAGACCCTGGTGCAGGCCCGGAAGAGAAAGCGAACTAGCATTGAGAACCGTGTGAGGTGG
AGTCTGGAGACCATGTTTCTGAAGTGCCCGAAGCCCTCCCTACAGCAGATCACTCACATCGCCAAT
CAGCTTGGGCTAGAGAAGGATGTGGTTCGAGTATGGTTCTGTAACCGGCGCCAGAAGGGCAAAAG
ATCAAGTATTGAGTATTCCCAACGAGAAGAGTATGAGGCTACAGGGACACCTTTCCCAGGGGGGG
CTGTATCCTTTCCTCTGCCCCCAGGTCCCCACTTTGGCACCCCAGGCTATGGAAGCCCCCACTTCAC
CACACTCTACTCAGTCCCTTTTCCTGAGGGCGAGGCCTTTCCCTCTGTTCCCGTCACTGCTCTGGGC
TCTCCCATGCATTCAAACGGAAGTGGCGTGAAACAGACTTTGAATTTTGACCTTCTCAAGTTGGCG
GGAGACGTGGAGTCCAACCCAGGGCCCATGGCTGTCAGCGACGCTCTGCTCCCGTCCTTCTCCACG
TTCGCGTCCGGCCCGGCGGGAAGGGAGAAGACACTGCGTCCAGCAGGTGCCCCGACTAACCGTTG
GCGTGAGGAACTCTCTCACATGAAGCGACTTCCCCCACTTCCCGGCCGCCCCTACGACCTGGCGGC
GACGGTGGCCACAGACCTGGAGAGTGGCGGAGCTGGTGCAGCTTGCAGCAGTAACAACCCGGCCC
TCCTAGCCCGGAGGGAGACCGAGGAGTTCAACGACCTCCTGGACCTAGACTTTATCCTTTCCAACT
CGCTAACCCACCAGGAATCGGTGGCCGCCACCGTGACCACCTCGGCGTCAGCTTCATCCTCGTCTT
CCCCAGCGAGCAGCGGCCCTGCCAGCGCGCCCTCCACCTGCAGCTTCAGCTATCCGATCCGGGCCG
GGGGTGACCCGGGCGTGGCTGCCAGCAACACAGGTGGAGGGCTCCTCTACAGCCGAGAATCTGCG
CCACCTCCCACGGCCCCCTTCAACCTGGCGGACATCAATGACGTGAGCCCCTCGGGCGGCTTCGTG
GCTGAGCTCCTGCGGCCGGAGTTGGACCCAGTATACATTCCGCCACAGCAGCCTCAGCCGCCAGGT
GGCGGGCTGATGGGCAAGTTTGTGCTGAAGGCGTCTCTGACCACCCCTGGCAGCGAGTACAGCAG
CCCTTCGGTCATCAGTGTTAGCAAAGGAAGCCCAGACGGCAGCCACCCCGTGGTAGTGGCGCCCT
ACAGCGGTGGCCCGCCGCGCATGTGCCCCAAGATTAAGCAAGAGGCGGTCCCGTCCTGCACGGTC
AGCCGGTCCCTAGAGGCCCATTTGAGCGCTGGACCCCAGCTCAGCAACGGCCACCGGCCCAACAC
ACACGACTTCCCCCTGGGGCGGCAGCTCCCCACCAGGACTACCCCTACACTGAGTCCCGAGGAACT
GCTGAACAGCAGGGACTGTCACCCTGGCCTGCCTCTTCCCCCAGGATTCCATCCCCATCCGGGGCC
CAACTACCCTCCTTTCCTGCCAGACCAGATGCAGTCACAAGTCCCCTCTCTCCATTATCAAGAGCTC
ATGCCACCGGGTTCCTGCCTGCCAGAGGAGCCCAAGCCAAAGAGGGGAAGAAGGTCGTGGCCCCG
GAAAAGAACAGCCACCCACACTTGTGACTATGCAGGCTGTGGCAAAACCTATACCAAGAGTTCTC
ATCTCAAGGCACACCTGCGAACTCACACAGGCGAGAAACCTTACCACTGTGACTGGGACGGCTGT
GGGTGGAAATTCGCCCGCTCCGATGAACTGACCAGGCACTACCGCAAACACACAGGGCACCGGCC
CTTTCAGTGCCAGAAGTGTGACAGGGCCTTTTCCAGGTCGGACCACCTTGCCTTACACATGAAGAG
GCACTTTTAAAGATCCCTCCCCCCCCCCTAACGTTACTGGCCGAAGCCGCTTGGAATAAGGCCGGT
GTGCGTTTGTCTATATGTTATTTTCCACCATATTGCCGTCTTTTGGCAATGTGAGGGCCCGGAAACC
TGGCCCTGTCTTCTTGACGAGCATTCCTAGGGGTCTTTCCCCTCTCGCCAAAGGAATGCAAGGTCT
GTTGAATGTCGTGAAGGAAGCAGTTCCTCTGGAAGCTTCTTGAAGACAAACAACGTCTGTAGCGA
CCCTTTGCAGGCAGCGGAACCCCCCACCTGGCGACAGGTGCCTCTGCGGCCAAAAGCCACGTGTA
TAAGATACACCTGCAAAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCCAGAAGGTACCCCATT
GTATGGGATCTGATCTGGGGCCTCGGTGCACATGCTTTACATGTGTTTAGTCGAGGTTAAAAAAAC
GTCTAGGCCCCCCGAACCACGGGGACGTGGTTTTCCTTTGAAAAACACGATGATAATATGGCCACA
CATATGATGTATAACATGATGGAGACGGAGCTGAAGCCGCCGGGCCCGCAGCAAGCTTCGGGGGG
CGGCGGCGGAGGAGGCAACGCCACGGCGGCGGCGACCGGCGGCAACCAGAAGAACAGCCCGGAC
CGCGTCAAGAGGCCCATGAACGCCTTCATGGTATGGTCCCGGGGGCAGCGGCGTAAGATGGCCCA
GGAGAACCCCAAGATGCACAACTCGGAGATCAGCAAGCGCCTGGGCGCGGAGTGGAAACTTTTGT
CCGAGACCGAGAAGCGGCCGTTCATCGACGAGGCCAAGCGGCTGCGCGCTCTGCACATGAAGGAG
CACCCGGATTATAAATACCGGCCGCGGCGGAAAACCAAGACGCTCATGAAGAAGGATAAGTACAC
GCTTCCCGGAGGCTTGCTGGCCCCCGGCGGGAACAGCATGGCGAGCGGGGTTGGGGTGGGCGCCG
GCCTGGGTGCGGGCGTGAACCAGCGCATGGACAGCTACGCGCACATGAACGGCTGGAGCAACGGC
AGCTACAGCATGATGCAGGAGCAGCTGGGCTACCCGCAGCACCCGGGCCTCAACGCTCACGGCGC
GGCACAGATGCAACCGATGCACCGCTACGACGTCAGCGCCCTGCAGTACAACTCCATGACCAGCT
CGCAGACCTACATGAACGGCTCGCCCACCTACAGCATGTCCTACTCGCAGCAGGGCACCCCCGGT
ATGGCGCTGGGCTCCATGGGCTCTGTGGTCAAGTCCGAGGCCAGCTCCAGCCCCCCCGTGGTTACC
TCTTCCTCCCACTCCAGGGCGCCCTGCCAGGCCGGGGACCTCCGGGACATGATCAGCATGTACCTC
CCCGGCGCCGAGGTGCCGGAGCCCGCTGCGCCCAGTAGACTGCACATGGCCCAGCACTACCAGAG
CGGCCCGGTGCCCGGCACGGCCATTAACGGCACACTGCCCCTGTCGCACATGGGTAGTGGGCAAT
GTACTAACTACGCTTTGTTGAAACTCGCTGGCGATGTTGAAAGTAACCCCGGTCCTATGGTGAGCA
AGGGCGAGGAGGATAACATGGCCATCATCAAGGAGTTCATGCGCTTCAAGGTGCACATGGAGGGC
TCCGTGAACGGCCACGAGTTCGAGATCGAGGGCGAGGGCGAGGGCCGCCCCTACGAGGGCACCCA
GACCGCCAAGCTGAAGGTGACCAAGGGTGGCCCCCTGCCCTTCGCCTGGGACATCCTGTCCCCTCA
GTTCATGTACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGACCGTGA
CCCAGGACTCCTCCCTGCAGGACGGCGAGTTCATCTACAAGGTGAAGCTGCGCGGCACCAACTTCC
CCTCCGACGGCCCCGTAATGCAGAAGAAGACCATGGGCTGGGAGGCCTCCTCCGAGCGGATGTAC
CCCGAGGACGGCGCCCTGAAGGGCGAGATCAAGCAGAGGCTGAAGCTGAAGGACGGCGGCCACT
ACGACGCTGAGGTCAAGACCACCTACAAGGCCAAGAAGCCCGTGCAGCTGCCCGGCGCCTACAAC
GTCAACATCAAGTTGGACATCACCTCCCACAACGAGGACTACACCATCGTGGAACAGTACGAACG
CGCCGAGGGCCGCCACTCCACCGGCGGCATGGACGAGCTGTACAAGTAA

### ADDITIONAL EMBODIMENTS

Embodiment 1. A method comprising:
inducing in a cell, tissue, and/or organ from the central nervous system, optionally wherein the cell, tissue, and/or organ is in a subject in need thereof:
   (i) OCT4 expression;
   (ii) SOX2 expression; and
   (iii) KLF4 expression;
in the absence of inducing c-MYC expression.

Embodiment 2. The method of embodiment 1, wherein OCT4 expression is induced by administering:
i) a first engineered nucleic acid encoding OCT4 or encoding a Cas9 fusion protein (CRISPR activator) and a guide RNA sequence targeting promoter or enhancer at endogenous locus of Oct4, optionally wherein the first nucleic acid (e.g., engineered nucleic acid) comprises RNA and/or DNA;
(ii) a chemical agent that induces OCT4 expression;
(iii) an antibody that induces OCT4 expression; or
(iv) an engineered protein encoding OCT4, optionally wherein OCT4 comprises a sequence that is at least 70% identical (e.g., at least 75%, 80%, 85%, 90%, 95%, 98%, 99%, or 100% identical) to SEQ ID NO: 2 or SEQ ID NO: 41.

Embodiment 3. The method of any one of embodiments 1-2, wherein SOX2 expression comprises administering:
(v) a second engineered nucleic acid encoding SOX2 encoding a Cas9 fusion protein (CRISPR activator) and a guide RNA sequence targeting promoter or enhancer at endogenous locus of SOX2, wherein the second engineered nucleic acid comprises RNA and/or DNA;
(vi) a chemical agent that induces SOX2 expression;
(vii) an antibody that induces SOX2 expression; or
(viii) an engineered protein encoding SOX2, optionally wherein SOX2 comprises a sequence that is at least 70% identical (e.g., at least 75%, 80%, 85%, 90%, 95%, 98%, 99%, or 100% identical) to SEQ ID NO: 4 or SEQ ID NO: 43.

Embodiment 4. The method of any one of embodiments 1-3, wherein KLF4 expression comprises administering:
(ix) a third engineered nucleic acid encoding KLF4 encoding a Cas9 fusion protein (CRISPR activator) and a guide RNA sequence targeting promoter or enhancer at endogenous locus of KLF4, wherein the third nucleic acid (e.g., engineered nucleic acid) comprises RNA and/or DNA;
(x) a chemical agent that induces KLF4 expression;
(xi) an antibody that induces KLF4 expression;
(xii) an engineered protein encoding KLF4, optionally wherein KLF4 comprises a sequence that is at least 70% identical (e.g., at least 75%, 80%, 85%, 90%, 95%, 98%, 99%, or 100% identical) to SEQ ID NO: 6 or SEQ ID NO: 45.

Embodiment 5. The method of any one of embodiments 2-4, wherein said first, second, third engineered nucleic acids, or a combination thereof are present on an expression vector or are not present on an expression vector, optionally wherein the first, second, third engineered nucleic acids are mRNA or plasmid DNA.

Embodiment 6. The method of embodiment 5, wherein two or three of said first, second and third engineered nucleic acids are present in the same expression vector.

Embodiment 7. The method of any one of embodiments 1-5, wherein said first, second and third engineered nucleic acids are present in separate expression vectors.

Embodiment 8. The method of any one of embodiments 5-7, wherein said expression vector(s) include an inducible promoter operably linked to the first, second, third engineered nucleic acids, or a combination thereof, optionally wherein said method further comprises administering an inducing agent.

Embodiment 9. The method of embodiment 8 wherein said promoter comprises a tetracycline response element (TRE).

Embodiment 10. The method of embodiment 9, wherein administration of the inducing agent comprises administering a protein or a fourth engineered nucleic acid encoding the inducing agent, optionally wherein the fourth engineered nucleic acid is introduced simultaneously as the first, second, and third engineered nucleic acids.

Embodiment 11. The method of embodiment 10, wherein the fourth engineered nucleic acid is present on a separate expression vector from the first, second, and third engineered nucleic acids.

Embodiment 12. The method of embodiment 10, wherein the fourth engineered nucleic acid is present on the same expression vector with at least one of the first, second, and third engineered nucleic acids.

Embodiment 13. The method of any one of embodiments 9-12, wherein the inducing agent is capable of inducing expression of the first, second, third engineered nucleic acids, or a combination thereof from the inducible promoter in the presence of a tetracycline and the method further comprises administering tetracycline and/or removing tetracycline, optionally wherein the tetracycline is doxycycline.

Embodiment 14. The method of embodiment 13, wherein the inducing agent is reverse tetracycline-controlled transactivator (rtTA).

Embodiment 15. The method of embodiment 14, wherein the rtTA is rtTA3, rtTA Advanced, or rtTA2S-M2.

Embodiment 16. The method of embodiment 15, wherein the rtTA3 comprises an amino acid sequence that is at least 70% identical (e.g., at least 75%, 80%, 85%, 90%, 95%, 98%, 99%, or 100% identical) to SEQ ID NO: 11, the rtTA Advanced comprises an amino acid sequence that is at least 70% identical (e.g., at least 75%, 80%, 85%, 90%, 95%, 98%, 99%, or 100% identical) to SEQ ID NO: 129, or the rtTA2S-M2 comprises an amino acid sequence that is at least 70% identical (e.g., at least 75%, 80%, 85%, 90%, 95%, 98%, 99%, or 100% identical) to SEQ ID NO: 15.

Embodiment 17. The method of any one of embodiments 9-12, wherein the inducing agent is capable of inducing expression of the first, second, third engineered nucleic acids, or a combination thereof from the inducible promoter in the absence of a tetracycline, optionally, wherein the tetracycline is doxycycline.

Embodiment 18. The method of embodiment 17, wherein the inducing agent is a temperature, a chemical, a pH, a nucleic acid, a protein, optionally wherein the protein is a tetracycline-controlled transactivator (tTA).

Embodiment 19. The method of any one of embodiments embodiment 11 or 13-18, wherein the first, second, and third engineered nucleic acids are present in a first expression vector and the fourth engineered nucleic acid is present in a second expression vector.

Embodiment 20. The method of any one of embodiments 9-19, wherein the promoter is a TRE3G, a TRE2 promoter, or a P tight promoter, optionally, wherein the promoter comprises a engineered nucleic acid sequence that is at least 70% identical (e.g., at least 75%, 80%, 85%, 90%, 95%, 98%, 99%, or 100% identical) to SEQ ID NO: 7, optionally, wherein the promoter comprises a engineered nucleic acid sequence that is at least 70% identical (e.g., at least 75%, 80%, 85%, 90%, 95%, 98%, 99%, or 100% identical) to SEQ ID NO: 23, and optionally wherein the promoter comprises a sequence that is at least 70% identical (e.g., at least 75%, 80%, 85%, 90%, 95%, 98%, 99%, or 100% identical) to SEQ ID NO: 24.

Embodiment 21. The method of any one of embodiments 1-7 or 10-20, wherein said expression vector(s) comprise a constitutive promoter operably linked to the first, second, third, fourth engineered nucleic acids, or any combination thereof.

Embodiment 22. The method of embodiment 21, wherein the constitutive promoter is operably linked to the fourth engineered nucleic acid but not to the first, second, or third engineered nucleic acids, optionally wherein the constitutive promoter is CP1, CMV, EF1 alpha, SV40, PGK1, Ubc, human beta actin, CAG, Ac5, polyhedrin, TEF1, GDS, CaM3 5S, Ubi, H1, and U6 promoter, or a tissue-specific promoter, optionally wherein the tissue-specific promoter is a CaMKIIα promoter, optionally wherein the CaMKIIα promoter comprises a sequence that is at least 70% identical (e.g., at least 75%, 80%, 85%, 90%, 95%, 98%, 99%, or 100% identical) to SEQ ID NO: 146, 149, or 154.

Embodiment 23. The method of embodiment 19-22, wherein the first expression vector comprises a sequence that is at least 70% identical (e.g., at least 75%, 80%, 85%, 90%, 95%, 98%, 99%, or 100% identical) to a sequence provided in SEQ ID NO: 16, SEQ ID NO: 33, SEQ ID NO: 38, SEQ ID NO: 105, SEQ ID NO: 106. SEQ ID NO: 121, or SEQ ID NO: 123, optionally wherein the second expression vector comprises a sequence that is at least 70% identical (e.g., at least 75%, 80%, 85%, 90%, 95%, 98%, 99%, or 100% identical) to a sequence provided in SEQ ID NO: 10, SEQ ID NO: 12, SEQ ID NO: 14, SEQ ID NO: 17, SEQ ID NO: 31, SEQ ID NO: 32, SEQ ID NO: 124, SEQ ID NO: 125, or SEQ ID NO: 126, optionally wherein the second expression vector does not comprise SEQ ID NO: 127.

Embodiment 24. The method of any one of embodiments 2-23, wherein at least one of (i)-(xii) is delivered in a viral vector or is delivered without a viral vector, wherein the viral vector is selected from the group consisting of a lentivirus, a retrovirus, an adenovirus, alphavirus, vaccinia virus, herpes virus, human papilloma virus, and an adeno-associated virus (AAV) vector, optionally wherein delivery without a viral vector comprises administration of a naked nucleic acid, electroporation, use of a nanoparticle, or use of liposomes.

Embodiment 25. The method of any one of embodiments 19-24, wherein the first expression vector is a first viral vector, and the second expression vector is a viral vector, optionally wherein the first and second viral vectors are AAV vectors.

Embodiment 26. The method of any one of embodiments 1-25 wherein at least one engineered nucleic acid comprises an SV40-derived sequence including a sequence that is at least 70% identical (e.g., at least 75%, 80%, 85%, 90%, 95%, 98%, 99%, or 100% identical) to SEQ ID NO: 8 or SEQ ID NO: 143 and/or a hGH pA terminator sequence, optionally wherein the hGH pA terminator sequence is at least 70% identical (e.g., at least 75%, 80%, 85%, 90%, 95%, 98%, 99%, or 100% identical) to SEQ ID NO: 139, 148, 153, 156, or 161.

Embodiment 27. The methods of any one of embodiments 1-26, wherein OCT4, KLF4, or SOX2 is a mammalian protein.

Embodiment 28. The method of any one of embodiments 1-27, wherein the cell or tissue is in a subject, wherein the subject has a condition, is suspected of having a condition, or at risk for a condition, optionally wherein the condition is selected from the group consisting of ocular disease, aging, cancer, musculoskeletal disease, age-related disease, a disease affecting a non-human animal and neurodegenerative disease.

Embodiment 29. The method of any one of embodiments 1-28, wherein the method further comprises regulating: cellular reprogramming, tissue repair, tissue survival, tissue regeneration, tissue growth, tissue function, organ regeneration, organ survival, organ function, disease, or any combination thereof, optionally wherein regulating comprises inducing cellular reprogramming, reversing aging, improving tissue function, improving organ function, tissue repair, tissue survival, tissue regeneration, tissue growth, promoting angiogenesis, treating a disease, reducing scar formation, reducing the appearance of aging, promoting organ regeneration, promoting organ survival, altering the taste and quality of agricultural products derived from animals, treating a disease, or any combination thereof, ex vivo or in vitro and optionally wherein treating a disease comprises inducing expression of OCT4, KLF4, and/or SOX2 prior to the onset of disease or wherein treating a disease a disease comprises inducing expression of OCT4, KLF4, and/or SOX2 after the onset of disease.

Embodiment 30. The method of embodiment 29, wherein the cell or tissue is from nerve tissue.

Embodiment 31. The method of any one of embodiments 1-30, wherein the engineered nucleic acid further comprises a self-cleaving peptide, optionally wherein the self-cleaving peptide is a 2A peptide that is at least 70% identical (e.g., at least 75%, 80%, 85%, 90%, 95%, 98%, 99%, or 100% identical) to SEQ ID NO: 9 or 118.

Embodiment 32. The method of any one of embodiments 1-31, wherein the engineered nucleic acid further comprises inverted terminal repeats (ITRs) flanking the first nucleic acid, the second nucleic acid, the third nucleic acid, or a combination thereof, optionally, wherein the distance between the ITRs is 4.7 kb or less.

Embodiment 33. An expression vector for rejuvenating a cell, a tissue, and/or an organ from the central nervous system comprising:
(i) a first engineered nucleic acid encoding OCT4;
(ii) a second engineered nucleic acid encoding SOX2; and
(iii) a third engineered nucleic acid encoding KLF4;
   in the absence of an engineered nucleic acid capable of expressing c-MYC.

Embodiment 34. The expression vector of embodiment 33, wherein the OCT4 protein comprises a sequence that is at least 70% identical (e.g., at least 75%, 80%, 85%, 90%, 95%, 98%, 99%, or 100% identical) to SEQ ID NO: 2 or SEQ ID NO: 41.

Embodiment 35. The expression vector of any one of embodiments 33-34, wherein the SOX2 protein comprises a sequence that is at least 70% identical (e.g., at least 75%, 80%, 85%, 90%, 95%, 98%, 99%, or 100% identical) to SEQ ID NO: 4 or SEQ ID NO: 43.

Embodiment 36. The expression vector of any one of embodiments 33-35, wherein the KLF4 protein comprises a sequence that is at least 70% identical (e.g., at least 75%, 80%, 85%, 90%, 95%, 98%, 99%, or 100% identical) to SEQ ID NO: 6 or SEQ ID NO: 45.

Embodiment 37. The expression vector of any one of embodiments 33-36, further comprising an inducible promoter operably linked to the first, second, third engineered nucleic acids, or any combination thereof.

Embodiment 38. The expression vector of embodiment 37, wherein an inducing agent is capable of inducing expression of the first, second, third engineered nucleic acids, or any combination thereof from the inducible promoter in the presence of a tetracycline, optionally wherein the tetracycline is doxycycline.

Embodiment 39. The expression vector of embodiment 38, wherein the inducing agent is reverse tetracycline-controlled transactivator (rtTA).

Embodiment 40. The expression vector of embodiment 39, wherein the rtTA is rtTA3, rtTA Advanced, or rtTA2S-M2.

Embodiment 41. The expression vector of embodiment 40, wherein the rtTA3 comprises an amino acid sequence that is at least 70% identical (e.g., at least 75%, 80%, 85%, 90%, 95%, 98%, 99%, or 100% identical) to SEQ ID NO: 11, or the rtTA Advanced comprises an amino acid sequence that is at least 70% identical (e.g., at least 75%, 80%, 85%, 90%, 95%, 98%, 99%, or 100% identical) to SEQ ID NO: 129, or the rtTA2S-M2 comprises an amino acid sequence that is at least 70% identical (e.g., at least 75%, 80%, 85%, 90%, 95%, 98%, 99%, or 100% identical) to SEQ ID NO: 15.

Embodiment 42. The expression vector of any one of embodiments 38-41, wherein the inducing agent is capable of inducing expression of the first, second, third engineered nucleic acids, or any combination thereof from the inducible promoter in the absence of a tetracycline, optionally, wherein the tetracycline is doxycycline.

Embodiment 43. The expression vector of embodiment 42, wherein the inducing agent is a tetracycline-controlled transactivator (ITA).

Embodiment 44. The expression vector of any one of embodiments 37-43, wherein the inducible promoter comprises a tetracycline-responsive element (TRE), optionally, wherein the promoter is a TRE3G promoter comprising a engineered nucleic acid sequence that is at least 70% identical (e.g., at least 75%, 80%, 85%, 90%, 95%, 98%, 99%, or 100% identical) to SEQ ID NO: 7, optionally, wherein the promoter comprises a engineered nucleic acid sequence that is at least 70% identical (e.g., at least 75%, 80%, 85%, 90%, 95%, 98%, 99%, or 100% identical) to SEQ ID NO: 23, and optionally wherein the promoter comprises a sequence that is at least 70% identical (e.g., at least 75%, 80%, 85%, 90%, 95%, 98%, 99%, or 100% identical) to SEQ ID NO: 24.

Embodiment 45. The expression vector of any one of embodiments 33-36, wherein said expression vector(s) comprise a constitutive promoter operably linked to the first, second, third engineered nucleic acids, or a combination thereof.

Embodiment 46. The expression vector of any one of embodiments 33-44, wherein the expression vector comprises the sequence provided in SEQ ID NO: 16.

Embodiment 47. The expression vector of any one of embodiments 33-46, wherein the expression vector is a viral vector, wherein the viral vector is selected from the group consisting of a lentivirus, alphavirus, vaccinia virus, a herpes virus, human papillomavirus, a retrovirus, an adenovirus, and an adeno-associated virus (AAV) vector.

Embodiment 48. The expression vector of any one of embodiments 33-47, wherein at least one engineered nucleic acid comprises an SV40-derived sequence including a sequence that is at least 70% identical (e.g., at least 75%, 80%, 85%, 90%, 95%, 98%, 99%, or 100% identical) to SEQ ID NO: 8 or 143.

Embodiment 49. The expression vectors of any one of embodiments 33-48, wherein OCT4, KLF4, or SOX2 is a mammalian protein.

Embodiment 50. The expression vector of any one of embodiments 33-49, wherein the expression vector further comprises a self-cleaving peptide, optionally wherein the self-cleaving peptide is 2A peptide, optionally wherein the 2A peptide comprises a sequence that is at least 70% identical (e.g., at least 75%, 80%, 85%, 90%, 95%, 98%, 99%, or 100% identical) to SEQ ID NO: 9 or 118.

Embodiment 51. The expression vector of any one of embodiments 37-44 and 46-50, wherein the expression vector comprises one inducible promoter.

Embodiment 52. The expression vector of any one of embodiments 45-50, wherein the expression vector comprises one constitutive promoter.

Embodiment 53. The expression vector of any one of embodiments 33-52, wherein the engineered nucleic acid further comprises inverted terminal repeats (ITRs) flanking the first nucleic acid, the second nucleic acid, the third nucleic acid, or a combination thereof.

Embodiment 54. The expression vector of embodiment 32, wherein the distance between the ITRs is 4.7 kb or less.

Embodiment 55. A recombinant virus comprising the expression vector of any one of embodiments 47-54, optionally wherein the recombinant virus is a retrovirus, an adenovirus, an AAV, alphavirus, vaccinia virus, a herpes virus, human papillomavirus, or a lentivirus, optionally wherein the recombinant virus is AAV.PHP.eB

Embodiment 56. An engineered cell produced by any one of the methods of embodiments 1-32, 63-66, 70-75, 81, and 85-87, optionally wherein the engineered cell comprises the expression vector of any one of embodiments 33-54, optionally wherein the engineered cell is a cell from the central nervous system, optionally wherein the engineered cell is a brain cell, optionally wherein the engineered cell is a nerve cell, optionally wherein the engineered cell is a neuron.

Embodiment 57. A composition comprising the expression vector of any one of embodiments 33-54, the recombinant virus of embodiment 55, the engineered cell of embodiment 56, a chemical agent that is capable of inducing OCT4, KLF4, and/or SOX2 expression, an engineered protein selected from the group consisting of OCT4, KLF4, and/or SOX2, an antibody capable of inducing expression of OCT4, KLF4, and/or SOX2, optionally wherein the composition comprises a pharmaceutically acceptable carrier.

Embodiment 58. The composition of embodiment 57, further comprising a second expression vector encoding an inducing agent, a second protein encoding an inducing agent, or a second recombinant virus encoding an inducing agent, optionally wherein the second expression vector is an AAV vector and/or the second recombinant virus is an AAV.

Embodiment 59. The composition of embodiment 58, wherein the inducing agent is reverse tetracycline transactivator (rtTA) or tetracycline transactivator (tTA).

Embodiment 60. The composition of any one of embodiments 58-59, wherein the inducing agent is encoded by a viral vector, optionally, wherein the viral vector is selected from the group consisting of a lentiviral vector, an adenoviral vector, an adeno-associated viral vector, and a retroviral vector.

Embodiment 61. The composition of embodiment 60, wherein the viral vector encoding the inducing agent comprises a sequence set forth in SEQ ID NO: 31 or SEQ ID NO: 32.

Embodiment 62. A kit comprising the expression vector of any one of embodiments 33-54, recombinant virus of embodiment 55, the engineered cell of embodiment 56, a chemical agent that is capable of inducing OCT4, KLF4, and/or SOX2 expression, an engineered protein selected from the group consisting of OCT4, KLF4, and/or SOX2, an antibody capable of inducing expression of OCT4, KLF4, and/or SOX2, or the composition of any one of embodiments 56-61.

Embodiment 63. A method of producing an engineered cell comprising the method of any one of embodiments 1-32, thereby producing the engineered cell optionally wherein the engineered cell is a cell from the central nervous system, optionally wherein the engineered cell is a brain cell, optionally wherein the engineered cell is a nerve cell, optionally wherein the engineered cell is a neuron.

Embodiment 64. The method of embodiment 63, wherein the engineered cell is an induced pluripotent stem cell-derived neuron.

Embodiment 65. The method of any one of embodiments 63-64, wherein the engineered cell is the cell of embodiment 56.

Embodiment 66. A method of producing an engineered cell, comprising the method of any one of embodiments 1-32 and 63-65, wherein the engineered cell is produced ex vivo.

Embodiment 67. The method of any one of embodiments 63-66, further comprising generating an engineered tissue or engineered organ.

Embodiment 68. The method of any one of embodiments 66-67, further comprising administering the engineered cell, engineered tissue, and/or engineered organ to a subject in need thereof, optionally wherein the cell, tissue, and/or organ is from the central nervous system.

Embodiment 69. The method of any one of embodiments 63-68, wherein the method further comprises treating a disease, optionally wherein the disease is a neurodegenerative diseases a neurological disease, a psychiatric disorder, a chronic disease, a cancer, aging, age-related diseases, and any disease affecting the central nervous system, optionally wherein the disease is a neurodegenerative disease.

Embodiment 70. A method comprising:
(i) activating OCT4;
(ii) activating SOX2; and
(iii) activating KLF4;
   in a cell, tissue, and/or organ from the central nervous system, and in the absence of activating c-Myc, optionally wherein the cell, tissue, and/or organ is within a subject.

Embodiment 71. The method of embodiment 71, wherein the activating in any one of (i)-(iii) comprises administering an antibody, protein, nucleic acid, or chemical agent.

Embodiment 72. The method of any one of embodiments 72, wherein the nucleic acid, antibody, protein, and/or chemical agent replaces OCT4, SOX2, and/or KLF4.

Embodiment 73. The method of embodiment 72, wherein the replacing comprises promoting cellular reprogramming.

Embodiment 74. The method of any one of embodiments 70-73, wherein activating of any one of (i)-(iii) comprises replacing OCT4, SOX2, and/or KLF4, selected from the group consisting of an antibody, a protein, a nucleic acid, and a chemical agent.

Embodiment 75. The method of embodiment 74, wherein the replacing of OCT4, SOX2, and/or KLF4 comprises administering a nucleic acid and/or protein encoding Tet1, NR5A-2, Sall4, E-cadherin, NKX3-1, NANOG, and/or Tet2.

Embodiment 76. The method of any one of embodiments 1-32 and 70-75, wherein the subject is healthy.

Embodiment 77. The method of any one of embodiments 1-32 and 70-76, wherein the subject is a pediatric subject.

Embodiment 78. The method of any one of embodiments 1-32 and 70-76, wherein the subject is an adult subject.

Embodiment 79. The method of any one of embodiments 28-32 and 70-78, wherein the subject has, is suspected of having, or at risk for a neurodegenerative disorder.

Embodiment 80. The method of any one of embodiments 28-32 and 70-79, wherein the subject has, is suspected of having, or at risk for age-related decline in brain function.

Embodiment 81. A method comprising administering a nucleic acid and/or protein encoding Tet1 or Tet2 to a cell, tissue, and/or organ from the central nervous system, optionally wherein the cell, tissue, and/or organ is within a subject.

Embodiment 82. The method of embodiment 81, wherein the subject has a disease.

Embodiment 83. The method of embodiment 82, wherein the disease is selected from acute injuries, neurodegenerative diseases, neurological diseases, chronic diseases, proliferative diseases, genetic diseases, inflammatory diseases, autoimmune diseases, neurological diseases, painful conditions, psychiatric disorders, chronic diseases, cancers, aging, age-related diseases, and diseases affecting any tissue in a subject.

Embodiment 84. The method of embodiment 83, wherein the disease is a neurodegenerative disease.

Embodiment 85. The method of any one of embodiments 1-32 and 63-84, further comprising activating an enhancer of reprogramming in the cell, tissue, and/or organ from the central nervous system, optionally, wherein the cell, tissue, and/or organ is within a subject in need thereof.

Embodiment 86. The method of any one of embodiments 1-32 and 63-85, further comprising inhibiting a barrier of reprogramming in the cell, tissue, organ and/or subject.

Embodiment 87. The method of embodiment 86, wherein the barrier of reprogramming is a DNA methyltransferase (DNMT) in the cell, tissue, organ and/or subject. Embodiment 88. A method comprising:
inducing in a subject with a neurological disorder:
   (i) OCT4 expression;
   (ii) SOX2 expression; and
   (iii) KLF4 expression;
in the absence of inducing c-MYC expression, wherein the subject has been treated with a chemotherapy drug.

Embodiment 89. The method of embodiment 89, wherein the chemotherapy drug is vincristine (VCS).

Embodiment 90. A method comprising inducing in a cell, tissue, and/or organ from the central nervous system, optionally wherein the cell, tissue, and/or organ is within a subject:
(i) OCT4 expression;
(ii) SOX2 expression; and
(iii) KLF4 expression;
   wherein OCT4, SOX2, and KLF4 is encoded by a nucleic acid and expression of OCT4, SOX2, and/or KLF4 is induced from a single promoter.

Embodiment 91. A method comprising:
inducing in a cell, tissue, and/or organ from the central nervous system, optionally wherein the cell, tissue, and/or organ is within a subject:
   (i) OCT4 expression;
   (ii) SOX2 expression;
   (iii) KLF4 expression; or
   (iv) any combination of (i)-(iii),
in the absence of inducing c-MYC expression.

Embodiment 92. The method of embodiment 91, wherein the combination of (i)-(iii) comprises (i) and (ii); (i) and (iii); (ii) and (iii); or (i), (ii), and (iii).
(40) Embodiment 93. An expression vector comprising (i) a first engineered nucleic acid encoding OCT4;
(ii) a second engineered nucleic acid encoding SOX2;
(iii) a third engineered nucleic acid encoding KLF4; or
(iv) any combination of (i)-(iii),
   in the absence of an engineered nucleic acid capable of inducing c-MYC expression, optionally wherein one or more of (i)-(iii) is operably linked to a CaMKIIα promoter, optionally wherein the CaMKIIα promoter comprises a sequence that is at least 70% identical (e.g., at least 75%, 80%, 85%, 90%, 95%, 98%, 99%, or 100% identical) to SEQ ID NO: 146, 149, or 154.

Embodiment 94. The expression vector of embodiment 93, wherein the combination of (i)-(iii) comprises (i) and (ii); (i) and (iii); (ii) and (iii); or (i), (ii), and (iii).

Embodiment 95. A recombinant virus comprising the expression vector of any one of embodiments 47-54 and 93-94, optionally wherein the recombinant virus is a retrovirus, an adenovirus, an AAV, alphavirus, vaccinia virus, a herpes virus, human papillomavirus, or a lentivirus.

Embodiment 96. An engineered cell produced by any one of the methods of embodiments 1-32, 63-66, 70-75, 81, 85-87, and 91-92, optionally wherein the engineered cell comprises the expression vector of any one of embodiments 33-54 and 93-94.

Embodiment 97. A composition comprising the expression vector of any one of embodiments 33-54 and 93-94, the recombinant virus of embodiment 55 or embodiment 95, the engineered cell of embodiment 56 or 96, a chemical agent that is capable of inducing expression of OCT4; KLF4; SOX2; or any combination thereof, an engineered protein selected from the group consisting of OCT4; KLF4; SOX2; or any combination thereof, an antibody capable of inducing expression of OCT4; KLF4; SOX2; or any combination thereof, optionally wherein the composition comprises a pharmaceutically acceptable carrier.

Embodiment 98. A kit comprising the expression vector of any one of embodiments 33-54 and 93-94, recombinant virus of embodiment 55 or 95, the engineered cell of embodiment 56 or 96, a chemical agent that is capable of inducing expression of OCT4; KLF4; SOX2; or any combination thereof, an engineered protein selected from the group consisting of OCT4; KLF4; SOX2; or any combination thereof, an antibody capable of inducing expression of OCT4; KLF4; SOX2; or any combination thereof, or the composition of any one of embodiments 56-61 or 97.

Embodiment 99. A method of producing an engineered cell comprising the method of any one of embodiments 1-32 and 91-92, thereby producing the engineered cell.

Embodiment 100. A method of producing an engineered cell, comprising the method of any one of embodiments 1-32, 63-65, 91-92, and 99, wherein the engineered cell is produced in vivo.

Embodiment 101. A method of producing an engineered cell, comprising the method of any one of embodiments 1-32, 63-65, 91-92, and 99, wherein the engineered cell is produced ex vivo.

Embodiment 102. A method comprising:
(i) activating OCT4;
(ii) activating SOX2;
(iii) activating KLF4; or
(iv) any combination of (i)-(iii),
   in a cell, tissue, and/or organ from the central nervous system, optionally wherein the cell, tissue, and/or organ is within a subject and in the absence of activating c-Myc above endogenous levels.

Embodiment 103. The method of embodiment 102, wherein the combination of (i)-(iii) comprises (i) and (ii); (i) and (iii); (ii) and (iii); or (i), (ii), and (iii).

Embodiment 104. A method comprising:
inducing in the central nervous system of a subject:
   (i) OCT4 expression;
   (ii) SOX2 expression;
   (iii) KLF4 expression; or
   (iv) any combination of (i)-(iii),
in the absence of inducing c-MYC expression, wherein the subject has been treated with a chemotherapy drug.

Embodiment 105. The method of embodiment 104, wherein the combination of (i)-(iii) comprises (i) and (ii); (i) and (iii); (ii) and (iii); or (i), (ii), and (iii).

Embodiment 106. A method comprising inducing in a cell, tissue, and/or organ from the central nervous system, optionally wherein the cell, tissue, and/or organ is with a subject:
(i) OCT4 expression;
(ii) SOX2 expression;
(iii) KLF4 expression; or
(iv) any combination of (i)-(iii),
   wherein OCT4, SOX2, KLF4, or any combination thereof is encoded by a nucleic acid and expression of OCT4, SOX2, KLF4, or any combination thereof is induced from a single promoter.

Embodiment 107. The method of embodiment 106, wherein the combination of (i)-(iii) comprises (i) and (ii); (i) and (iii); (ii) and (iii); or (i), (ii), and (iii).

Embodiment 108. The method of any one of embodiments 1-32, 68-92, or 102-107 wherein the subject is a human.

Embodiment 109. The method of any one of embodiments 1-32, 68-92, or 102-108, wherein the method does not induce teratoma formation.

Embodiment 110. The method of any one of embodiments 1-32, 68-92, or 102-109, wherein the method does not induce tumor formation or tumor growth.

Embodiment 111. The method of embodiment 110, wherein the method reduces tumor formation or tumor growth.

Embodiment 112. The method of any one of embodiments 1-32, 68-92, or 102-111, wherein the method increases cognitive function in the subject.

Embodiment 113. The method of any one of embodiments 1-32, 68-92, or 102-112, wherein the method does not induce cancer.

Embodiment 114. The method of any one of embodiments 1-32, 68-92, or 102-113, wherein the method does not induce glaucoma brain tumor.

Embodiment 115. The method of any one of embodiments 1-32, 68-92, or 102-114, wherein the method reverses the epigenetic clock of the cell, the tissue, the organ, the subject, or any combination thereof.

Embodiment 116. The method of embodiment 115, wherein the epigenetic clock is determined using a DNA methylation-based (DNAm) age estimator.

Embodiment 117. The method of any one of embodiments 1-32, 68-92, or 102-116, wherein the method alters the expression of one or more genes associated with ageing.

Embodiment 118. The method of embodiment 117, wherein the method reduces expression of one or more genes associated with ageing.

Embodiment 119. The method of embodiment 118, wherein the one or more genes associated with ageing is a central nervous system gene.

Embodiment 120. The method of embodiment 118, wherein the one or more genes associated with ageing is a brain gene.

Embodiment 121. The method of embodiment 117, wherein the method increases expression of one or more genes associated with ageing.

Embodiment 122. The method of embodiment 121, where the one or more genes associated with ageing is a central nervous system gene.

Embodiment 123. The method of embodiment 121, wherein the one or more genes associated with ageing is a brain gene.

Embodiment 124. The method of any one of embodiments 118-123, wherein the one or more genes is selected from the group consisting of RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

Embodiment 125. A method of reprogramming comprising rejuvenating the epigenetic clock of a cell, tissue, and/or organ of the central nervous system, optionally wherein the cell, tissue, and/or organ is within a subject.

Embodiment 126. The method of embodiment 125, wherein rejuvenating the epigenetic clock of a cell, tissue, organ, subject, or any combination thereof comprises introducing, activating, and/or expressing OCT4, KLF4, SOX2, or any combination thereof.

Embodiment 127. The method of any one of embodiments 126, wherein the epigenetic clock of a cell, tissue, organ, subject, or any combination thereof is rejuvenated to that of a young cell, tissue, organ, subject, or any combination thereof.

Embodiment 128. The method of any one of embodiments 125-127, wherein rejuvenating the epigenetic clock comprises altering expression of one or more genes associated with ageing in the cell, tissue, organ, subject, or the combination thereof.

Embodiment 129. The method of embodiment 128, wherein the method comprises reducing expression of one or more genes associated with ageing.

Embodiment 130. The method of embodiment 129, wherein the one or more genes associated with ageing is a central nervous system gene.

Embodiment 131. The method of embodiment 129, wherein the one or more genes associated with ageing is a brain gene.

Embodiment 132. The method of embodiment 128, wherein the method increases expression of one or more genes associated with ageing.

Embodiment 133. The method of embodiment 132, where the one or more genes associated with ageing is a central nervous system gene.

Embodiment 134. The method of embodiment 132, wherein the one or more genes associated with ageing is a brain gene.

Embodiment 135. The method of any one of embodiments 128-134, wherein the one or more genes is selected from the group consisting of RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

Embodiment 136. A method of reprogramming comprising altering the expression of one or more genes associated with ageing of the central nervous system.

Embodiment 137. The method of embodiment 136, comprising increasing expression of OCT4, KLF4, SOX2, or any combination thereof.

Embodiment 138. The method of any one of embodiments 136-137, wherein the method rejuvenates the epigenetic clock of a cell, tissue, organ, subject, or any combination thereof.

Embodiment 139. The method of any one of embodiments embodiment 136-138, wherein the method comprises reducing expression of one or more genes associated with ageing.

Embodiment 140. The method of embodiment 139, wherein the one or more genes associated with ageing is a central nervous system gene.

Embodiment 141. The method of embodiment 139, wherein the one or more genes associated with ageing is a brain gene.

Embodiment 142. The method of embodiment 138, wherein the method increases expression of one or more genes associated with ageing.

Embodiment 143. The method of embodiment 142, where the one or more genes associated with ageing is a central nervous system gene.

Embodiment 144. The method of embodiment 142, wherein the one or more genes associated with ageing is a brain gene.

Embodiment 145. The method of any one of embodiments 139-144, wherein the one or more genes is selected from the group consisting of RE1 Silencing Transcription Factor (REST), (Tumor Protein P73) TP73, Glial Fibrillary Acidic Protein (GFAP), Intercellular Adhesion Molecule 2 (ICAM2), and Thioredoxin Interacting Protein (TXNIP).

Embodiment 146. A method comprising resetting the transcriptional profile of old cells from the central nervous system in vitro.

Embodiment 147. A method comprising resetting the transcriptional profile of old cells in the central nervous system of a subject.

Embodiment 148. A method comprising inducing in the central nervous system of a subject:
(i) OCT4 expression;
(ii) SOX2 expression; and/or
(iii) KLF4 expression;
   in the absence of inducing c-MYC expression, wherein the subject has, is at risk for, or is suspected of having a condition that increases the DNA methylation-based age of a cell, of a tissue, and/or of an organ within the subject, as compared to a control cell, a control tissue, and/or of a control organ of a control subject that does not have the condition.

Embodiment 149. The method of embodiment 148, wherein the method reduces the DNA methylation-based age of the cell, the tissue, the organ, and/or the subject.

Embodiment 150. A method of transdifferentiation comprising inducing in one type of cell:
(i) OCT4 expression;
(ii) SOX2 expression;
(iii) KLF4 expression; and
(iv) expression of a lineage determining factor,
   wherein (i)-(iii) are expressed from a single vector, thereby transdifferentiating the cell into a cell of the central nervous system.

Embodiment 151. A method of transdifferentiation into a cell of the central nervous system comprising inducing in a cell:
(i) OCT4 expression;
(ii) SOX2 expression; and
(iii) KLF4 expression; and
   reducing expression of a lineage determining factor, wherein (i)-(iii) are expressed from a single vector.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A composition for use in rejuvenating a cell, tissue, or organ comprising:
a) an agent that induces OCT4 expression;
b) an agent that induce SOX2 expression; and
c) an agent that induces KLF4 expression,
wherein the composition does not comprise an agent that induces c-MYC expression, wherein the cell, tissue, or organ is a central nervous system cell, central nervous system tissue, or central nervous system organ, optionally wherein the central nervous system does not include the retina, optionally wherein the cell, tissue, or organ is a brain cell, brain tissue, or brain.

2. A method of rejuvenating a cell, tissue, and/or organ, comprising administering to the cell, tissue, or organ a composition comprising:
a) an agent that induces OCT4 expression;
b) an agent that induce SOX2 expression; and
c) an agent that induces KLF4 expression,
wherein the composition does not comprise an agent that induces c-MYC expression, wherein the cell, tissue, or organ is a central nervous system cell, central nervous system tissue, or central nervous system organ, optionally wherein the central nervous system does not include the retina, optionally wherein the cell, tissue, or organ is a brain cell, brain tissue, or brain.

3. The composition or method of claim 1 or 2, wherein the brain cell is a neuron or glial cell.

4. The composition or method of claim 3, wherein the neuron is an excitatory neuron.

5. The composition or method of any one of claims 1-4, wherein the brain tissue is nervous tissue.

6. The composition or method of any one of claims 1-5, wherein the cell, tissue, or organ is in a subject, optionally wherein the composition is administered to a subject in need thereof, optionally wherein the subject has a neurological disorder.

7. The composition or method of any one of claims 1-6, wherein the composition or does not induce OCT4, SOX2, or KLF4 expression in the retina.

8. The composition or method of any one of claims 1-7, wherein the composition induces the expression of OCT4, SOX2, and KLF4 for a time period that is sufficient to rejuvenate the cell, tissue, and/or organ.

9. The composition or method of any one of claims 1-8, wherein the time period sufficient to rejuvenate the cell, tissue, or organ is approximately one month.

10. The composition or method of any one of claims 1-9, wherein the expression of OCT4, SOX2, and KLF4 is induced for less than two months.

11. The composition or method of any one of claims 1-10, wherein the expression of OCT4, SOX2, and KLF4 is induced for at most one month.

12. The composition or method of any one of claims 1-11, wherein rejuvenating the cell, tissue, or organ comprises restoring epigenetic information in the cell, tissue, and/or organ.

13. The composition or method of any one of claims 1-12, wherein rejuvenating the cell, tissue, or organ comprises restoring epigenetic information lost due to aging, injury, disease, or any combination thereof in the cell, tissue, or organ.

14. The composition or method of any one of claims 1-13, wherein rejuvenating the cell, tissue, or organ comprises reestablishing the epigenetic status of the cell, tissue, or organ an epigenetic status that is similar to the status formed soon after fertilization or final differentiation.

15. The composition or method of any one of claims 1-14, wherein each agent is independently a nucleic acid, a small molecule, or a polypeptide, optionally wherein the polypeptide is an antibody.

16. The composition or method of any one of claims 1-15, wherein at least one agent comprises a nanoparticle.

17. The composition or method of any one of claims 15-16, wherein at least one agent is encapsulated in at least one nanoparticle.

18. The composition or method of any one of claims 15-17, wherein the nucleic acid is DNA or RNA.

19. The composition or method of claim 18, wherein the DNA is plasmid DNA.

20. The composition or method of claim 18, wherein the RNA is mRNA.

21. The composition or method of any one of claims 15-20, wherein the agent that induces OCT4 expression is an engineered nucleic acid encoding OCT4.

22. The composition or method of any one of claims 15-21, wherein the agent that induces SOX2 expression is an engineered nucleic acid encoding SOX2.

23. The composition or method of any one of claims 15-22, wherein the agent that induces KLF4 expression is an engineered nucleic acid encoding KLF4.

24. The composition or method of any one of claims 15-20, wherein the agent that induces OCT4 expression is an engineered nucleic acid encoding OCT4, the agent that induces SOX2 expression is an engineered nucleic acid encoding SOX2, and the agent that induces KLF4 expression is an engineered nucleic acid encoding KLF4.

25. The composition or method of claim 21, 22, or 23, wherein the engineered nucleic acids are present on one or more expression vectors.

26. The composition or method of claim 25, wherein the engineered nucleic acids are present on the same expression vector.

27. The composition or method of any one of claim 25 or 26, wherein the one or more expression vectors include an inducible promoter operably linked to any one of the engineered nucleic acids or a combination thereof.

28. The composition or method of claim 27, wherein the promoter is a TRE3G, a TRE2 promoter, or a P tight promoter.

29. The composition or method of claim 27 or claim 28, wherein said promoter comprises a tetracycline response element (TRE).

30. The composition or method of any one of claims 25-29, wherein the expression vector comprises a hGH pA terminator sequence, optionally wherein the hGH pA terminator sequence comprises a sequence that is at least 70% identical to SEQ ID NO: 139, 148, 153, 156, or 161.

31. The composition or method of any one of claims 25-30, wherein the expression vector comprises a WPRE sequence.

32. The composition or method of any one of claims 25-31, wherein the expression vector comprises a self-cleaving peptide.

33. The composition or method of claim 32, wherein the self-cleaving peptide is a 2A peptide, optionally wherein the 2A peptide sequence comprises a sequence that is at least 70% identical to SEQ ID NO: 118 and/or is encoded by a nucleic acid comprising a sequence that is at least 70% identical to SEQ ID NO: 144.

34. The composition or method of any one of claims 25-33, wherein the expression vector comprises inverted terminal repeats (ITRs) flanking the first nucleic acid, the second nucleic acid, the third nucleic acid, or a combination thereof, and wherein the distance between the ITRs is 4.7 kb or less.

35. The composition or method of any one of the preceding claims, wherein the composition further comprises an inducing agent, or wherein the method further comprises administering to said subject an inducing agent.

36. The composition or method of claim 35, wherein the inducing agent comprises a tetracycline, a tetracycline transactivator (tTA), and/or a reverse tetracycline-controlled transactivator (rtTA), optionally wherein the tTA comprises a sequence that is at least 70% identical to 138 or 159, optionally wherein the tTA is encoded by a sequence that is at least 70% identical to 137 or 158, optionally wherein the rtTA comprises a sequence that is at least 70% identical to SEQ ID NO: 11, 129, 13, or 15, optionally wherein the rtTA is encoded by a sequence that is at least 70% identical to SEQ ID NO: 10, 12, 14, or 128.

37. The composition or method of claim 36, wherein the tetracycline is doxycycline.

38. The composition or method of claim 36 or claim 37, wherein the composition comprises an expression vector with an engineered nucleic acid that encodes the tTA and/or rtTA, optionally wherein the engineered nucleic acid that encodes the tTA and/or rtTA comprises a WPRE sequence and/or an hGH pA sequence, optionally wherein the WPRE sequence is at least 70% identical to SEQ ID NO: 21, 135, 147, 152, 155, or 160 and/or the hGH pA sequence is at least 70% identical to SEQ ID NO: 139, 148, 153, 156, or 161.

39. The composition or method of claim 38, wherein the expression vector encoding the rTA and/or rtTA is the same expression vector or is a different expression vector as the engineered nucleic acids encoding OCT4, SOX2, and/or KLF4.

40. The composition or method of any one of claims 36-39, wherein the rtTA is rtTA3, rtTA Advanced, rtTA2S-M2, or rtTA4, optionally wherein the rtTA comprises a sequence that is at least 70% identical to a sequence selected from the group consisting of SEQ ID NOs: 11, 13, 15, and 129.

41. The composition or method of any one of claims 25-40, wherein at least one expression vector is a viral vector, optionally wherein at least one expression vector is packaged in a recombinant virus.

42. The composition or method of claim 41, wherein the viral vector is a lentivirus, a retrovirus, an adenovirus, alphavirus, vaccinia virus, human papillomavirus, or an adeno-associated virus (AAV) vector.

43. The composition or method of claim 41 or claim 42, wherein the AAV vector is packaged in AAV-PHP.eB, AAV-PHP.b, AAV.CAP-B10, or AAV.CAP-B22 virus.

44. The composition or method of any one of the preceding claims, wherein the AAV vector is not AAV2 or AAV9.

45. The composition or method of any one of the preceding claims, wherein the subject is a human or non-human mammal.

46. The composition or method of any one of claims 38-45, wherein the expression vector with the engineered nucleic acid that encodes the tTA and/or rtTA comprises a promoter operably linked to the nucleic acid that encodes the tTA and/or rtTA.

47. The composition or method of claim 46, wherein the promoter operably linked to the engineered nucleic acid that encodes the tTa or rtTA is a ubiquitous promoter.

48. The composition or method of claim 47, wherein the ubiquitous promoter is UBC, CMV, PGK1, CAG, optionally wherein the ubiquitous promoter comprises a sequence that is at least 70% identical to 48, 132, 130, 136, or 162.

49. The composition or method of claim 46, wherein the promoter operably linked to the engineered nucleic acid that encodes the tTa or rtTA is a neuron-specific promoter.

50. The composition or method of claim 49, wherein the neuron-specific promoter is CaMKIIα, optionally wherein the CaMKIIα promoter comprises a sequence that is at least 70% identical to SEQ ID NO: 146, 149, or 154.

51. The composition or method of any one of claims 46-50, wherein the promoter operably linked to the engineered nucleic acid that encodes tTA and/or rtTA is not a Synapsin-I promoter, is not a CaMKII-gamma promoter, or a combination thereof, optionally wherein the Synapsin-I promoter comprises a sequence that is at least 70% identical to SEQ ID NO: 157.

52. The composition or method of any one of the preceding claims, wherein the composition is administered through retro-orbital venous injection.

53. The composition or method of any one of claims 1-51, wherein the composition is administered via intrathecal administration.

54. The composition or method of any one of claims 1-51, wherein the composition is systemically administered, optionally wherein the systemic injection is intravenous injection.

55. The composition or method of any one of the preceding claims, wherein the composition is not administered to the retina of the subject.

56. The composition or method of any one of the preceding claims, wherein the composition is used to improve cognitive function of the subject.

57. The composition or method of any one of the preceding claims, wherein the composition is used to improve the memory of the subject.

58. The composition or method of any one of the preceding claims, wherein the composition does not comprise a nucleic acid with a Synapsin-I promoter, does not comprise a CaMKII-gamma promoter, or a combination thereof, optionally wherein the Synapsin-I promoter comprises a sequence that is at least 70% identical to SEQ ID NO: 157.

59. The composition or method of any one of the preceding claims, wherein the composition is administered to a subject who has or is suspected of having a neurological disorder.

60. The composition or method of claim 59, wherein the neurological disorder is a neurodegenerative disorder.

61. The composition or method of claim 60, wherein the neurodegenerative disorder is Alzheimer's Disease, Parkinson's Disease, dementia, Friedreich ataxia, amyotrophic lateral sclerosis, or vascular dementia.

62. The composition or method of any preceding claim, wherein the composition is a pharmaceutical composition.

63. The composition or method of any preceding claim, wherein the composition comprises:
(a) an viral vector comprising a nucleic acid encoding a tetracycline-controlled transactivator (tTA) or reverse tetracycline-controlled transactivator (rtTA), wherein the nucleic acid encoding the tTA or rtTA is operably linked to a CaMKIIα promoter, optionally wherein the viral vector is an AAV vector, optionally wherein the AAV vector is packaged in AAV-PHP.eB virus; and
(b) an viral vector comprising a first nucleic acid encoding OCT4, a second nucleic acid encoding SOX2, and a third nucleic acid encoding KLF4, wherein the first, second, and third nucleic acids are operably linked to a promoter comprising a tetracycline response element (TRE), optionally wherein the viral vector is an AAV vector, optionally wherein the AAV vector is packaged in AAV-PHP.eB virus.

64. The composition or method of claim 63, wherein the vector in (a) comprises a WPRE sequence and/or a hGH pA terminator sequence, optionally wherein the WPRE sequence comprises a sequence that is at least 70% identical to SEQ ID NO: 21, 135, 147, 152, 155, or 160 and/or wherein the hGH pA terminator sequence comprises a sequence that is at least 70% identical to SEQ ID NO: 139, 148, 153, 156, or 161.

65. The composition or method of claim 63, wherein the viral vector in (a) is the same viral vector as in (b).

66. The composition or method of any preceding claim, wherein the composition comprises a sequence that is at least 70% identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163.

67. The composition or method of any one of claims 1-29, 32-33, 35-42, 45-48, 51-62, and 65, wherein the composition comprises a sequence that is at least 70% identical to SEQ ID NO: 123.

68. The composition or method of any one of claims 1-66, wherein the composition comprises a sequence that is at least 70% identical to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126, optionally wherein the composition does not comprise a sequence that is at least 70% identical to SEQ ID NO: 127.

69. The composition or method of any one of claims 63-65, wherein the viral vector in part (a) comprises a sequence that is at least 70% identical to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126.

70. The composition or method of any one of claims 63-65 and 69, wherein the viral vector in part (b) comprises a sequence that is at least 70% identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163.

71. The composition or method of any preceding claim, wherein the composition further comprises a pharmaceutically acceptable carrier.

72. An expression vector comprising a sequence that is at least 70% identical to a sequence selected from SEQ ID NOs: 123-127.

73. A recombinant virus comprising the expression vector of claim 72.

74. An engineered cell, tissue, or organ of a central nervous system comprising the composition of any one of claims 1, 3-51, 58, and 62-71, the expression vector of claim 72, or the recombinant virus of claim 73.

75. A kit comprising:
(a) a container housing the composition of any one of claims 1, 3-51, 58 and 62-68, the expression vector of claim 72, or the recombinant virus of claim 73, and
(b) instructions for rejuvenating a cell, a tissue, or an organ of a central nervous system, optionally instructions for rejuvenating the cell, tissue, or organ of a subject in need thereof, optionally wherein the cell, tissue, or organ from the central nervous system is a brain cell, brain tissue, or brain.

76. A kit comprising:
(a) a first container housing a viral vector comprising a nucleic acid encoding a tetracycline-controlled transactivator (tTA) or a reverse tetracycline-controlled transactivator (rtTA), wherein the nucleic acid encoding the tTA or rtTA is operably linked to a CaMKIIα promoter;
(b) a second container housing a viral vector comprising a first nucleic acid encoding OCT4, a second nucleic acid encoding SOX2, and a third nucleic acid encoding KLF4, wherein the first, second, and third nucleic acids are operably linked to a promoter comprising a tetracycline response element (TRE), and
(c) instructions for rejuvenating a cell, tissue, or organ, optionally instructions for rejuvenating the cell, tissue, or organ of a subject in need thereof, optionally wherein the viral vector in (a) and/or (b) is an AAV vector, optionally wherein the AAV vector is packaged in AAV-PHP.b virus, optionally wherein the AAV-PHP.b virus is AAV.PHP.eB virus, optionally wherein the cell, tissue, or organ from the central nervous system is a brain cell, brain tissue, or brain.

77. The kit of claim 76, wherein the viral vector in part (a) comprises a sequence that is at least 70% identical to SEQ ID NO: 10, SEQ ID NO: 12, SEQ ID NO: 14, SEQ ID NO: 137, SEQ ID NO: 158, SEQ ID NO: 17, SEQ ID NO: 31, SEQ ID NO: 32, SEQ ID NO: 123, SEQ ID NO: 124, SEQ ID NO: 125, SEQ ID NO: 146, 149, or 154, SEQ ID NO: 135, 147, 152, 155, or 160, SEQ ID NO: 139, 148, 153, 156, or 161, or SEQ ID NO: 126.

78. The kit of claim 76 or 77 wherein the viral vector in part (b) comprises a sequence that is at least 70% identical to SEQ ID NO: 16, SEQ ID NO: 33, SEQ ID NO: 38, SEQ ID NO: 40, SEQ ID NO: 42, SEQ ID NO: 44, SEQ ID NO: 105, SEQ ID NO: 106, SEQ ID NO: 121, SEQ ID NO: 123, or SEQ ID NO: 163.

79. The composition, method, expression vector, recombinant virus, engineered cell, tissue, or organ, or kit of any one of claims 1-78, wherein the KLF4 comprises a sequence that is at least 70% identical to SEQ ID NO: 6 or SEQ ID NO: 45.

80. The composition, method, expression vector, recombinant virus, engineered cell, tissue, or organ, or kit of any one of claims 1-79, wherein the SOX2 comprises a sequence that is at least 70% identical to SEQ ID NO: 4 or SEQ ID NO: 43.

81. The composition, method, expression vector, recombinant virus, engineered cell, tissue, or organ, or kit of any one of claims 1-60, wherein the OCT4 comprises a sequence that is at least 70% identical to SEQ ID NO: 2 or SEQ ID NO: 41.

82. The composition, method, expression vector, recombinant virus, engineered cell, tissue, or organ, or kit of any one of claims 1-81, wherein the composition, expression vector, recombinant virus, engineered cell, tissue, or organ or kit comprises a nucleic acid with:
(a) a nucleic acid sequence that encodes OCT4, KLF4, and SOX2 operably linked to a TRE promoter; and
(b) a nucleic acid sequence that encodes rtTA operably linked to a UbC promoter.

83. The composition, method, expression vector, recombinant virus, engineered cell, tissue, or organ, or kit of claim 82, wherein the nucleic acid sequence that encodes OCT4, KLF4, and SOX2 further encodes a 2A peptide.

84. The composition, method, expression vector, recombinant virus, engineered cell, tissue, or organ, or kit of claim 82 or 83, wherein nucleic acid with (a) and (b) further encodes a neomycin resistance gene and/or comprises a WPRE sequence.

85. The composition, method, expression vector, recombinant virus, engineered cell, tissue, or organ, or kit of claim 84, wherein the neomycin resistance gene is operably linked to a PGK promoter.

86. The composition, method, expression vector, recombinant virus, engineered cell, tissue, or organ, or kit of any one of claims 81-85, wherein the nucleic acid with (a) and (b) encodes at least one protein sequence that is at least 70% identical to a sequence selected from:
(a) rtTA Advanced (SEQ ID NO: 129);
(b) human OCT4 (SEQ ID NO: 41);
(c) P2A (SEQ ID NO: 118);
(d) human SOX2 (SEQ ID NO: 43);
(e) T2A (SEQ ID NO: 9);
(f) human KLF4 (SEQ ID NO: 45); and
(g) neomycin resistance gene (SEQ ID NO: 134).

87. The composition, method, expression vector, recombinant virus, engineered cell, tissue, or organ, or kit of any one of claims 81-86, wherein the nucleic acid with (a) and (b) comprises at least one sequence that is at least 70% identical to a sequence selected from:
(a) rtTA Advanced in reverse complement (SEQ ID NO: 128);
(b) UbC promoter in reverse complement (SEQ ID NO: 130);
(c) P tight TRE promoter (SEQ ID NO: 24);
(d) human OCT4 (SEQ ID NO: 40);
(e) P2A (SEQ ID NO: 119);
(f) human SOX2 (SEQ ID NO: 42);
(g) T2A (SEQ ID NO: 120);
(h) human KLF4 (SEQ ID NO: 131);
(i) PGK promoter (SEQ ID NO: 132);
(j) Neomycin resistance gene (SEQ ID NO: 133); and
(k) WPRE (SEQ ID NO: 135).

88. The composition, method, expression vector, recombinant virus, engineered cell, tissue, or organ, or kit of any one of claims 81-86, wherein the nucleic acid with (a) and (b) comprises a sequence that is at least 70% identical to pLVX-rtTA-hOSK-all-in-one (human) (SEQ ID NO: 123).

89. The composition, method, expression vector, recombinant virus, engineered cell, tissue, or organ, or kit of any one of claims 1-81, wherein the composition, expression vector, recombinant virus, engineered cell, tissue, or organ or kit comprises a nucleic acid encoding a tTA, wherein the tTA comprises a sequence that is at least 70% identical to SEQ ID NO: 138.

90. The composition, method, expression vector, recombinant virus, engineered cell, tissue, or organ, or kit of claim 89, wherein the nucleic acid encoding the tTA is at least 70% identical to SEQ ID NO: 137.

91. The composition, method, expression vector, recombinant virus, engineered cell, tissue, or organ, or kit of claim 88 or 89, wherein the nucleic acid encoding the tTA comprises a hGH pA sequence and/or a CMV promoter.

92. The composition, method, expression vector, recombinant virus, engineered cell, tissue, or organ, or kit of claim 91, wherein the hGH pA sequence is at least 70% identical to SEQ ID NO: 139.

93. The composition, method, expression vector, recombinant virus, engineered cell, tissue, or organ, or kit of claim 91 or 92, wherein the CMV promoter is at least 70% identical to SEQ ID NO: 136.

94. The composition, method, expression vector, recombinant virus, engineered cell, tissue, or organ, or kit of any one of claims 89-93, wherein the nucleic acid encoding a tTA comprises a sequence that is at least 70% identical to pAAV-CMV-tTA (Advanced) (SEQ ID NO: 32).

95. The composition, method, expression vector, recombinant virus, engineered cell, tissue, or organ, or kit of any one of claims 1-81 and 89-94, wherein the composition, expression vector, recombinant virus, engineered cell, tissue, or organ or kit comprises a nucleic acid encoding OCT4, SOX2, and KLF4 operably linked to a TRE promoter.

96. The composition, method, expression vector, recombinant virus, engineered cell, tissue, or organ, or kit of claim 95, wherein the nucleic acid sequence encoding OCT4, SOX2, and KLF4 operably linked to a TRE promoter further encodes a 2A peptide and/or SV40 pA.

97. The composition, method, expression vector, recombinant virus, engineered cell, tissue, or organ, or kit of claim 96, wherein the 2A peptide comprises a sequence that is at least 70% identical to T2A (SEQ ID NO: 9) or P2A (SEQ ID NO: 118).

98. The composition, method, expression vector, recombinant virus, engineered cell, tissue, or organ, or kit of claim 97, wherein the nucleic acid sequence encoding OCT4, SOX2, and KLF4 encodes at least one sequence that is 70% identical to a sequence selected from:
(a) mouse OCT4 (SEQ ID NO: 2);
(b) human OCT4 (SEQ ID NO: 40);
(c) mouse SOX2 (SEQ ID NO: 4);
(d) human SOX2 (SEQ ID NO: 42);
(e) human KLF4 (SEQ ID NO: 131); and
(f) mouse KLF4 (SEQ ID NO: 6).

99. The composition, method, expression vector, recombinant virus, engineered cell, tissue, or organ, or kit of any one of claims 95-98, wherein the TRE promoter operably linked to the nucleic acid sequence encoding OCT4, SOX2, and KLF4 is at least 70% identical to SEQ ID NO: 7.

100. The composition, method, expression vector, recombinant virus, engineered cell, tissue, or organ, or kit of any one of claims 95-99, wherein the nucleic acid sequence encoding OCT4, SOX2, and KLF4 operably linked to the TRE promoter comprises at least one sequence that is 70% identical to a sequence selected from:
(a) TRE3G (SEQ ID NO: 7):
(b) mouse Oct4 (SEQ ID NO: 1):
(c) P2A (SEQ ID NO: 144);
(d) mouse Klf4 (SEQ ID NO: 145);
(e) SV40 pA (SEQ ID NO: 143);
(f) mouse Sox2 (SEQ ID NO: 3); and
(g) T2A (SEQ ID NO: 120),
optionally wherein the sequence is at least 70% identical to pAAV-TRE3G-OSK (mouse) SEQ ID NO: 16.

101. The composition, method, expression vector, recombinant virus, engineered cell, tissue, or organ, or kit of any one of claims 1-81 and 89-100, wherein the composition, expression vector, recombinant virus, engineered cell, tissue, or organ or kit comprises a nucleic acid encoding an inducing agent operably linked to a CaMKIIα promoter.

102. The composition, method, expression vector, recombinant virus, engineered cell, tissue, or organ, or kit of claim 101, wherein the inducing agent is a tTA or rtTA.

103. The composition, method, expression vector, recombinant virus, engineered cell, tissue, or organ, or kit of claim 102, wherein the inducing agent comprises a sequence that is at least 70% identical to tTA Advanced (SEQ ID NO: 138), rtTA2S-M2 (SEQ ID NO: 15), or rtTA3 (SEQ ID NO: 11).

104. The composition, method, expression vector, recombinant virus, engineered cell, tissue, or organ, or kit of any one of claims 101-103, wherein the CaMKIIα promoter comprises a sequence that is at least 70% identical to SEQ ID NO: 146, SEQ ID NO: 149, or SEQ ID NO: 154.

105. The composition, method, expression vector, recombinant virus, engineered cell, tissue, or organ, or kit of any one of claims 101-104, wherein the nucleic acid encoding the inducing agent further comprises a WPRE and/or hGH pA sequence.

106. The composition, method, expression vector, recombinant virus, engineered cell, tissue, or organ, or kit of any one of claim 105, wherein the WPRE sequence is at least 70% identical to SEQ ID NO: 147, 152, or 155.

107. The composition, method, expression vector, recombinant virus, engineered cell, tissue, or organ, or kit of claim 105 or 106, wherein the hGH pA sequence is at least 70% identical to SEQ ID NO: 148, 153, or 156.

108. The composition, method, expression vector, recombinant virus, engineered cell, tissue, or organ, or kit of any one of claims 101-107, wherein the nucleic acid encoding the inducing agent comprises a sequence that is at least 70% identical to a sequence selected from tTA-Advanced (SEQ ID NO: 137), rtTA2S-M2 (SEQ ID NO: 14), or rtTA3 (SEQ ID NO: 10).

109. The composition, method, expression vector, recombinant virus, engineered cell, tissue, or organ, or kit of any one of claims 101-108, wherein the nucleic acid encoding the inducing agent comprises a sequence that is at least 70% identical to a sequence selected from pAAV-CaMKIIα-tTA2 (SEQ ID NO: 124), pAAV-CaMKIIα-rtTA2S-M2 (SEQ ID NO: 125, or pAAV-CaMKIIα-rtTA3 (SEQ ID NO: 126).

110. The composition, method, expression vector, recombinant virus, engineered cell, tissue, or organ, or kit of any one of claims 1-109, wherein the composition, expression vector, recombinant virus, engineered cell, tissue, or organ or kit does not comprise a Synapsin-I promoter operably linked to a nucleic acid sequence encoding an inducing agent.

111. The composition, method, expression vector, recombinant virus, engineered cell, tissue, or organ, or kit of claim 110, wherein the composition, expression vector, recombinant virus, engineered cell, tissue, or organ or kit does not comprise a sequence that is at least 70% identical to SEQ ID NO: 157.

112. The composition, method, expression vector, recombinant virus, engineered cell, tissue, or organ, or kit of claim 111, wherein the composition, expression vector, recombinant virus, engineered cell, tissue, or organ or kit does not comprise a sequence that is at least 70% identical to pAAV-ihSyn1-tTA (SEQ ID NO: 127).

113. An animal comprising a central nervous system cell, central nervous system tissue, or central nervous system organ, wherein the nervous system cell, central nervous system tissue, or central nervous system organ comprises:
a) an agent that induces OCT4 expression;
b) an agent that induce SOX2 expression; and
c) an agent that induces KLF4 expression,
wherein the nervous system cell, central nervous system tissue, or central nervous system organ does not comprise an agent that induces c-MYC expression.

114. A method of rejuvenating a cell, tissue, and/or organ, comprising administering to the cell, tissue, or organ a composition comprising:
(a) an agent that induces OCT4 expression;
(b) an agent that induce SOX2 expression; and
(c) an agent that induces KLF4 expression,
wherein the composition does not comprise an agent that induces c-MYC expression, wherein the cell, tissue, or organ is a central nervous system cell, central nervous system tissue, or central nervous system organ, optionally wherein the central nervous system does not include the retina, optionally wherein the cell, tissue, or organ is a brain cell, brain tissue, or brain,
wherein the method increases the expression of one or more of the following gene sets:
associative learning, excitatory synapse assembly, central nervous system neuron axonogenesis, central nervous system neuron development, memory, regulation of synaptic transmission GABAergic, regulation of postsynapse organization, learning, regulation of neurogenesis, central nervous system neuron differentiation and/or synapse maturation.

115. A method of rejuvenating a cell, tissue, and/or organ, comprising administering to the cell, tissue, or organ a composition comprising:
(a) an agent that induces OCT4 expression;
(b) an agent that induce SOX2 expression; and
(c) an agent that induces KLF4 expression,
wherein the composition does not comprise an agent that induces c-MYC expression,
wherein the cell, tissue, or organ is a central nervous system cell, central nervous system tissue, or central nervous system organ, optionally wherein the central nervous system does not include the retina, optionally wherein the cell, tissue, or organ is a brain cell, brain tissue, or brain,
wherein the method increases hypo-methylated CpG sites at development-related loci associated with positive regulation of epidermis development, midgut development, negative regulation of chromatin organization, negative regulation of histone methylation, lens development in camera-type eye, negative regulation of fat cell differentiation, negative regulation of histone modification, lung morphogenesis, DNA methylation on cytosine, and/or neural precursor cell proliferation and/or
wherein the method increases hyper-methylated CpG sites at development-related loci associated with skeletal muscle cell differentiation, heart looping, determination of heart left/right asymmetry, uterus morphogenesis, regulation of Notch signaling pathway, embryonic cranial skeleton morphogenesis, animal organ formation, tricuspid valve morphogenesis, lens fiber cell differentiation, and/or positive regulation of BMP signaling pathway.


SEQUENCE LISTING (163 sequences)

SEQ ID NO: 1 - DNA, 1056 bp, Mus musculus
  source 1..1056: mol_type = genomic DNA
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaac

SEQ ID NO: 2 - protein, 352 aa, Mus musculus
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 3 - DNA, 957 bp, Mus musculus
  source 1..957: mol_type = genomic DNA
atgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatg

SEQ ID NO: 4 - protein, 319 aa, Mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 5 - DNA, 1446 bp, Mus musculus
  source 1..1446: mol_type = genomic DNA
atgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcac

SEQ ID NO: 6 - protein, 482 aa, Mus musculus
MRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRH

SEQ ID NO: 7 - DNA, 376 bp, Synthetic construct
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 8 - DNA, 169 bp, Synthetic construct
  source 1..169: mol_type = other DNA
tgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccg

SEQ ID NO: 9 - protein, 21 aa, Synthetic construct
GSGEGRGSLLTCGDVEENPGP

SEQ ID NO: 10 - DNA, 708 bp, Synthetic construct
  source 1..708: mol_type = other DNA
atgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccgggtaa

SEQ ID NO: 11 - protein, 235 aa, Synthetic construct
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPG

SEQ ID NO: 12 - DNA, 747 bp, Synthetic construct
  source 1..747: mol_type = other DNA
atgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaa

SEQ ID NO: 13 - protein, 248 aa, Synthetic construct
MSRLDKSKVINSALELLNGVGIEGLTTRKLAQKLGVEQPTLYWHVKNKRALLDALPIEMLDRHHTHSCPLEVESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 14 - DNA, 747 bp, Synthetic construct
  source 1..747: mol_type = other DNA
atgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 15 - protein, 248 aa, Synthetic construct
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 16 - DNA, 7408 bp, Synthetic construct
  source 1..7408: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcactaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 17 - DNA, 5657 bp, Synthetic construct
  source 1..5657: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcctgatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcggccgcattaaacgccaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaaactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 18 - DNA, 1215 bp, Synthetic construct
  source 1..1215: mol_type = other DNA
gatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 19 - DNA, 19 bp, Synthetic construct
  source 1..19: mol_type = other DNA
tccctatcagtgatagaga

SEQ ID NO: 20 - DNA, 68 bp, Synthetic construct
  source 1..68: mol_type = other DNA
gctttaggcgtgtacggtgggcgcctataaaagcagagctcgtttagtgaaccgtcagatcgcctgga

SEQ ID NO: 21 - DNA, 248 bp, Synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 22 - DNA, 141 bp, Synthetic construct
  source 1..141: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 23 - DNA, 438 bp, Synthetic construct
  source 1..438: mol_type = other DNA
aattcgtacacgcctacctcgacccatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctggatcgc

SEQ ID NO: 24 - DNA, 315 bp, Synthetic construct
  source 1..315: mol_type = other DNA
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 25 - DNA, 618 bp, Synthetic construct
  source 1..618: mol_type = other DNA
atggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtggg

SEQ ID NO: 26 - protein, 206 aa, Synthetic construct
MARLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESG

SEQ ID NO: 27 - DNA, 1008 bp, Synthetic construct
  source 1..1008: mol_type = other DNA
atggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaa

SEQ ID NO: 28 - protein, 335 aa, Synthetic construct
MARLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 29 - DNA, 977 bp, Synthetic construct
  source 1..977: mol_type = other DNA
accttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgcc

SEQ ID NO: 30 - DNA, 5428 bp, Synthetic construct
  source 1..5428: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcctagatctaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgccaagcttgcggccgcattaaacgccaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaaactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 31 - DNA, 5332 bp, Synthetic construct
  source 1..5332: mol_type = other DNA
aaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccata

SEQ ID NO: 32 - DNA, 5392 bp, Synthetic construct
  source 1..5392: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggatgaggatcctctagagtcgacctgcagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 33 - DNA, 7732 bp, Synthetic construct
  source 1..7732: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgtacacgcctacctcgacccatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctggatcgcggccgcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcactaaatgactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 34 - DNA, 186 bp, Synthetic construct
  source 1..186: mol_type = other DNA
gaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctaga

SEQ ID NO: 35 - DNA, 783 bp, Synthetic construct
  source 1..783: mol_type = other DNA
agccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 36 - DNA, 570 bp, Synthetic construct
  source 1..570: mol_type = other DNA
cgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 37 - DNA, 1416 bp, Synthetic construct
  source 1..1416: mol_type = other DNA
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 38 - DNA, 3594 bp, Synthetic construct
  source 1..3594: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 39 - protein, 1198 aa, Synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNASGSGATNFSLLKQAGDVEENPGPACMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMACGSGEGRGSLLTCGDVEENPGPLEMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 40 - DNA, 1080 bp, Homo sapiens
  source 1..1080: mol_type = genomic DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaac

SEQ ID NO: 41 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 42 - DNA, 951 bp, Homo sapiens
  source 1..951: mol_type = genomic DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 43 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 44 - DNA, 1410 bp, Homo sapiens
  source 1..1410: mol_type = genomic DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 45 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 46 - DNA, 235 bp, Synthetic construct
  source 1..235: mol_type = other DNA
attttaatctcactagggttctgggagcacccccccccaccgctcccgccctccacaaagctcctgggcccctcctcccttcaaggattgcgaagagctggtcgcaaatcctcctaagccaccagcatctcggtcttcagctcacaccagccttgagcccagcctgcggccaggggaccacgcacgtcccacccacccagcgactccccagccgctgcccactcttcctcactca

SEQ ID NO: 47 - DNA, 229 bp, Synthetic construct
  source 1..229: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 48 - DNA, 617 bp, Synthetic construct
  source 1..617: mol_type = other DNA
cattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgc

SEQ ID NO: 49 - DNA, 252 bp, Synthetic construct
  source 1..252: mol_type = other DNA
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgg

SEQ ID NO: 50 - DNA, 292 bp, Homo sapiens
  source 1..292: mol_type = genomic DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttcgcctggtgctgtgtcagccccggtctcccaggggcttcccagtggtccccaggaaccctcgacagggcccggtctctctcgtccagcaagggcagggacgggccacaggccaagggc

SEQ ID NO: 51 - DNA, 113 bp, Homo sapiens
  source 1..113: mol_type = genomic DNA
gcctgtagccttaatctctcctagcagggggtttgggggagggaggaggagaaagaaagggccccttatggctgagacacaatgacccagccacaaggagggattaccgggcg

SEQ ID NO: 52 - DNA, 280 bp, Homo sapiens
  source 1..280: mol_type = genomic DNA
aggtaggaagtggcctttaactccatagaccctatttaaacagcttcggacaggtttaaacatctccttggataattcctagtatccctgttcccactcctactcagggatgatagctctaagaggtgttaggggattaggctgaaaatgtaggtcacccctcagccatctgggaactagaatgagtgagagaggagagaggggcagagacacacacattcgcatattaaggtgacgcgtgtggcctcgaacaccgagcgaccctgcagcgacccgctta

SEQ ID NO: 53-100 - skipped (48 entries)

SEQ ID NO: 101 - DNA, 492 bp, Homo sapiens
  source 1..492: mol_type = genomic DNA
gatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagc

SEQ ID NO: 102 - DNA, 900 bp, Homo sapiens
  source 1..900: mol_type = genomic DNA
agttaatgattaacccgccatgctacttatctacgtagccatgctctaggaagatcggaattcgcccttaagctagcagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatggatcctgagtaccttctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcccctgaattctgca

SEQ ID NO: 103 - DNA, 521 bp, Mus musculus
  source 1..521: mol_type = genomic DNA
ggttcttcccattttggctacatggtctttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctattcttgttctctgactcccactaatctacacattcagaggattgtggatataagaggctgggaggccagcttagcaaccagagctggagg

SEQ ID NO: 104 - DNA, 292 bp, Homo sapiens
  source 1..292: mol_type = genomic DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggc

SEQ ID NO: 105 - DNA, 7390 bp, Homo sapiens
  source 1..7390: mol_type = genomic DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 106 - DNA, 7265 bp, Homo sapiens
  source 1..7265: mol_type = genomic DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 107 - DNA, 5437 bp, Synthetic construct
  source 1..5437: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 108 - DNA, 18 bp, Synthetic construct
  source 1..18: mol_type = other DNA
cacaagtcaaacctttat

SEQ ID NO: 109 - DNA, 21 bp, Synthetic construct
  source 1..21: mol_type = other DNA
ggacgtaatccagaaagaaga

SEQ ID NO: 110 - DNA, 21 bp, Synthetic construct
  source 1..21: mol_type = other DNA
ttgtgcctctggaggttataa

SEQ ID NO: 111 - DNA, 21 bp, Synthetic construct
  source 1..21: mol_type = other DNA
ggaaataaaggctggtgaagg

SEQ ID NO: 112 - DNA, 21 bp, Synthetic construct
  source 1..21: mol_type = other DNA
gaaagatgaaggtccatatta

SEQ ID NO: 113 - DNA, 21 bp, Synthetic construct
  source 1..21: mol_type = other DNA
gcagatggccgtgacacaaat

SEQ ID NO: 114 - DNA, 21 bp, Synthetic construct
  source 1..21: mol_type = other DNA
gctcatggagactaggtttgg

SEQ ID NO: 115 - DNA, 21 bp, Synthetic construct
  source 1..21: mol_type = other DNA
ggatgtaagtttgccagaagc

SEQ ID NO: 116 - DNA, 21 bp, Synthetic construct
  source 1..21: mol_type = other DNA
gctccaacgagaagctatttg

SEQ ID NO: 117 - DNA, 19 bp, Synthetic construct
  source 1..19: mol_type = other DNA
gttcagatgtgcggcgagt

SEQ ID NO: 118 - protein, 22 aa, Synthetic construct
GSGATNFSLLKQAGDVEENPGP

SEQ ID NO: 119 - DNA, 66 bp, Synthetic construct
  source 1..66: mol_type = other DNA
ggcagcggcgccacgaacttctctctgttaaagcaagcaggagatgttgaagaaaaccccgggcct

SEQ ID NO: 120 - DNA, 63 bp, Synthetic construct
  source 1..63: mol_type = other DNA
ggctccggcgagggcaggggaagtcttctaacatgcggggacgtggaggaaaatcccggccca

SEQ ID NO: 121 - DNA, 7390 bp, Synthetic construct; identical to SEQ ID NO: 105
  source 1..7390: mol_type = other DNA

SEQ ID NO: 122 - DNA, 6586 bp, Synthetic construct
  source 1..6586: mol_type = other DNA
aattcagagaccgggaaccaaactagcctttaaaaaacataagtacaggagccagcaagatggctcagtgggtaaaggtgcctaccagcaagcctgacagcctgagttcagtccccacgaactacgtggtaggagaggaccaaccaactctggaaatctgttctgcaaacacatgctcacacacacacacacaaatagtataaacaattttaaatttcatttaaaaataatttgtaaacaaaatcattagcacaggttttagaaagagcctcttggtgacatcaagttgatgctgtagatggggtatcattcctgaggacccaaaaccgggtctcagcctttccccattctgagagttctctcttttctcagccactagctgaagagtagagtggctcagcactgggctcttgagttcccaagtcctacaactggtcagcctgactactaaccagccatgaagaaacaaggagtggatgggctgagtctgctgggatgggagtggagttagtaagtggccatggatgtaatgaccccagcaatgctggctagaaggcatgcctcctttccttgtctggagacggaacgggagggatcatcttgtactcacagaagggagaacattctagctggttgggccaaaatgtgcaagttcacctggaggtggtggtgcatgcttttaactccagtactcaggaggcagggccaggtggatctctgtgagttcaagaccagcctgcactatggagagagttttgggacagccagagttacacagaaaaatcctggtggaaaatctgaaagaaagagagaaagaaagaaagaaagaaaggaagaaagaaagaaagagtggcaggcaggcaggcaggaggaaggaaggaaggaaggaaggaaggaaggaaggaaggaaggaaaataggtgcgacttcaagatccggagttacaagcagaatgcactgtttccctaacagggccaagtgttttgagtaactgaaggtgggcatgatgcctgggaagcagaaacaagccaggcagatgcaccccttgccttgcttccgaagggctgcagtagcatggaaaacatggaaaacaaccaatccattccctttgctgatataacaggctccaaagccaaaacctgtcactggaggctcaagagcagatctccagccaagaggcaaaggaatgggggaagctggagggcctccctctggttatccaggcttctgaaggttcaagcaaagaaagggttacaaccttaaaaggagagcgtcccggggtatgggtagaagactgctccaccccgacccccagggtccctaaccgtcttttccctgggcgagtcagcccaatcacaggactgagagtgcctctttagtagcagcaagccacttcggacacccaaatggaacacctccagtcagccctcgccgaccaccccaccccctccatccttttccctcagcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgaggatgcaagccttccccagctacagttttgggaaaggataccagggcgctcctatatgggggcgcgggaactggggaaagaaggtgctcccaggtcgaggtgggagaggaaggcagtgcggggtcacgggctttctccctgctaacggacgctttcgaagagtgggtgccggaggagaaccatgaggaaggacatcaaggacagcctttggtccccaagctcaaatcgctttagtggtgcgaatagagggaggaggtgggtggcaaactggagggagtccccagcgggtgacctcgtggctggctgggtgcggggcaccgcaggtaagaaaaccgcaatgttgcgggaggggactgggtggcaggcgcgggggaggggaaagctagaaaggatgcgagggagcggaggggggagggagcgggagaatctcaactggtagaggaagattaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgccaggcttctccagccccagccccaattcttttctctagtgttcccccttccctcccctgaatctcaagcccacactccctcctccataacccactgttatcaaatctaagtcatttgccacccaacaaccatcaggaggcggaagcagacgggaggagtttgagatcaacttgggctacatcacgagttccaggctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcgcagactgtgcctctcttgggcaaggacaggctagacagcctgccggtgtgttgagctagggcactgtggggaaggcagagaacctgtgcagggcagcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaacctcatttatccggtaaatgtcttttactctgctctcagggagctgaggcaggacatcctgagatacattgggagaggagatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctgcctctgcctctctgcctctctgcctctctgcctctctctctgcctctctctgcctctctctgcccctctctgcccctctctgcccctctctgccgccctctgccttttgccctctgccctctgttctctggcctctgccctctgccctctggcctctggcctctgcctctgcctcttgagtgctggaatcaaaggtgtgagctctgtaggtcttaagttccagaagaaagtaatgaagtcacccagcagggaggtgctcagggacagcacagacacacacccaggacataggctcccacttccttggctttctctgagtggcaaaggaccttaggcagtgtcactccctaagagaaggggataaagagaggggctgaggtattcatcatgtgctccgtggatctcaagccctcaaggtaaatggggacccacctgtcctaccagctggctgacctgtagctttccccaccacagaatccaagtcggaactcttggcacctagaggatctcgaggtccttcctctgcagaggtcttgcttctcccggtcagctgactccctccccaagtccttcaaatatctcagaacatggggagaaacggggaccttgtccctcctaaggaaccccagtgctgcatgccatcatcccccccaccctcgcccccacccccgccacttctccctccatgcataccactagctgtcattttgtactctgtatttattccagggctgcttctgattatttagtttgttctttccctggagacctgttagaacataagggcgtatggtgggtaggggaggcaggatatcagtccctggggcgagttcctccctgccaaccaagccagatgcctgaaagagatatggatgagggaagttggactgtgcctgtacctggtacagtcatactctgttgaaagaatcatcggggaggggggggggctcaagaggggagagctctgctgagcctttgtggaccatccaatgaggatgagggcttagattctaccaggtcattctcagccaccacacacaagcgctctgccatcactgaagaagccccctagggctcttgggccagggcacactcagtaaagatgcaggttcagtcagggaatgatggggaaaggggtaggaggtgggggagggatcaccccctcctctaaaacacgagcctgctgtctccaaaggcctctgcctgtagtgagggtggcagaagaagacaaggagccagaactctgactccaggatctaagtccgtgcaggaaggggatcctagaaccatctggttggacccagcttaccaagggagagcctttattcttctttcccttgcccctctgtgccagcccctcttgctgtccctgatcccccagacagcgagagtcttgcaacctgcctcttccaagacctcctaatctcaggggcaggcggtggagtgagatccggcgtgcacactttttggaagatagctttcccaaggatcctctcccccactggcagctctgcctgtcccatcaccatgtataataccaccactgctacagcatctcaccgaggaaagaaaactgcacaataaaaccaagcctctggagtgtgtcctggtgtctgtctcttctgtgtcctggcgtctgtctcttctgtgttcttccaaggtcagaaacaaaaaccacacacttcaacctggatggctcggctgagcacttctgtgtgcagaaggtccaaccagactctggggtaccccggccctccctattcccttgcctcctgtctcccgctttttatagctccctatgctgggcttctctggagagtgaaatctttgcccaaatcaatgcgcattctctctgctgagtcatctggcgacagcagttgagttcacccgccaacacatgggcccagctatgtagccgaaccctggctctggaagtgccagggactttgtgcataagtatgtaccatgcccttttttcacagtcctagctctgcagaagtgcagcctgaaggcctgtctgctgagaggacatgccctggagccctgaaacaggcacagtgggaggaggaacggaggatgacaggcatcaggccctcagtccaaaagcaaccacttgagaatgggctggagtacgaaacatggggtcccgtccctggatccctcctcaaagagtaataagtaaaatataaacaggtaccccaggccgttctgggtttgggttgtaatgggatccatttgcagagaactattgagacagcccagccgtactgtgacaggcaatgtgggggaggaggttgaatcacttggtatttagcatgaatagaataattccctgaacatttttcttaaacatccatatctaaattaccaccactcgctcccagtcttcctgcctttgcgccagcctcctgtctggccatgcctgaagaaggctggagaagccacccacctcaggccatgacactgccagccacttggcaggtgcagccaaacctgagctgtcccagaaagggacattctcaagacccaggcaccctgatcagcactgacttggagctacaagtgtcatgccagaaaagtctctaagaaaaccttttcagggaaaagggggtgactcaacaccgggcaagtttgggaagccccacccttcgagtgatggaagagcagataggaagcctcagaagagagacaccggcacccaggtaacgttcctcatgtggtctctgtcacactaggtgctcttccctggacatctccgtgaccacactctcagttcttagggagatgcgggtgctctctgaggctatctcagagttgcagattctgaggcctagagtgactacagtcagcctaggaagccacagaggactgtggaccaggagggcagaagaggagaagggaagaaaaaccatcagataggacttgcaatgaaactaacccaagacaatcataatgcagacaggaatgttaaaggcgttcagcagc

SEQ ID NO: 123 - DNA, 13577 bp, Synthetic construct
  source 1..13577: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgattagttacccgggagcatgtcaaggtcaaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgccggtcgaacagctcaattgcttgtctcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcgccgtttatgactttgctcttgtccagtctagacatggtggatccgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccagatcggcgcatcgatgaggccctttcgtcttcactcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaaggatccgcggccgcgccgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaatgaacgcgtgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttacccgtacgtgtacagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgataacctgcaggacctggtgcatgacccgcaagcccggtgcctgacgggcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 124 - DNA, 6108 bp, Synthetic construct
  source 1..6108: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggatgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 125 - DNA, 6108 bp, Synthetic construct
  source 1..6108: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 126 - DNA, 6069 bp, Synthetic construct
  source 1..6069: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccgccaccatgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 127 - DNA, 5455 bp, Synthetic construct
  source 1..5455: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaacagatctctaaaacaggtaagtcccattaatctccctatcagtgatagagaaggtctgaagagtttactccctatcagtgatagagattaattcctctactaaccttgttcatcttttctttttttttctacaggtcctgggtgattaacagcttaagggcccggatccggtaccgccaccatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 128 - DNA, 750 bp, Synthetic construct
  source 1..750: mol_type = other DNA
ttagttacccgggagcatgtcaaggtcaaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgccggtcgaacagctcaattgcttgtctcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcgccgtttatgactttgctcttgtccagtctagacat

SEQ ID NO: 129 - protein, 249 aa, Synthetic construct
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGN

SEQ ID NO: 130 - DNA, 1212 bp, Synthetic construct
  source 1..1212: mol_type = other DNA
gtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggcc

SEQ ID NO: 131 - DNA, 1413 bp, Synthetic construct
  source 1..1413: mol_type = other DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 132 - DNA, 500 bp, Synthetic construct
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 133 - DNA, 795 bp, Synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 134 - protein, 264 aa, Synthetic construct
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 135 - DNA, 589 bp, Synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 136 - DNA, 641 bp, Synthetic construct
  source 1..641: mol_type = other DNA
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcct

SEQ ID NO: 137 - DNA, 747 bp, Synthetic construct
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggatga

SEQ ID NO: 138 - protein, 248 aa, Synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 139 - DNA, 479 bp, Synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 140 - DNA, 4517 bp, Synthetic construct
  source 1..4517: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgctcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 141 - DNA, 720 bp, Synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 142 - protein, 239 aa, Synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 143 - DNA, 177 bp, Synthetic construct
  source 1..177: mol_type = other DNA
ctagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatc

SEQ ID NO: 144 - DNA, 67 bp, Synthetic construct
  source 1..67: mol_type = other DNA
ggcagcggcgccacgaacttctctctgttaaagcaagcaggagatgttgaagaaaaccccgggcctg

SEQ ID NO: 145 - DNA, 1449 bp, Synthetic construct
  source 1..1449: mol_type = other DNA
atgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcactaa

SEQ ID NO: 146 - DNA, 1322 bp, Synthetic construct
  source 1..1322: mol_type = other DNA
tgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtacc

SEQ ID NO: 147 - DNA, 589 bp, Synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 148 - DNA, 477 bp, Synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 149 - DNA, 1322 bp, Synthetic construct; identical to SEQ ID NO: 146
  source 1..1322: mol_type = other DNA

SEQ ID NO: 150-151 - skipped (2 entries)

SEQ ID NO: 152 - DNA, 589 bp, Synthetic construct; identical to SEQ ID NO: 147
  source 1..589: mol_type = other DNA

SEQ ID NO: 153 - DNA, 477 bp, Synthetic construct; identical to SEQ ID NO: 148
  source 1..477: mol_type = other DNA

SEQ ID NO: 154 - DNA, 1322 bp, Synthetic construct; identical to SEQ ID NO: 146
  source 1..1322: mol_type = other DNA

SEQ ID NO: 155 - DNA, 589 bp, Synthetic construct; identical to SEQ ID NO: 147
  source 1..589: mol_type = other DNA

SEQ ID NO: 156 - DNA, 477 bp, Synthetic construct; identical to SEQ ID NO: 148
  source 1..477: mol_type = other DNA

SEQ ID NO: 157 - DNA, 448 bp, Synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 158 - DNA, 747 bp, Synthetic construct
  source 1..747: mol_type = other DNA
atgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 159 - protein, 248 aa, Synthetic construct
MSRLDKSKVINGALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 160 - DNA, 589 bp, Synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 161 - DNA, 477 bp, Synthetic construct; identical to SEQ ID NO: 148
  source 1..477: mol_type = other DNA

SEQ ID NO: 162 - DNA, 1715 bp, Synthetic construct
  source 1..1715: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 163 - DNA, 4880 bp, synthetic construct
  source 1..4880: mol_type = other DNA
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacggaagtggcgtgaaacagactttgaattttgaccttctcaagttggcgggagacgtggagtccaacccagggcccatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaaagatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatgatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgggtagtgggcaatgtactaactacgctttgttgaaactcgctggcgatgttgaaagtaaccccggtcctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa